(19) United States
(12) Patent Application Publication
(10) Pub. No.: US 2026/0184693 A1
(43) Pub. Date: Jul. 2, 2026

(54) INHIBITORS OF SOLUTE CARRIER FAMILY 6A MEMBER 19 (SLC6A19) AND METHODS OF USE THEREOF

(71) Applicant: Maze Therapeutics, Inc., South San Francisco, CA (US)

(72) Inventors: Jennifer PITZEN, Oakland, CA (US); Maximiliano DE LA HIGUERA MACIAS, San Francisco, CA (US); Nicole COOPER, San Bruno, CA (US); Christopher Joseph SINZ, Walnut Creek, CA (US); Kevin MELLEM, Redwood City, CA (US); Patrick Sang Tae LEE, Walnut Creek, CA (US); Nathan FASTMAN, Palo Alto, CA (US); Christos TZITZILONIS, Emeryville, CA (US); Yuxi LIU, San Mateo, CA (US); David John MORGANS, JR., Los Altos, CA (US); Jessica WAHLERS, South San Francisco, CA (US)

(21) Appl. No.: 19/131,916

(22) PCT Filed: Nov. 21, 2023
(86) PCT No.: PCT/US2023/080815

§ 371 Date: May 21, 2025

Related U.S. Application Data
(60) Provisional application No. 63/427,275, filed on Nov. 22, 2022.

(51) Int. Cl. C07D 401/14; A61K 31/454; A61K 31/4545; A61K 31/497; A61K 31/501; A61K 31/506; A61K 31/513; A61K 31/519; A61K 31/5377; C07D 401/12; C07D 405/14; C07D 413/14; C07D 417/14; C07D 471/04; C07D 487/04

(52) U.S. Cl. CPC C07D 401/14; A61K 31/454; A61K 31/4545; A61K 31/497; A61K 31/501; A61K 31/506; A61K 31/513; A61K 31/519; A61K 31/5377; C07D 401/12; C07D 405/14; C07D 413/14; C07D 417/14; C07D 471/04; C07D 487/04

(57) ABSTRACT

Provided herein are compounds of formula (I): or a stereoisomer or tautomer thereof, or a pharmaceutically acceptable salt of any of the foregoing, wherein n, r, p, R^{1a}, R^{1b}, R², R³, R⁴, and R⁵ are as defined elsewhere herein. Also provided herein are methods of preparing compounds of formula (I). Also provided herein are methods of inhibiting SL-C6A19 and methods of treating a SLC6A19-mediated disease, disorder, or condition in an individual in need thereof.

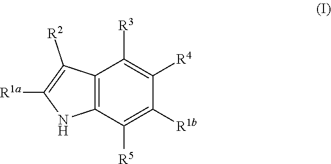

### CROSS-REFERENCE TO RELATED APPLICATIONS

This application claims priority to U.S. Provisional Application Ser. No. 63/427,275, filed Nov. 22, 2022, which is hereby incorporated herein by reference in its entirety.

### BACKGROUND OF THE INVENTION

Solute carrier family 6A member 19 (SLC6A19), also known as BOAT1, is a sodium-dependent neutral amino acid (NAA) transporter that is predominantly expressed on the apical membranes of renal and intestinal epithelial cells (Fairweather et al. J Biol. Chem (2015) 290, 24308-24325). In the intestines, surface expression of SLC6A19 is dependent on the chaperone protein ACE2, for amino acid uptake (Singer et al Am. J. Physiol. Gastrointest. Liver Physiol (2012) 303, G686-G695). In the kidneys, SLC6A19 requires TMEM27 for surface expression to reabsorb amino acids (Verrey et al. Arch. Eur. J. Physiol. (2009) 458, 53-60).

Phenylketonuria (PKU) is caused by mutations in the enzyme phenylalanine hydroxylase (PAH), the key enzyme in the metabolism of phenylalanine (Phe) to tyrosine (Tyr), both of which are NAA. PKU patients have toxic buildup of Phe in the blood and other tissues, leading to neurologic alterations (Scriver C. R Hum. Mutat. (2007) 28, 831-843). Treatment options for PKU include severely limiting protein intake, which has poor compliance, and the 2 FDA-approved drugs, Palynziq and Kuvan, both of which are limited to a subset of patients; significant unmet medical need still remains (Strisciuglio et. al. Metabolites (2014) 4, 1007-1017; Gentile et. al. Mol. Genet. and Metab. (2010) 99, S64-S67). Because the symptoms of PKU are a result of systemic buildup of Phe, inhibition of SLC6A19 to limit the intestinal absorption of Phe and its reuptake in the kidneys can be a viable approach to treating PKU. Indeed, loss of SLC6A19 in a mouse model of PKU significantly increased the excretion of Phe in urine and dramatically reduced plasma Phe and normalized neurotransmitter levels (Belanger et al. JCI Insight (2018) 3, e121762).

The prevalence of chronic kidney disease (CKD) is rapidly increasing and an estimated 15% of the US adult population have CKD (CDC 2021). An association of loss-of-function splice (c.1173+2T>G) (Sveinbjornsson G. et al. Hum. Mol. Genet. (2014) 23, 6935-6943) and missense (D173N) variants in SLC6A19 with protection from CKD has been reported (Sinnott-Amstrong N. et al. Nat. Genet. (2021) 53, 185-194).

Metabolic syndrome, and related diseases such as diabetes, is a global epidemic with an estimated one third of US adults having metabolic syndrome (Saklayen M. Curr Hypertens Rep (2018) 20, 12). Mice with deletion of SLC6A19 have a number of improved metabolic outcomes, including resistance to weight gain on a high-fat diet, improved glucose tolerance and insulin sensitivity, and increased energy expenditure (Jiang et al. Mol. Metab. (2015) 4, 406-417). These positive outcomes are likely due to increased secretion of FGF21 and GLP-1, two hormones that play important roles in regulating energy metabolism, (Geng et al. Nat Rev Endo. (2020) 16, 654-667; Baggio et al. Mol Metab (2021) 46). Inhibiting SLC6A19, therefore, may be an effective approach to treat metabolic diseases.

In humans, SLC6A19 biallelic loss of function results in a rare disease called Hartnup disorder, which is predominantly asymptomatic in patients with adequate nutrition (Seow et al. Nat. Genet. (2004) 36, 1003-1007. Azmanov et al. Hum. Mutat. (2008) 29, 1217-1221). Since the NAA tryptophan is essential for the synthesis of nicotinamide, Harnup patients may develop niacin deficiency and its associated symptoms, which include dermatitis, photosensitivity, and psychosis, but these symptoms are well-controlled with niacin supplementation (Hashmi et al. StatPearls (2021)). Similarly, mice with complete loss of SLC6A19 are viable and fertile (Jiang et al. Mol. Metab. (2015) 4, 406-417). Thus, inhibition of SLC6A19 is believed to be a viable therapeutic approach.

There remains a need for therapies to treat such diseases/disorders.

### BRIEF SUMMARY OF THE INVENTION

In one aspect, provided herein is a compound of formula (I):

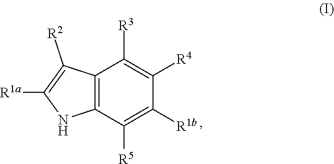

or a stereoisomer or tautomer thereof, or a pharmaceutically acceptable salt of any of the foregoing, wherein
one of R^{1a} and R^{1b} is

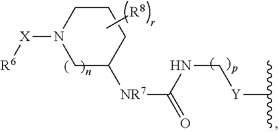

and
the other of R^{1a} and R^{1b} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein the C₁₋₆alkyl of R³ and R⁵ is optionally substituted with one or more —OH;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
   the C₁₋₆alkyl, C₆₋₂₀aryl, and 5-20 membered heteroaryl of R⁶ are each independently optionally substituted with one or more R^{a}, and
   the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
X is absent or —C(O)—;
Y is absent or

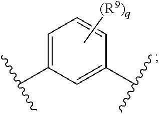

n is an integer from 1 to 2;
p is an integer from 1 to 4;
q is an integer from 0 to 4; and
r is an integer from 0 to 11.

In one aspect, provided herein is a compound of formula (I-A):

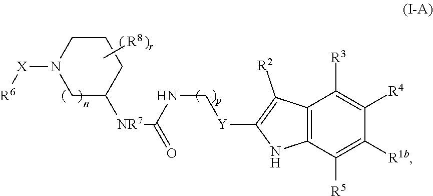

or a stereoisomer or tautomer thereof, or a pharmaceutically acceptable salt of any of the foregoing, wherein n, r, p, R^{1b}, R², R³, R⁴, R⁵, R⁶, R⁷, R⁸, X, and Y are as defined elsewhere herein.

In one aspect, provided herein is a compound of formula (I-B):

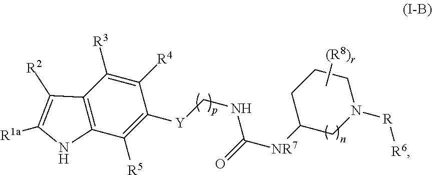

or a stereoisomer or tautomer thereof, or a pharmaceutically acceptable salt of any of the foregoing, wherein n, r, p, R^{1a}, R², R³, R⁴, R⁵, R⁶, R⁷, R⁸, X, and Y are as defined elsewhere herein.

In one aspect, provided herein is a compound of formula (I-C):

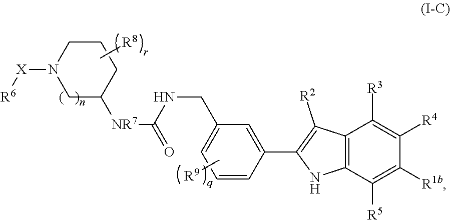

or a stereoisomer or tautomer thereof, or a pharmaceutically acceptable salt of any of the foregoing, wherein n, r, p, q, R^{1b}, R², R³, R⁴, R⁵, R⁶, R⁷, R⁸, R⁹, and X are as defined elsewhere herein.

In one aspect, provided herein is a compound of formula (I-D):

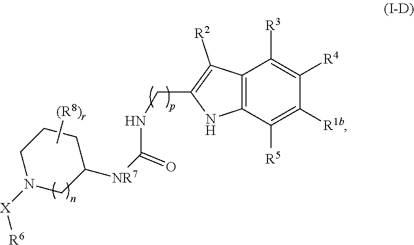

or a stereoisomer or tautomer thereof, or a pharmaceutically acceptable salt of any of the foregoing, wherein n, r, p, R^{1b}, R², R³, R⁴, R⁵, R⁶, R⁷, R⁸, and X are as defined elsewhere herein.

In one aspect, provided herein is a pharmaceutical composition, comprising (i) a compound of formula (I), or any variation or embodiment thereof, or a stereoisomer or tautomer thereof, or a pharmaceutically acceptable salt of any of the foregoing, and (ii) one or more pharmaceutically acceptable excipients.

In one aspect, provided herein is a method of modulating SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of inhibiting SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of modulating SLC6A19 in a cell of an individual in need thereof, comprising administering to the individual an effective amount of (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a SLC6A19-mediated disease, disorder, or condition in an individual in need thereof, comprising administering to the individual an effective amount of (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a kit, comprising (i) an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an SLC6A19-mediated disease, disorder, or condition in an individual in need thereof.

In some aspects, provided herein are methods of preparing a compound of formula (I), or any embodiment or variation thereof, such as a compound of formula (I), (I-A), (I-A1), (I-A2), (I-A3), (I-A4), (I-A5), (I-A6), (I-B), (I-B1), (I-B32), (I-B33), (I-B34), (I-C), (I-C1), (I-C2), (I-D), (I-D1), (I-D2), (I-D3), (I-D4), (I-D5), (I-D6), or a stereoisomer or tautomer thereof, or a pharmaceutically acceptable salt of any of the foregoing.

Any embodiments provided herein of a compound of formula (I), (I-A), (I-A1), (I-A2), (I-A3), (I-A4), (I-A5), (I-A6), (I-B), (I-B1), (I-B32), (I-B33), (I-B34), (I-C), (I-C1), (I-C2), (I-D), (I-D1), (I-D2), (I-D3), (I-D4), (I-D5), (I-D6), or a stereoisomer or tautomer thereof, or a pharmaceutically acceptable salt of any of the foregoing, or any variation or embodiment thereof, are also embodiments of a compound of formula (I), or a stereoisomer or tautomer thereof, or a pharmaceutically acceptable salt of any of the foregoing, or any variation or embodiment thereof.

### DETAILED DESCRIPTION OF THE INVENTION

“Individual” refers to mammals and includes humans and non-human mammals. Examples of individuals include, but are not limited to, mice, rats, hamsters, guinea pigs, pigs, rabbits, cats, dogs, goats, sheep, cows, and humans. In some embodiments, individual refers to a human.

As used herein, “about” a parameter or value includes and describes that parameter or value per se. For example, “about X” includes and describes X per se.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated univalent hydrocarbon chain. As used herein, alkyl has 1-20 carbons (i.e., C₁₋₂₀alkyl), 1-16 carbons (i.e., C₁₋₁₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl).

Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkenyl”, as used herein, refers to a branched or unbranched univalent hydrocarbon chain comprising at least one carbon-carbon double bond. As used herein, alkenyl has 2-20 carbons (i.e., C₂₋₂₀alkenyl), 2-16 carbons (i.e., C₂₋₁₆alkenyl), 2-12 carbons (i.e., C₂₋₁₂alkenyl), 2-10 carbons (i.e., C₂₋₁₀alkenyl), 2-8 carbons (i.e., C₂₋₈alkenyl), 2-6 carbons (i.e., C₂₋₆alkenyl), 2-4 carbons (i.e., C₂₋₄alkenyl), or 2-3 carbons (i.e., C₂₋₃alkenyl). Examples of alkenyl include, but are not limited to, ethenyl, prop-1-enyl, prop-2-enyl 1,2-butadienyl, and 1,3-butadienyl. When an alkenyl residue having a specific number of carbons is named by chemical name or molecular formula, all positional isomers having that number of carbon atoms may be encompassed—for example, “propenyl” includes prop-1-enyl and prop-2-enyl. Certain commonly used alternative names may be used and will be understood by those of ordinary skill in the art. For instance, a divalent group, such as a divalent “alkenyl” group, may be referred to as an “alkenylene”.

The term “alkynyl”, as used herein, refers to a branched or unbranched univalent hydrocarbon chain comprising at least one carbon-carbon triple bond. As used herein, alkynyl has 2-20 carbons (i.e., C₂₋₂₀alkynyl), 2-16 carbons (i.e., C₂₋₁₆alkynyl), 2-12 carbons (i.e., C₂₋₁₂alkynyl), 2-10 carbons (i.e., C₂₋₁₀alkynyl), 2-8 carbons (i.e., C₂₋₈-alkynyl), 2-6 carbons (i.e., C₂₋₆alkynyl), 2-4 carbons (i.e., C₂₋₄alkynyl), or 2-3 carbons (i.e., C₂₋₃alkynyl). Examples of alkynyl include, but are not limited to, ethynyl, prop-1-ynyl, prop-2-ynyl, but-1-ynyl, but-2-ynyl, and but-3-ynyl. When an alkynyl residue having a specific number of carbons is named by chemical name or molecular formula, all positional isomers having that number of carbon atoms may be encompassed for example, “propynyl” includes prop-1-ynyl and prop-2-ynyl. Certain commonly used alternative names may be used and will be understood by those of ordinary skill in the art. For instance, a divalent group, such as a divalent “alkynyl” group, may be referred to as an “alkynylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond. Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbonyl, decalinyl, 7,7-dimethyl-bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo). Additionally, terms such as “haloalkyl” are meant to include monohaloalkyl and polyhaloalkyl. For example, the term “C1-C4 haloalkyl” is mean to include trifluoromethyl, 2,2,2-trifluoroethyl, 4-chlorobutyl, 3-bromopropyl, difluoromethyl, and the like.

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The term “oxo”, as used herein, refers to a ═O moiety.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference in its entirety. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²p, ³⁵S, ¹⁸F, ³⁶Cl, ¹²³I, and ¹²⁵I, respectively. In some embodiments, a compound of formula (I) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers.

Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference in its entirety.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)-(or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

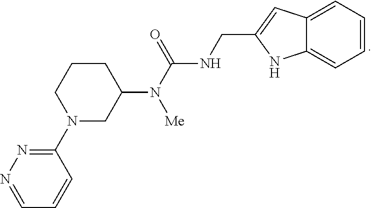 [SMILES: CN(C(=O)NCc1cc2ccccc2[nH]1)C1CCCN(c2cccnn2)C1]

Where enantiomeric and/or diastereomeric forms exist of a given structure with two stereocenters, wedged and/or dashed bonds and the presence of two “or 1” symbols indicate the composition is made up of at least 90%, by weight, a single stereoisomer with known relative stereochemistry but unknown absolute stereochemistry, e.g.,

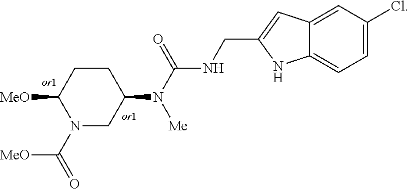 [SMILES: COC(=O)N1C[C@H](N(C)C(=O)NCc2cc3cc(Cl)ccc3[nH]2)CC[C@@H]1OC]

Where enantiomeric and/or diastereomeric forms exist of a given structure with two stereocenters, wedged and/or dashed bonds and the presence of two “& 1” symbols indicate the composition is made up of a pair of enantiomers with known relative stereochemistry, e.g.,

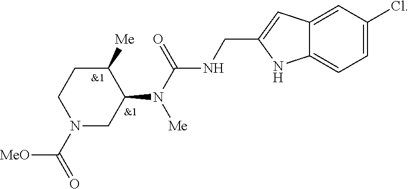 [SMILES: COC(=O)N1CC[C@@H](C)[C@@H](N(C)C(=O)NCc2cc3cc(Cl)ccc3[nH]2)C1]

### Compounds

In one aspect, provided herein is a compound of formula (I):

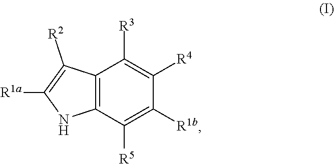

or a stereoisomer or tautomer thereof, or a pharmaceutically acceptable salt of any of the foregoing, wherein
one of R^{1a} and R^{1b} is

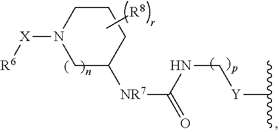

and
the other of R^{1a} and R^{1b} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein the C₁₋₆alkyl of R³ and R⁵ is optionally substituted with one or more —OH;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
X is absent or —C(O)—;

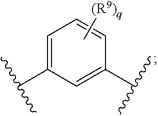

Y is absent or;
n is an integer from 1 to 2;
p is an integer from 1 to 4;
q is an integer from 0 to 4; and
r is an integer from 0 to 11.

In some embodiments of a compound of formula (I), or a stereoisomer or tautomer thereof, or a pharmaceutically acceptable salt of any of the foregoing, one of R^{1a} and R^{1b} is

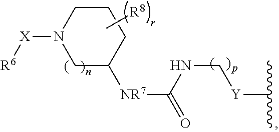

and the other of R^{1a} and R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R³ and R⁵ are each independently H, —OH, halo, —N(R^{w1})(R^{w2}) or C₁₋₃alkyl, wherein the C₁₋₃alkyl of R³ and R⁵ is optionally substituted with one or more —OH; R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-6 membered heterocyclyl of R⁶ are each independently optionally substituted with one or more R^{b}; R⁷ is C₁₋₃alkyl, C₁₋₃cycloalkyl, or C₁₋₃haloalkyl; R⁸ is independently at each occurrence, C₁₋₃alkyl; R⁹ is independently at each occurrence, halo; each R^{a} is independently —OH, halo, —CN, C₁₋₃alkyl, C₁₋₃alkoxy, —N(R^{w1})(R^{w2}), —OR^{x}, —C(O)R^{y}, —SO₂R^{z}, C₃₋₆cycloalkyl, C₆₋₁₀aryl, 5-10 membered heteroaryl, or 3-6 membered heterocyclyl, wherein the C₁₋₃alkyl, C₃₋₆cycloalkyl, and 3-6 membered heterocyclyl of R^{a} are each optionally substituted with one or more R^{b}, and the C₆₋₁₀aryl, and 5-10 membered heteroaryl of R^{a} are each optionally substituted with one or more R^{c}; each R^{b} is independently —OH, oxo, halo, C₁₋₃alkyl, C₁₋₃alkoxy, —C(O)R^{y}, or —SO₂R^{z}, wherein the C₁₋₃alkyl is optionally substituted with one or more R^{c}; each R^{c} is independently —OH, halo, —N(R^{w1})(R^{w2}), C₁₋₃alkyl, —C₁₋₃alkylN(R^{w1})(R^{w2}), or C₁₋₃alkoxy; R^{w1} and R^{w2} are independently at each occurrence H, C₁₋₃alkyl, C₁₋₃haloalkyl, C₃₋₆cycloalkyl, or —C(O)R^{y}; R^{x} is independently at each occurrence, 3-6 membered heterocyclyl optionally substituted with one or more oxo or C₁₋₃alkyl; R^{y} is independently at each occurrence, C₁₋₃alkyl, C₁₋₃haloalkyl; —NH₂, —NH(C₁₋₃alkyl), or —N(C₁₋₃alkyl)₂; R^{z} is independently at each occurrence, C₁₋₃alkyl; X is absent or —C(O)—; Y is absent or

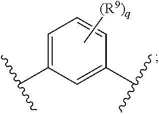

n is an integer from 1 to 2; p is an integer from 1 to 4; q is an integer from 0 to 4; and r is an integer from 0 to 11.

In some embodiments of a compound of formula (I), or a stereoisomer or tautomer thereof, or a pharmaceutically acceptable salt of any of the foregoing, one of R^{1a} and R^{1b} is

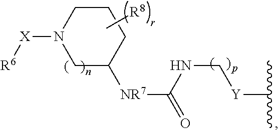

and the other of R^{1a} and R^{1b} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a}. In some embodiments, R^{1a} is

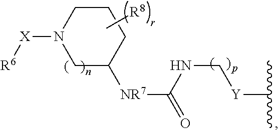

and R^{1a} bis H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a}. In some embodiments R^{1b} is

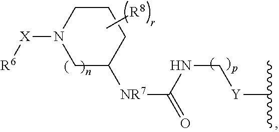

and R^{1a} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a}.

In some embodiments of a compound of formula (I), or a stereoisomer or tautomer thereof, or a pharmaceutically acceptable salt of any of the foregoing, R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}. In some embodiments, R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R⁷ is C₁₋₆cycloalkyl, X is —C(O)—, n is 1, p is 1, and Y is absent.

In some embodiments of a compound of formula (I), or a stereoisomer or tautomer thereof, or a pharmaceutically acceptable salt of any of the foregoing, R² is H, halo, —CN, C₁₋₆alkyl, C₁₋₆cycloalkyl, C₁₋₆haloalkyl, or C₁₋₇₆alkoxy, wherein the C₁₋₆alkoxy of R² is optionally substituted with one or more R^{a}. In some embodiments, R² is H, halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}. In some embodiments, R² is H, or C₁₋₃alkyl. In some embodiments, R² is H. In some embodiments, R² is C₁₋₃alkyl. In some embodiments, R² is methyl.

In some embodiments of a compound of formula (I), or a stereoisomer or tautomer thereof, or a pharmaceutically acceptable salt of any of the foregoing, R⁴ is H, halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}. In some embodiments, R⁴ is H, halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}. In some embodiments, R⁴ is H or halo. In some embodiments, R⁴ is H. In some embodiments, R⁴ is F or Cl. In some embodiments, R⁴ is F. In some embodiments, R⁴ is Cl. In some embodiments, R⁴ is H or halo, R⁷ is C₁₋₆cycloalkyl, X is —C(O)—, n is 1, p is 1, and Y is absent.

In some embodiments of a compound of formula (I), or a stereoisomer or tautomer thereof, or a pharmaceutically acceptable salt of any of the foregoing, R³ and R⁵ are each independently H, —OH, halo, —N(R^{w1})(R^{w2}), or C₁₋₆alkyl, wherein the C₁₋₆alkyl of R³ and R⁵ is optionally substituted with one or more —OH. In some embodiments, R³ and Rare each independently H, —OH, halo, —N(R^{w1})(R^{w2}), or C₁₋₃alkyl, wherein the C₁₋₃alkyl of R³ and R⁵ is optionally substituted with one or more —OH.

In some embodiments of a compound of formula (I), or a stereoisomer or tautomer thereof, or a pharmaceutically acceptable salt of any of the foregoing, R³ is H.

In some embodiments of a compound of formula (I), or a stereoisomer or tautomer thereof, or a pharmaceutically acceptable salt of any of the foregoing, R⁵ is H, —N(R^{w1})(R^{w2}), or C₁₋₃alkyl, wherein the C₁₋₃alkyl of R⁵ is optionally substituted with one or more —OH. In some embodiments, R⁵ is H. In some embodiments, R⁵ is —N(R^{w1})(R^{w2}), wherein R^{w1} and R^{w2} are independently at each occurrence H, or C₁₋₃alkyl. In some embodiments, R⁵ is —NHCH₃. In some embodiments, R⁵ is C₁₋₃alkyl, wherein the C₁₋₃alkyl of R⁵ is optionally substituted with one or more —OH. In some embodiments, R⁵ is CH₂OH.

In some embodiments of a compound of formula (I), or a stereoisomer or tautomer thereof, or a pharmaceutically acceptable salt of any of the foregoing, R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl and 3-10 membered heterocyclyl of R⁶ are each independently optionally substituted with one or more R^{b}.

In some embodiments of a compound of formula (I), or a stereoisomer or tautomer thereof, or a pharmaceutically acceptable salt of any of the foregoing, R⁶ is —N(R^{w1})(R^{w2}) wherein R^{w1} and R^{w2} are independently at each occurrence H, C₁₋₆alkyl, C₁₋₆haloalkyl, C₃₋₁₀-cycloalkyl, or —C(O)R^{y}. In some embodiments, R^{w1} and R^{w2} are independently at each occurrence H, C₁₋₃alkyl, C₁₋₃haloalkyl, C₃₋₆cycloalkyl, or —C(O)R^{y}. In some embodiments, R^{w1} and R^{w2} are independently at each occurrence H, methyl, ethyl, isopropyl or cyclobutyl. In some embodiments, R⁶ is selected from the group consisting of

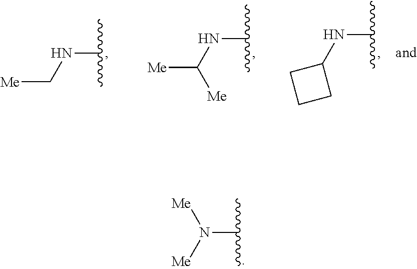

In some embodiments of a compound of formula (I), or a stereoisomer or tautomer thereof, or a pharmaceutically acceptable salt of any of the foregoing, R⁶ is 5-20 membered heteroaryl, wherein the 5-20 membered heteroaryl is optionally substituted with one or more R^{a}. In some embodiments, R⁶ is 3-10 membered heterocyclyl, wherein the 3-10 membered heterocyclyl is optionally substituted with one or more R^{b}. In some embodiments, R⁶ is C₆₋₂₀aryl, wherein the C₆₋₂₀aryl is optionally substituted with one or more R^{a}. In some embodiments, R⁶ is C₃₋₁₀cycloalkyl, wherein the C₃₋₁₀cycloalkyl is optionally substituted with one or more R^{b}. In some embodiments, R⁶ is C₁₋₆alkyl, wherein the C₁₋₆alkyl is optionally substituted with one or more R^{a}.

In some embodiments of a compound of formula (I), or a stereoisomer or tautomer thereof, or a pharmaceutically acceptable salt of any of the foregoing, R⁶ is 5-20 membered heteroaryl, wherein the 5-20 membered heteroaryl is optionally substituted with one or more R^{a}. In some embodiments, R⁶ is selected from the group consisting of

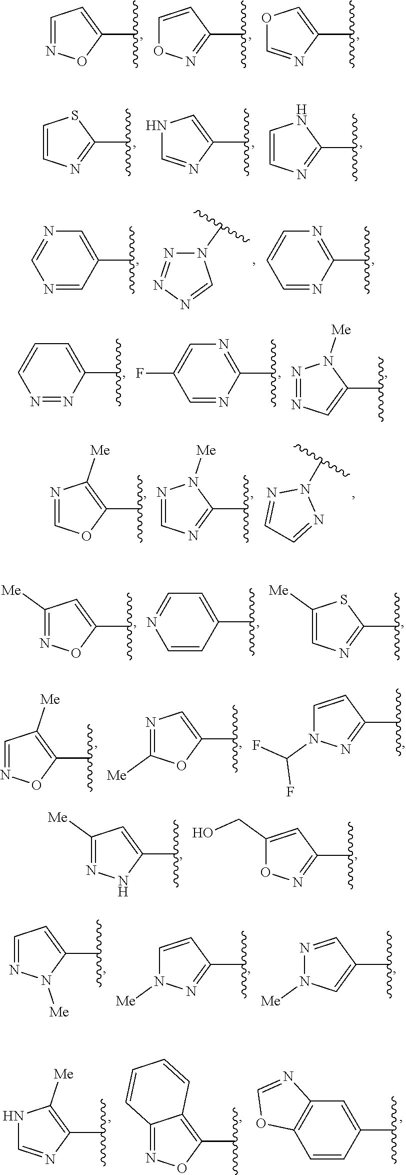

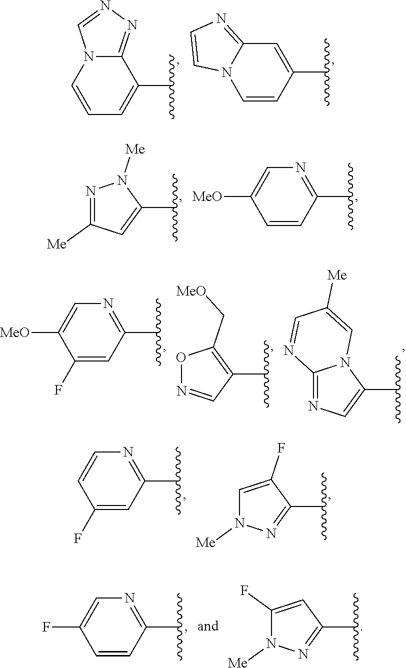

In some embodiments, R⁶ is selected from the group consisting of

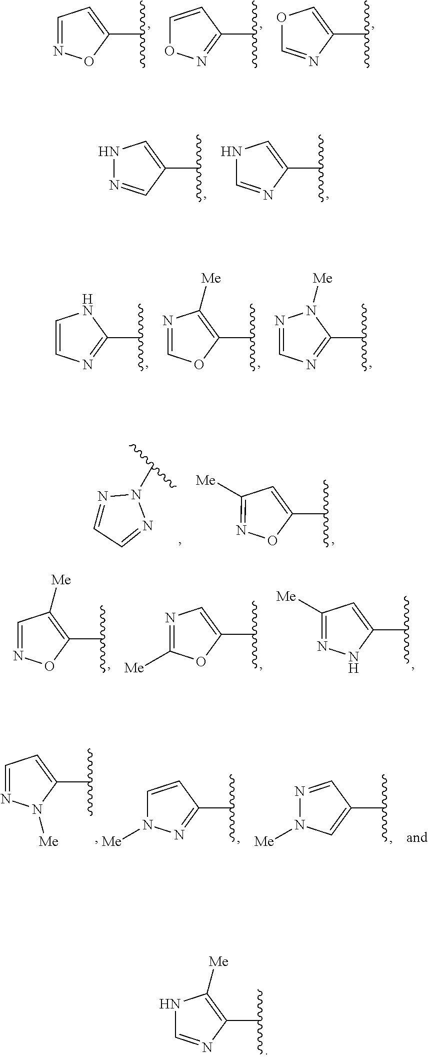

In some embodiments, R⁶ is selected from the group consisting of

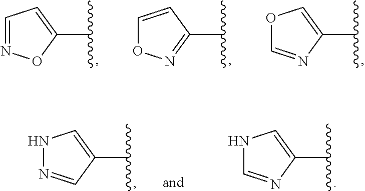 [SMILES: *c1ccno1 ; *c1ccon1 ; *c1cocn1 ; *c1cn[nH]c1 ; *c1c[nH]cn1]

In some embodiments of a compound of formula (I), or a stereoisomer or tautomer thereof, or a pharmaceutically acceptable salt of any of the foregoing, R⁶ is 3-10 membered heterocyclyl, wherein the 3-10 membered heterocyclyl is optionally substituted with one or more R^{b}. In some embodiments, R⁶ is selected from the group consisting of

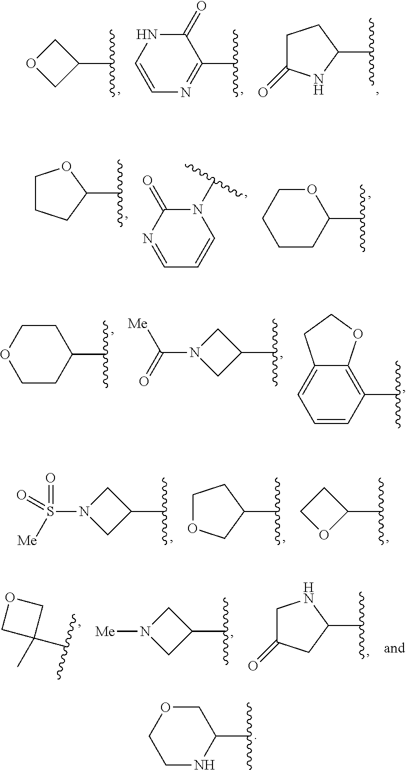

In some embodiments, R⁶ is selected from the group consisting of

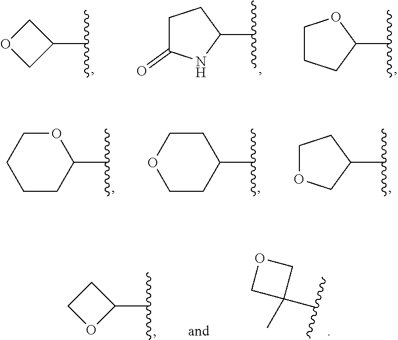

In some embodiments of a compound of formula (I), or a stereoisomer or tautomer thereof, or a pharmaceutically acceptable salt of any of the foregoing, R⁶ is C₆₋₂₀aryl, wherein the C₆₋₂₀aryl is optionally substituted with one or more R^{a}. In some embodiments, R⁶ is selected from the group consisting of

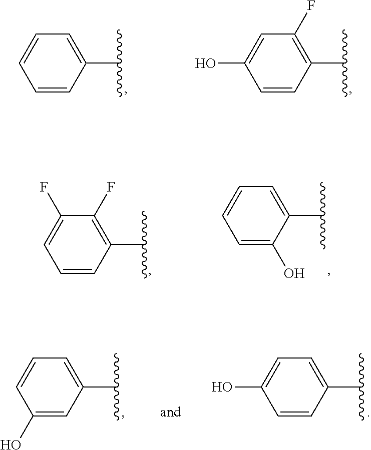 [SMILES: *c1ccccc1 ; *c1ccc(O)cc1F ; *c1cccc(F)c1F ; *c1ccccc1O ; *c1cccc(O)c1 ; *c1ccc(O)cc1]

In some embodiments of a compound of formula (I), or a stereoisomer or tautomer thereof, or a pharmaceutically acceptable salt of any of the foregoing, R⁶ is C₃₋₁₀cycloalkyl, wherein the C₃₋₁₀cycloalkyl is optionally substituted with one or more R^{b}. In some embodiments, R⁶ is selected from the group consisting of

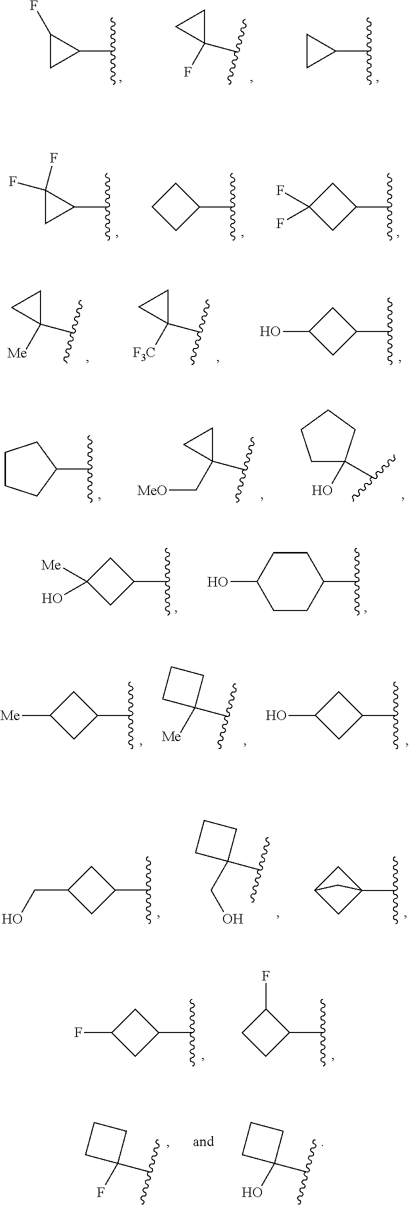

In some embodiments of a compound of formula (I), or a setereoisomer or tautomer thereof, or a pharmaceutically acceptable salt of any of the foregoing, R⁶ is C₁₋₆alkyl, wherein the C₁₋₆alkyl is optionally substituted with one or more R^{a}. In some embodiments, R⁶ is selected from the group consisting of

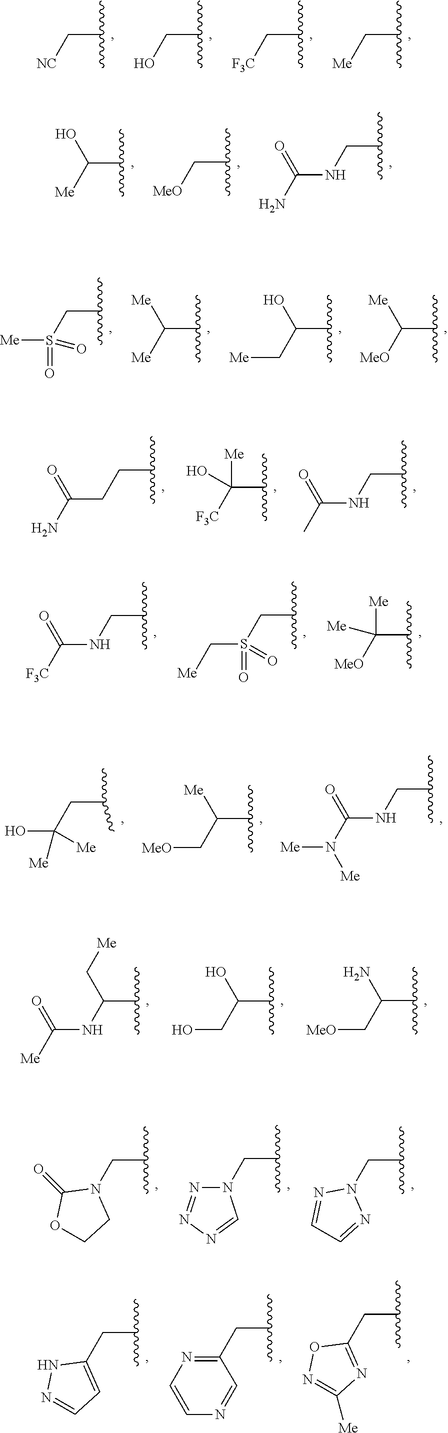

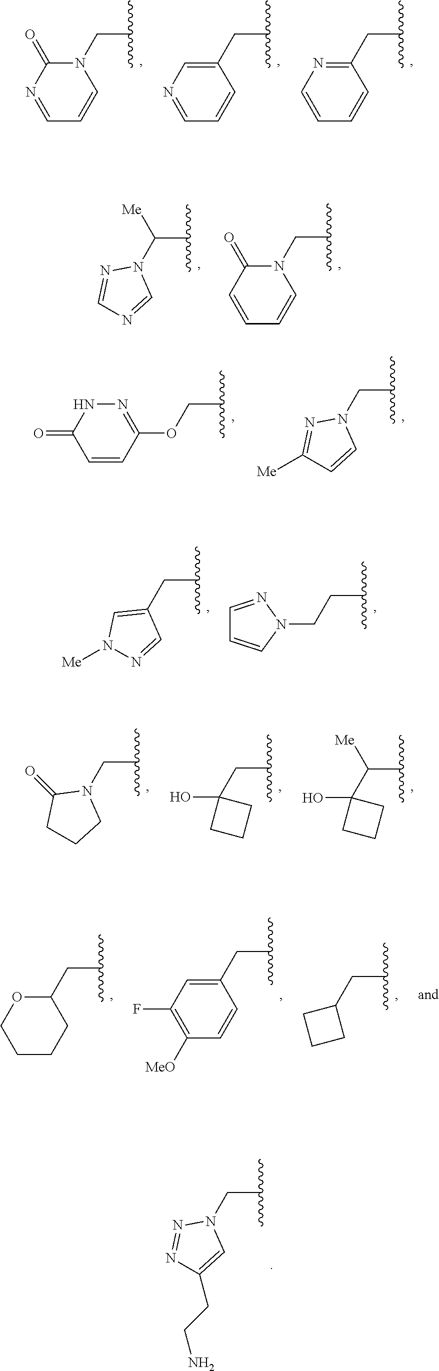

In some embodiments of a compound of formula (I), or a stereoisomer or tautomer thereof, or a pharmaceutically acceptable salt of any of the foregoing, R⁷ is C₁₋₆alkyl, C₁₋₆cycloalkyl, or C₁₋₆haloalkyl. In some embodiments, R⁷ is C₁₋₆alkyl. In some embodiments, R⁷ is C₁₋₆haloalkyl. In some embodiments, R⁷ is C₁₋₆cycloalkyl. In some embodiments, R⁷ is C₁₋₃alkyl, C₁₋₃cycloalkyl, or C₁₋₃haloalkyl. In some embodiments, R⁷ is C₁₋₃alkyl, or C₁₋₃cycloalkyl. In some embodiments, R⁷ is C₁₋₃alkyl. In some embodiments, R⁷ is methyl, ethyl, or isopropyl. In some embodiments, R⁷ is C₁₋₃cycloalkyl. In some embodiments, R⁷ is cyclopropyl.

In some embodiments of a compound of formula (I), or a stereoisomer or tautomer thereof, or a pharmaceutically acceptable salt of any of the foregoing, R⁸ is independently at each occurrence, C₁₋₆alkyl. In some embodiments, R⁸ is independently at each occurrence, C₁₋₃alkyl. In some embodiments, R⁸ is independently at each occurrence, methyl.

In some embodiments of a compound of formula (I), or a stereoisomer or tautomer thereof, or a pharmaceutically acceptable salt of any of the foregoing, r is an integer from 0-11. In some embodiments, r is an integer from 0-9. In some embodiments, r is an integer from 0-4. In some embodiments, r is 1. In some embodiments, r is 1 and R⁸ is methyl.

In some embodiments of a compound of formula (I), or a stereoisomer or tautomer thereof, or a pharmaceutically acceptable salt of any of the foregoing, Y is absent.

In some embodiments of a compound of formula (I), or a stereoisomer or tautomer thereof, or a pharmaceutically acceptable salt of any of the foregoing, embodiments Y is

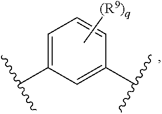

wherein q is an integer from 0-4, and R⁹ is independently at each occurrence, halo. In some embodiments, q is an integer from 0-2. In some embodiments, q is 0. In some embodiments, q is 1. In some embodiments, q is 2. In some embodiments, R⁹ is F or Cl. In some embodiments, R⁹ is F. In some embodiments, R⁹ is Cl.

In some embodiments of a compound of formula (I), or a stereoisomer or tautomer thereof, or a pharmaceutically acceptable salt of any of the foregoing, X is —C(O)—. In some embodiments, X is absent.

In some embodiments of a compound of formula (I), or a stereoisomer or tautomer thereof, or a pharmaceutically acceptable salt of any of the foregoing, n is an integer from 1 to 2. In some embodiments, n is 1. In some embodiments, n is 2.

In some embodiments of a compound of formula (I), or a stereoisomer or tautomer thereof, or a pharmaceutically acceptable salt of any of the foregoing, n is an integer from 1 to 4. In some embodiments, p is 1 or 2. In some embodiments, p is 1. In some embodiments, p is 2.

In some embodiments of a compound of formula (I), or a stereoisomer or tautomer thereof, or a pharmaceutically acceptable salt of any of the foregoing, X is —C(O)—, n is 1, p is 1 and Y is absent. In some embodiments, X is —C(O)—, n is 1, p is 1 and Y is

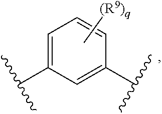

wherein q is 0. In some embodiments, X is —C(O)—, n is 1, p is 1 and Y is

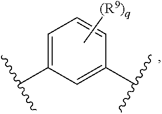

wherein q is 1. In some embodiments, X is —C(O)—, n is 1, p is 1 and Y is

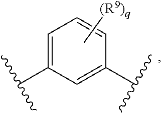

wherein q is 2.

In some embodiments of a compound of formula (I), or a stereoisomer or tautomer thereof, or a pharmaceutically acceptable salt of any of the foregoing, R⁷ is C₁₋₆alkyl, X is —C(O)—, n is 1, p is 1, and Y is absent. In some embodiments, R⁷ is C₁₋₆cycloalkyl, and X is —C(O)—, n is 1, p is 1, and Y is absent. In some embodiments, R⁷ is C₁₋₆haloalkyl and X is —C(O)—, n is 1, p is 1, and Y is absent.

In some embodiments of a compound of formula (I), or a stereoisomer or tautomer thereof, or a pharmaceutically acceptable salt of any of the foregoing, X is —C(O)—; R⁷ is methyl; p is 1; n is 1; Y is absent; and R⁶ is selected from the group consisting of

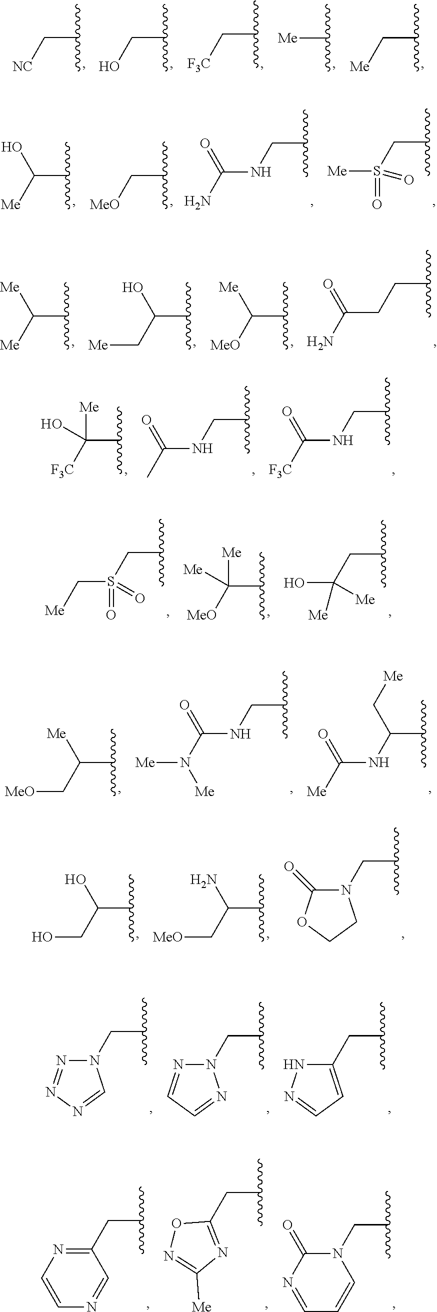

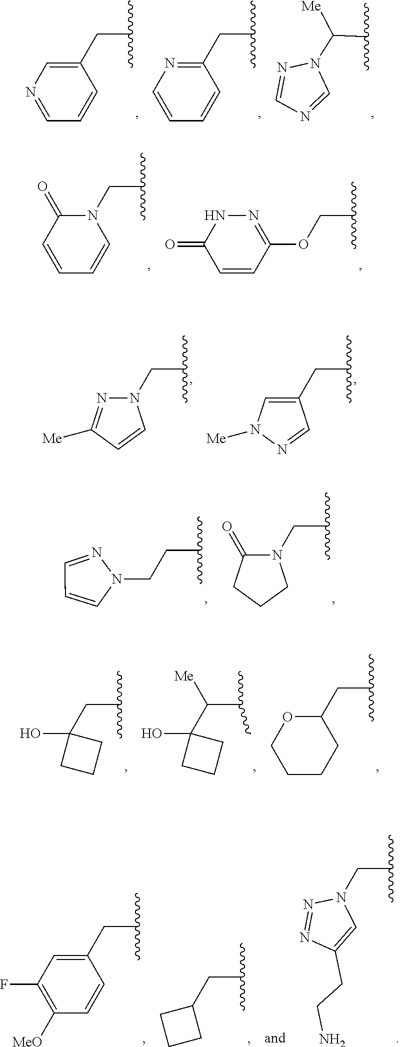

In some embodiments of a compound of formula (I), or a stereoisomer or tautomer thereof, or a pharmaceutically acceptable salt of any of the foregoing, X is —C(O)—; R⁷ is methyl; p is 1; n is 1; Y is absent; and R⁶ is selected from the group consisting of

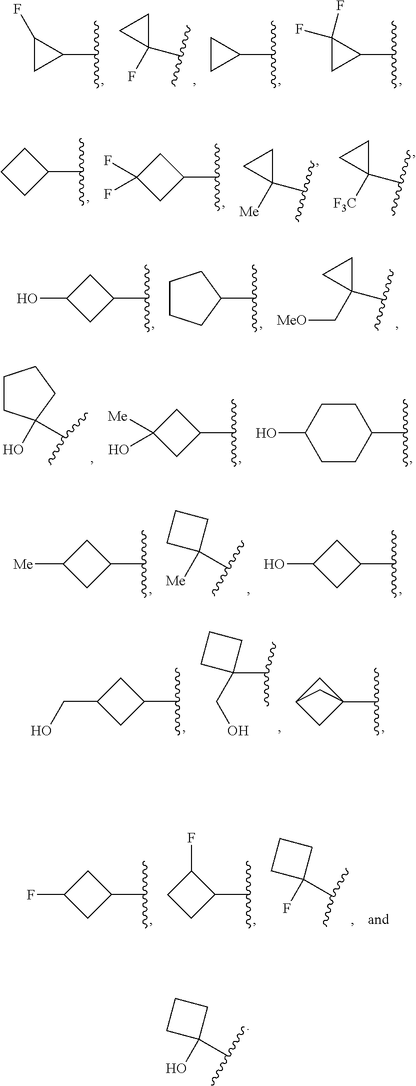

In some embodiments of a compound of formula (I), or a stereoisomer or tautomer thereof, or a pharmaceutically acceptable salt of any of the foregoing, X is —C(O)—; R⁷ is methyl; p is 1; n is 1; Y is absent; and R⁶ is selected from the group consisting of

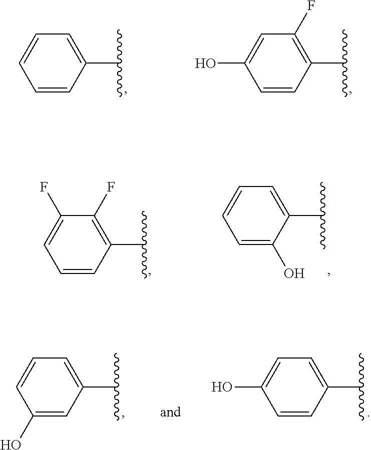 [SMILES: *c1ccccc1 ; *c1ccc(O)cc1F ; *c1cccc(F)c1F ; *c1ccccc1O ; *c1cccc(O)c1 ; *c1ccc(O)cc1]

In some embodiments of a compound of formula (I), or a stereoisomer or tautomer thereof, or a pharmaceutically acceptable salt of any of the foregoing, X is —C(O)—; R⁷ is methyl; p is 1; n is 1; Y is absent; and R⁶ is selected from the group consisting of

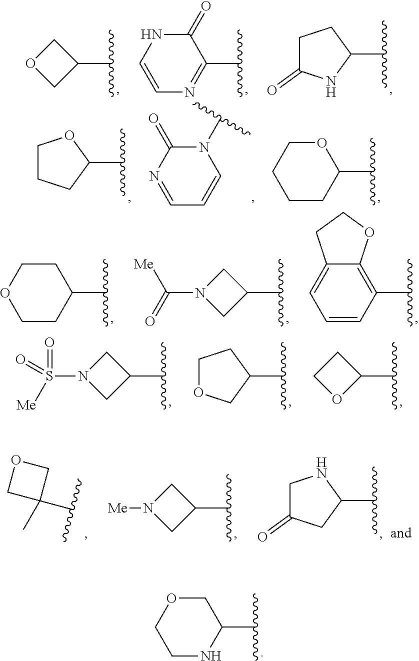

In some embodiments of a compound of formula (I), or a stereoisomer or tautomer thereof, or a pharmaceutically acceptable salt of any of the foregoing, X is —C(O)—; R⁷ is methyl; p is 1; n is 1; Y is absent; and R⁶ is selected from the group consisting of

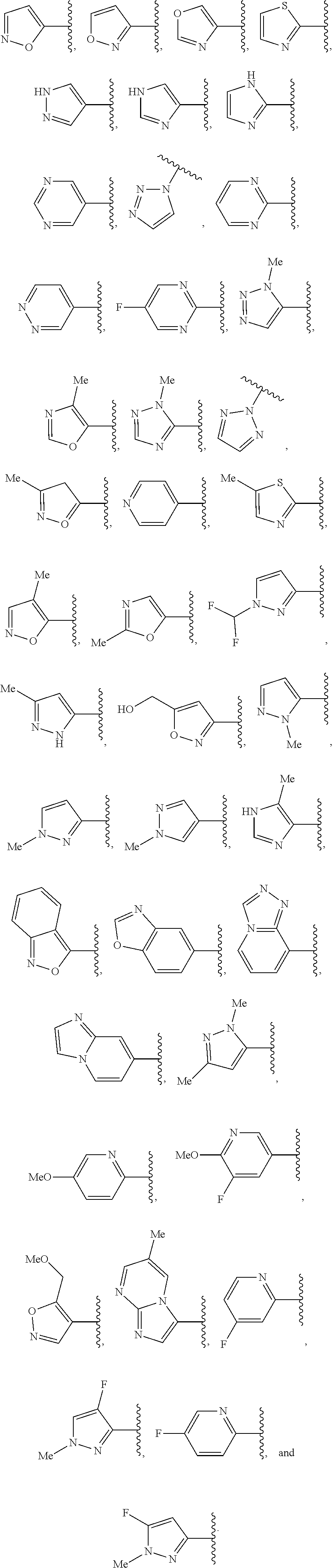

In some embodiments of a compound of formula (I), or a stereoisomer or tautomer thereof, or a pharmaceutically acceptable salt of any of the foregoing, R⁶ is C₁₋₆alkyl, wherein the C₁₋₆alkyl is optionally substituted with one or more R^{a}; each R^{a} is independently —OH, halo, —CN, C₁₋₆alkyl, C₁₋₆alkoxy, —N(R^{w1})(R^{w2}), —C(O)R^{y}, or —SO₂R^{z}; X is —C(O)—; Y is absent; p is 1; and n is 1. In some embodiments, the moiety represented by

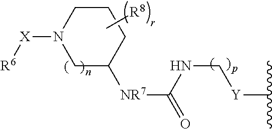

selected from the group consisting of

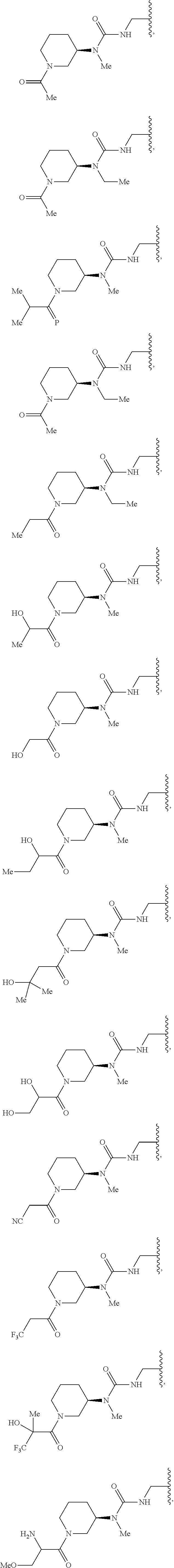

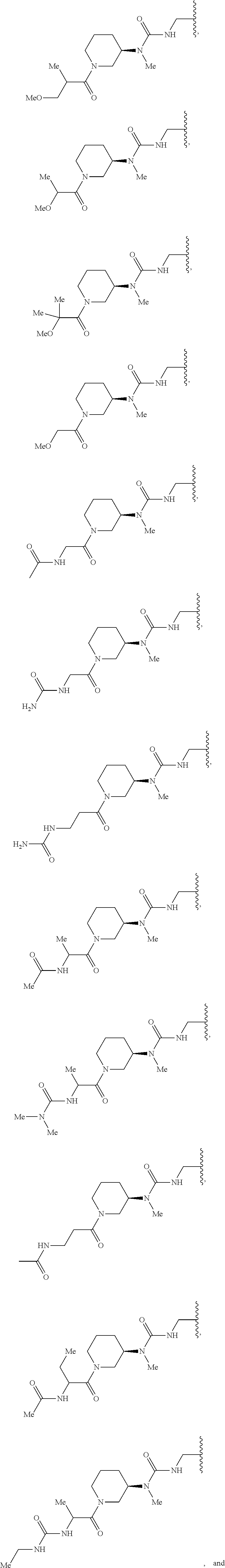

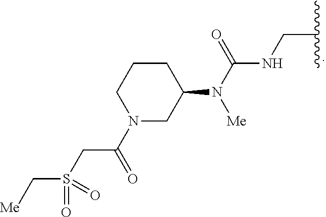 [SMILES: *CNC(=O)N(C)[C@@H]1CCCN(C(=O)CS(=O)(=O)CC)C1]

In some embodiments of a compound of formula (I), or a stereoisomer or tautomer thereof, or a pharmaceutically acceptable salt of any of the foregoing, R⁶ is C₁₋₆alkyl, wherein the C₁₋₆alkyl is optionally substituted with one or more R^{a}; each R^{a} is independently 3-10 membered heterocyclyl optionally substituted with one or more R^{b}; X is —C(O)—; Y is absent; p is 1; and n is 1. In some embodiments, the moiety represented by

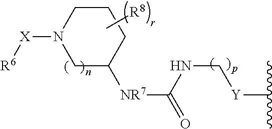

is selected from the group consisting of

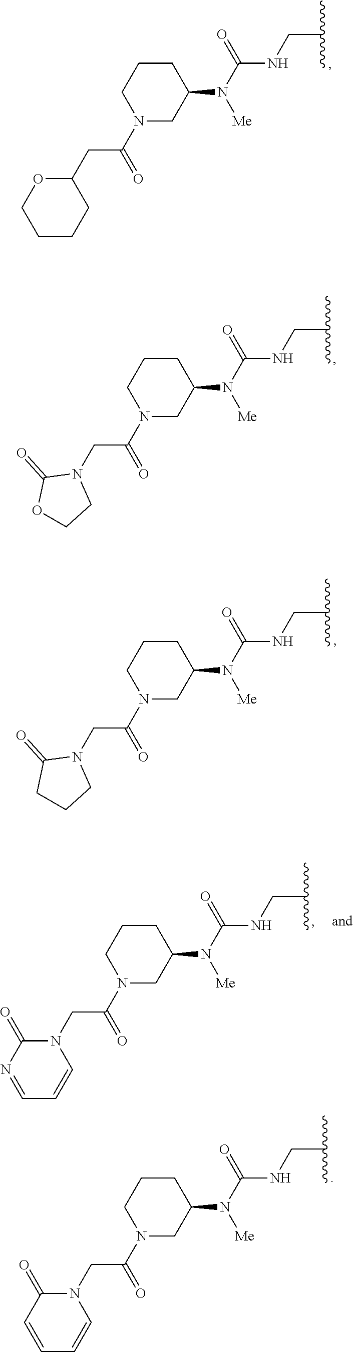 [SMILES: *CNC(=O)N(C)[C@@H]1CCCN(C(=O)CC2CCCCO2)C1 ; *CNC(=O)N(C)[C@@H]1CCCN(C(=O)CN2CCOC2=O)C1 ; *CNC(=O)N(C)[C@@H]1CCCN(C(=O)CN2CCCC2=O)C1 ; *CNC(=O)N(C)[C@@H]1CCCN(C(=O)Cn2cccnc2=O)C1 ; *CNC(=O)N(C)[C@@H]1CCCN(C(=O)Cn2ccccc2=O)C1]

In some embodiments of a compound of formula (I), or a stereoisomer or tautomer thereof, or a pharmaceutically acceptable salt of any of the foregoing, R⁶ is C₁₋₆alkyl, wherein the C₁₋₆alkyl is optionally substituted with one or more R^{a}; each R^{a} is independently 5-20 membered heteroaryl optionally substituted with one or more R^{c}; X is —C(O)—; Y is absent; p is 1; and n is 1. In some embodiments, the moiety represented by

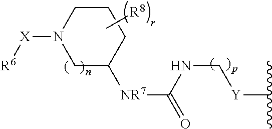

is selected from the group consisting of

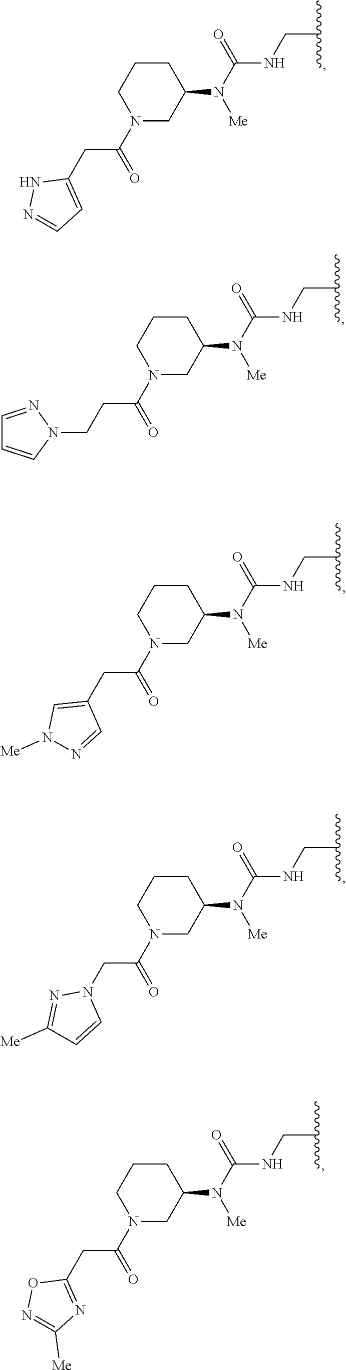 [SMILES: *CNC(=O)N(C)[C@@H]1CCCN(C(=O)Cc2ccn[nH]2)C1 ; *CNC(=O)N(C)[C@@H]1CCCN(C(=O)CCn2cccn2)C1 ; *CNC(=O)N(C)[C@@H]1CCCN(C(=O)Cc2cnn(C)c2)C1 ; *CNC(=O)N(C)[C@@H]1CCCN(C(=O)Cn2ccc(C)n2)C1 ; *CNC(=O)N(C)[C@@H]1CCCN(C(=O)Cc2nc(C)no2)C1]

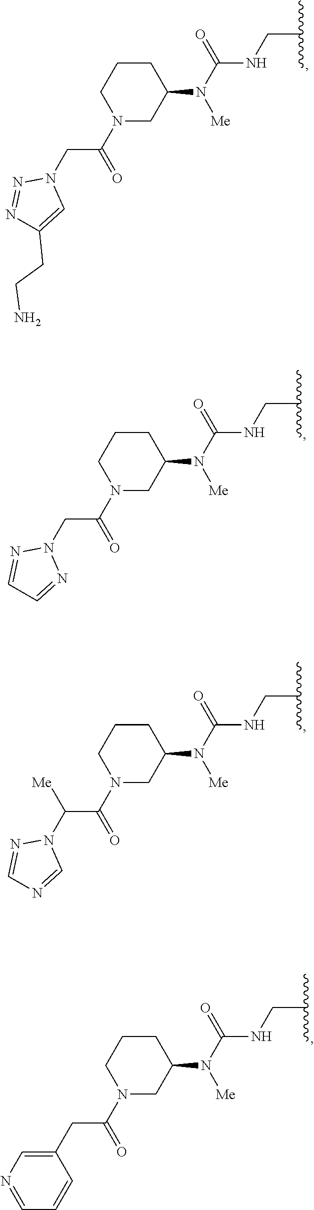 [SMILES: *CNC(=O)N(C)[C@@H]1CCCN(C(=O)Cn2cc(CCN)nn2)C1 ; *CNC(=O)N(C)[C@@H]1CCCN(C(=O)Cn2nccn2)C1 ; *CNC(=O)N(C)[C@@H]1CCCN(C(=O)C(C)n2cncn2)C1 ; *CNC(=O)N(C)[C@@H]1CCCN(C(=O)Cc2cccnc2)C1]

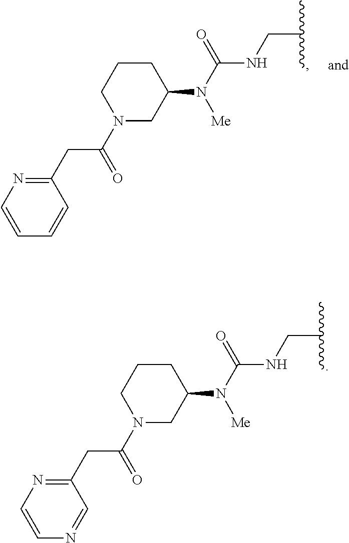 [SMILES: *CNC(=O)N(C)[C@@H]1CCCN(C(=O)Cc2ccccn2)C1 ; *CNC(=O)N(C)[C@@H]1CCCN(C(=O)Cc2cnccn2)C1]

In some embodiments of a compound of formula (I), or a stereoisomer or tautomer thereof, or a pharmaceutically acceptable salt of any of the foregoing, R⁶ is C₁₋₆alkyl, wherein the C₁₋₆alkyl is optionally substituted with one or more R^{a}; each R^{a} is independently —OR^{x}; X is —C(O)—; Y is absent; p is 1; and n is 1. In some embodiments, the moiety represented by

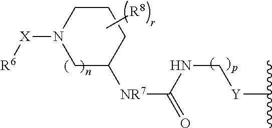

is

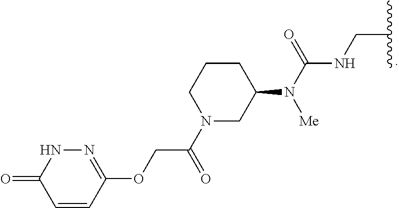 [SMILES: *CNC(=O)N(C)[C@@H]1CCCN(C(=O)COc2ccc(=O)[nH]n2)C1]

In some embodiments of a compound of formula (I), or a stereoisomer or tautomer thereof, or a pharmaceutically acceptable salt of any of the foregoing, R⁶ is C₁₋₆alkyl, wherein the C₁₋₆alkyl is optionally substituted with one or more R^{a}; each R^{a} is independently C₃₋₁₀-cycloalkyl optionally substituted with one or more R^{b}; X is —C(O)—; Y is absent; and n is 1. In some embodiments, the moiety represented by

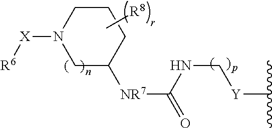

is selected from the group consisting of

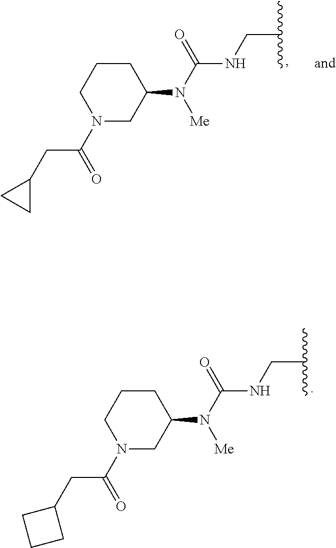 [SMILES: *CNC(=O)N(C)[C@@H]1CCCN(C(=O)CC2CC2)C1 ; *CNC(=O)N(C)[C@@H]1CCCN(C(=O)CC2CCC2)C1]

In some embodiments of a compound of formula (I), or a stereoisomer or tautomer thereof, or a pharmaceutically acceptable salt of any of the foregoing, R⁶ is 5-20 membered heteroaryl optionally substituted with one or more R^{a}; X is —C(O)—; Y is absent; p is 1; and n is 1. In some embodiments, the moiety represented by

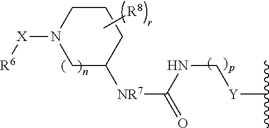

is selected from the group consisting of

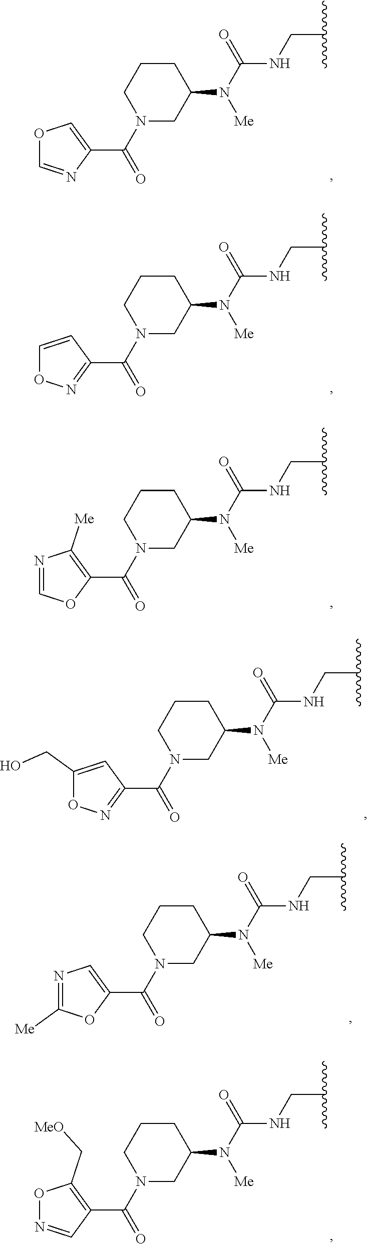 [SMILES: *CNC(=O)N(C)[C@@H]1CCCN(C(=O)c2cocn2)C1 ; *CNC(=O)N(C)[C@@H]1CCCN(C(=O)c2ccon2)C1 ; *CNC(=O)N(C)[C@@H]1CCCN(C(=O)c2ocnc2C)C1 ; *CNC(=O)N(C)[C@@H]1CCCN(C(=O)c2cc(CO)on2)C1 ; *CNC(=O)N(C)[C@@H]1CCCN(C(=O)c2cnc(C)o2)C1 ; *CNC(=O)N(C)[C@@H]1CCCN(C(=O)c2cnoc2COC)C1]

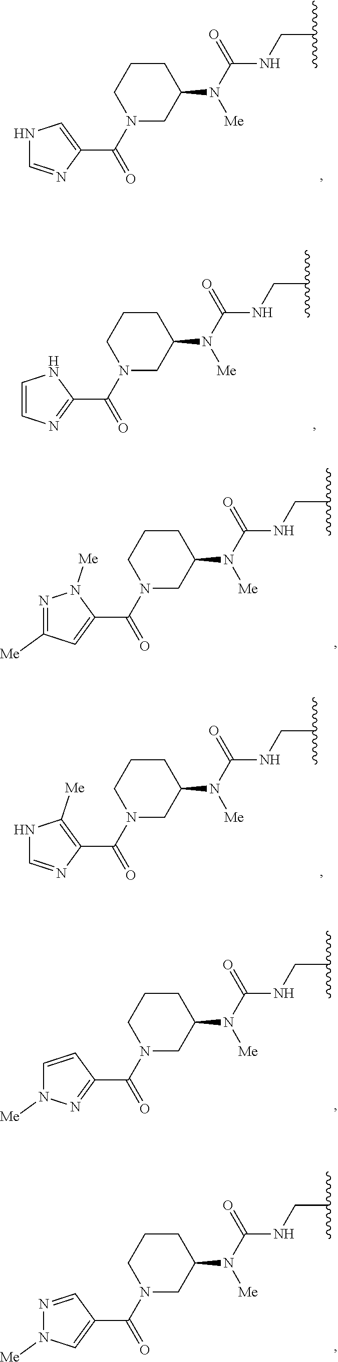 [SMILES: *CNC(=O)N(C)[C@@H]1CCCN(C(=O)c2c[nH]cn2)C1 ; *CNC(=O)N(C)[C@@H]1CCCN(C(=O)c2ncc[nH]2)C1 ; *CNC(=O)N(C)[C@@H]1CCCN(C(=O)c2cc(C)nn2C)C1 ; *CNC(=O)N(C)[C@@H]1CCCN(C(=O)c2nc[nH]c2C)C1 ; *CNC(=O)N(C)[C@@H]1CCCN(C(=O)c2ccn(C)n2)C1 ; *CNC(=O)N(C)[C@@H]1CCCN(C(=O)c2cnn(C)c2)C1]

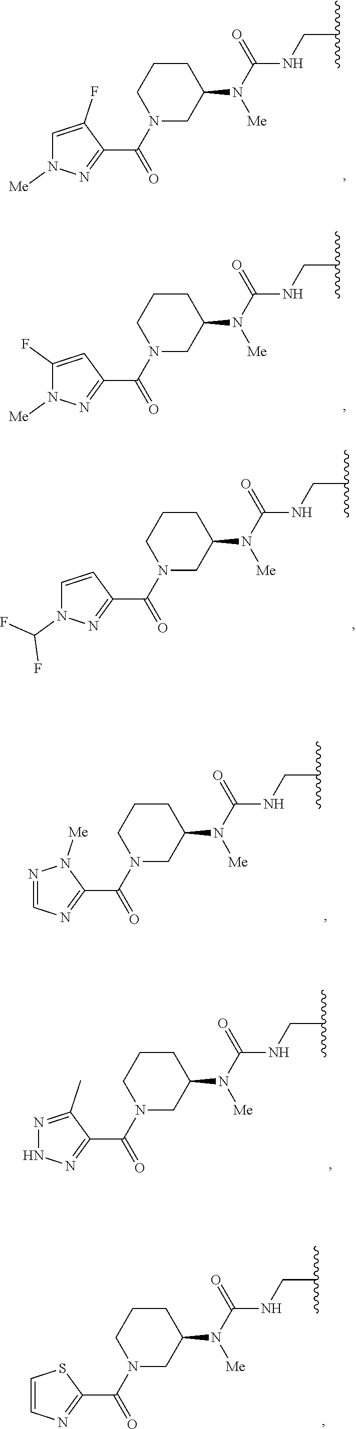 [SMILES: *CNC(=O)N(C)[C@@H]1CCCN(C(=O)c2nn(C)cc2F)C1 ; *CNC(=O)N(C)[C@@H]1CCCN(C(=O)c2cc(F)n(C)n2)C1 ; *CNC(=O)N(C)[C@@H]1CCCN(C(=O)c2ccn(C(F)F)n2)C1 ; *CNC(=O)N(C)[C@@H]1CCCN(C(=O)c2ncnn2C)C1 ; *CNC(=O)N(C)[C@@H]1CCCN(C(=O)c2n[nH]nc2C)C1 ; *CNC(=O)N(C)[C@@H]1CCCN(C(=O)c2nccs2)C1]

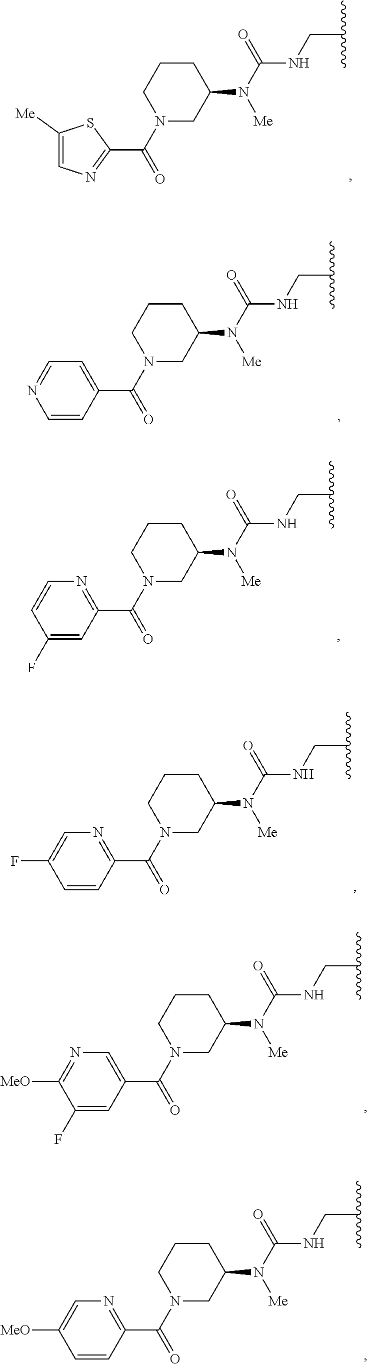 [SMILES: *CNC(=O)N(C)[C@@H]1CCCN(C(=O)c2ncc(C)s2)C1 ; *CNC(=O)N(C)[C@@H]1CCCN(C(=O)c2ccncc2)C1 ; *CNC(=O)N(C)[C@@H]1CCCN(C(=O)c2cc(F)ccn2)C1 ; *CNC(=O)N(C)[C@@H]1CCCN(C(=O)c2ccc(F)cn2)C1 ; *CNC(=O)N(C)[C@@H]1CCCN(C(=O)c2cnc(OC)c(F)c2)C1 ; *CNC(=O)N(C)[C@@H]1CCCN(C(=O)c2ccc(OC)cn2)C1]

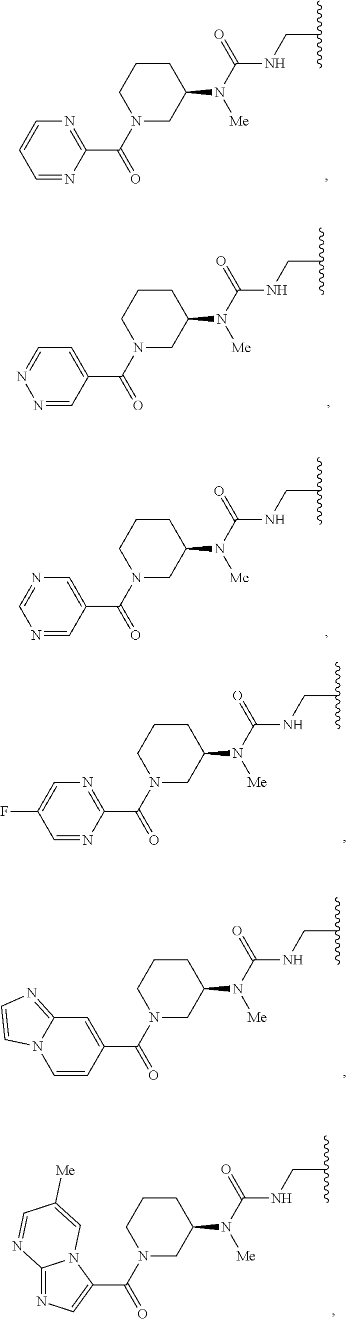 [SMILES: *CNC(=O)N(C)[C@@H]1CCCN(C(=O)c2ncccn2)C1 ; *CNC(=O)N(C)[C@@H]1CCCN(C(=O)c2ccnnc2)C1 ; *CNC(=O)N(C)[C@@H]1CCCN(C(=O)c2cncnc2)C1 ; *CNC(=O)N(C)[C@@H]1CCCN(C(=O)c2ncc(F)cn2)C1 ; *CNC(=O)N(C)[C@@H]1CCCN(C(=O)c2ccn3ccnc3c2)C1 ; *CNC(=O)N(C)[C@@H]1CCCN(C(=O)c2cnc3ncc(C)cn23)C1]

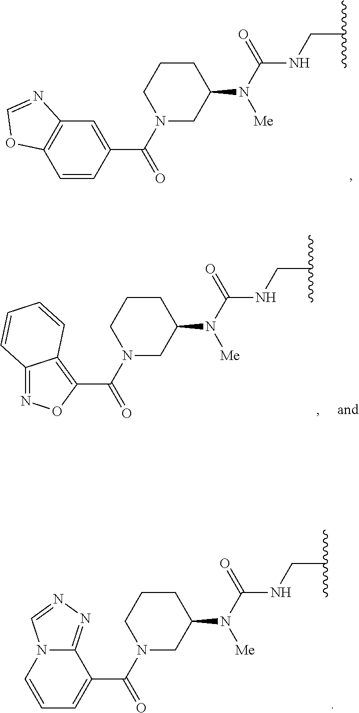 [SMILES: *CNC(=O)N(C)[C@@H]1CCCN(C(=O)c2ccc3ocnc3c2)C1 ; *CNC(=O)N(C)[C@@H]1CCCN(C(=O)c2onc3ccccc23)C1 ; *CNC(=O)N(C)[C@@H]1CCCN(C(=O)c2cccn3cnnc23)C1]

In some embodiments of a compound of formula (I), or a stereoisomer or tautomer thereof, or a pharmaceutically acceptable salt of any of the foregoing, R⁶ is 3-10 membered heterocyclyl optionally substituted with one or more R^{b}; X is —C(O)—; Y is absent; p is 1; and n is 1. In some embodiments, the moiety represented by

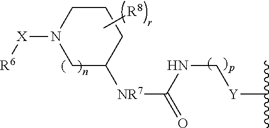

is selected from the group consisting of

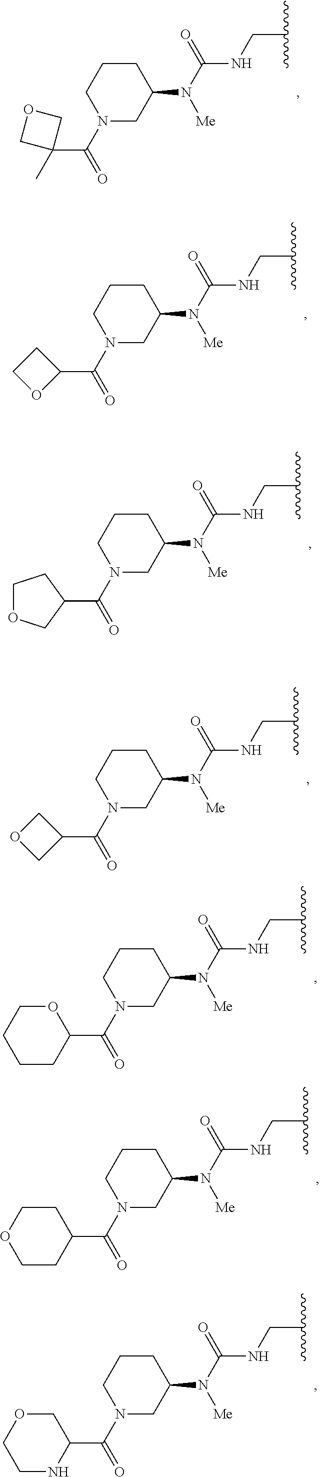

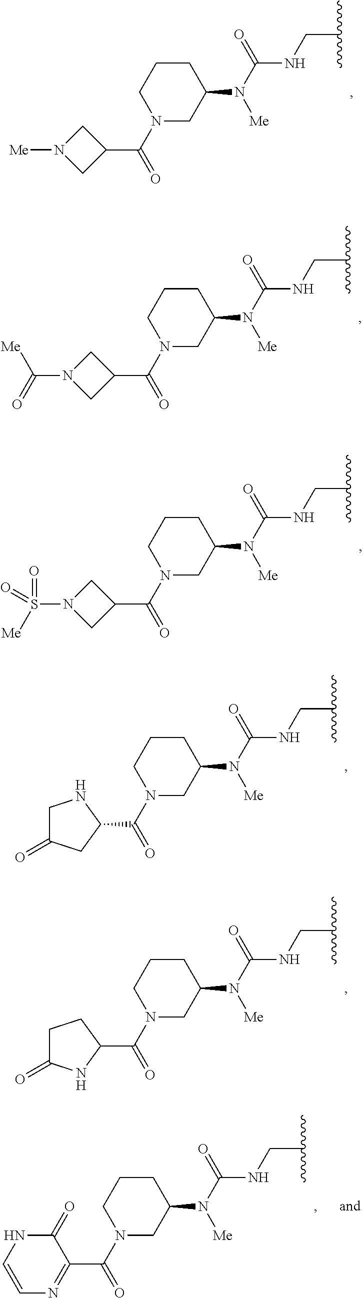 [SMILES: *CNC(=O)N(C)[C@@H]1CCCN(C(=O)C2CN(C)C2)C1 ; *CNC(=O)N(C)[C@@H]1CCCN(C(=O)C2CN(C(C)=O)C2)C1 ; *CNC(=O)N(C)[C@@H]1CCCN(C(=O)C2CN(S(C)(=O)=O)C2)C1 ; *CNC(=O)N(C)[C@@H]1CCCN(C(=O)[C@@H]2CC(=O)CN2)C1 ; *CNC(=O)N(C)[C@@H]1CCCN(C(=O)C2CCC(=O)N2)C1 ; *CNC(=O)N(C)[C@@H]1CCCN(C(=O)c2ncc[nH]c2=O)C1]

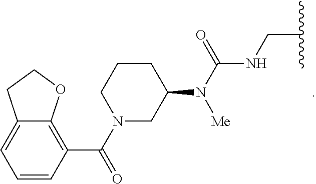 [SMILES: *CNC(=O)N(C)[C@@H]1CCCN(C(=O)c2cccc3c2OCC3)C1]

In some embodiments of a compound of formula (I), or a stereoisomer or tautomer thereof, or a pharmaceutically acceptable salt of any of the foregoing, R⁶ is C₆₋₂₀aryl optionally substituted with one or more R^{a}; X is —C(O)—; Y is absent; p is 1; and n is 1. In some embodiments, the moiety represented by

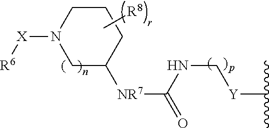

selected from the group consisting of

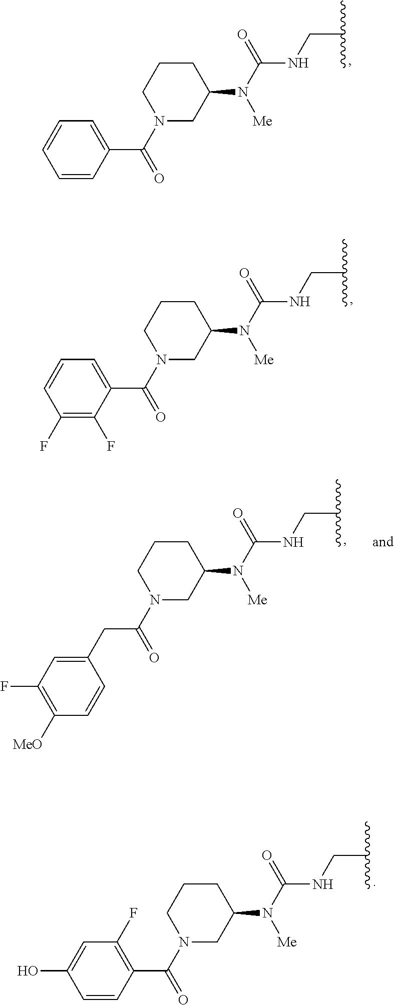 [SMILES: *CNC(=O)N(C)[C@@H]1CCCN(C(=O)c2ccccc2)C1 ; *CNC(=O)N(C)[C@@H]1CCCN(C(=O)c2cccc(F)c2F)C1 ; *CNC(=O)N(C)[C@@H]1CCCN(C(=O)Cc2ccc(OC)c(F)c2)C1 ; *CNC(=O)N(C)[C@@H]1CCCN(C(=O)c2ccc(O)cc2F)C1]

In some embodiments of a compound of formula (I), or a stereoisomer or tautomer thereof, or a pharmaceutically acceptable salt of any of the foregoing, R⁶ is C₃₋₁₀cycloalkyl optionally substituted with one or more R^{b}; X is —C(O)—; Y is absent; p is 1; and n is 1. In some embodiments, the moiety represented by

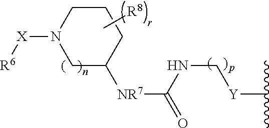

is selected from the group consisting of

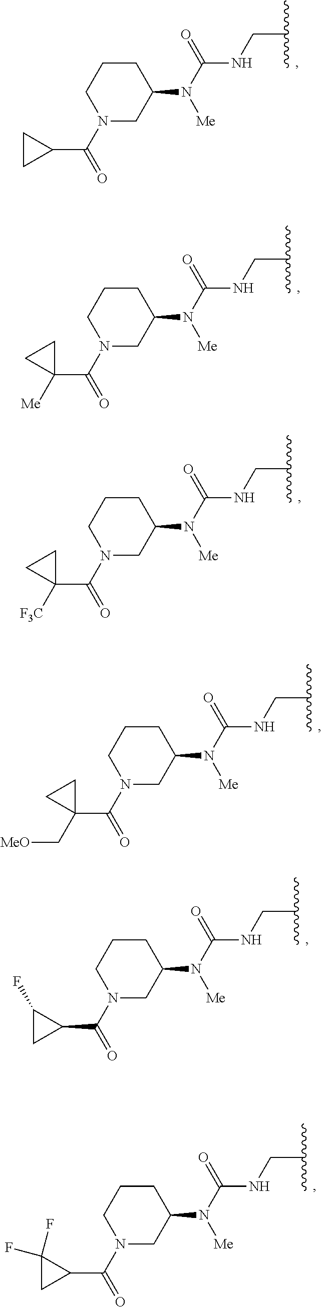 [SMILES: *CNC(=O)N(C)[C@@H]1CCCN(C(=O)C2CC2)C1 ; *CNC(=O)N(C)[C@@H]1CCCN(C(=O)C2(C)CC2)C1 ; *CNC(=O)N(C)[C@@H]1CCCN(C(=O)C2(C(F)(F)F)CC2)C1 ; *CNC(=O)N(C)[C@@H]1CCCN(C(=O)C2(COC)CC2)C1 ; *CNC(=O)N(C)[C@@H]1CCCN(C(=O)[C@H]2C[C@@H]2F)C1 ; *CNC(=O)N(C)[C@@H]1CCCN(C(=O)C2CC2(F)F)C1]

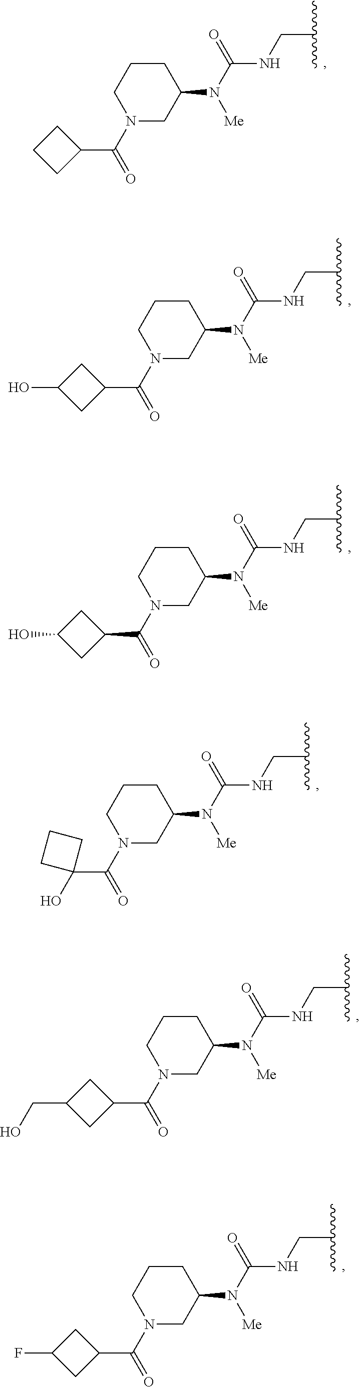 [SMILES: *CNC(=O)N(C)[C@@H]1CCCN(C(=O)C2CCC2)C1 ; *CNC(=O)N(C)[C@@H]1CCCN(C(=O)C2CC(O)C2)C1 ; *CNC(=O)N(C)[C@@H]1CCCN(C(=O)[C@H]2C[C@H](O)C2)C1 ; *CNC(=O)N(C)[C@@H]1CCCN(C(=O)C2(O)CCC2)C1 ; *CNC(=O)N(C)[C@@H]1CCCN(C(=O)C2CC(CO)C2)C1 ; *CNC(=O)N(C)[C@@H]1CCCN(C(=O)C2CC(F)C2)C1]

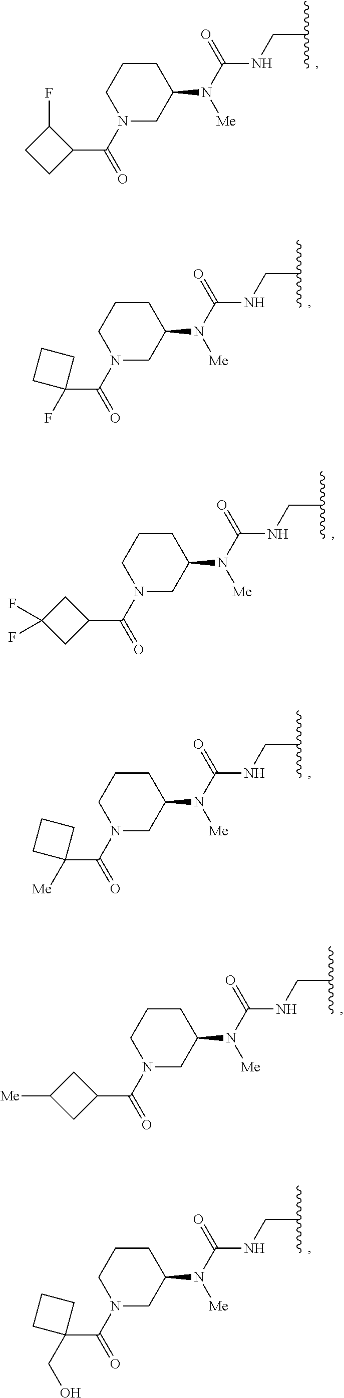 [SMILES: *CNC(=O)N(C)[C@@H]1CCCN(C(=O)C2CCC2F)C1 ; *CNC(=O)N(C)[C@@H]1CCCN(C(=O)C2(F)CCC2)C1 ; *CNC(=O)N(C)[C@@H]1CCCN(C(=O)C2CC(F)(F)C2)C1 ; *CNC(=O)N(C)[C@@H]1CCCN(C(=O)C2(C)CCC2)C1 ; *CNC(=O)N(C)[C@@H]1CCCN(C(=O)C2CC(C)C2)C1 ; *CNC(=O)N(C)[C@@H]1CCCN(C(=O)C2(CO)CCC2)C1]

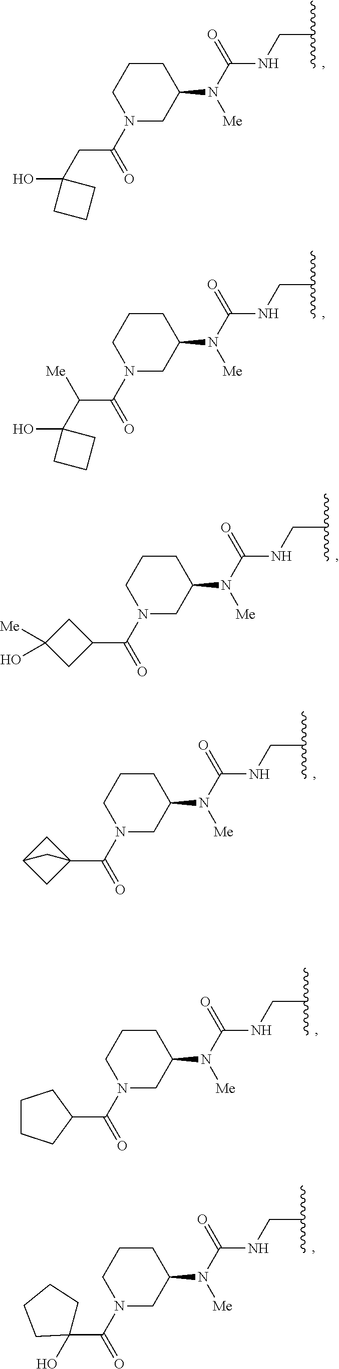 [SMILES: *CNC(=O)N(C)[C@@H]1CCCN(C(=O)CC2(O)CCC2)C1 ; *CNC(=O)N(C)[C@@H]1CCCN(C(=O)C(C)C2(O)CCC2)C1 ; *CNC(=O)N(C)[C@@H]1CCCN(C(=O)C2CC(C)(O)C2)C1 ; *CNC(=O)N(C)[C@@H]1CCCN(C(=O)C23CC(C2)C3)C1 ; *CNC(=O)N(C)[C@@H]1CCCN(C(=O)C2CCCC2)C1 ; *CNC(=O)N(C)[C@@H]1CCCN(C(=O)C2(O)CCCC2)C1]

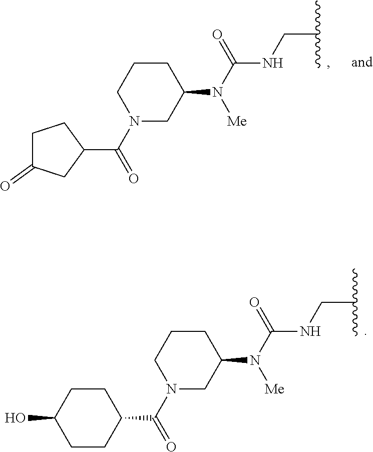 [SMILES: *CNC(=O)N(C)[C@@H]1CCCN(C(=O)C2CCC(=O)C2)C1 ; *CNC(=O)N(C)[C@@H]1CCCN(C(=O)[C@H]2CC[C@H](O)CC2)C1]

In some embodiments of a compound of formula (I), or a stereoisomer or tautomer thereof, or a pharmaceutically acceptable salt of any of the foregoing, R⁶ is 5-20 membered heteroaryl optionally substituted with one or more R^{a}; X is absent; Y is absent; p is 1; and n is 1. In some embodiments, the moiety represented by

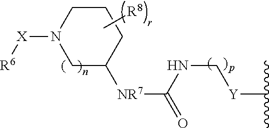

is selected from the group consisting of

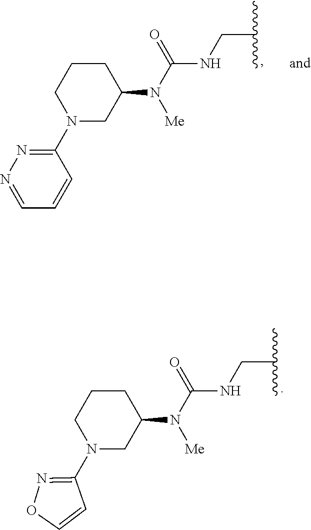 [SMILES: *CNC(=O)N(C)[C@@H]1CCCN(c2cccnn2)C1 ; *CNC(=O)N(C)[C@@H]1CCCN(c2ccon2)C1]

In some embodiments of a compound of formula (I), or a stereoisomer or tautomer thereof, or a pharmaceutically acceptable salt of any of the foregoing, Y is absent; p is 1; and n is 1. In some embodiments, the moiety represented by

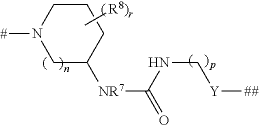

is selected from the group consisting of

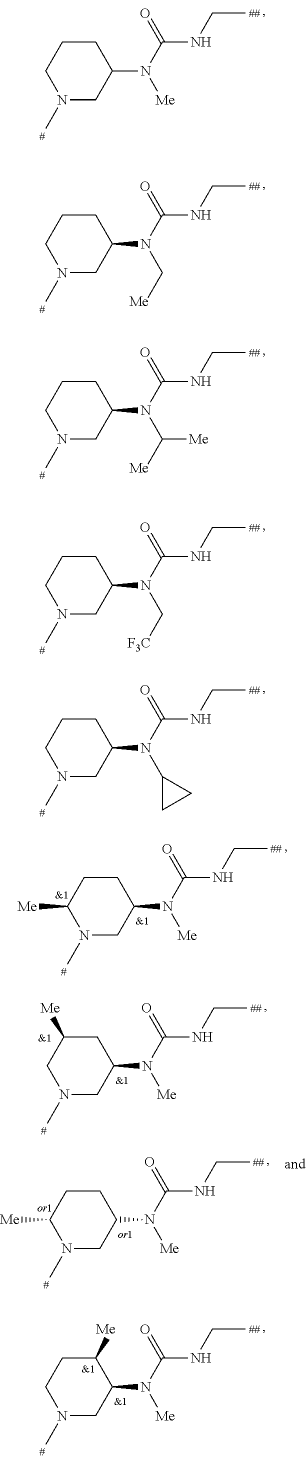

wherein # represents the point of attachment to X, and ## represents the point of attachment to the rest of the molecule.

In some embodiments of a compound of formula (I), or a stereoisomer or tautomer thereof, or a pharmaceutically acceptable salt of any of the foregoing, Y is

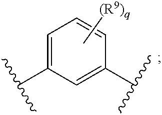

p is 2; and n is 1. In some embodiments, the moiety represented by

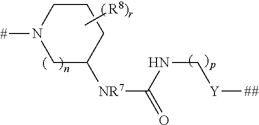

is selected from the group consisting of

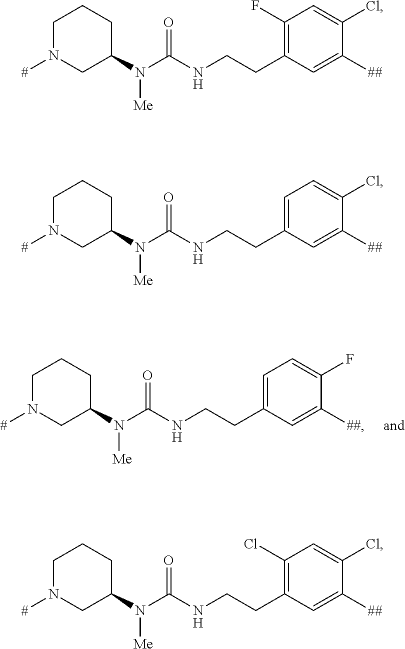

wherein # represents the point of attachment to X, and ## represents the point of attachment to the rest of the molecule.

In some embodiments of a compound of formula (I), or a stereoisomer or tautomer thereof, or a pharmaceutically acceptable salt of any of the foregoing, the moiety represented by

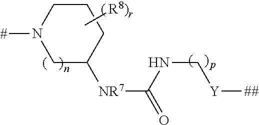

is of the configuration

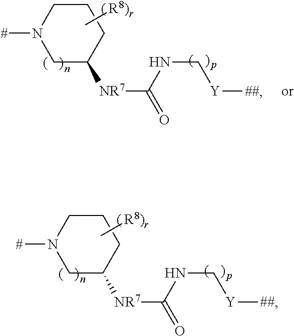

wherein # represents the point of attachment to X, and ## represents the point of attachment to the rest of the molecule. In some embodiments, the moiety represented by

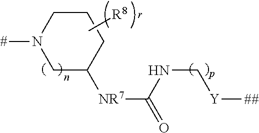

is of the configuration

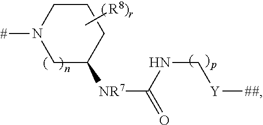

wherein # represents the point of attachment to X, and ## represents the point of attachment to the rest of the molecule. In some embodiments, the moiety represented by

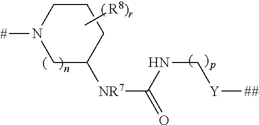

is of the configuration

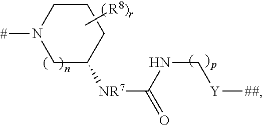

wherein # represents the point of attachment to X, and ## represents the point of attachment to the rest of the molecule.

In some embodiments of a compound of formula (I), or a stereoisomer or tautomer thereof, or a pharmaceutically acceptable salt of any of the foregoing, the moiety represented by

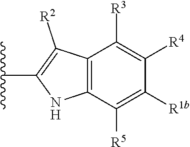

is a 2-substituted indole such as

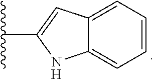 [SMILES: *c1cc2ccccc2[nH]1]

In some embodiments, the moiety represented by

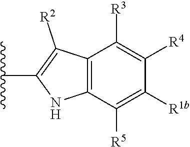

comprises a 2-substituted indole such as

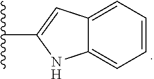 [SMILES: *c1cc2ccccc2[nH]1]

In some embodiments of a compound of formula (I), or a stereoisomer or tautomer thereof, or a pharmaceutically acceptable salt of any of the foregoing, the moiety represented by

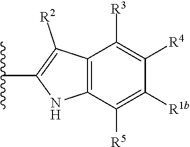

is a 2,5-substituted indole selected from the group consisting of

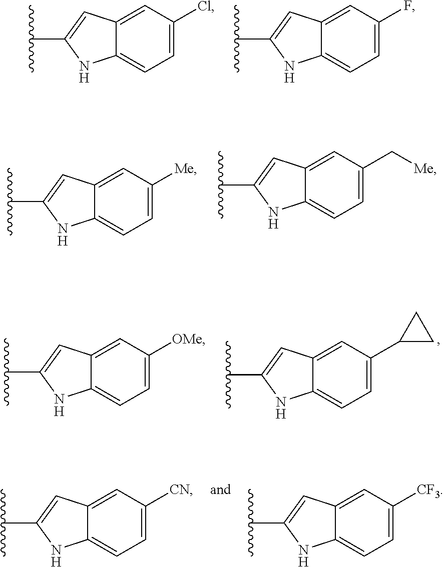

In some embodiments of a compound of formula (I), or a stereoisomer or tautomer thereof, or a pharmaceutically acceptable salt of any of the foregoing, the moiety represented by

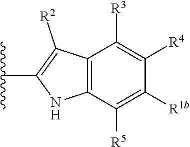

is a 2,6-substituted indole selected from the group consisting of

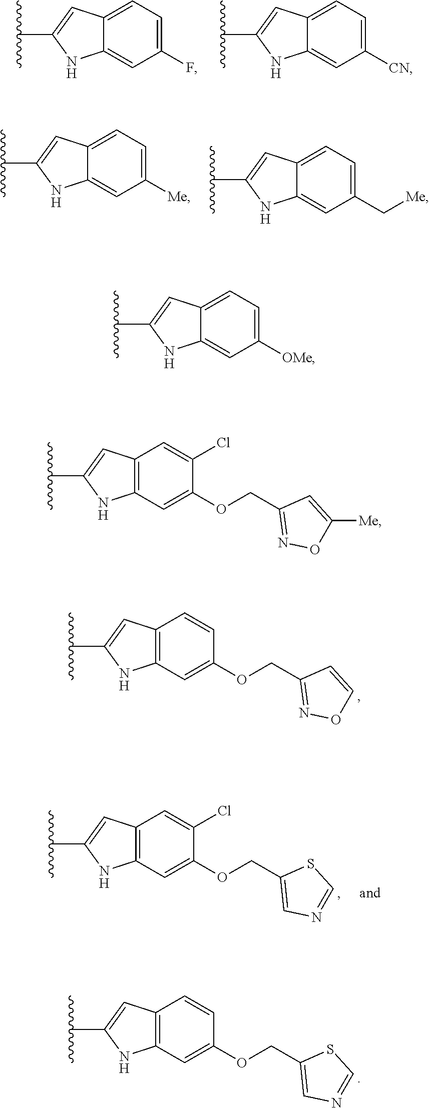

In some embodiments of a compound of formula (I), or a stereoisomer or tautomer thereof, or a pharmaceutically acceptable salt of any of the foregoing, the moiety represented by

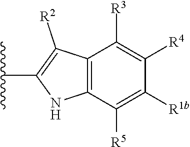

is a 2,5,6-substituted indole selected from the group consisting of

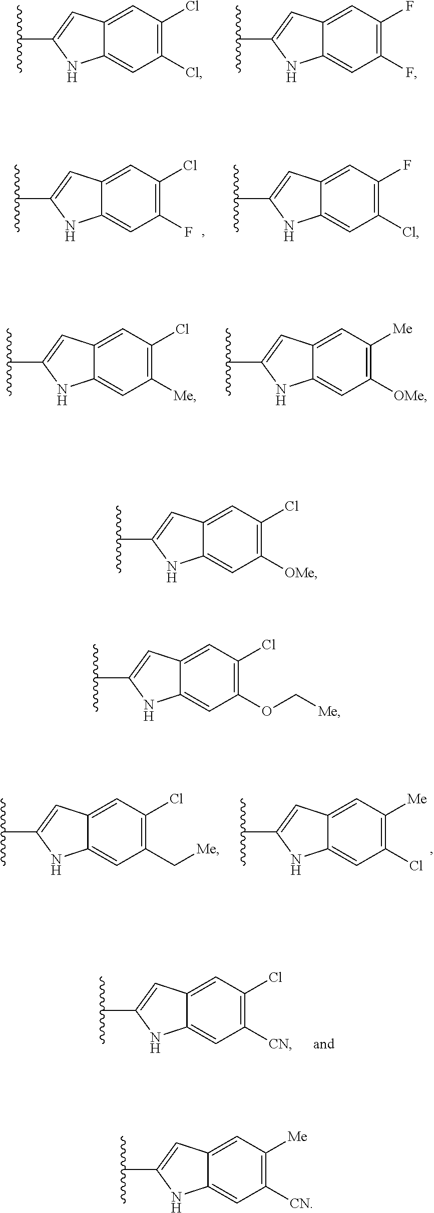

In some embodiments of a compound of formula (I), or a stereoisomer or tautomer thereof, or a pharmaceutically acceptable salt of any of the foregoing, the moiety represented by

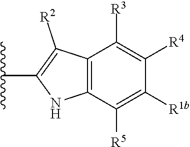

is a 2,3,5-substituted indole selected from the group consisting of

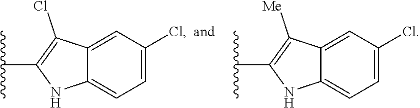

In some embodiments of a compound of formula (I), or a stereoisomer or tautomer thereof, or a pharmaceutically acceptable salt of any of the foregoing, the moiety represented by

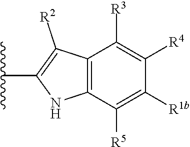

is a 2,5,7-substituted indole selected from the group consisting of

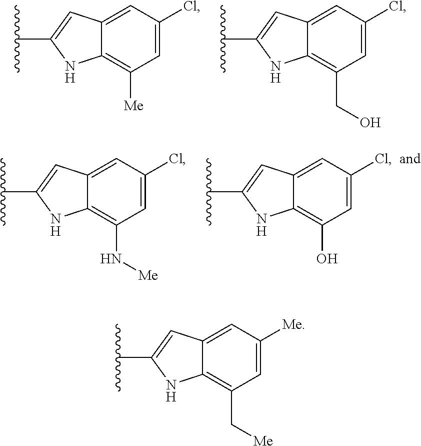

In some embodiments of a compound of formula (I), or a stereoisomer or tautomer thereof, or a pharmaceutically acceptable salt of any of the foregoing, the moiety represented by

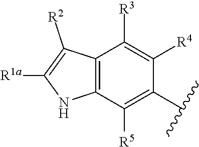

is a 5,6-substituted indole selected from the group consisting of

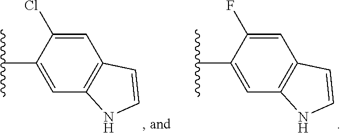 [SMILES: *c1cc2[nH]ccc2cc1Cl ; *c1cc2[nH]ccc2cc1F]

In some embodiments of a compound of formula (I), or a stereoisomer or tautomer thereof, or a pharmaceutically acceptable salt of any of the foregoing, the moiety represented by

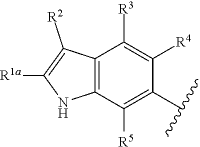

is a 3,6-substituted indole such as

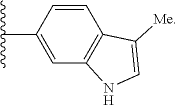 [SMILES: *c1ccc2c(C)c[nH]c2c1]

In some embodiments of a compound of formula (I), or a stereoisomer or tautomer thereof, or a pharmaceutically acceptable salt of any of the foregoing, the moiety represented by

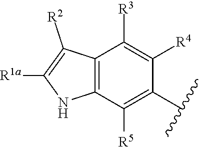

is a 3,5,6-substituted indole selected from the group consisting of

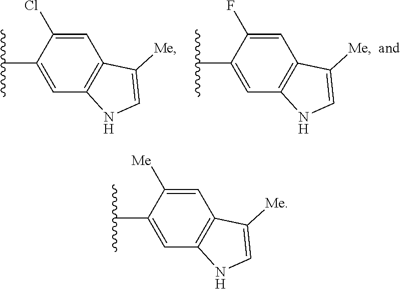

In some embodiments of a compound of formula (I), or a stereoisomer or tautomer thereof, or a pharmaceutically acceptable salt of any of the foregoing, the moiety represented by

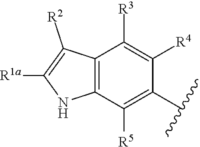

is a 2,3,6-substituted indole such as

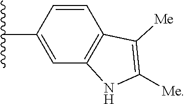 [SMILES: *c1ccc2c(C)c(C)[nH]c2c1]

In one aspect, provided herein is a compound of formula (I), such as a compound of formula (I-A):

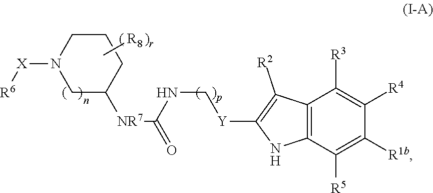

or a stereoisomer or tautomer thereof, or a pharmaceutically acceptable salt of any of the foregoing, wherein
R^{1b} is H, halo, —CN, C₁₋₆alkyl, C₁₋₆haloalkyl, C₁₋₆cycloalkyl, or C₁₋₆alkoxy, wherein the C₁₋₆alkoxy is optionally substituted with one or more R^{a}; and
n, r, p, R^{1b}, R², R³, R⁴, R⁵, R⁶, R⁷, R⁸, X, and Y are as defined for a compound of formula (I).

In some embodiments of a compound of formula (I), or (I-A), or a stereoisomer or tautomer thereof, or a pharmaceutically acceptable salt of any of the foregoing, R^{1b} is H, halo, —CN, C₁₋₆alkyl, C₁₋₆haloalkyl, C₁₋₆cycloalkyl, or C₁₋₆alkoxy, wherein the C₁₋₆alkoxy is optionally substituted with one or more R^{a}; R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; R³ and R⁵ are each independently H, —OH, halo, —N(R^{w1})(R^{w2}) or C₁₋₆alkyl, wherein the C₁₋₆alkyl of R³ and R⁵ is optionally substituted with one or more —OH; R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}; R⁷ is C₁₋₆alkyl, C₁₋₆cycloalkyl, or C₁₋₆haloalkyl; R⁸ is independently at each occurrence, C₁₋₆alkyl; R⁹ is independently at each occurrence, halo; each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c}; each R^{b} is independently —OH, oxo, halo, C₁₋₆alkyl, C₁₋₆alkoxy, —C(O)R^{y}, or —SO₂R^{z}, wherein the C₁₋₆alkyl is optionally substituted with one or more R^{c}; each R^{c} is independently —OH, halo, —N(R^{w1})(R^{w2}), C₁₋₆alkyl, —C₁₋₆alkylN(R^{w1})(R^{w2}), or C₁₋₆alkoxy; R^{w1} and R^{w2} are independently at each occurrence H, C₁₋₆alkyl, C₁₋₆haloalkyl, C₃₋₁₀-cycloalkyl, or —C(O)R^{y}; R^{x} is independently at each occurrence, 3-10 membered heterocyclyl optionally substituted with one or more oxo or C₁₋₆alkyl; R is independently at each occurrence, C₁₋₆alkyl, C₁₋₆haloalkyl; —NH₂, —NH(C₁₋₆alkyl), or —N(C₁₋₆alkyl)₂; R^{z} is independently at each occurrence, C₁₋₆alkyl; X is absent or —C(O)—; Y is absent or

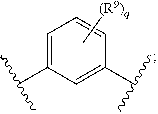

n is an integer from 1 to 2; p is an integer from 1 to 4; q is an integer from 0 to 4; and r is an integer from 0 to 11.

In some embodiments of a compound of formula (I), or (I-A), or a stereoisomer or tautomer thereof, or a pharmaceutically acceptable salt of any of the foregoing, R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R³ and R⁵ are each independently H, —OH, halo, —N(R^{w1})(R^{w2}), or C₁₋₃alkyl, wherein the C₁₋₃alkyl of R³ and R⁵ is optionally substituted with one or more —OH; R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-6 membered heterocyclyl of R⁶ are each independently optionally substituted with one or more R^{b}; R⁷ is C₁₋₃alkyl, C₁₋₃cycloalkyl, or C₁₋₃haloalkyl; R⁸ is independently at each occurrence, C₁₋₃alkyl; R⁹ is independently at each occurrence, halo; each R^{a} is independently —OH, halo, —CN, C₁₋₃alkyl, C₁₋₃alkoxy, —N(R^{w1})(R^{w2}), —OR^{x}, —C(O)R^{y}, —SO₂R^{z}, C₃₋₆cycloalkyl, C₆₋₁₀aryl, 5-10 membered heteroaryl, or 3-6 membered heterocyclyl, wherein the C₁₋₃alkyl, C₃₋₆cycloalkyl, and 3-6 membered heterocyclyl of R^{a} are each optionally substituted with one or more R^{b}, and the C₆₋₁₀aryl, and 5-10 membered heteroaryl of R^{a} are each optionally substituted with one or more R^{c}; each R^{b} is independently —OH, oxo, halo, C₁₋₃alkyl, C₁₋₃alkoxy, —C(O)R^{y}, or —SO₂R^{z}, wherein the C₁₋₃alkyl is optionally substituted with one or more R^{c}; each R^{c} is independently —OH, halo, —N(R^{w1})(R^{w2}), C₁₋₃alkyl, —C₁₋₃alkylN(R^{w1})(R^{w2}), or C₁₋₃alkoxy; R^{w1} and R^{w2} are independently at each occurrence H, C₁₋₃alkyl, C₁₋₃haloalkyl, C₃₋₆cycloalkyl, or —C(O)R^{y}; R^{x} is independently at each occurrence, 3-6 membered heterocyclyl optionally substituted with one or more oxo or C₁₋₃alkyl; R^{y} is independently at each occurrence, C₁₋₃alkyl, C₁₋₃haloalkyl; —NH₂, —NH(C₁₋₃alkyl), or —N(C₁₋₃alkyl)₂; R^{z} is independently at each occurrence, C₁₋₃alkyl; X is absent or —C(O)—; Y is absent or

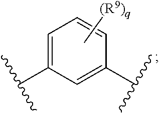

n is an integer from 1 to 2; p is an integer from 1 to 4; q is an integer from 0 to 4; and r is an integer from 0 to 11.

In some embodiments of a compound of formula (I), or (I-A), or a stereoisomer or tautomer thereof, or a pharmaceutically acceptable salt of any of the foregoing, R^{1b} is H, halo, —CN, C₁₋₆alkyl, C₁₋₆haloalkyl, C₁₋₆cycloalkyl, or C₁₋₆alkoxy, wherein R^{1b} is optionally substituted with one or more R^{a}. In some embodiments, R^{1b} is H, halo, —CN, C₁₋₃alkyl, C₁₋₃haloalkyl, C₁₋₃cycloalkyl, or C₁₋₃alkoxy, wherein R^{1b} is optionally substituted with one or more R^{a}. In some embodiments, R^{1b} is H, halo, —CN, C₁₋₃alkyl, C₁₋₃haloalkyl, C₁₋₃cycloalkyl, or C₁₋₃alkoxy. In some embodiments, R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}. In some embodiments, R^{1b} is H, halo, or C₁₋₃alkyl. In some embodiments, R^{1b} is H, F, Cl, methyl, ethyl, or isopropyl.

In some embodiments of a compound of formula (I), or (I-A), or a stereoisomer or tautomer thereof, or a pharmaceutically acceptable salt of any of the foregoing, R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R⁷ is C₁₋₆cycloalkyl; X is —C(O)—; n is 1; p is 1; and Y is absent.

In some embodiments of a compound of formula (I), or (I-A), or a stereoisomer or tautomer thereof, or a pharmaceutically acceptable salt of any of the foregoing, R⁴ is H or halo. In some embodiments, R⁴ is H. In some embodiments, R⁴ is F or Cl. In some embodiments, R⁴ is F. In some embodiments, R⁴ is Cl. In some embodiments, R⁴ is H or halo, R⁷ is C₁₋₆cycloalkyl, X is —C(O)—, n is 1, p is 1, and Y is absent.

In some embodiments of a compound of formula (I), or (I-A), or a stereoisomer or tautomer thereof, or a pharmaceutically acceptable salt of any of the foregoing, R⁷ is C₁₋₆alkyl, C₁₋₆cycloalkyl, or C₁₋₆haloalkyl. In some embodiments, R⁷ is C₁₋₃alkyl, C₁₋₃cycloalkyl, or C₁₋₃haloalkyl. In some embodiments, R⁷ is H, methyl, ethyl, or —CF₃.

In some embodiments of a compound of formula (I), or (I-A), or a stereoisomer or tautomer thereof, or a pharmaceutically acceptable salt of any of the foregoing, R⁷ is C₁₋₆alkyl. In some embodiments, R⁷ is C₁₋₆haloalkyl. In some embodiments, R⁷ is C₁₋₆cycloalkyl. In some embodiments, R⁷ is C₁₋₆alkyl, X is —C(O)—, n is 1, p is 1, and Y is absent. In some embodiments, R⁷ is C₁₋₆cycloalkyl, X is —C(O)—, n is 1, p is 1, and Y is absent. In some embodiments, R⁷ is C₁₋₆haloalkyl, X is —C(O)—, n is 1, p is 1, and Y is absent.

In some embodiments of a compound of formula (I), or (I-A), or a stereoisomer or tautomer thereof, or a pharmaceutically acceptable salt of any of the foregoing, X is absent or —C(O)—. In some embodiments, X is absent. In some embodiments, X is —C(O)—.

In some embodiments of a compound of formula (I), or (I-A), or a stereoisomer or tautomer thereof, or a pharmaceutically acceptable salt of any of the foregoing, Y is absent.

In some embodiments of a compound of formula (I), or (I-A), or a stereoisomer or tautomer thereof, or a pharmaceutically acceptable salt of any of the foregoing, embodiments Y is

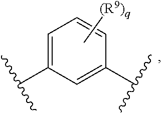

wherein q is an integer from 0-4, and R⁹ is independently at each occurrence, halo. In some embodiments, q is an integer from 0-2. In some embodiments, q is 0. In some embodiments, q is 1. In some embodiments, q is 2. In some embodiments, R⁹ is F or Cl. In some embodiments, R⁹ is F. In some embodiments, R⁹ is Cl.

In some embodiments of a compound of formula (I), or (I-A), or a stereoisomer or tautomer thereof, or a pharmaceutically acceptable salt of any of the foregoing, X is —C(O)—, n is 1, p is 1, and Y is absent. In some embodiments, X is —C(O)—, n is 1, p is 1, and Y is

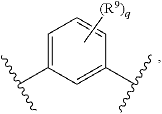

wherein q is 0. In some embodiments, X is —C(O)—, n is 1, p is 1, and Y is

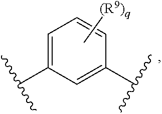

wherein q is 1. In some embodiments, X is —C(O)—, n is 1, p is 1, and Y is

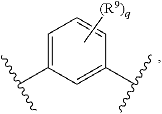

wherein q is 2.

In some embodiments of a compound of formula (I), or (I-A), or a stereoisomer or tautomer thereof, or a pharmaceutically acceptable salt of any of the foregoing, n is an integer from 1-2. In some embodiments, n is 1. In some embodiments, n is 2.

In some embodiments of a compound of formula (I), or (I-A), or a stereoisomer or tautomer thereof, or a pharmaceutically acceptable salt of any of the foregoing, p is an integer from 1-4. In some embodiments, p is 1. In some embodiments, p is 2. In some embodiments, p is 3. In some embodiments, p is 4.

In some embodiments of a compound of formula (I), or (I-A), or a stereoisomer or tautomer thereof, or a pharmaceutically acceptable salt of any of the foregoing, r is an integer from 0-13, and n is an integer from 1-2. In some embodiments, r is an integer from 0-6, and n is an integer from 1-2. In some embodiments, r is 0, and n is 1. In some embodiments, r is 0, and n is 2. In some embodiments, r is 1, and n is 1.

In some embodiments of a compound of formula (I), or (I-A), or a stereoisomer or tautomer thereof, or a pharmaceutically acceptable salt of any of the foregoing, q is an integer from 0-4. In some embodiments, q is 0. In some embodiments, q is 1. In some embodiments, q is 2. In some embodiments, q is 3. In some embodiments, q is 4.

In some embodiments of a compound of formula (I), or (I-A), or a stereoisomer or tautomer thereof, or a pharmaceutically acceptable salt of any of the foregoing, X is —C(O)—; Y is absent; n is 1; R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; R^{1b} is H, halo, —CN, C₁₋₆alkyl, C₁₋₆haloalkyl, C₁₋₆cycloalkyl, or C₁₋₆alkoxy, wherein
the C₁₋₆alkoxy of R^{1b} is optionally substituted with one or more R^{a}; R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH; and R⁶ is C₁₋₆alkyl, C₁₋₆haloalkyl, C₁₋₆alkoxy, C₃₋₁₀ cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), or a stereoisomer or tautomer thereof, or a pharmaceutically acceptable salt of any of the foregoing, X is —C(O)—; Y is absent; n is 1; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R³ and R⁵ are each independently H, —OH, halo, —N(R^{w1})(R^{w2}), or C₁₋₃alkyl, wherein C₁₋₃alkyl of R³ and R⁵ is optionally substituted with one or more —OH; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments, X is —C(O)—; Y is absent; n is 1; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R³ and R⁵ are each independently H, —OH, halo, —N(R^{w1})(R^{w2}), or C₁₋₃alkyl, wherein C₁₋₃alkyl of R³ and R⁵ is optionally substituted with one or more —OH; and R⁶ is selected from the group consisting of

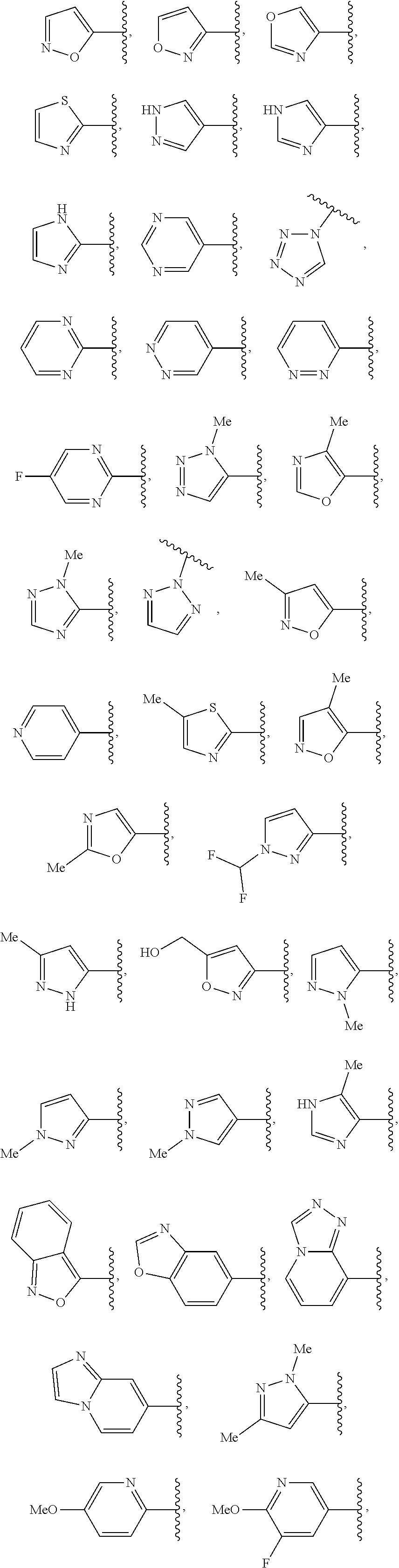

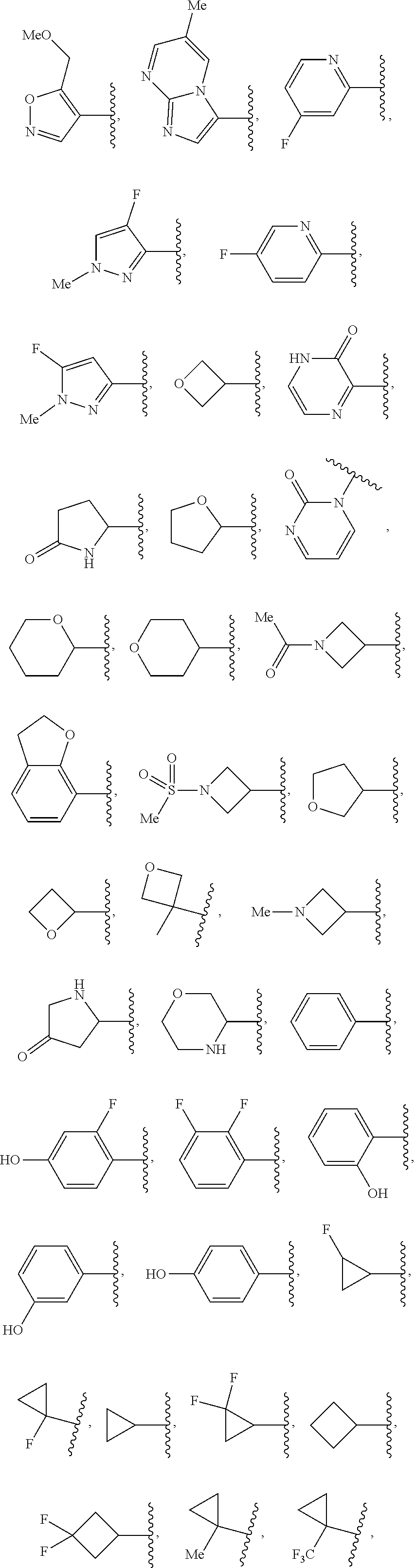

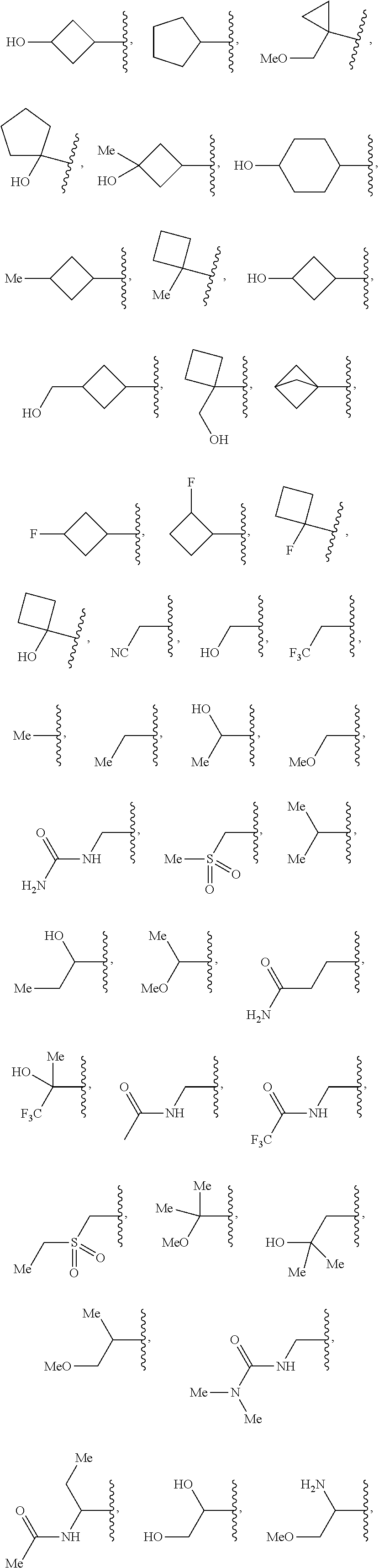

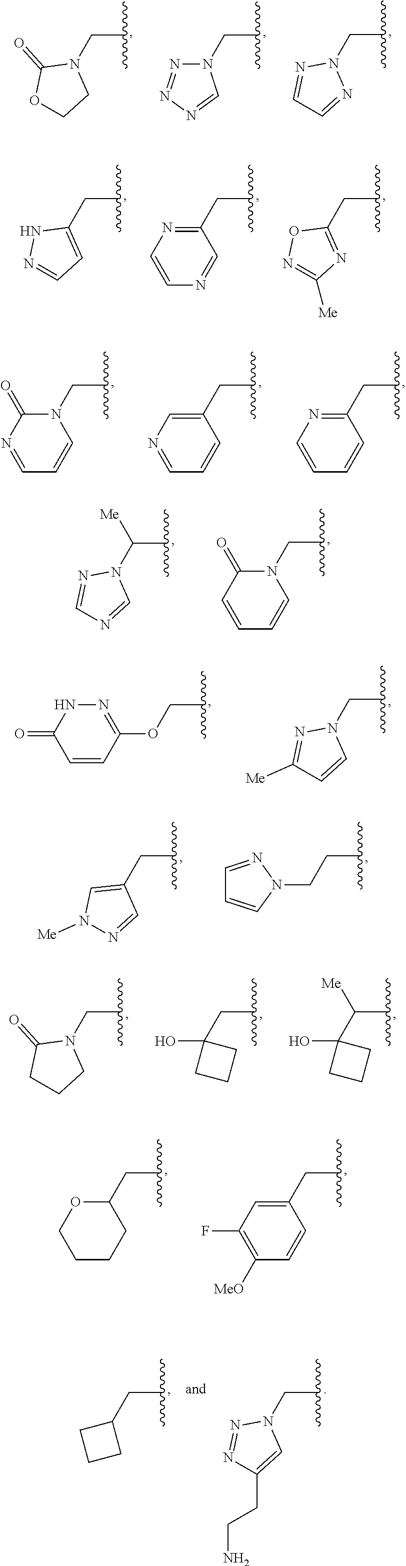

In one aspect, provided herein is a compound of formula (I), or (I-A), such as a compound of formula (I-A1):

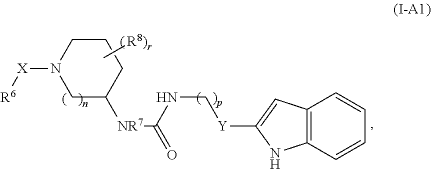

or a stereoisomer or tautomer thereof, or a pharmaceutically acceptable salt of any of the foregoing, wherein n, p, r, R⁶, R⁷, R⁸, X, and Y are as defined for a compound of formula (I).

In some embodiments of a compound of formula (I), or (I-A), such as a compound of formula (I-A1), or a stereoisomer or tautomer thereof, or a pharmaceutically acceptable salt of any of the foregoing, X is absent; Y is absent; n is 1; and R⁶ is 5-10 membered heteroaryl. In some embodiments, X is absent; Y is absent; n is 1; R⁶ is

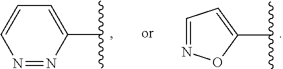 [SMILES: *c1cccnn1 ; *c1ccno1]

In some embodiments of a compound of formula (I), or (I-A), such as a compound of formula (I-A1), or a stereoisomer or tautomer thereof, or a pharmaceutically acceptable salt of any of the foregoing, X is —C(O)—; Y is absent; n is 1; and R⁶ is C₁₋₃alkyl. In some embodiments, X is —C(O)—; Y is absent; n is 1; and R⁶ is methyl.

In some embodiments of a compound of formula (I), or (I-A), such as a compound of formula (I-A1), or a stereoisomer or tautomer thereof, or a pharmaceutically acceptable salt of any of the foregoing, X is —C(O)—; Y is

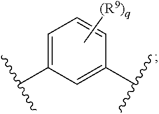

n is 2; and R⁶ is C₁₋₃alkyl. In some embodiments, X is —C(O)—; Y is

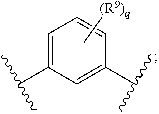

n is 2; and R⁶ is methyl.

In one aspect, provided herein is a compound of formula (I), or (I-A), such as a compound of formula (I-A2):

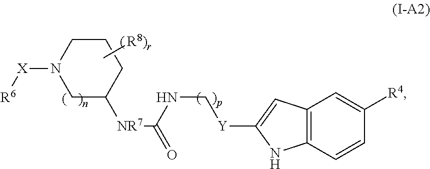

or a stereoisomer or tautomer thereof, or a pharmaceutically acceptable salt of any of the foregoing, wherein n, p, r, R⁴, R⁶, R⁷, R⁸, X, and Y are as defined for a compound of formula (I).

In some embodiments of a compound of formula (I), or (I-A), such as a compound of formula (I-A2), or a stereoisomer or tautomer thereof, or a pharmaceutically acceptable salt of any of the foregoing, X is —C(O)—; Y is absent; n is 1; R⁴ is halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₆alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), such as a compound of formula (I-A2), or a stereoisomer or tautomer thereof, or a pharmaceutically acceptable salt of any of the foregoing, wherein X is —C(O)—; Y is absent; n is 1; R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}) C₆₋₁₀aryl, 3-10 membered heterocyclyl, or 5-10 membered heteroaryl, wherein the C₁₋₃alkyl, C₆₋₁₀ aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A2), or a stereoisomer or tautomer thereof, or a pharmaceutically acceptable salt of any of the foregoing, wherein X is —C(O)—; Y is absent; n is 1; R⁴ is halo, —CN, C₁₋₃alkyl, C₁₋₃cycloalkyl, C₁₋₃haloalkyl, or C₁₋₃alkoxy; and R⁶ is C₁₋₃alkyl, C₁₋₃haloalkyl, C₁₋₃alkoxy, C₃₋₆-cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A2), or a stereoisomer or tautomer thereof, or a pharmaceutically acceptable salt of any of the foregoing, X is absent; Y is absent; n is 1; R⁴ is halo; and R⁶ is C₁₋₃alkyl. In some embodiments, X is absent; Y is absent; n is 1; R⁴ is halo; and R⁶ is methyl.

In some embodiments of a compound of formula (I), or (I-A), such as a compound of formula (I-A2), or a stereoisomer or tautomer thereof, or a pharmaceutically acceptable salt of any of the foregoing, R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}.

In one aspect, provided herein is a compound of formula (I), or (I-A), such as a compound of formula (I-A3):

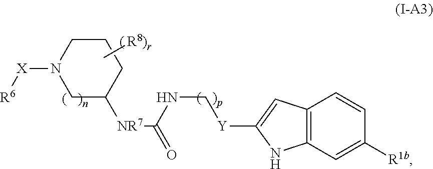

or a stereoisomer or tautomer thereof, or a pharmaceutically acceptable salt of any of the foregoing, wherein n, p, r, R^{1b}, R⁶, R⁷, R⁸, X, and Y are as defined for a compound of formula (I).

In some embodiments of a compound of formula (I), or (I-A), such as a compound of formula (I-A3), or a stereoisomer or tautomer thereof, or a pharmaceutically acceptable salt of any of the foregoing, X is —C(O)—; Y is absent; n is 1; R^{1b} is halo, —CN, C₁₋₆alkyl, C₁₋₆haloalkyl, C₁₋₆cycloalkyl, or C₁₋₆alkoxy, wherein the C₁₋₆alkoxy of R^{1b} is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₆alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), such as a compound of formula (I-A3), or a stereoisomer or tautomer thereof, or a pharmaceutically acceptable salt of any of the foregoing, wherein X is —C(O)—; Y is absent; n is 1; R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}) C₆₋₁₀aryl, 3-10 membered heterocyclyl, or 5-10 membered heteroaryl, wherein the C₁₋₃alkyl, C₆₋₁₀ aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A3), or a stereoisomer or tautomer thereof, or a pharmaceutically acceptable salt of any of the foregoing, wherein X is —C(O)—; Y is absent; n is 1; R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; and R⁶ is C₁₋₃alkyl.

In some embodiments of a compound of formula (I), or (I-A), such as a compound of formula (I-A3), or a stereoisomer or tautomer thereof, or a pharmaceutically acceptable salt of any of the foregoing, X is absent; Y is absent; n is 1; R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; and R⁶ is C₁₋₃alkyl.

In some embodiments of a compound of formula (I), or (I-A), such as a compound of formula (I-A3), or a stereoisomer or tautomer thereof, or a pharmaceutically acceptable salt of any of the foregoing, R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}.

In one aspect, provided herein is a compound of formula (I), or (I-A), such as a compound of formula (I-A4):

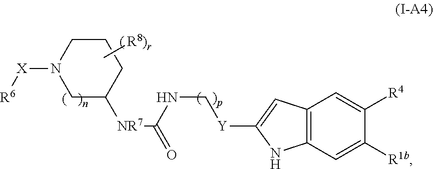

or a stereoisomer or tautomer thereof, or a pharmaceutically acceptable salt of any of the foregoing, wherein n, p, r, R^{1b}, R⁴, R⁶, R⁷, R⁸, X, and Y are as defined for a compound of formula (I).

In some embodiments of a compound of formula (I), or (I-A), such as a compound of formula (I-A4), or a stereoisomer or tautomer thereof, or a pharmaceutically acceptable salt of any of the foregoing, X is —C(O)—; Y is absent; n is 1; R^{1b} is halo, —CN, C₁₋₆alkyl, C₁₋₆haloalkyl, C₁₋₆cycloalkyl, or C₁₋₆alkoxy, wherein the C₁₋₆alkoxy of R^{1b} is optionally substituted with one or more R^{a}; R⁴ is halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₆alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), such as a compound of formula (I-A4), or a stereoisomer or tautomer thereof, or a pharmaceutically acceptable salt of any of the foregoing, wherein X is —C(O)—; Y is absent; n is 1; R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A4), or a stereoisomer or tautomer thereof, or a pharmaceutically acceptable salt of any of the foregoing, wherein X is —C(O)—; Y is absent; n is 1; R^{1b} is halo, —CN, C₁₋₃alkyl, or C₁₋₃alkoxy, wherein the C₁₋₃alkoxy of R^{1b} is optionally substituted with one or more R^{a}; R⁴ is halo, or C₁₋₃alkyl; and R⁶ is C₁₋₃alkyl.

In some embodiments of a compound of formula (I), or (I-A), such as a compound of formula (I-A4), or a stereoisomer or tautomer thereof, or a pharmaceutically acceptable salt of any of the foregoing, X is absent; Y is absent; n is 1; R^{1b} is C₁₋₃alkoxy, wherein the C₁₋₃alkoxy of R^{1b} is optionally substituted with one or more R^{a}; R⁴ is halo; and R⁶ is 5-10 membered heteroaryl.

In some embodiments of a compound of formula (I), or (I-A), such as a compound of formula (I-A4), or a stereoisomer or tautomer thereof, or a pharmaceutically acceptable salt of any of the foregoing, R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; and R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}.

In one aspect, provided herein is a compound of formula (I), or (I-A), such as a compound of formula (I-A5):

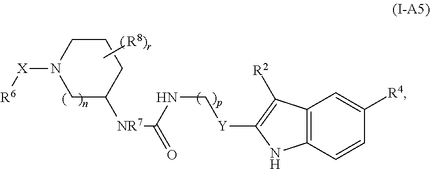

or a stereoisomer or tautomer thereof, or a pharmaceutically acceptable salt of any of the foregoing, wherein n, p, r, R², R⁴, R⁶, R⁷, R⁸, X, and Y are as defined for a compound of formula (I).

In some embodiments of a compound of formula (I), or (I-A), such as a compound of formula (I-A5), or a stereoisomer or tautomer thereof, or a pharmaceutically acceptable salt of any of the foregoing, X is —C(O)—; Y is absent; n is 1; R² and R⁴ are each independently halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; and R⁶ is C₁₋₆alkyl, C₁₋₆haloalkyl, C₁₋₆alkoxy, C₃₋₁₀ cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), such as a compound of formula (I-A5), or a stereoisomer or tautomer thereof, or a pharmaceutically acceptable salt of any of the foregoing, wherein X is —C(O)—; Y is absent; n is 1; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A5), or a stereoisomer or tautomer thereof, or a pharmaceutically acceptable salt of any of the foregoing, wherein X is —C(O)—; Y is absent; n is 1; R² is halo, or C₁₋₃alkyl; R⁴ is halo; and R⁶ is C₁₋₃alkyl.

In some embodiments of a compound of formula (I), or (I-A), such as a compound of formula (I-A5), or a stereoisomer or tautomer thereof, or a pharmaceutically acceptable salt of any of the foregoing, R² and R⁴ are each independently halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R.

In one aspect, provided herein is a compound of formula (I), or (I-A), such as a compound of formula (I-A6):

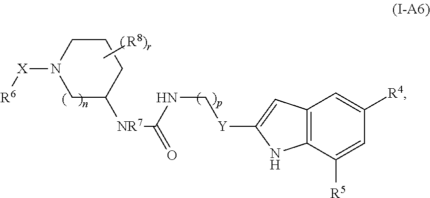

or a stereoisomer or tautomer thereof, or a pharmaceutically acceptable salt of any of the foregoing, wherein n, p, r, R⁴, R⁵, R⁶, R⁷, R⁸, X, and Y are as defined for a compound of formula (I).

In some embodiments of a compound of formula (I), or (I-A), such as a compound of formula (I-A6), or a stereoisomer or tautomer thereof, or a pharmaceutically acceptable salt of any of the foregoing, X is —C(O)—; Y is absent; n is 1; R⁴ is halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}; R⁵ is —OH, halo, —N(R^{w1})(R^{w2}), or C₁₋₆alkyl, wherein the C₁₋₆alkyl of R⁵ is optionally substituted with one or more —OH; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₆alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-A), such as a compound of formula (I-A6), or a stereoisomer or tautomer thereof, or a pharmaceutically acceptable salt of any of the foregoing, wherein X is —C(O)—; Y is absent; n is 1; R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}; R⁵ is —OH, halo, —N(R^{w1})(R^{w2}), or C₁₋₃alkyl, wherein the C₁₋₃alkyl of R⁵ is optionally substituted with one or more —OH; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-A), such as a compound of formula (I-A6), or a stereoisomer or tautomer thereof, or a pharmaceutically acceptable salt of any of the foregoing, wherein X is —C(O)—; Y is absent; n is 1; R⁴ is halo, or C₁₋₃alkyl; R⁵ is —OH, —N(R^{w1})(R^{w2}), or C₁₋₃alkyl, wherein the C₁₋₃alkyl of R⁵ is optionally substituted with one or more —OH; and R⁶ is C₁₋₃alkyl.

In some embodiments of a compound of formula (I), or (I-A), such as a compound of formula (I-A6), or a stereoisomer or tautomer thereof, or a pharmaceutically acceptable salt of any of the foregoing, R⁴ is halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}; and R⁵ is —OH, halo, —N(R^{w1})(R^{w2}), or C₁₋₆alkyl, wherein the C₁₋₆alkyl of R⁵ is optionally substituted with one or more —OH.

In one aspect, provided herein is a compound of formula (I), such as a compound of formula (I-B):

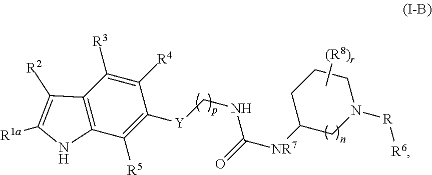

or a stereoisomer or tautomer thereof, or a pharmaceutically acceptable salt of any of the foregoing, wherein n, p, r, R^{1a}, R², R³, R⁴, R⁵, R⁶, R⁷, R⁸, X, and Y are as defined for a compound of formula (I).

In some embodiments of a compound of formula (I), or (I-B), or a stereoisomer or tautomer thereof, or a pharmaceutically acceptable salt of any of the foregoing, R^{1a} is H, halo, —CN, C₁₋₆alkyl, C₁₋₆haloalkyl, C₁₋₆cycloalkyl, or C₁₋₆alkoxy, wherein the C₁₋₆alkoxy is optionally substituted with one or more R^{a}; R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; R³ and R⁵ are each independently H, —OH, halo, —N(R^{w1})(R^{w2}) or C₁₋₆alkyl, wherein the C₁₋₆alkyl of R³ and R⁵ is optionally substituted with one or more —OH; R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}; R⁷ is C₁₋₆alkyl, C₁₋₆cycloalkyl, or C₁₋₆haloalkyl; R⁸ is independently at each occurrence, C₁₋₆alkyl; R⁹ is independently at each occurrence, halo; each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c}; each R^{b} is independently —OH, oxo, halo, C₁₋₆alkyl, C₁₋₆alkoxy, —C(O)R^{y}, or —SO₂R^{z}, wherein the C₁₋₆alkyl is optionally substituted with one or more R^{c}; each R^{c} is independently —OH, halo, —N(R^{w1})(R^{w2}), C₁₋₆alkyl, —C₁₋₆alkylN(R^{w1})(R^{w2}), or C₁₋₆alkoxy; R^{w1} and R^{w2} are independently at each occurrence H, C₁₋₆alkyl, C₁₋₆haloalkyl, C₃₋₁₀-cycloalkyl, or —C(O)R^{y}; R^{x} is independently at each occurrence, 3-10 membered heterocyclyl optionally substituted with one or more oxo or C₁₋₆alkyl; R is independently at each occurrence, C₁₋₆alkyl, C₁₋₆haloalkyl; —NH₂, —NH(C₁₋₆alkyl), or —N(C₁₋₆alkyl)₂; R^{z} is independently at each occurrence, C₁₋₆alkyl; X is absent or —C(O)—; Y is absent or

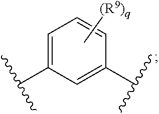

n is an integer from 1 to 2; p is an integer from 1 to 4; q is an integer from 0 to 4; and r is an integer from 0 to 11.

In some embodiments of a compound of formula (I), or (I-B), or a stereoisomer or tautomer thereof, or a pharmaceutically acceptable salt of any of the foregoing, R^{1a} is H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R³ and R⁵ are each independently H, —OH, halo, —N(R^{w1})(R^{w2}), or C₁₋₃alkyl, wherein the C₁₋₃alkyl of R³ and R⁵ is optionally substituted with one or more —OH; R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-6 membered heterocyclyl of R⁶ are each independently optionally substituted with one or more R^{b}; R⁷ is C₁₋₃alkyl, C₁₋₃cycloalkyl, or C₁₋₃haloalkyl; R⁸ is independently at each occurrence, C₁₋₃alkyl; R⁹ is independently at each occurrence, halo; each R^{a} is independently —OH, halo, —CN, C₁₋₃alkyl, C₁₋₃alkoxy, —N(R^{w1})(R^{w2}), —OR^{x}, —C(O)R^{y}, —SO₂R^{z}, C₃₋₆cycloalkyl, C₆₋₁₀aryl, 5-10 membered heteroaryl, or 3-6 membered heterocyclyl, wherein the C₁₋₃alkyl, C₃₋₆cycloalkyl, and 3-6 membered heterocyclyl of R^{a} are each optionally substituted with one or more R^{b}, and the C₆₋₁₀aryl, and 5-10 membered heteroaryl of R^{a} are each optionally substituted with one or more R^{c}; each R^{b} is independently —OH, oxo, halo, C₁₋₃alkyl, C₁₋₃alkoxy, —C(O)R^{y}, or —SO₂R^{z}, wherein the C₁₋₃alkyl is optionally substituted with one or more R^{c}; each R^{c} is independently —OH, halo, —N(R^{w1})(R^{w2}), C₁₋₃alkyl, —C₁₋₃alkylN(R^{w1})(R^{w2}), or C₁₋₃alkoxy; R^{w1} and R^{w2} are independently at each occurrence H, C₁₋₃alkyl, C₁₋₃haloalkyl, C₃₋₆cycloalkyl, or —C(O)R^{y}; R^{x} is independently at each occurrence, 3-6 membered heterocyclyl optionally substituted with one or more oxo or C₁₋₃alkyl; R^{y} is independently at each occurrence, C₁₋₃alkyl, C₁₋₃haloalkyl; —NH₂, —NH(C₁₋₃alkyl), or —N(C₁₋₃alkyl)₂; R^{z} is independently at each occurrence, C₁₋₃alkyl; X is absent or —C(O)—; Y is absent or

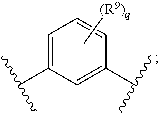

n is an integer from 1 to 2; p is an integer from 1 to 4; q is an integer from 0 to 4; and r is an integer from 0 to 11.

In some embodiments of a compound of formula (I), or (I-B), or a stereoisomer or tautomer thereof, or a pharmaceutically acceptable salt of any of the foregoing, X is —C(O)—.

In some embodiments of a compound of formula (I), or (I-B), or a stereoisomer or tautomer thereof, or a pharmaceutically acceptable salt of any of the foregoing, n is 1 or 2. In some embodiments, n is 1. In some embodiments, p is 1 or 2. In some embodiments, p is 1.

In some embodiments of a compound of formula (I), or (I-B), or a stereoisomer or tautomer thereof, or a pharmaceutically acceptable salt of any of the foregoing, X is —C(O)—, n is 1, p is 1 and Y is absent.

In some embodiments of a compound of formula (I), or (I-B), or a stereoisomer or tautomer thereof, or a pharmaceutically acceptable salt of any of the foregoing, R⁷ is C₁₋₆alkyl. In some embodiments, R⁷ is C₁₋₆alkyl, X is —C(O)—, n is 1, p is 1 and Y is absent.

In some embodiments of a compound of formula (I), or (I-B), or a stereoisomer or tautomer thereof, or a pharmaceutically acceptable salt of any of the foregoing, R⁴ is H or halo.

In some embodiments, R⁴ is H. In some embodiments, R⁴ is F or Cl. In some embodiments, R⁴ is F. In some embodiments, R⁴ is Cl. In some embodiments, R⁴ is H or halo, R⁷ is C₁₋₆alkyl, X is —C(O)—, n is 1, p is 1 and Y is absent.

In some embodiments of a compound of formula (I), or (I-B), or a stereoisomer or tautomer thereof, or a pharmaceutically acceptable salt of any of the foregoing, R² and R⁴ are each independently H, halo, —CN, C₁₋₃alkyl, C₁₋₃cycloalkyl, C₁₋₃haloalkyl, or C₁₋₃alkoxy, wherein the C₁₋₆alkoxy of R² and R⁴ is optionally substituted with one or more R^{a}; R⁷ is C₁₋₆alkyl; X is —C(O)—; n is 1; p is 1; and Y is absent.

In one aspect, provided herein is a compound of formula (I), or (I-B), such as a compound of formula (I-B1):

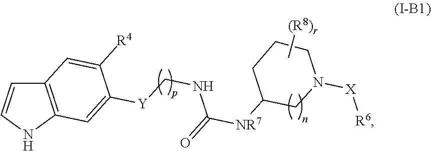

or a stereoisomer or tautomer thereof, or a pharmaceutically acceptable salt of any of the foregoing, wherein n, r, p, R⁴, R⁶, R⁷, R⁸, X and Y are as defined for a compound of formula (I).

In some embodiments of a compound of formula (I), or (I-B), such as a compound of formula (I-B1), or a stereoisomer or tautomer thereof, or a pharmaceutically acceptable salt of any of the foregoing, X is —C(O)—; Y is absent; n is 1; R⁴ is halo, —CN, C₁₋₆alkyl, C₁₋₆cycloalkyl, C₁₋₆haloalkyl, or C₁₋₆alkoxy, wherein the C₁₋₆alkoxy of R⁴ is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₃alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-B), such as a compound of formula (I-B1), or a stereoisomer or tautomer thereof, or a pharmaceutically acceptable salt of any of the foregoing, wherein X is —C(O)—; Y is absent; n is 1; R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}) C₆₋₁₀aryl, 3-10 membered heterocyclyl, or 5-10 membered heteroaryl, wherein the C₁₋₃alkyl, C₆₋₁₀ aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-B), such as a compound of formula (I-B1), or a stereoisomer or tautomer thereof, or a pharmaceutically acceptable salt of any of the foregoing, wherein X is —C(O)—; Y is absent; n is 1; R⁴ is halo; and R⁶ is C₁₋₃alkyl.

In some embodiments of a compound of formula (I), or (I-B), such as a compound of formula (I-B1), or a stereoisomer or tautomer thereof, or a pharmaceutically acceptable salt of any of the foregoing, R⁴ is halo, —CN, C₁₋₃alkyl, C₁₋₃cycloalkyl, C₁₋₃haloalkyl, or C₁₋₃alkoxy, wherein the C₁₋₃alkoxy of R⁴ is optionally substituted with one or more R^{a}.

In one aspect, provided herein is a compound of formula (I), or (I-B), such as a compound of formula (I-B2):

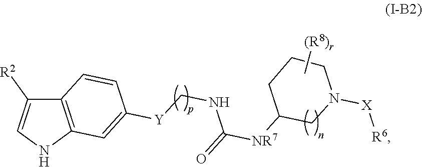

or a stereoisomer or tautomer thereof, or a pharmaceutically acceptable salt of any of the foregoing, wherein n, r, p, R², R⁶, R⁷, R⁸, X and Y are as defined for a compound of formula (I).

In some embodiments of a compound of formula (I), or (I-B), such as a compound of formula (I-B2), or a stereoisomer or tautomer thereof, or a pharmaceutically acceptable salt of any of the foregoing, X is —C(O)—; Y is absent; n is 1; R² is halo, —CN, C₁₋₆alkyl, C₁₋₆cycloalkyl, C₁₋₆haloalkyl, or C₁₋₆alkoxy, wherein the C₁₋₆alkoxy of R² is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-B), such as a compound of formula (I-B2), or a stereoisomer or tautomer thereof, or a pharmaceutically acceptable salt of any of the foregoing, wherein X is —C(O)—; Y is absent; n is 1; R² is halo, —CN, C₁₋₃alkyl, C₁-3cycloalkyl, C₁₋₃haloalkyl, or C₁₋₃alkoxy, wherein the C₁₋₃alkoxy of R² is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}) C₆₋₁₀aryl, 3-10 membered heterocyclyl, or 5-10 membered heteroaryl, wherein the C₁₋₃alkyl, C₆₋₁₀ aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-B), such as a compound of formula (I-B2), or a stereoisomer or tautomer thereof, or a pharmaceutically acceptable salt of any of the foregoing, wherein X is —C(O)—; Y is absent; n is 1; R² is C₁₋₃alkyl; and R⁶ is C₁₋₃alkyl.

In some embodiments of a compound of formula (I), or (I-B), such as a compound of formula (I-B2), or a stereoisomer or tautomer thereof, or a pharmaceutically acceptable salt of any of the foregoing, R² is halo, —CN, C₁₋₆alkyl, C₁₋₆cycloalkyl, C₁₋₆haloalkyl, or C₁₋₆alkoxy, wherein the C₁₋₆alkoxy of R² is optionally substituted with one or more R^{a}.

In one aspect, provided herein is a compound of formula (I), or (I-B), such as a compound of formula (I-B3):

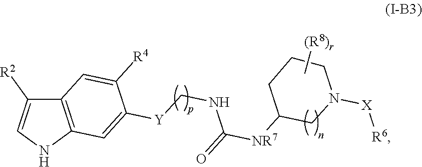

or a stereoisomer or tautomer thereof, or a pharmaceutically acceptable salt of any of the foregoing, wherein n, r, p, R², R⁴, R⁶, R⁷, R⁸, X and Y are as defined for a compound of formula (I).

In some embodiments of a compound of formula (I), or (I-B), such as a compound of formula (I-B3), or a stereoisomer or tautomer thereof, or a pharmaceutically acceptable salt of any of the foregoing, X is —C(O)—; Y is absent; n is 1; R² and R⁴ are each independently halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; and R⁶ is C₁₋₆alkyl, C₁₋₆haloalkyl, C₁₋₆alkoxy, C₃₋₁₀ cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-B), such as a compound of formula (I-B3), or a stereoisomer or tautomer thereof, or a pharmaceutically acceptable salt of any of the foregoing, wherein X is —C(O)—; Y is absent; n is 1; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-B), such as a compound of formula (I-B3), or a stereoisomer or tautomer thereof, or a pharmaceutically acceptable salt of any of the foregoing, wherein X is —C(O)—; Y is absent; n is 1; R² is C₁₋₃alkyl; R⁴ is halo, or C₁₋₃alkyl; and R⁶ is C₁₋₃alkyl.

In some embodiments of a compound of formula (I), or (I-B), such as a compound of formula (I-B3), or a stereoisomer or tautomer thereof, or a pharmaceutically acceptable salt of any of the foregoing, R² and R⁴ are each independently halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R.

In one aspect, provided herein is a compound of formula (I), or (I-B), such as a compound of formula (I-B4):

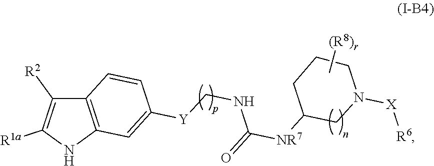

or a stereoisomer or tautomer thereof, or a pharmaceutically acceptable salt of any of the foregoing, wherein n, r, p, R^{1a}, R², R⁶, R⁷, R⁸, X and Y are as defined for a compound of formula (I).

In some embodiments of a compound of formula (I), or (I-B), such as a compound of formula (I-B4), or a stereoisomer or tautomer thereof, or a pharmaceutically acceptable salt of any of the foregoing, X is —C(O)—; Y is absent; n is 1; R^{1a} is halo, —CN, C₁₋₆alkyl, C₁₋₆haloalkyl, C₁₋₆cycloalkyl, or C₁₋₆alkoxy, wherein the C₁₋₆alkoxy of R^{1a} is optionally substituted with one or more R^{a}; R² is halo, —CN, C₁₋₆alkyl, C₁₋₆cycloalkyl, C₁₋₆haloalkyl, or C₁₋₆alkoxy, wherein the C₁₋₆alkoxy of R² is optionally substituted with one or more R^{a}; and R⁶ is C₁₋₆alkyl, C₁₋₆haloalkyl, C₁₋₆alkoxy, C₃₋₁₀cycloalkyl, —N(R^{w1})(R^{w2}), C₆₋₁₀aryl, 3-10 membered heterocyclyl, or 5-20 membered heteroaryl, wherein the C₁₋₆alkyl, C₆₋₁₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}.

In some embodiments of a compound of formula (I), or (I-B), such as a compound of formula (I-B4), or a stereoisomer or tautomer thereof, or a pharmaceutically acceptable salt of any of the foregoing, wherein X is —C(O)—; Y is absent; n is 1; R^{1a} is halo, —CN, C₁₋₃alkyl, C₁₋₃haloalkyl, C₁₋₃cycloalkyl, or C₁₋₃alkoxy, wherein the C₁₋₃alkoxy of R^{1a} is optionally substituted with one or more R^{a}; R² is halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-B), such as a compound of formula (I-B4), or a stereoisomer or tautomer thereof, or a pharmaceutically acceptable salt of any of the foregoing, wherein X is —C(O)—; Y is absent; n is 1; R^{1a} is C₁₋₃alkyl; R² is C₁₋₃alkyl; and R⁶ is C₁₋₃alkyl.

In some embodiments of a compound of formula (I), or (I-B), such as a compound of formula (I-B4), or a stereoisomer or tautomer thereof, or a pharmaceutically acceptable salt of any of the foregoing, X is absent; Y is absent; n is 1; R^{1a} is C₁₋₃alkoxy, wherein the C₁₋₃alkoxy of R^{1a} is optionally substituted with one or more R^{a}; R⁴ is halo; and R⁶ is 5-10 membered heteroaryl.

In some embodiments of a compound of formula (I), or (I-B), such as a compound of formula (I-B4), or a stereoisomer or tautomer thereof, or a pharmaceutically acceptable salt of any of the foregoing, R^{1b} is halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; and R² is halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}.

In one aspect, provided herein is a compound of formula (I), such as a compound of formula (I-C):

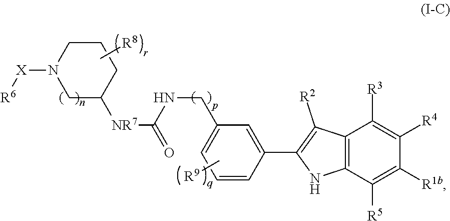

or a stereoisomer or tautomer thereof, or a pharmaceutically acceptable salt of any of the foregoing, wherein n, p, q, r, R^{1b}, R², R³, R⁴, R⁵, R⁶, R⁷, R⁸, R⁹ and X are as defined for a compound of formula (I).

In some embodiments of a compound of formula (I), or (I-C), or a stereoisomer or tautomer thereof, or a pharmaceutically acceptable salt of any of the foregoing, R^{1b} is H, halo, —CN, C₁₋₆alkyl, C₁₋₆haloalkyl, C₁₋₆cycloalkyl, or C₁₋₆alkoxy, wherein the C₁₋₆alkoxy is optionally substituted with one or more R^{a}; R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; R³ and R⁵ are each independently H, —OH, halo, —N(R^{w1})(R^{w2}) or C₁₋₆alkyl, wherein the C₁₋₆alkyl of R³ and R⁵ is optionally substituted with one or more —OH; R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}; R⁷ is C₁₋₆alkyl, C₁₋₆cycloalkyl, or C₁₋₆haloalkyl; R⁸ is independently at each occurrence, C₁₋₆alkyl; R⁹ is independently at each occurrence, halo; each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c}; each R^{b} is independently —OH, oxo, halo, C₁₋₆alkyl, C₁₋₆alkoxy, —C(O)R^{y}, or —SO₂R^{z}, wherein the C₁₋₆alkyl is optionally substituted with one or more R^{c}; each R^{c} is independently —OH, halo, —N(R^{w1})(R^{w2}), C₁₋₆alkyl, —C₁₋₆alkylN(R^{w1})(R^{w2}), or C₁₋₆alkoxy; R^{w1} and R^{w2} are independently at each occurrence H, C₁₋₆alkyl, C₁₋₆haloalkyl, C₃₋₁₀-cycloalkyl, or —C(O)R^{y}; R^{x} is independently at each occurrence, 3-10 membered heterocyclyl optionally substituted with one or more oxo or C₁₋₆alkyl; R is independently at each occurrence, C₁₋₆alkyl, C₁₋₆haloalkyl; —NH₂, —NH(C₁₋₆alkyl), or —N(C₁₋₆alkyl)₂; R^{z} is independently at each occurrence, C₁₋₆alkyl; X is absent or —C(O)—; n is an integer from 1 to 2; p is an integer from 1 to 4; q is an integer from 0 to 4; and r is an integer from 0 to 11.

In some embodiments of a compound of formula (I), or (I-C), or a stereoisomer or tautomer thereof, or a pharmaceutically acceptable salt of any of the foregoing, R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R³ and R⁵ are each independently H, —OH, halo, —N(R^{w1})(R^{w2}), or C₁₋₃alkyl, wherein the C₁₋₃alkyl of R³ and R⁵ is optionally substituted with one or more —OH; R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-6 membered heterocyclyl of R⁶ are each independently optionally substituted with one or more R^{b}; R⁷ is C₁₋₃alkyl, C₁₋₃cycloalkyl, or C₁₋₃haloalkyl; R⁸ is independently at each occurrence, C₁₋₃alkyl; R⁹ is independently at each occurrence, halo; each R^{a} is independently —OH, halo, —CN, C₁₋₃alkyl, C₁₋₃alkoxy, —N(R^{w1})(R^{w2}), —OR^{x}, —C(O)R^{y}, —SO₂R^{z}, C₃₋₆cycloalkyl, C₆₋₁₀aryl, 5-10 membered heteroaryl, or 3-6 membered heterocyclyl, wherein the C₁₋₃alkyl, C₃₋₆cycloalkyl, and 3-6 membered heterocyclyl of R^{a} are each optionally substituted with one or more R^{b}, and the C₆₋₁₀aryl, and 5-10 membered heteroaryl of R^{a} are each optionally substituted with one or more R^{c}; each R^{b} is independently —OH, oxo, halo, C₁₋₃alkyl, C₁₋₃alkoxy, —C(O)R^{y}, or —SO₂R^{z}, wherein the C₁₋₃alkyl is optionally substituted with one or more R^{c}; each R^{c} is independently —OH, halo, —N(R^{w1})(R^{w2}), C₁₋₃alkyl, —C₁₋₃alkylN(R^{w1})(R^{w2}), or C₁₋₃alkoxy; R^{w1} and R^{w2} are independently at each occurrence H, C₁₋₃alkyl, C₁₋₃haloalkyl, C₃₋₆cycloalkyl, or —C(O)R^{y}; R^{x} is independently at each occurrence, 3-6 membered heterocyclyl optionally substituted with one or more oxo or C₁₋₃alkyl; R^{y} is independently at each occurrence, C₁₋₃alkyl, C₁₋₃haloalkyl, —NH₂, —NH(C₁₋₃alkyl), or —N(C₁₋₃alkyl)₂; R^{z} is independently at each occurrence, C₁₋₃alkyl; X is absent or —C(O)—; n is an integer from 1 to 2; p is an integer from 1 to 4; q is an integer from 0 to 4; and r is an integer from 0 to 11.

In some embodiments of a compound of formula (I), or (I-C), or a stereoisomer or tautomer thereof, or a pharmaceutically acceptable salt of any of the foregoing, R^{1b} is H; R² and R⁴ are each independently H; R³ and R⁵ are each independently H; R⁶ is C₁₋₃alkyl; R⁷ is C₁₋₃alkyl; R⁹ is independently at each occurrence, halo; X is —C(O)—; n is 1; p is 2; q is an integer from 1 to 2; and r is 0.

In some embodiments of a compound of formula (I), or (I-C), or a stereoisomer or tautomer thereof, or a pharmaceutically acceptable salt of any of the foregoing, X is —C(O)—. In some embodiments, X is absent. In some embodiments, q is 1. In some embodiments, q is 2. In some embodiments, q is 3. In some embodiments, q is 4. In some embodiments, R⁹ is F or Cl. In some embodiments, R⁹ is F. In some embodiments, R⁹ is Cl.

In some embodiments of a compound of formula (I), or (I-C), or a stereoisomer or tautomer thereof, or a pharmaceutically acceptable salt of any of the foregoing, n is 1. In some embodiments, n is 2. In some embodiments, p is 1 or 2. In some embodiments, p is 1. In some embodiments, p is 2.

In some embodiments of a compound of formula (I), or (I-C), or a stereoisomer or tautomer thereof, or a pharmaceutically acceptable salt of any of the foregoing, X is —C(O)—; n is 1; p is 1; q is 0; and R⁹ is Cl or F. In some embodiments, X is —C(O)—; n is 1; p is 1; q is 1; and R⁹ is Cl or F. In some embodiments, X is —C(O)—; n is 1; p is 1; q is 2; and R⁹ is Cl or F. In some embodiments, X is —C(O)—; n is 1; p is 1; q is 3; and R⁹ is Cl or F. In some embodiments, X is —C(O)—; n is 1; p is 1; q is 4; and R⁹ is Cl or F.

In some embodiments of a compound of formula (I), or (I-C), or a stereoisomer or tautomer thereof, or a pharmaceutically acceptable salt of any of the foregoing, R⁷ is C₁₋₆alkyl. In some embodiments, R⁷ is C₁₋₆haloalkyl. In some embodiments, R⁷ is C₁₋₆cycloalkyl. In some embodiments, R⁷ is C₁₋₆alkyl; X is —C(O)—; n is 1; p is 1; and q is an integer from 0-2. In some embodiments, R⁷ is C₁₋₆cycloalkyl; X is —C(O)—; n is 1; p is 1; and q is an integer from 0-2. In some embodiments, R⁷ is C₁₋₆haloalkyl; X is —C(O)—; n is 1; p is 1; and q is an integer from 0-2.

In some embodiments of a compound of formula(I), or (I-C), or a stereoisomer or tautomer thereof, or a pharmaceutically acceptable salt of any of the foregoing, R⁴ is H or halo.

In some embodiments, R⁴ is H. In some embodiments, R⁴ is F or Cl. In some embodiments, R⁴ is F. In some embodiments, R⁴ is Cl. In some embodiments, R⁴ is H or Halo; R⁷ is C₁₋₆cycloalkyl; X is —C(O)—; n is 1; p is 1; and q is an integer from 0-2.

In some embodiments of a compound of formula (I), or (I-C), or a stereoisomer or tautomer thereof, or a pharmaceutically acceptable salt of any of the foregoing, R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R⁷ is C₁₋₆cycloalkyl; X is —C(O)—; n is 1; p is 1; and q is an integer from 0-2.

In one aspect, provided herein is a compound of formula (I), or (I-C), such as a compound of formula (I-C1):

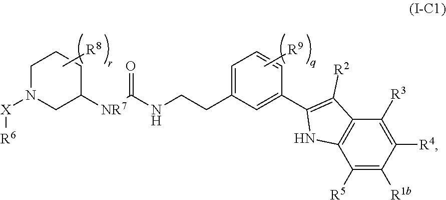

or a stereoisomer or tautomer thereof, or a pharmaceutically acceptable salt of any of the foregoing, wherein n, p, q, r, R^{1b}, R², R³, R⁴, R⁵, R⁶, R⁷, R⁸, R⁹ and X are as defined for a compound of formula (I).

In some embodiments of a compound of formula (I) or (I-C), such as a compound of formula (I-C₁), or a stereoisomer or tautomer thereof, or a pharmaceutically acceptable salt of any of the foregoing, q is an integer from 0 to 2. In some embodiments, q is 1 and R⁹ is halo. In some embodiments, q is 2 and R⁹ is halo.

In one aspect, provided herein is a compound of formula (I), (I-C), or (I-C1), such as a compound of formula (I-C2):

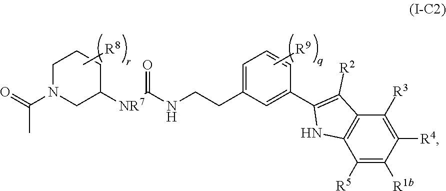

or a stereoisomer or tautomer thereof, or a pharmaceutically acceptable salt of any of the foregoing, wherein n, p, q, r, R^{1b}, R², R³, R⁴, R⁵, R⁷, R⁸, and R⁹ are as defined for a compound of formula (I).

In one aspect, provided herein is a compound of formula (I), such as a compound of formula (I-D):

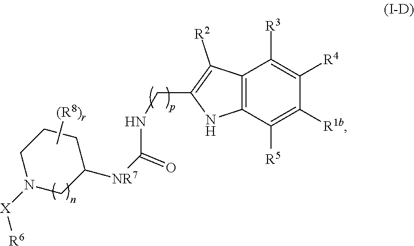

or a stereoisomer or tautomer thereof, or a pharmaceutically acceptable salt of any of the foregoing, wherein n, p, r, R^{1b}, R², R³, R⁴, R⁵, R⁶, R⁷, R⁸, R⁹ and X are as defined for a compound of formula (I).

In some embodiments of a compound of formula (I), or (I-D), or a stereoisomer or tautomer thereof, or a pharmaceutically acceptable salt of any of the foregoing, R^{1b} is H, halo, —CN, C₁₋₆alkyl, C₁₋₆haloalkyl, C₁₋₆cycloalkyl, or C₁₋₆alkoxy, wherein the C₁₋₆alkoxy is optionally substituted with one or more R^{a}; R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a}; R³ and R⁵ are each independently H, —OH, halo, —N(R^{w1})(R^{w2}) or C₁₋₆alkyl, wherein the C₁₋₆alkyl of R³ and R⁵ is optionally substituted with one or more —OH; R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein the C₁₋₆alkyl, C₆₋₂₀aryl, and 5-20 membered heteroaryl of R⁶ are each independently optionally substituted with one or more R^{a}, and the C₃₋₁₀cycloalkyl, and 3-10 membered heterocyclyl of R⁶ are each independently optionally substituted with one or more R^{b}; R⁷ is C₁₋₆alkyl, C₁₋₆cycloalkyl, or C₁₋₆haloalkyl; R⁸ is independently at each occurrence, C₁₋₆alkyl; each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c} each R^{b} is independently —OH, oxo, halo, C₁₋₆alkyl, C₁₋₆alkoxy, —C(O)R^{y}, or —SO₂R^{z}, wherein the C₁₋₆alkyl is optionally substituted with one or more R^{c}; each R^{c} is independently —OH, halo, —N(R^{w1})(R^{w2}), C₁₋₆alkyl, —C₁₋₆alkylN(R^{w1})(R^{w2}), or C₁₋₆alkoxy; R^{w1} and R^{w2} are independently at each occurrence H, C₁₋₆alkyl, C₁₋₆haloalkyl, C₃₋₁₀cycloalkyl, or —C(O)R^{y}; R^{x} is independently at each occurrence, 3-10 membered heterocyclyl optionally substituted with one or more oxo or C₁₋₆alkyl; R^{y} is independently at each occurrence, C₁₋₆alkyl, C₁₋₆haloalkyl; —NH₂, —NH(C₁₋₆alkyl), or —N(C₁₋₆alkyl)₂; R^{z} is independently at each occurrence, C₁₋₆alkyl; X is absent or —C(O)—; n is an integer from 1 to 2; p is an integer from 1 to 4; and r is an integer from 0 to 11.

In some embodiments of a compound of formula (I), or (I-D), or a stereoisomer or tautomer thereof, or a pharmaceutically acceptable salt of any of the foregoing, R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}; R² and R⁴ are each independently H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}; R³ and R⁵ are each independently H, —OH, halo, —N(R^{w1})(R^{w2}), or C₁₋₃alkyl, wherein the C₁₋₃alkyl of R³ and R⁵ is optionally substituted with one or more —OH; R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-6 membered heterocyclyl of R⁶ are each independently optionally substituted with one or more R^{b}; R⁷ is C₁₋₃alkyl, C₁₋₃cycloalkyl, or C₁₋₃haloalkyl; R⁸ is independently at each occurrence, C₁₋₃alkyl; each R^{a} is independently —OH, halo, —CN, C₁₋₃alkyl, C₁₋₃alkoxy, —N(R^{w1})(R^{w2}), —OR^{x}, —C(O)R^{y}, —SO₂R^{z}, C₃₋₆cycloalkyl, C₆₋₁₀aryl, 5-10 membered heteroaryl, or 3-6 membered heterocyclyl, wherein the C₁₋₃alkyl, C₃₋₆cycloalkyl, and 3-6 membered heterocyclyl of R^{a} are each optionally substituted with one or more R^{b}, and the C₆₋₁₀ aryl, and 5-10 membered heteroaryl of R^{a} are each optionally substituted with one or more R^{c}; each R^{b} is independently —OH, oxo, halo, C₁₋₃alkyl, C₁₋₃alkoxy, —C(O)R^{y}, or —SO₂R^{z}, wherein the C₁₋₃alkyl is optionally substituted with one or more R^{c}; each R^{c} is independently —OH, halo, —N(R^{w1})(R^{w2}), C₁₋₃alkyl, —C₁₋₃alkylN(R^{w1})(R^{w2}), or C₁₋₃alkoxy; R^{w1} and R^{w2} are independently at each occurrence H, C₁₋₃alkyl, C₁₋₃haloalkyl, C₃₋₆cycloalkyl, or —C(O)R^{y}; R^{x} is independently at each occurrence, 3-6 membered heterocyclyl optionally substituted with one or more oxo or C₁₋₃alkyl; R^{y} is independently at each occurrence, C₁₋₃alkyl, C₁₋₃haloalkyl; —NH₂, —NH(C₁₋₃alkyl), or —N(C₁₋₃alkyl)₂; R^{z} is independently at each occurrence, C₁₋₃alkyl; X is absent or —C(O)—; n is an integer from 1 to 2; p is an integer from 1 to 4; and r is an integer from 0 to 11.

In some embodiments of a compound of formula (I), or (I-D), or a stereoisomer or tautomer thereof, or a pharmaceutically acceptable salt of any of the foregoing, X is —C(O)—. In some embodiments, X is absent.

In some embodiments of a compound of formula (I) or (I-D), or a stereoisomer or tautomer thereof, or a pharmaceutically acceptable salt of any of the foregoing, n is 1. In some embodiments, n is 2. In some embodiments, p is a integer from 1-3. In some embodiments, p is 1. In some embodiments, p is 2. In some embodiments, p is 1. In some embodiments, p is 3.

In some embodiments of a compound of formula (I), or (I-D), or a stereoisomer or tautomer thereof, or a pharmaceutically acceptable salt of any of the foregoing, X is —C(O)—; n is 1; and p is 1. In some embodiments, X is —C(O)—; n is 1; and p is 2. In some embodiments, X is —C(O)—; n is 1; and p is 3.

In some embodiments of a compound of formula (I), or (I-D), or a stereoisomer or tautomer thereof, or a pharmaceutically acceptable salt of any of the foregoing, R⁷ is C₁₋₆alkyl. In some embodiments, R⁷ is C₁₋₆haloalkyl. In some embodiments, R⁷ is C₁₋₆cycloalkyl. In some embodiments, R⁷ is C₁₋₆alkyl; X is —C(O)—; n is 1; and p is 1. In some embodiments, R⁷ is C₁₋₆cycloalkyl; X is —C(O)—; n is 1; and p is 1. In some embodiments, R⁷ is C₁₋₆haloalkyl; X is —C(O)—; n is 1; and p is 1. In some embodiments, R⁷ is C₁₋₆cycloalkyl. In some embodiments, R⁷ is C₁₋₆alkyl; X is —C(O)—; n is 1; and p is 2. In some embodiments, R⁷ is C₁₋₆cycloalkyl; X is —C(O)—; n is 1; and p is 2. In some embodiments, R⁷ is C₁₋₆haloalkyl; X is —C(O)—; n is 1; and p is 2.

In some embodiments of a compound of formula (I), or (I-D), or a stereoisomer or tautomer thereof, or a pharmaceutically acceptable salt of any of the foregoing, R⁴ is H or halo. In some embodiments, R⁴ is H. In some embodiments, R⁴ is F or Cl. In some embodiments, R⁴ is F. In some embodiments, R⁴ is Cl. In some embodiments, R⁴ is H or halo, R⁷ is C₁₋₆cycloalkyl, and X is —C(O)—; n is 1; and p is 1. In some embodiments, R⁴ is H or halo; R⁷ is C₁₋₆cycloalkyl; X is —C(O)—; n is 1; and p is 2.

In some embodiments of a compound of formula (I), or (I-D), or a stereoisomer or tautomer thereof, or a pharmaceutically acceptable salt of any of the foregoing, R² and R⁴ are each independently H, halo, —CN, C₁₋₃alkyl, C₁₋₃cycloalkyl, C₁₋₃haloalkyl, or C₁₋₃alkoxy, wherein the C₁₋₆alkoxy of R² and R⁴ is optionally substituted with one or more R^{a}; R⁷ is C₁₋₆cycloalkyl; X is —C(O)—; n is 1; and p is an integer from 1 to 3.

In some embodiments of a compound of formula (I), or (I-D), or a stereoisomer or tautomer thereof, or a pharmaceutically acceptable salt of any of the foregoing, wherein X is —C(O)—; n is 1; R² is H, halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}; R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R³ and R⁵ are each independently H, —OH, halo, —N(R^{w1})(R^{w2}), or C₁₋₃alkyl, wherein C₁₋₃alkyl of R³ and R⁵ is optionally substituted with one or more —OH; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-D), or a stereoisomer or tautomer thereof, or a pharmaceutically acceptable salt of any of the foregoing, wherein X is —C(O)—, n is 1, R² is H, halo, —CN, C₁₋₃alkyl, C₁₋₃cycloalkyl, C₁₋₃haloalkyl; R^{1b} is H, halo, —CN, C₁₋₃alkyl, C₁₋₃haloalkyl, C₁₋₃cycloalkyl, or C₁₋₃alkoxy; R³ and R⁵ are each independently H, —OH, halo, —N(R^{w1})(R^{w2}), or C₁₋₃alkyl, wherein C₁₋₃alkyl of R³ and R⁵ is optionally substituted with one or more —OH; and R⁶ is C₁₋₃alkyl, C₁₋₃haloalkyl, C₁₋₃alkoxy, C₃₋₆cycloalkyl, —N(R^{w1})(R^{w2}), C₆₋₁₀ aryl, 3-10 membered heterocyclyl, or 5-10 membered heteroaryl, wherein the C₁₋₃alkyl, C₆₋₁₀ aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}.

In some embodiments of a compound of formula (I), or (I-D), or a stereoisomer or tautomer thereof, or a pharmaceutically acceptable salt of any of the foregoing, wherein X is —C(O)—; n is 1; R² is H, halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}; R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R³ and R⁵ are each independently H, —OH, halo, —N(R^{w1})(R^{w2}) or C₁₋₃alkyl, wherein C₁₋₃alkyl of R³ and R⁵ is optionally substituted with one or more —OH; and R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-10 membered heterocyclyl, or 5-10 membered heteroaryl, wherein the C₁₋₃alkyl, C₆₋₁₀aryl, and 5-10 membered heteroaryl of R⁶ are each independently optionally substituted with one or more R^{a}, and the C₃₋₆cycloalkyl, and 3-10 membered heterocyclyl of R⁶ are each independently optionally substituted with one or more R^{b}. In some embodiments, X is —C(O)—; n is 1; R² is H, halo, —CN, C₁₋₃alkyl, C₁₋₃cycloalkyl, C₁₋₃haloalkyl, or C₁₋₃alkoxy, wherein the C₁₋₃alkoxy of R² is optionally substituted with one or more R^{a}; R^{1b} is H, halo, —CN, C₁₋₃alkyl, C₁₋₃haloalkyl, C₁₋₃cycloalkyl, or C₁₋₃alkoxy, wherein the C₁₋₃alkoxy of R^{1b} is optionally substituted with one or more R^{a}; R³ and R⁵ are each independently H, —OH, halo, —N(R^{w1})(R^{w2}), or C₁₋₃alkyl, wherein C₁₋₃alkyl of R³ and R⁵ is optionally substituted with one or more —OH; and R⁶ is selected from the group consisting of

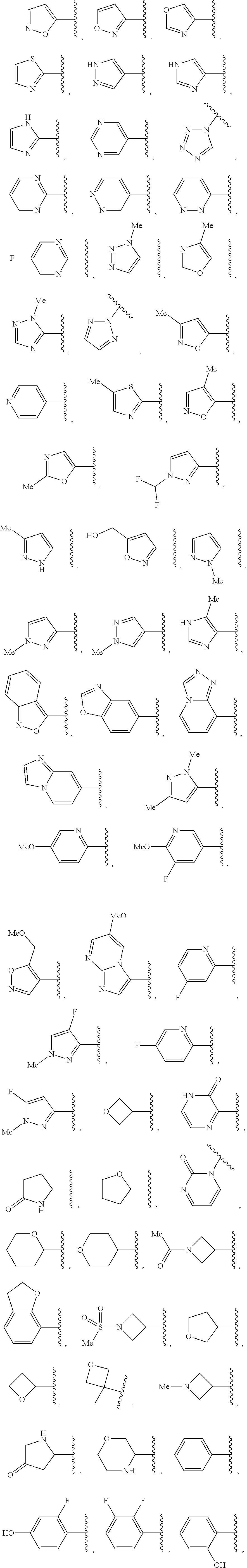

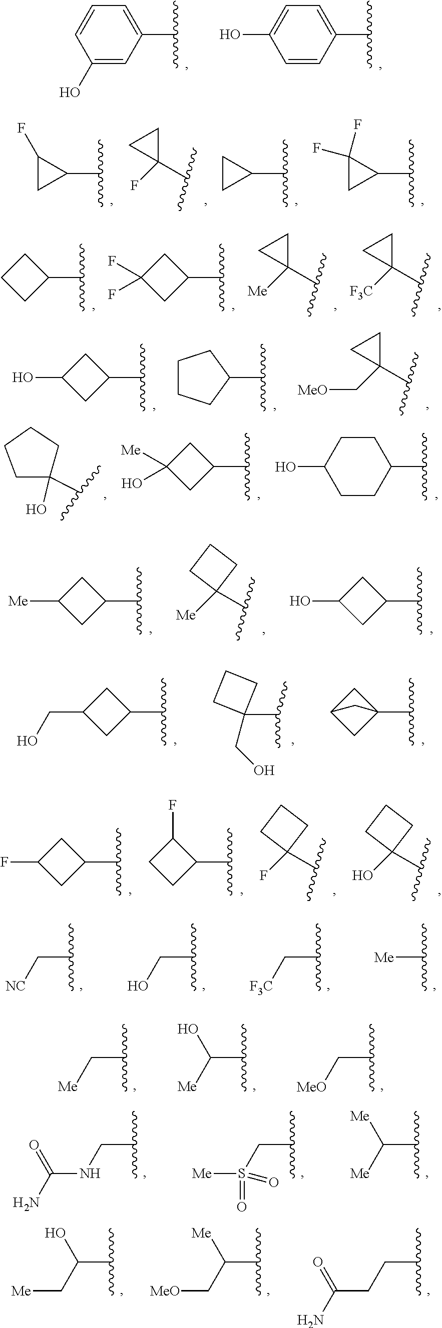

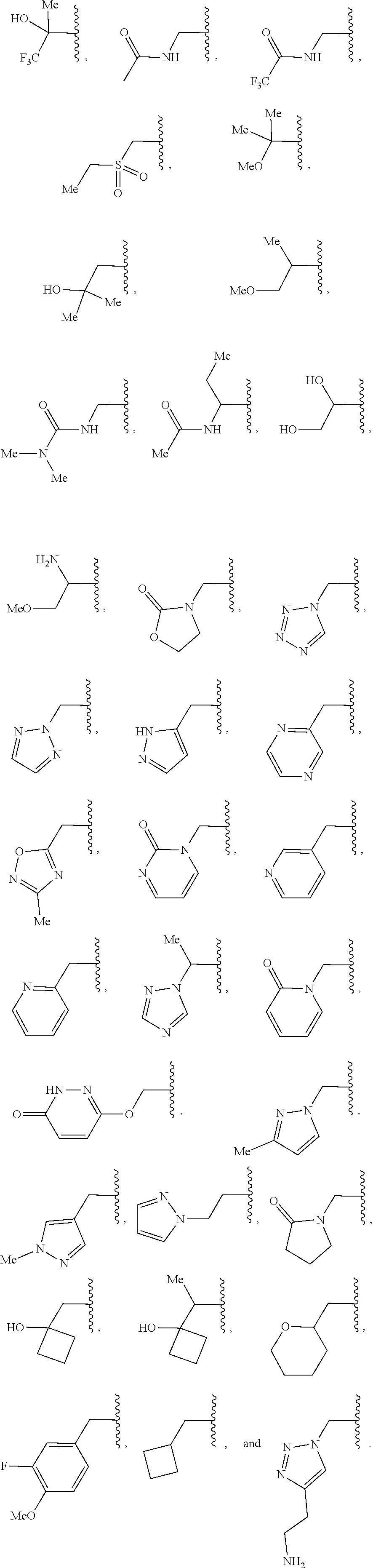

In one aspect, provided herein is a compound of formula (I), or (I-A), (I-A1), or (I-D), such as a compound of formula (I-D1):

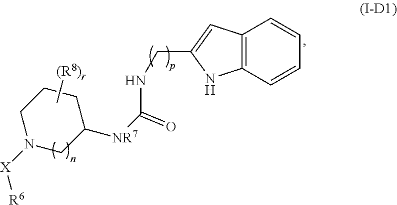

or a stereoisomer or tautomer thereof, or a pharmaceutically acceptable salt of any of the foregoing, wherein n, p, r, R⁶, R⁷, R⁸, and X are as defined for a compound of formula (I).

In one aspect, provided herein is a compound of formula (I), (I-A), (I-A2), or (I-D), such as a compound of formula (I-D2):

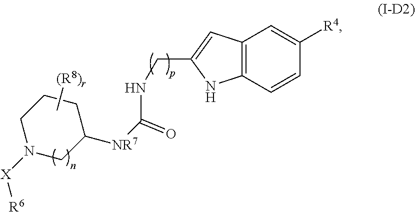

or a stereoisomer or tautomer thereof, or a pharmaceutically acceptable salt of any of the foregoing, wherein n, p, r, R⁴, R⁶, R⁷, R⁸, and X are as defined for a compound of formula (I).

In one aspect, provided herein is a compound of formula (I), (I-A), (I-A3), or (I-D), such as a compound of formula (I-D3):

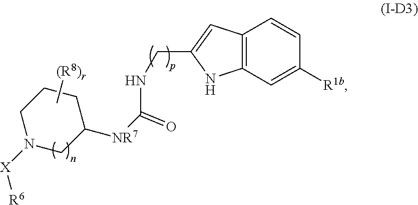

or a stereoisomer or tautomer thereof, or a pharmaceutically acceptable salt of any of the foregoing, wherein n, p, r, R^{1b}, R⁶, R⁷, R⁸, and X are as defined for a compound of formula (I).

In one aspect, provided herein is a compound of formula (I), (I-A), (I-A4), or (I-D), such as a compound of formula (I-D4):

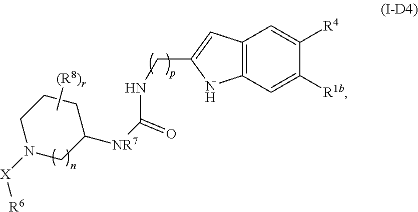

or a stereoisomer or tautomer thereof, or a pharmaceutically acceptable salt of any of the foregoing, wherein n, p, r, R^{1b}, R⁴, R⁶, R⁷, R⁸, and X are as defined for a compound of formula (I).

In one aspect, provided herein is a compound of formula (I), (I-A), (I-A5), or (I-D), such as a compound of formula (I-D5):

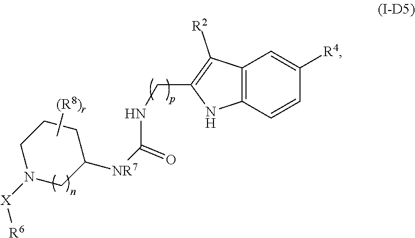

or a stereoisomer or tautomer thereof, or a pharmaceutically acceptable salt of any of the foregoing, wherein n, p, r, R², R⁴, R⁶, R⁷, R⁸, and X are as defined for a compound of formula (I).

In one aspect, provided herein is a compound of formula (I), (I-A), (I-A6), or (I-D), such as a compound of formula (I-D6):

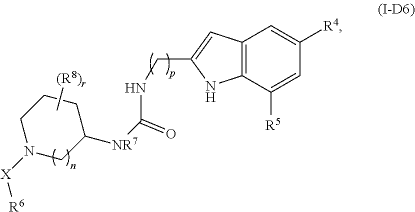

or a stereoisomer or tautomer thereof, or a pharmaceutically acceptable salt of any of the foregoing, wherein n, p, r, R⁴, R⁵, R⁶, R⁷, R⁸, and X are as defined for a compound of formula (I).

In some embodiments of a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1.

Compound Names included in Table 1 and for all intermediates and compounds were generated using ChemDraw© Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.1.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1

| Compound | | |
|---|---|---|
| No. | Structure | IUPAC name |
| 1 | 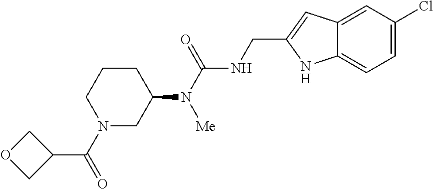 [SMILES: CN(C(=O)NCc1cc2cc(Cl)ccc2[nH]1)[C@@H]1CCCN(C(=O)C2COC2)C1] | (R)-3-((5-chloro-1H-indol-2- yl)methyl)-1-methyl-1-(1- (oxetane-3-carbonyl)piperidin-3- yl)urea |
| 2 | 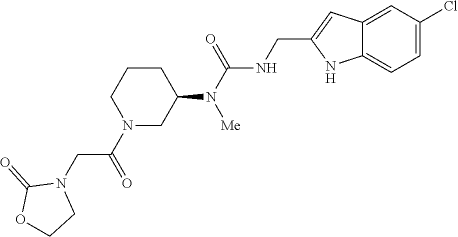 [SMILES: CN(C(=O)NCc1cc2cc(Cl)ccc2[nH]1)[C@@H]1CCCN(C(=O)CN2CCOC2=O)C1] | (R)-3-((5-chloro-1H-indol-2- yl)methyl)-1-methyl-1-(1-(2-(2- oxooxazolidin-3- yl)acetyl)piperidin-3-yl)urea |
| 3 | 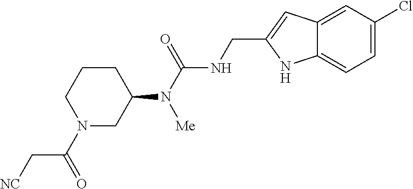 [SMILES: CN(C(=O)NCc1cc2cc(Cl)ccc2[nH]1)[C@@H]1CCCN(C(=O)CC#N)C1] | (R)-3-((5-chloro-1H-indol-2- yl)methyl)-1-(1-(2- cyanoacetyl)piperidin-3-yl)-1- methylurea |
| 4 | 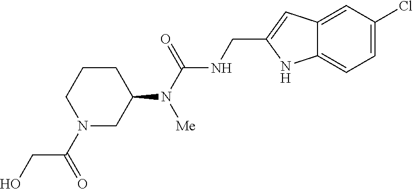 [SMILES: CN(C(=O)NCc1cc2cc(Cl)ccc2[nH]1)[C@@H]1CCCN(C(=O)CO)C1] | (R)-3-((5-chloro-1H-indol-2- yl)methyl)-1-(1-(2- hydroxyacetyl)piperidin-3-yl)-1- methylurea |
| 5 | 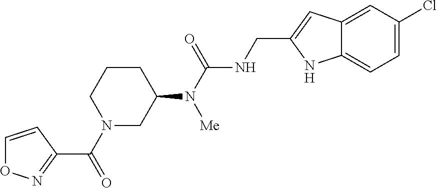 [SMILES: CN(C(=O)NCc1cc2cc(Cl)ccc2[nH]1)[C@@H]1CCCN(C(=O)c2ccon2)C1] | (R)-3-((5-chloro-1H-indol-2- yl)methyl)-1-(1-(isoxazole-3- carbonyl)piperidin-3-yl)-1- methylurea |
| 6 | 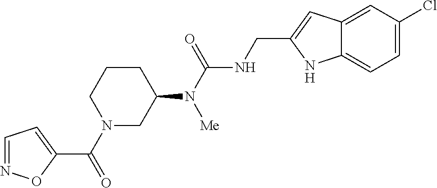 [SMILES: CN(C(=O)NCc1cc2cc(Cl)ccc2[nH]1)[C@@H]1CCCN(C(=O)c2ccno2)C1] | (R)-3-((5-chloro-1H-indol-2- yl)methyl)-1-(1-(isoxazole-5- carbonyl)piperidin-3-yl)-1- methylurea |
| 7 | 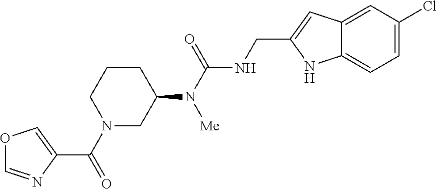 [SMILES: CN(C(=O)NCc1cc2cc(Cl)ccc2[nH]1)[C@@H]1CCCN(C(=O)c2cocn2)C1] | (R)-3-((5-chloro-1H-indol-2- yl)methyl)-1-methyl-1-(1- (oxazole-4-carbonyl)piperidin-3- yl)urea |
| 8 | 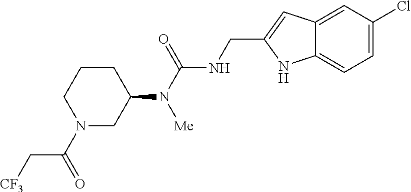 [SMILES: CN(C(=O)NCc1cc2cc(Cl)ccc2[nH]1)[C@@H]1CCCN(C(=O)CC(F)(F)F)C1] | (R)-3-((5-chloro-1H-indol-2- yl)methyl)-1-methyl-1-(1-(3,3,3- trifluoropropanoyl)piperidin-3- yl)urea |
| 9 | 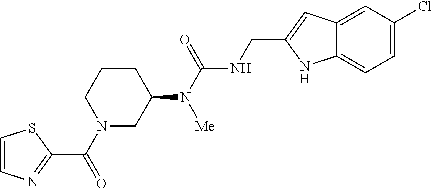 [SMILES: CN(C(=O)NCc1cc2cc(Cl)ccc2[nH]1)[C@@H]1CCCN(C(=O)c2nccs2)C1] | (R)-3-((5-chloro-1H-indol-2- yl)methyl)-1-methyl-1-(1- (thiazole-2-carbonyl)piperidin-3- yl)urea |
| 10 | 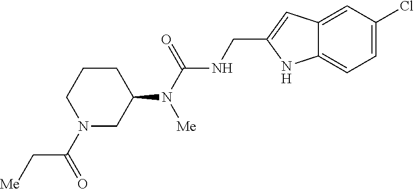 [SMILES: CCC(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1- propionylpiperidin-3-yl)urea |
| 11 | 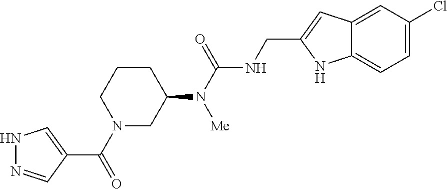 [SMILES: CN(C(=O)NCc1cc2cc(Cl)ccc2[nH]1)[C@@H]1CCCN(C(=O)c2cn[nH]c2)C1] | (R)-1-(1-(1H-pyrazole-4- carbonyl)piperidin-3-yl)-3-((5- chloro-1H-indol-2-yl)methyl)-1- methylurea |
| 12 | 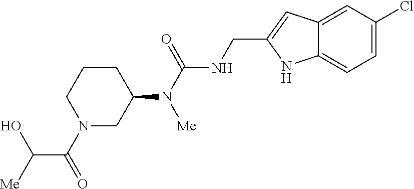 [SMILES: CC(O)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(2- hydroxypropanoyl)piperidin-3-yl)- 1-methylurea |
| 13 | 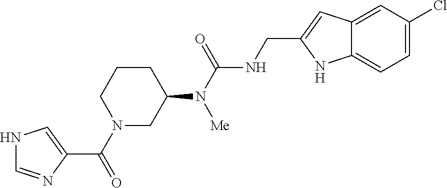 [SMILES: CN(C(=O)NCc1cc2cc(Cl)ccc2[nH]1)[C@@H]1CCCN(C(=O)c2c[nH]cn2)C1] | (R)-1-(1-(1H-imidazole-4- carbonyl)piperidin-3-yl)-3-((5- chloro-1H-indol-2-yl)methyl)-1- methylurea |
| 14 | 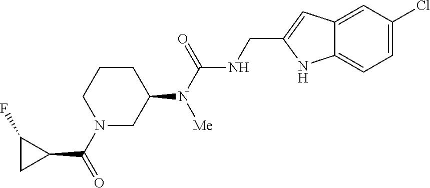 [SMILES: CN(C(=O)NCc1cc2cc(Cl)ccc2[nH]1)[C@@H]1CCCN(C(=O)[C@H]2C[C@@H]2F)C1] | 3-((5-chloro-1H-indol-2- yl)methyl)-1-((R)-1-((1R,2S)-2- fluorocyclopropane-1- carbonyl)piperidin-3-yl)-1- methylurea |
| 15 | 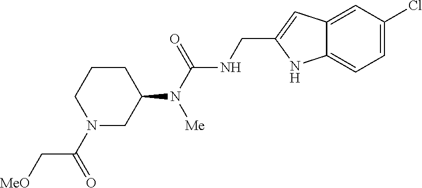 [SMILES: COCC(=O)N1CCC[C@@H](N(C)C(=O)NCc2cc3cc(Cl)ccc3[nH]2)C1] | (R)-3-((5-chloro-1H-indol-2- yl)methyl)-1-(1-(2- methoxyacetyl)piperidin-3-yl)-1- methylurea |
| 16 | 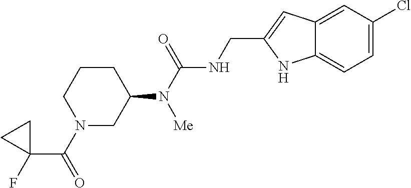 [SMILES: CN(C(=O)NCc1cc2cc(Cl)ccc2[nH]1)[C@@H]1CCCN(C(=O)C2(F)CC2)C1] | (R)-3-((5-chloro-1H-indol-2- yl)methyl)-1-(1-(1- fluorocyclopropane-1- carbonyl)piperidin-3-yl)-1- methylurea |
| 17 | 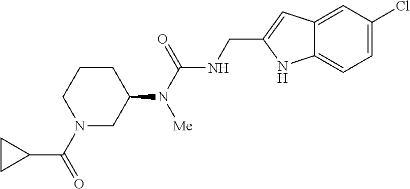 [SMILES: CN(C(=O)NCc1cc2cc(Cl)ccc2[nH]1)[C@@H]1CCCN(C(=O)C2CC2)C1] | (R)-3-((5-chloro-1H-indol-2- yl)methyl)-1-(1- (cyclopropanecarbonyl)piperidin- 3-yl)-1-methylurea |
| 18 | 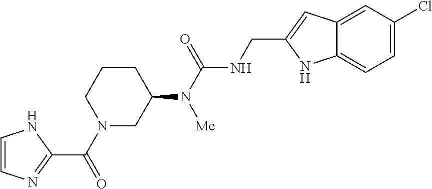 [SMILES: CN(C(=O)NCc1cc2cc(Cl)ccc2[nH]1)[C@@H]1CCCN(C(=O)c2ncc[nH]2)C1] | (R)-1-(1-(1H-imidazole-2- carbonyl)piperidin-3-yl)-3-((5- chloro-1H-indol-2-yl)methyl)-1- methylurea |
| 19 | 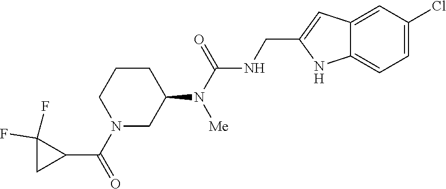 [SMILES: CN(C(=O)NCc1cc2cc(Cl)ccc2[nH]1)[C@@H]1CCCN(C(=O)C2CC2(F)F)C1] | 3-((5-chloro-1H-indol-2- yl)methyl)-1-((3R)-1-(2,2- difluorocyclopropane-1- carbonyl)piperidin-3-yl)-1- methylurea |
| 20 | 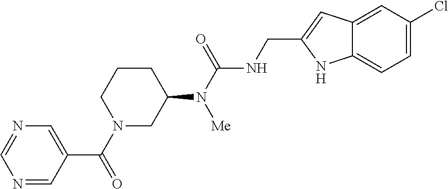 [SMILES: CN(C(=O)NCc1cc2cc(Cl)ccc2[nH]1)[C@@H]1CCCN(C(=O)c2cncnc2)C1] | (R)-3-((5-chloro-1H-indol-2- yl)methyl)-1-methyl-1-(1- (pyrimidine-5-carbonyl)piperidin- 3-yl)urea |
| 21 | 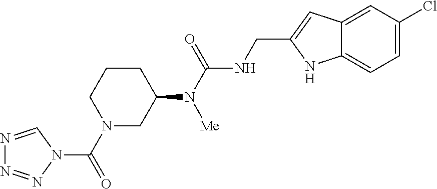 [SMILES: CN(C(=O)NCc1cc2cc(Cl)ccc2[nH]1)[C@@H]1CCCN(C(=O)n2cnnn2)C1] | (R)-1-(1-(2-(1H-tetrazol-1- yl)acetyl)piperidin-3-yl)-3-((5- chloro-1H-indol-2-yl)methyl)-1- methylurea |
| 22 | 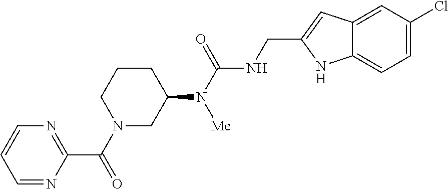 [SMILES: CN(C(=O)NCc1cc2cc(Cl)ccc2[nH]1)[C@@H]1CCCN(C(=O)c2ncccn2)C1] | (R)-3-((5-chloro-1H-indol-2- yl)methyl)-1-methyl-1-(1- (pyrimidine-2-carbonyl)piperidin- 3-yl)urea |
| 23 | 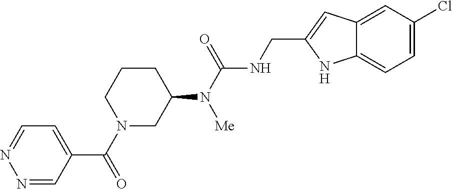 [SMILES: CN(C(=O)NCc1cc2cc(Cl)ccc2[nH]1)[C@@H]1CCCN(C(=O)c2ccnnc2)C1] | (R)-3-((5-chloro-1H-indol-2- yl)methyl)-1-methyl-1-(1- (pyridazine-4-carbonyl)piperidin- 3-yl)urea |
| 24 | 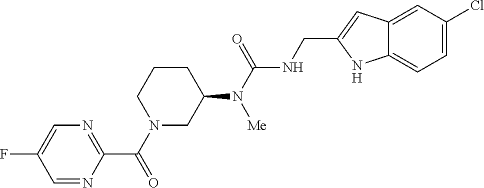 [SMILES: CN(C(=O)NCc1cc2cc(Cl)ccc2[nH]1)[C@@H]1CCCN(C(=O)c2ncc(F)cn2)C1] | (R)-3-((5-chloro-1H-indol-2- yl)methyl)-1-(1-(5- fluoropyrimidine-2- carbonyl)piperidin-3-yl)-1- methylurea |
| 25 | 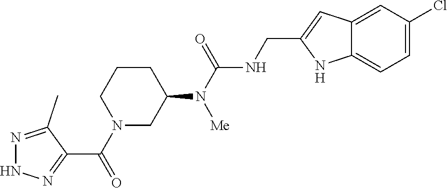 [SMILES: Cc1n[nH]nc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(5- methyl-2H-1,2,3-triazole-4- carbonyl)piperidin-3-yl)urea |
| 26 | 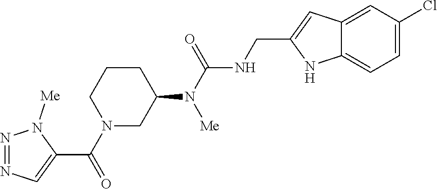 [SMILES: CN(C(=O)NCc1cc2cc(Cl)ccc2[nH]1)[C@@H]1CCCN(C(=O)c2cnnn2C)C1] | (R)-3-((5-chloro-1H-indol-2- yl)methyl)-1-methyl-1-(1-(1- methyl-1H-1,2,3-triazole-5- carbonyl)piperidin-3-yl)urea |
| 27 | 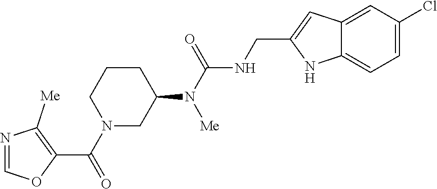 [SMILES: Cc1ncoc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(4- methyloxazole-5- carbonyl)piperidin-3-yl)urea |
| 28 | 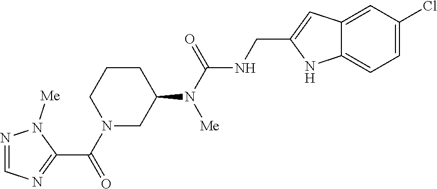 [SMILES: CN(C(=O)NCc1cc2cc(Cl)ccc2[nH]1)[C@@H]1CCCN(C(=O)c2ncnn2C)C1] | (R)-3-((5-chloro-1H-indol-2- yl)methyl)-1-methyl-1-(1-(1- methyl-1H-1,2,4-triazole-5- carbonyl)piperidin-3-yl)urea |
| 29 | 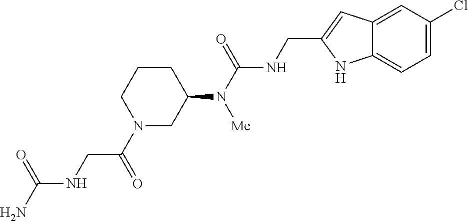 [SMILES: CN(C(=O)NCc1cc2cc(Cl)ccc2[nH]1)[C@@H]1CCCN(C(=O)CNC(N)=O)C1] | (R)-1-(1- (carbamoylglycyl)piperidin-3-yl)- 3-((5-chloro-1H-indol-2- yl)methyl)-1-methylurea |
| 30 | 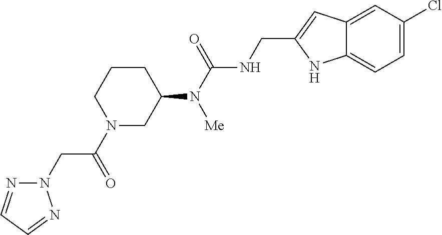 [SMILES: CN(C(=O)NCc1cc2cc(Cl)ccc2[nH]1)[C@@H]1CCCN(C(=O)Cn2nccn2)C1] | (R)-1-(1-(2-(2H-1,2,3-triazol-2- yl)acetyl)piperidin-3-yl)-3-((5- chloro-1H-indol-2-yl)methyl)-1- methylurea |
| 31 | 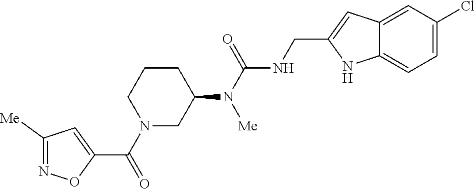 [SMILES: Cc1cc(C(=O)N2CCC[C@@H](N(C)C(=O)NCc3cc4cc(Cl)ccc4[nH]3)C2)on1] | (R)-3-((5-chloro-1H-indol-2- yl)methyl)-1-methyl-1-(1-(3- methylisoxazole-5- carbonyl)piperidin-3-yl)urea |
| 32 | 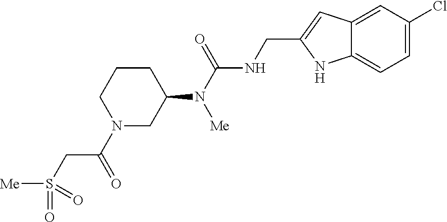 [SMILES: CN(C(=O)NCc1cc2cc(Cl)ccc2[nH]1)[C@@H]1CCCN(C(=O)CS(C)(=O)=O)C1] | (R)-3-((5-chloro-1H-indol-2- yl)methyl)-1-methyl-1-(1-(2- (methylsulfonyl)acetyl)piperidin- 3-yl)urea |
| 33 | 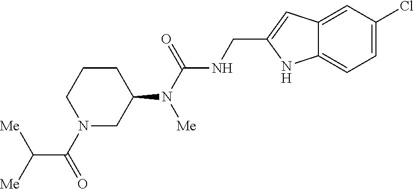 [SMILES: CC(C)C(=O)N1CCC[C@@H](N(C)C(=O)NCc2cc3cc(Cl)ccc3[nH]2)C1] | (R)-3-((5-chloro-1H-indol-2- yl)methyl)-1-(1- isobutyrylpiperidin-3-yl)-1- methylurea |
| 34 | 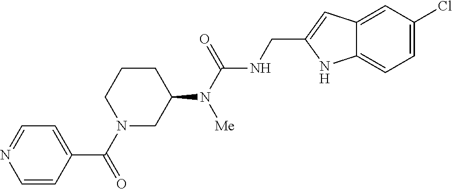 [SMILES: CN(C(=O)NCc1cc2cc(Cl)ccc2[nH]1)[C@@H]1CCCN(C(=O)c2ccncc2)C1] | (R)-3-((5-chloro-1H-indol-2- yl)methyl)-1-(1- isonicotinoylpiperidin-3-yl)-1- methylurea |
| 35 | 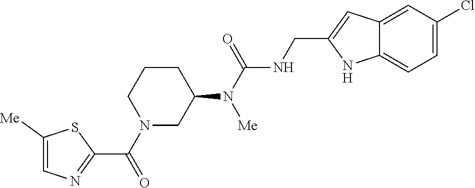 [SMILES: Cc1cnc(C(=O)N2CCC[C@@H](N(C)C(=O)NCc3cc4cc(Cl)ccc4[nH]3)C2)s1] | (R)-3-((5-chloro-1H-indol-2- yl)methyl)-1-methyl-1-(1-(5- methylthiazole-2- carbonyl)piperidin-3-yl)urea |
| 36 | 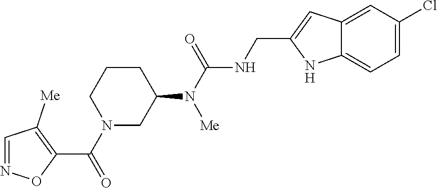 [SMILES: Cc1cnoc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(4- methylisoxazole-5- carbonyl)piperidin-3-yl)urea |
| 37 | 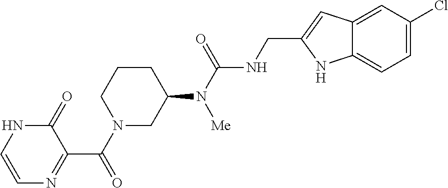 [SMILES: CN(C(=O)NCc1cc2cc(Cl)ccc2[nH]1)[C@@H]1CCCN(C(=O)c2ncc[nH]c2=O)C1] | (R)-3-((5-chloro-1H-indol-2- yl)methyl)-1-methyl-1-(1-(3-oxo- 3,4-dihydropyrazine-2- carbonyl)piperidin-3-yl)urea |
| 38 | 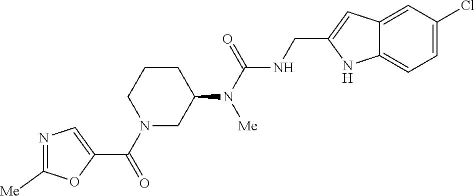 [SMILES: Cc1ncc(C(=O)N2CCC[C@@H](N(C)C(=O)NCc3cc4cc(Cl)ccc4[nH]3)C2)o1] | (R)-3-((5-chloro-1H-indol-2- yl)methyl)-1-methyl-1-(1-(2- methyloxazole-5- carbonyl)piperidin-3-yl)urea |
| 39 | 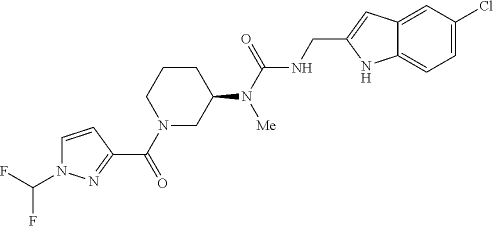 [SMILES: CN(C(=O)NCc1cc2cc(Cl)ccc2[nH]1)[C@@H]1CCCN(C(=O)c2ccn(C(F)F)n2)C1] | (R)-3-((5-chloro-1H-indol-2- yl)methyl)-1-(1-(1- (difluoromethyl)-1H-pyrazole-3- carbonyl)piperidin-3-yl)-1- methylurea |
| 40 | 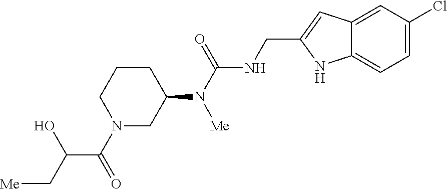 [SMILES: CCC(O)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(2- hydroxybutanoyl)piperidin-3-yl)- 1-methylurea |
| 41 | 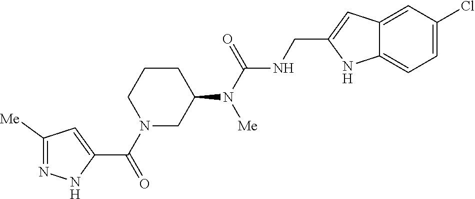 [SMILES: Cc1cc(C(=O)N2CCC[C@@H](N(C)C(=O)NCc3cc4cc(Cl)ccc4[nH]3)C2)[nH]n1] | (R)-3-((5-chloro-1H-indol-2- yl)methyl)-1-methyl-1-(1-(3- methyl-1H-pyrazole-5- carbonyl)piperidin-3-yl)urea |
| 42 | 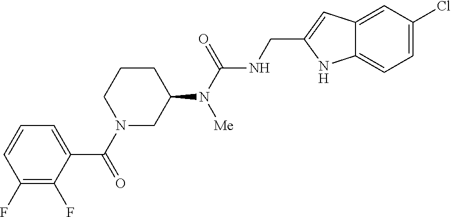 [SMILES: CN(C(=O)NCc1cc2cc(Cl)ccc2[nH]1)[C@@H]1CCCN(C(=O)c2cccc(F)c2F)C1] | (R)-3-((5-chloro-1H-indol-2- yl)methyl)-1-(1-(2,3- difluorobenzoyl)piperidin-3-yl)-1- methylurea |
| 43 | 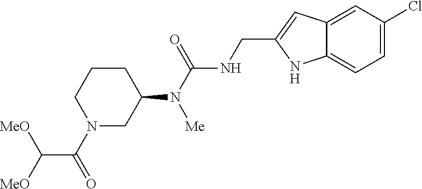 [SMILES: COC(OC)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(2- methoxypropanoyl)piperidin-3- yl)-1-methylurea |
| 44 | 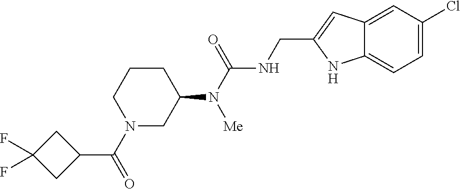 [SMILES: CN(C(=O)NCc1cc2cc(Cl)ccc2[nH]1)[C@@H]1CCCN(C(=O)C2CC(F)(F)C2)C1] | (R)-3-((5-chloro-1H-indol-2- yl)methyl)-1-(1-(3,3- difluorocyclobutane-1- carbonyl)piperidin-3-yl)-1- methylurea |
| 45 | 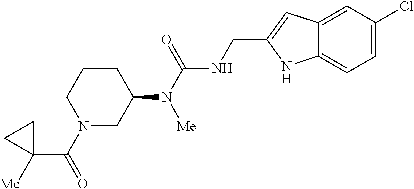 [SMILES: CN(C(=O)NCc1cc2cc(Cl)ccc2[nH]1)[C@@H]1CCCN(C(=O)C2(C)CC2)C1] | (R)-3-((5-chloro-1H-indol-2- yl)methyl)-1-methyl-1-(1-(1- methylcyclopropane-1- carbonyl)piperidin-3-yl)urea |
| 46 | 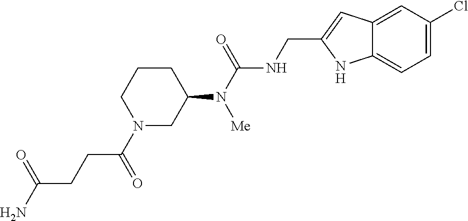 [SMILES: CN(C(=O)NCc1cc2cc(Cl)ccc2[nH]1)[C@@H]1CCCN(C(=O)CCC(N)=O)C1] | (R)-4-(3-(3-((5-chloro-1H-indol-2- yl)methyl)-1- methylureido)piperidin-1-yl)-4- oxobutanamide |
| 47 | 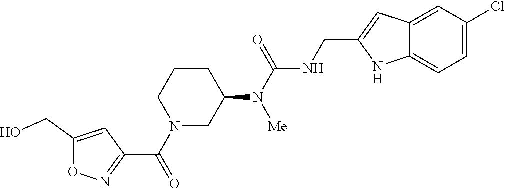 [SMILES: CN(C(=O)NCc1cc2cc(Cl)ccc2[nH]1)[C@@H]1CCCN(C(=O)c2cc(CO)on2)C1] | (R)-3-((5-chloro-1H-indol-2- yl)methyl)-1-(1-(5- (hydroxymethyl)isoxazole-3- carbonyl)piperidin-3-yl)-1- methylurea |
| 48 | 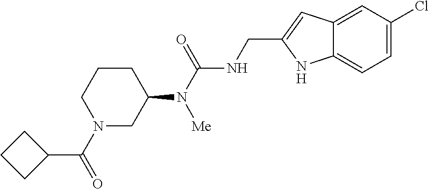 [SMILES: CN(C(=O)NCc1cc2cc(Cl)ccc2[nH]1)[C@@H]1CCCN(C(=O)C2CCC2)C1] | (R)-3-((5-chloro-1H-indol-2- yl)methyl)-1-(1- (cyclobutanecarbonyl)piperidin-3- yl)-1-methylurea |
| 49 | 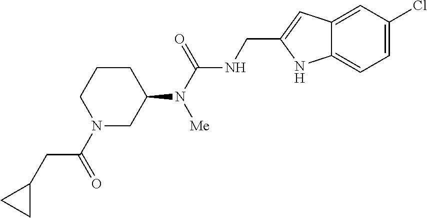 [SMILES: CN(C(=O)NCc1cc2cc(Cl)ccc2[nH]1)[C@@H]1CCCN(C(=O)CC2CC2)C1] | (R)-3-((5-chloro-1H-indol-2- yl)methyl)-1-(1-(2- cyclopropylacetyl)piperidin-3-yl)- 1-methylurea |
| 50 | 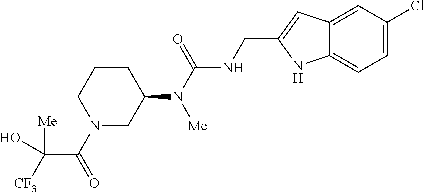 [SMILES: CN(C(=O)NCc1cc2cc(Cl)ccc2[nH]1)[C@@H]1CCCN(C(=O)C(C)(O)C(F)(F)F)C1] | 3-((5-chloro-1H-indol-2- yl)methyl)-1-methyl-1-((3R)-1- (3,3,3-trifluoro-2-hydroxy-2- methylpropanoyl)piperidin-3- yl)urea |
| 51 | 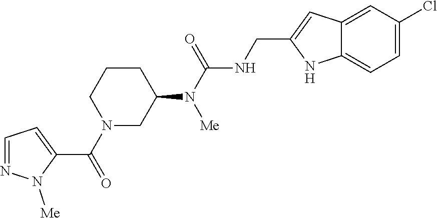 [SMILES: CN(C(=O)NCc1cc2cc(Cl)ccc2[nH]1)[C@@H]1CCCN(C(=O)c2ccnn2C)C1] | (R)-3-((5-chloro-1H-indol-2- yl)methyl)-1-methyl-1-(1-(1- methyl-1H-pyrazole-5- carbonyl)piperidin-3-yl)urea |
| 52 | 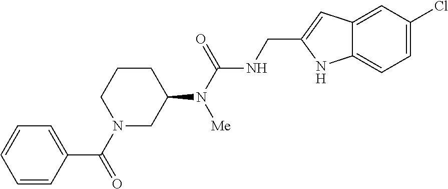 [SMILES: CN(C(=O)NCc1cc2cc(Cl)ccc2[nH]1)[C@@H]1CCCN(C(=O)c2ccccc2)C1] | (R)-1-(1-benzoylpiperidin-3-yl)-3- ((5-chloro-1H-indol-2-yl)methyl)- 1-methylurea |
| 53 | 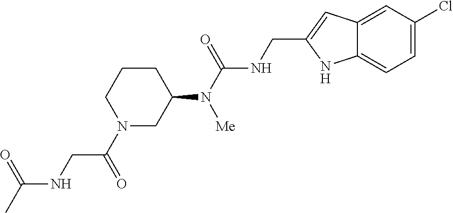 [SMILES: CC(=O)NCC(=O)N1CCC[C@@H](N(C)C(=O)NCc2cc3cc(Cl)ccc3[nH]2)C1] | (R)-N-(2-(3-(3-((5-chloro-1H- indol-2-yl)methyl)-1- methylureido)piperidin-1-yl)-2- oxoethyl)acetamide |
| 54 | 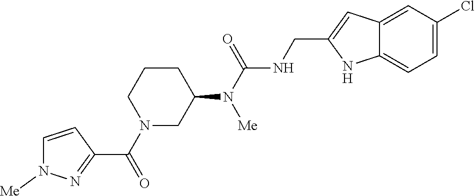 [SMILES: CN(C(=O)NCc1cc2cc(Cl)ccc2[nH]1)[C@@H]1CCCN(C(=O)c2ccn(C)n2)C1] | (R)-3-((5-chloro-1H-indol-2- yl)methyl)-1-methyl-1-(1-(1- methyl-1H-pyrazole-3- carbonyl)piperidin-3-yl)urea |
| 55 | 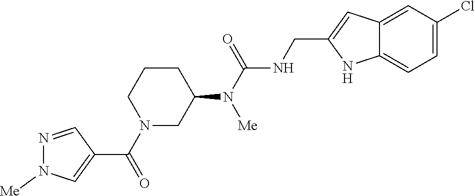 [SMILES: CN(C(=O)NCc1cc2cc(Cl)ccc2[nH]1)[C@@H]1CCCN(C(=O)c2cnn(C)c2)C1] | (R)-3-((5-chloro-1H-indol-2- yl)methyl)-1-methyl-1-(1-(1- methyl-1H-pyrazole-4- carbonyl)piperidin-3-yl)urea |
| 56 | 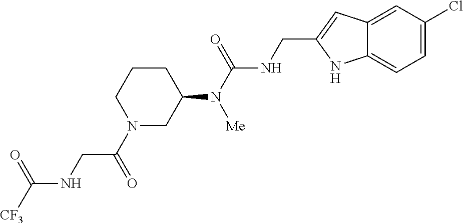 [SMILES: CN(C(=O)NCc1cc2cc(Cl)ccc2[nH]1)[C@@H]1CCCN(C(=O)CNC(=O)C(F)(F)F)C1] | (R)-N-(2-(3-(3-((5-chloro-1H- indol-2-yl)methyl)-1- methylureido)piperidin-1-yl)-2- oxoethyl)-2,2,2-trifluoroacetamide |
| 57 | 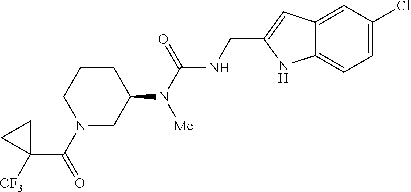 [SMILES: CN(C(=O)NCc1cc2cc(Cl)ccc2[nH]1)[C@@H]1CCCN(C(=O)C2(C(F)(F)F)CC2)C1] | (R)-3-((5-chloro-1H-indol-2- yl)methyl)-1-methyl-1-(1-(1- (trifluoromethyl)cyclopropane-1- carbonyl)piperidin-3-yl)urea |
| 58 | 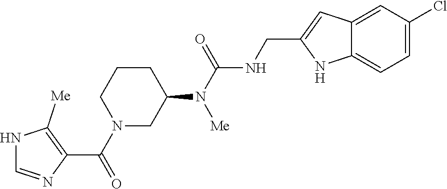 [SMILES: Cc1[nH]cnc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(5- methyl-1H-imidazole-4- carbonyl)piperidin-3-yl)urea |
| 59 | 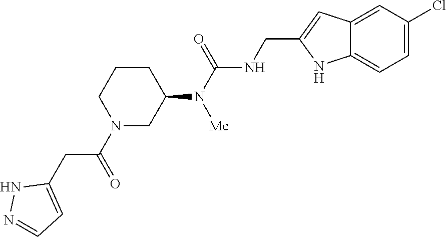 [SMILES: CN(C(=O)NCc1cc2cc(Cl)ccc2[nH]1)[C@@H]1CCCN(C(=O)Cc2ccn[nH]2)C1] | (R)-1-(1-(2-(1H-pyrazol-5- yl)acetyl)piperidin-3-yl)-3-((5- chloro-1H-indol-2-yl)methyl)-1- methylurea |
| 60 | 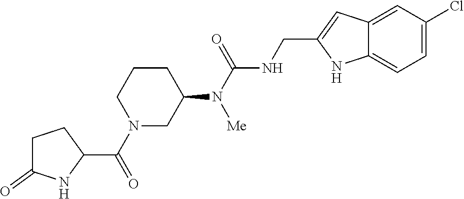 [SMILES: CN(C(=O)NCc1cc2cc(Cl)ccc2[nH]1)[C@@H]1CCCN(C(=O)C2CCC(=O)N2)C1] | 3-((5-chloro-1H-indol-2- yl)methyl)-1-methyl-1-((3R)-1-(5- oxopyrrolidine-2- carbonyl)piperidin-3-yl)urea |
| 61 | 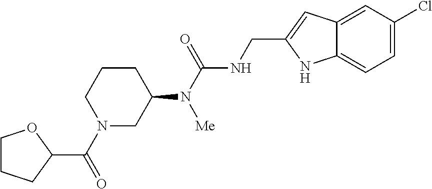 [SMILES: CN(C(=O)NCc1cc2cc(Cl)ccc2[nH]1)[C@@H]1CCCN(C(=O)C2CCCO2)C1] | 3-((5-chloro-1H-indol-2- yl)methyl)-1-methyl-1-((3R)-1- (tetrahydrofuran-2- carbonyl)piperidin-3-yl)urea |
| 62 | 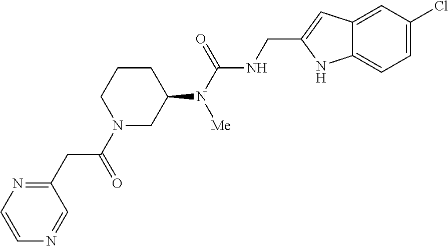 [SMILES: CN(C(=O)NCc1cc2cc(Cl)ccc2[nH]1)[C@@H]1CCCN(C(=O)Cc2cnccn2)C1] | (R)-3-((5-chloro-1H-indol-2- yl)methyl)-1-methyl-1-(1-(2- (pyrazin-2-yl)acetyl)piperidin-3- yl)urea |
| 63 | 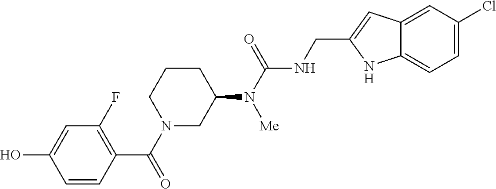 [SMILES: CN(C(=O)NCc1cc2cc(Cl)ccc2[nH]1)[C@@H]1CCCN(C(=O)c2ccc(O)cc2F)C1] | (R)-3-((5-chloro-1H-indol-2- yl)methyl)-1-(1-(2-fluoro-4- hydroxybenzoyl)piperidin-3-yl)-1- methylurea |
| 64 | 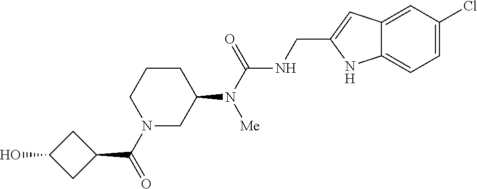 [SMILES: CN(C(=O)NCc1cc2cc(Cl)ccc2[nH]1)[C@@H]1CCCN(C(=O)[C@H]2C[C@H](O)C2)C1] | trans-(R)-3-((5-chloro-1H-indol-2- yl)methyl)-1-(1-(3- hydroxycyclobutane-1- carbonyl)piperidin-3-yl)-1- methylurea |
| 65 | 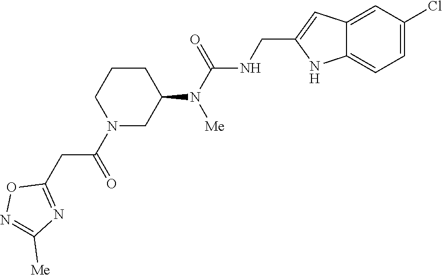 [SMILES: Cc1noc(CC(=O)N2CCC[C@@H](N(C)C(=O)NCc3cc4cc(Cl)ccc4[nH]3)C2)n1] | (R)-3-((5-chloro-1H-indol-2- yl)methyl)-1-methyl-1-(1-(2-(3- methyl-1,2,4-oxadiazol-5- yl)acetyl)piperidin-3-yl)urea |
| 66 | 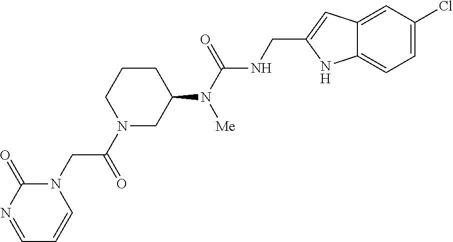 [SMILES: CN(C(=O)NCc1cc2cc(Cl)ccc2[nH]1)[C@@H]1CCCN(C(=O)Cn2cccnc2=O)C1] | (R)-3-((5-chloro-1H-indol-2- yl)methyl)-1-methyl-1-(1-(2-(2- oxopyrimidin-1(2H)- yl)acetyl)piperidin-3-yl)urea |
| 67 | 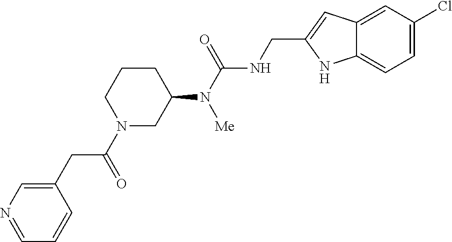 [SMILES: CN(C(=O)NCc1cc2cc(Cl)ccc2[nH]1)[C@@H]1CCCN(C(=O)Cc2cccnc2)C1] | (R)-3-((5-chloro-1H-indol-2- yl)methyl)-1-methyl-1-(1-(2- (pyridin-3-yl)acetyl)piperidin-3- yl)urea |
| 68 | 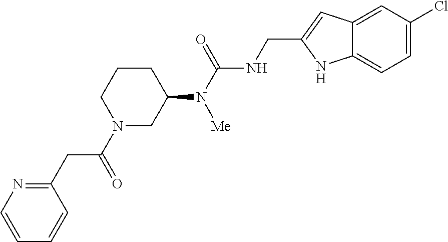 [SMILES: CN(C(=O)NCc1cc2cc(Cl)ccc2[nH]1)[C@@H]1CCCN(C(=O)Cc2ccccn2)C1] | (R)-3-((5-chloro-1H-indol-2- yl)methyl)-1-methyl-1-(1-(2- (pyridin-2-yl)acetyl)piperidin-3- yl)urea |
| 69 | 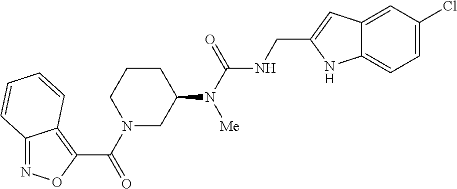 [SMILES: CN(C(=O)NCc1cc2cc(Cl)ccc2[nH]1)[C@@H]1CCCN(C(=O)c2onc3ccccc23)C1] | (R)-1-(1-(benzo[c]isoxazole-3- carbonyl)piperidin-3-yl)-3-((5- chloro-1H-indol-2-yl)methyl)-1- methylurea |
| 70 | 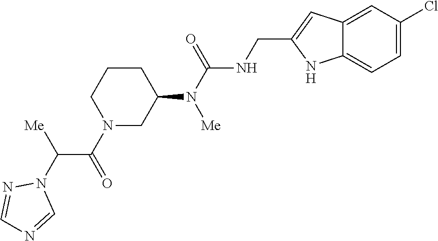 [SMILES: CC(C(=O)N1CCC[C@@H](N(C)C(=O)NCc2cc3cc(Cl)ccc3[nH]2)C1)n1cncn1] | 1-((3R)-1-(2-(1H-1,2,4-triazol-1- yl)propanoyl)piperidin-3-yl)-3-((5- chloro-1H-indol-2-yl)methyl)-1- methylurea |
| 71 | 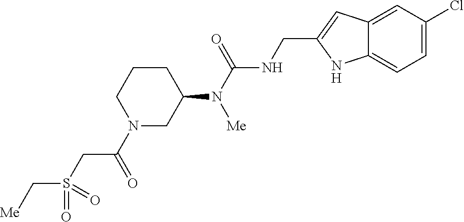 [SMILES: CCS(=O)(=O)CC(=O)N1CCC[C@@H](N(C)C(=O)NCc2cc3cc(Cl)ccc3[nH]2)C1] | (R)-3-((5-chloro-1H-indol-2- yl)methyl)-1-(1-(2- (ethylsulfonyl)acetyl)piperidin-3- yl)-1-methylurea |
| 72 | 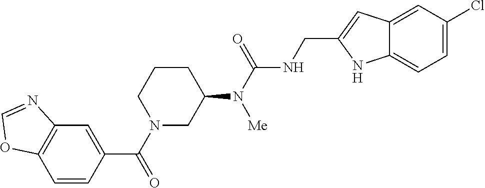 [SMILES: CN(C(=O)NCc1cc2cc(Cl)ccc2[nH]1)[C@@H]1CCCN(C(=O)c2ccc3ocnc3c2)C1] | (R)-1-(1-(benzo[d]oxazole-5- carbonyl)piperidin-3-yl)-3-((5- chloro-1H-indol-2-yl)methyl)-1- methylurea |
| 73 | 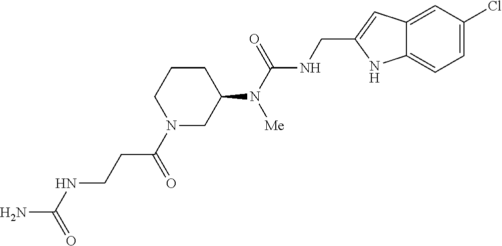 [SMILES: CN(C(=O)NCc1cc2cc(Cl)ccc2[nH]1)[C@@H]1CCCN(C(=O)CCNC(N)=O)C1] | (R)-3-((5-chloro-1H-indol-2- yl)methyl)-1-methyl-1-(1-(3- ureidopropanoyl)piperidin-3- yl)urea |
| 74 | 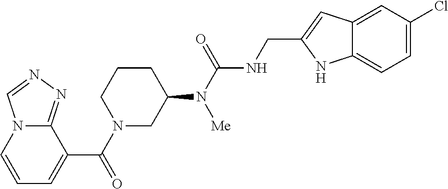 [SMILES: CN(C(=O)NCc1cc2cc(Cl)ccc2[nH]1)[C@@H]1CCCN(C(=O)c2cccn3cnnc23)C1] | (R)-1-(1-([1,2,4]triazolo[4,3- a]pyridine-8-carbonyl)piperidin-3- yl)-3-((5-chloro-1H-indol-2- yl)methyl)-1-methylurea |
| 75 | 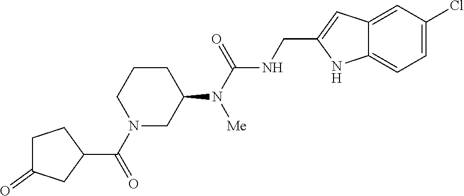 [SMILES: CN(C(=O)NCc1cc2cc(Cl)ccc2[nH]1)[C@@H]1CCCN(C(=O)C2CCC(=O)C2)C1] | 3-((5-chloro-1H-indol-2- yl)methyl)-1-methyl-1-((3R)-1-(3- oxocyclopentane-1- carbonyl)piperidin-3-yl)urea |
| 76 | 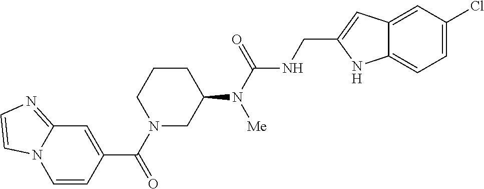 [SMILES: CN(C(=O)NCc1cc2cc(Cl)ccc2[nH]1)[C@@H]1CCCN(C(=O)c2ccn3ccnc3c2)C1] | (R)-3-((5-chloro-1H-indol-2- yl)methyl)-1-(1-(imidazo[1,2- a]pyridine-7-carbonyl)piperidin-3- yl)-1-methylurea |
| 77 | 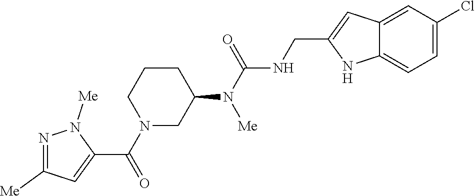 [SMILES: Cc1cc(C(=O)N2CCC[C@@H](N(C)C(=O)NCc3cc4cc(Cl)ccc4[nH]3)C2)n(C)n1] | (R)-3-((5-chloro-1H-indol-2- yl)methyl)-1-(1-(1,3-dimethyl-1H- pyrazole-5-carbonyl)piperidin-3- yl)-1-methylurea |
| 78 | 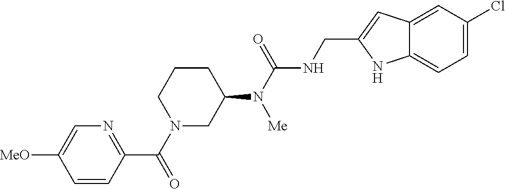 [SMILES: COc1ccc(C(=O)N2CCC[C@@H](N(C)C(=O)NCc3cc4cc(Cl)ccc4[nH]3)C2)nc1] | (R)-3-((5-chloro-1H-indol-2- yl)methyl)-1-(1-(5- methoxypicolinoyl)piperidin-3- yl)-1-methylurea |
| 79 | 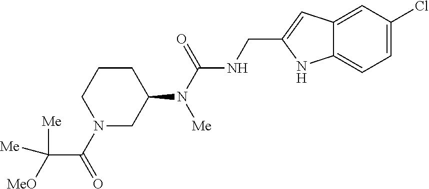 [SMILES: COC(C)(C)C(=O)N1CCC[C@@H](N(C)C(=O)NCc2cc3cc(Cl)ccc3[nH]2)C1] | (R)-3-((5-chloro-1H-indol-2- yl)methyl)-1-(1-(2-methoxy-2- methylpropanoyl)piperidin-3-yl)- 1-methylurea |
| 80 | 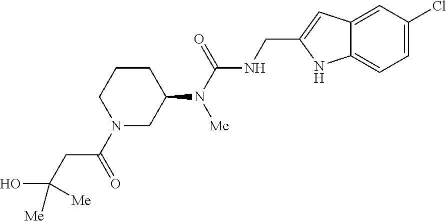 [SMILES: CN(C(=O)NCc1cc2cc(Cl)ccc2[nH]1)[C@@H]1CCCN(C(=O)CC(C)(C)O)C1] | (R)-3-((5-chloro-1H-indol-2- yl)methyl)-1-(1-(3-hydroxy-3- methylbutanoyl)piperidin-3-yl)-1- methylurea |
| 81 | 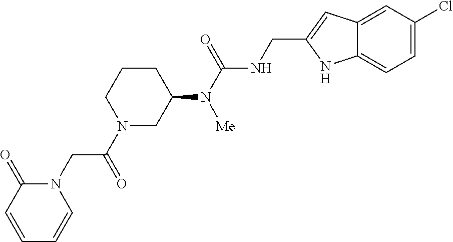 [SMILES: CN(C(=O)NCc1cc2cc(Cl)ccc2[nH]1)[C@@H]1CCCN(C(=O)Cn2ccccc2=O)C1] | (R)-3-((5-chloro-1H-indol-2- yl)methyl)-1-methyl-1-(1-(2-(2- oxopyridin-1(2H)- yl)acetyl)piperidin-3-yl)urea |
| 82 | 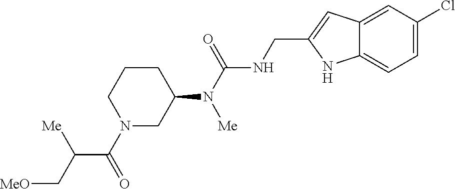 [SMILES: COCC(C)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(3-methoxy- 2-methylpropanoyl)piperidin-3- yl)-1-methylurea |
| 83 | 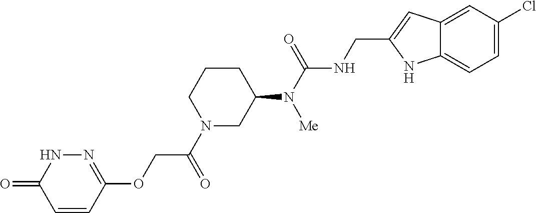 [SMILES: CN(C(=O)NCc1cc2cc(Cl)ccc2[nH]1)[C@@H]1CCCN(C(=O)COc2ccc(=O)[nH]n2)C1] | (R)-3-((5-chloro-1H-indol-2- yl)methyl)-1-methyl-1-(1-(2-((6- oxo-1,6-dihydropyridazin-3- yl)oxy)acetyl)piperidin-3-yl)urea |
| 84 | 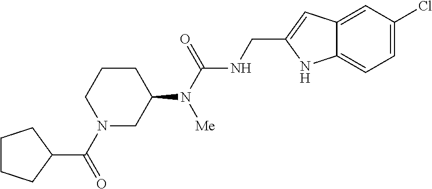 [SMILES: CN(C(=O)NCc1cc2cc(Cl)ccc2[nH]1)[C@@H]1CCCN(C(=O)C2CCCC2)C1] | (R)-3-((5-chloro-1H-indol-2- yl)methyl)-1-(1- (cyclopentanecarbonyl)piperidin- 3-yl)-1-methylurea |
| 85 | 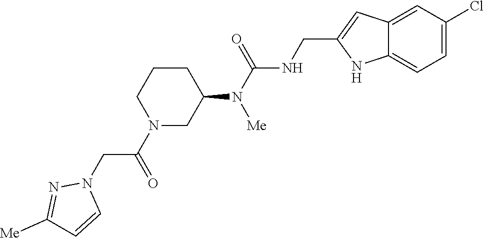 [SMILES: Cc1ccn(CC(=O)N2CCC[C@@H](N(C)C(=O)NCc3cc4cc(Cl)ccc4[nH]3)C2)n1] | (R)-3-((5-chloro-1H-indol-2- yl)methyl)-1-methyl-1-(1-(2-(3- methyl-1H-pyrazol-1- yl)acetyl)piperidin-3-yl)urea |
| 86 | 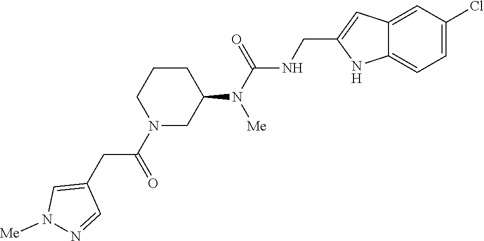 [SMILES: CN(C(=O)NCc1cc2cc(Cl)ccc2[nH]1)[C@@H]1CCCN(C(=O)Cc2cnn(C)c2)C1] | (R)-3-((5-chloro-1H-indol-2- yl)methyl)-1-methyl-1-(1-(2-(1- methyl-1H-pyrazol-4- yl)acetyl)piperidin-3-yl)urea |
| 87 | 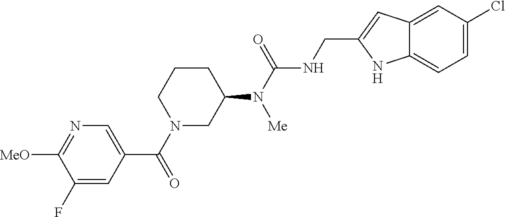 [SMILES: COc1ncc(C(=O)N2CCC[C@@H](N(C)C(=O)NCc3cc4cc(Cl)ccc4[nH]3)C2)cc1F] | (R)-3-((5-chloro-1H-indol-2- yl)methyl)-1-(1-(5-fluoro-6- methoxynicotinoyl)piperidin-3- yl)-1-methylurea |
| 88 | 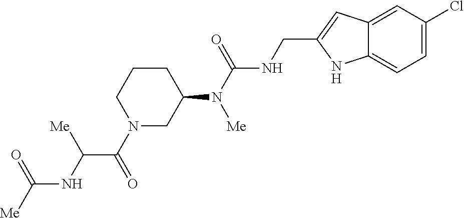 [SMILES: CC(=O)NC(C)C(=O)N1CCC[C@@H](N(C)C(=O)NCc2cc3cc(Cl)ccc3[nH]2)C1] | N-(1-((R)-3-(3-((5-chloro-1H- indol-2-yl)methyl)-1- methylureido)piperidin-1-yl)-1- oxopropan-2-yl)acetamide |
| 89 | 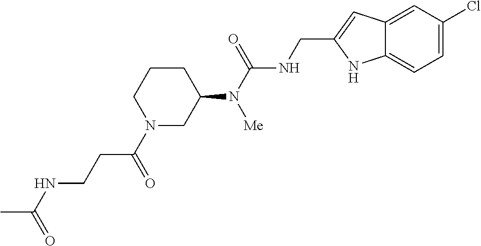 [SMILES: CC(=O)NCCC(=O)N1CCC[C@@H](N(C)C(=O)NCc2cc3cc(Cl)ccc3[nH]2)C1] | (R)-N-(3-(3-(3-((5-chloro-1H- indol-2-yl)methyl)-1- methylureido)piperidin-1-yl)-3- oxopropyl)acetamide |
| 90 | 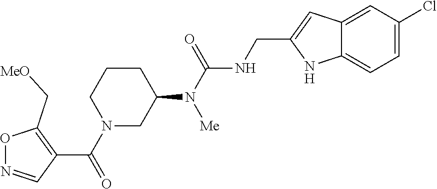 [SMILES: COCc1oncc1C(=O)N1CCC[C@@H](N(C)C(=O)NCc2cc3cc(Cl)ccc3[nH]2)C1] | (R)-3-((5-chloro-1H-indol-2- yl)methyl)-1-(1-(5- (methoxymethyl)isoxazole-4- carbonyl)piperidin-3-yl)-1- methylurea |
| 91 | 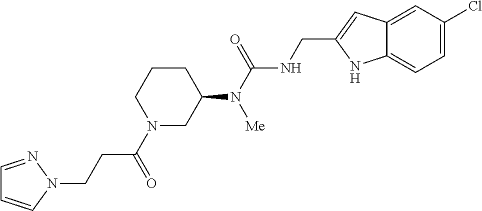 [SMILES: CN(C(=O)NCc1cc2cc(Cl)ccc2[nH]1)[C@@H]1CCCN(C(=O)CCn2cccn2)C1] | (R)-1-(1-(3-(1H-pyrazol-1- yl)propanoyl)piperidin-3-yl)-3-((5- chloro-1H-indol-2-yl)methyl)-1- methylurea |
| 92 | 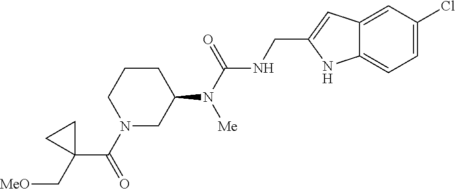 [SMILES: COCC1(C(=O)N2CCC[C@@H](N(C)C(=O)NCc3cc4cc(Cl)ccc4[nH]3)C2)CC1] | (R)-3-((5-chloro-1H-indol-2- yl)methyl)-1-(1-(1- (methoxymethyl)cyclopropane-1- carbonyl)piperidin-3-yl)-1- methylurea |
| 93 | 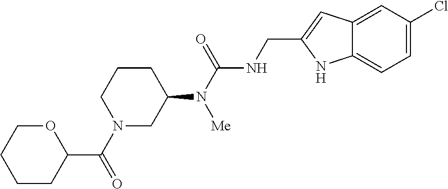 [SMILES: CN(C(=O)NCc1cc2cc(Cl)ccc2[nH]1)[C@@H]1CCCN(C(=O)C2CCCCO2)C1] | 3-((5-chloro-1H-indol-2- yl)methyl)-1-methyl-1-((3R)-1- (tetrahydro-2H-pyran-2- carbonyl)piperidin-3-yl)urea |
| 94 | 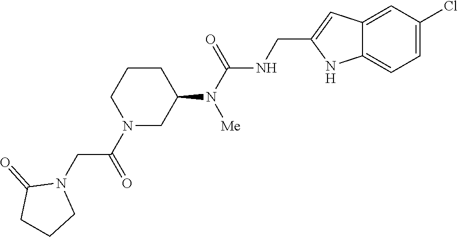 [SMILES: CN(C(=O)NCc1cc2cc(Cl)ccc2[nH]1)[C@@H]1CCCN(C(=O)CN2CCCC2=O)C1] | (R)-3-((5-chloro-1H-indol-2- yl)methyl)-1-methyl-1-(1-(2-(2- oxopyrrolidin-1- yl)acetyl)piperidin-3-yl)urea |
| 95 | 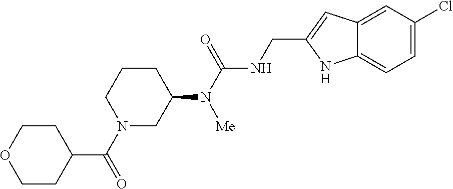 [SMILES: CN(C(=O)NCc1cc2cc(Cl)ccc2[nH]1)[C@@H]1CCCN(C(=O)C2CCOCC2)C1] | (R)-3-((5-chloro-1H-indol-2- yl)methyl)-1-methyl-1-(1- (tetrahydro-2H-pyran-4- carbonyl)piperidin-3-yl)urea |
| 96 | 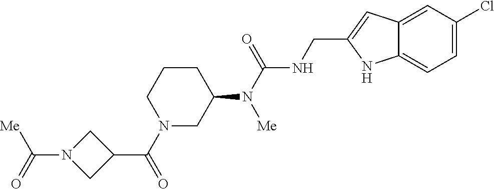 [SMILES: CC(=O)N1CC(C(=O)N2CCC[C@@H](N(C)C(=O)NCc3cc4cc(Cl)ccc4[nH]3)C2)C1] | (R)-1-(1-(1-acetylazetidine-3- carbonyl)piperidin-3-yl)-3-((5- chloro-1H-indol-2-yl)methyl)-1- methylurea |
| 97 | 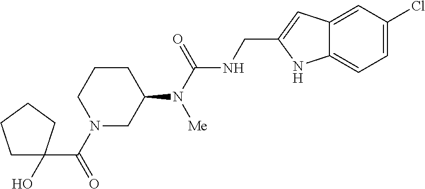 [SMILES: CN(C(=O)NCc1cc2cc(Cl)ccc2[nH]1)[C@@H]1CCCN(C(=O)C2(O)CCCC2)C1] | (R)-3-((5-chloro-1H-indol-2- yl)methyl)-1-(1-(1- hydroxycyclopentane-1- carbonyl)piperidin-3-yl)-1- methylurea |
| 98 | 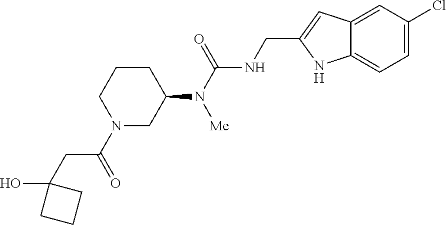 [SMILES: CN(C(=O)NCc1cc2cc(Cl)ccc2[nH]1)[C@@H]1CCCN(C(=O)CC2(O)CCC2)C1] | (R)-3-((5-chloro-1H-indol-2- yl)methyl)-1-(1-(2-(1- hydroxycyclobutyl)acetyl)piperidin- 3-yl)-1-methylurea |
| 99 | 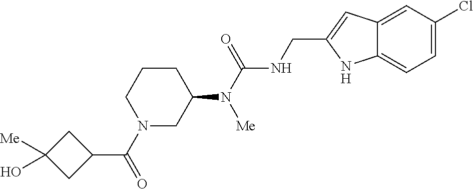 [SMILES: CN(C(=O)NCc1cc2cc(Cl)ccc2[nH]1)[C@@H]1CCCN(C(=O)C2CC(C)(O)C2)C1] | (R)-3-((5-chloro-1H-indol-2- yl)methyl)-1-(1-(3-hydroxy-3- methylcyclobutane-1- carbonyl)piperidin-3-yl)-1- methylurea |
| 100 | 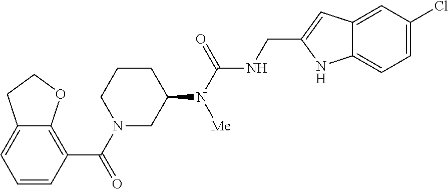 [SMILES: CN(C(=O)NCc1cc2cc(Cl)ccc2[nH]1)[C@@H]1CCCN(C(=O)c2cccc3c2OCC3)C1] | (R)-3-((5-chloro-1H-indol-2- yl)methyl)-1-(1-(2,3- dihydrobenzofuran-7- carbonyl)piperidin-3-yl)-1- methylurea |
| 101 | 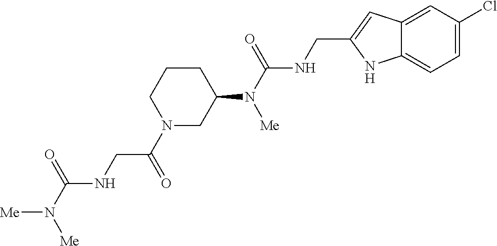 [SMILES: CN(C)C(=O)NCC(=O)N1CCC[C@@H](N(C)C(=O)NCc2cc3cc(Cl)ccc3[nH]2)C1] | (R)-3-((5-chloro-1H-indol-2- yl)methyl)-1-(1- ((dimethylcarbamoyl)glycyl) piperidin-3-yl)-1-methylurea |
| 102 | 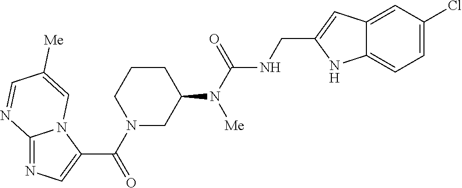 [SMILES: Cc1cnc2ncc(C(=O)N3CCC[C@@H](N(C)C(=O)NCc4cc5cc(Cl)ccc5[nH]4)C3)n2c1] | (R)-3-((5-chloro-1H-indol-2- yl)methyl)-1-methyl-1-(1-(6- methylimidazo[1,2-a]pyrimidine- 3-carbonyl)piperidin-3-yl)urea |
| 103 | 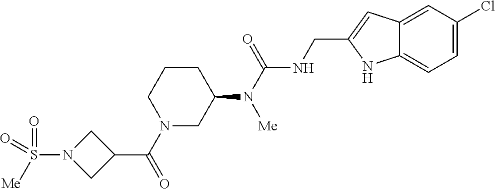 [SMILES: CN(C(=O)NCc1cc2cc(Cl)ccc2[nH]1)[C@@H]1CCCN(C(=O)C2CN(S(C)(=O)=O)C2)C1] | (R)-3-((5-chloro-1H-indol-2- yl)methyl)-1-methyl-1-(1-(1- (methylsulfonyl)azetidine-3- carbonyl)piperidin-3-yl)urea |
| 104 | 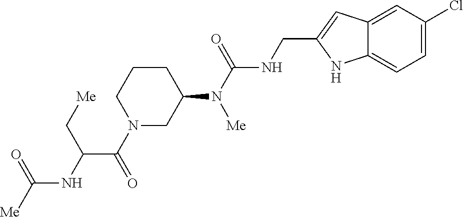 [SMILES: CCC(NC(C)=O)C(=O)N1CCC[C@@H](N(C)C(=O)NCc2cc3cc(Cl)ccc3[nH]2)C1] | N-(1-((R)-3-(3-((5-chloro-1H- indol-2-yl)methyl)-1- methylureido)piperidin-1-yl)-1- oxobutan-2-yl)acetamide |
| 105 | 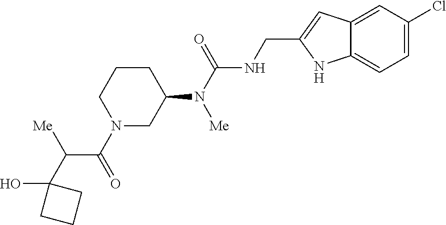 [SMILES: CC(C(=O)N1CCC[C@@H](N(C)C(=O)NCc2cc3cc(Cl)ccc3[nH]2)C1)C1(O)CCC1] | 3-((5-chloro-1H-indol-2- yl)methyl)-1-((3R)-1-(2-(1- hydroxycyclobutyl)propanoyl) piperidin-3-yl)-1-methylurea |
| 106 | 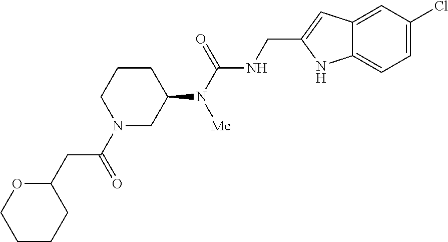 [SMILES: CN(C(=O)NCc1cc2cc(Cl)ccc2[nH]1)[C@@H]1CCCN(C(=O)CC2CCCCO2)C1] | 3-((5-chloro-1H-indol-2- yl)methyl)-1-methyl-1-((3R)-1-(2- (tetrahydro-2H-pyran-2- yl)acetyl)piperidin-3-yl)urea |
| 107 | 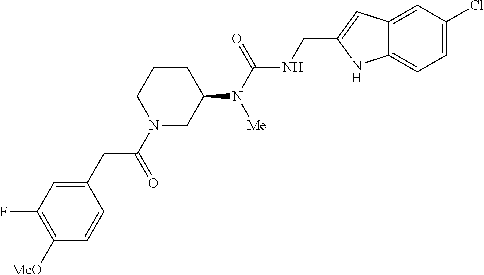 [SMILES: COc1ccc(CC(=O)N2CCC[C@@H](N(C)C(=O)NCc3cc4cc(Cl)ccc4[nH]3)C2)cc1F] | (R)-3-((5-chloro-1H-indol-2- yl)methyl)-1-(1-(2-(3-fluoro-4- methoxyphenyl)acetyl)piperidin-3- yl)-1-methylurea |
| 108 | 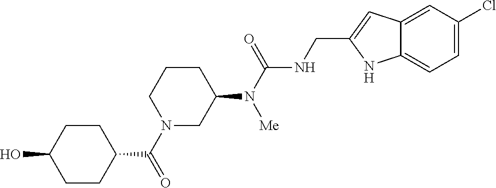 [SMILES: CN(C(=O)NCc1cc2cc(Cl)ccc2[nH]1)[C@@H]1CCCN(C(=O)[C@H]2CC[C@H](O)CC2)C1] | trans-3-((5-chloro-1H-indol-2- yl)methyl)-1-((R)-1-((4- hydroxycyclohexane-1- carbonyl)piperidin-3-yl)-1- methylurea |
| 109 | 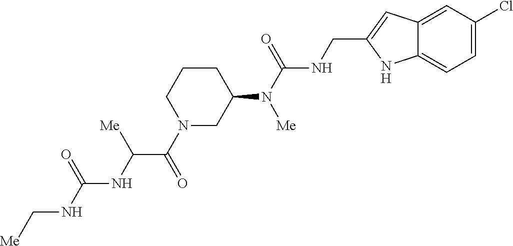 [SMILES: CCNC(=O)NC(C)C(=O)N1CCC[C@@H](N(C)C(=O)NCc2cc3cc(Cl)ccc3[nH]2)C1] | 3-((5-chloro-1H-indol-2- yl)methyl)-1-((3R)-1- ((ethylcarbamoyl)alanyl)piperidin- 3-yl)-1-methylurea |
| 110 | 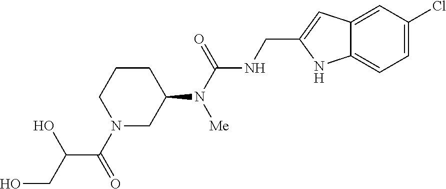 [SMILES: CN(C(=O)NCc1cc2cc(Cl)ccc2[nH]1)[C@@H]1CCCN(C(=O)C(O)CO)C1] | 3-((5-chloro-1H-indol-2- yl)methyl)-1-((3R)-1-(2,3- dihydroxypropanoyl)piperidin-3- yl)-1-methylurea |
| 111 | 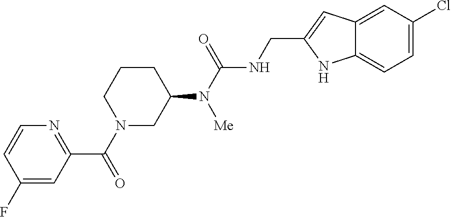 [SMILES: CN(C(=O)NCc1cc2cc(Cl)ccc2[nH]1)[C@@H]1CCCN(C(=O)c2cc(F)ccn2)C1] | (R)-3-((5-chloro-1H-indol-2- yl)methyl)-1-(1-(4- fluoropicolinoyl)piperidin-3-yl)-1- methylurea |
| 112 | 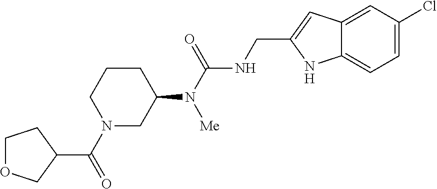 [SMILES: CN(C(=O)NCc1cc2cc(Cl)ccc2[nH]1)[C@@H]1CCCN(C(=O)C2CCOC2)C1] | 3-((5-chloro-1H-indol-2- yl)methyl)-1-methyl-1-((3R)-1- (tetrahydrofuran-3- carbonyl)piperidin-3-yl)urea |
| 113 | 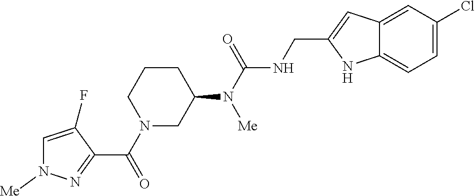 [SMILES: CN(C(=O)NCc1cc2cc(Cl)ccc2[nH]1)[C@@H]1CCCN(C(=O)c2nn(C)cc2F)C1] | (R)-3-((5-chloro-1H-indol-2- yl)methyl)-1-(1-(4-fluoro-1- methyl-1H-pyrazole-3- carbonyl)piperidin-3-yl)-1- methylurea |
| 114 | 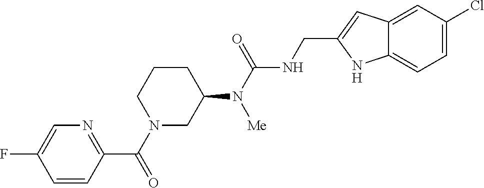 [SMILES: CN(C(=O)NCc1cc2cc(Cl)ccc2[nH]1)[C@@H]1CCCN(C(=O)c2ccc(F)cn2)C1] | (R)-3-((5-chloro-1H-indol-2- yl)methyl)-1-(1-(5- fluoropicolinoyl)piperidin-3-yl)-1- methylurea |
| 115 | 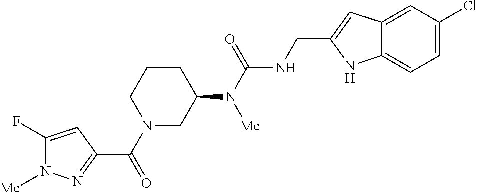 [SMILES: CN(C(=O)NCc1cc2cc(Cl)ccc2[nH]1)[C@@H]1CCCN(C(=O)c2cc(F)n(C)n2)C1] | (R)-3-((5-chloro-1H-indol-2- yl)methyl)-1-(1-(5-fluoro-1- methyl-1H-pyrazole-3- carbonyl)piperidin-3-yl)-1- methylurea |
| 116 | 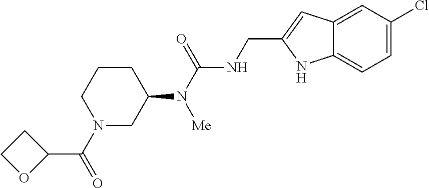 [SMILES: CN(C(=O)NCc1cc2cc(Cl)ccc2[nH]1)[C@@H]1CCCN(C(=O)C2CCO2)C1] | 3-((5-chloro-1H-indol-2- yl)methyl)-1-methyl-1-((3R)-1- (oxetane-2-carbonyl)piperidin-3- yl)urea |
| 117 | 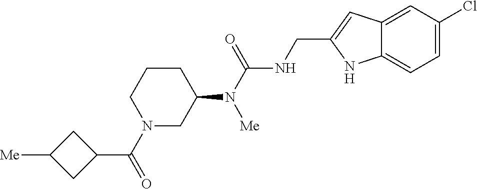 [SMILES: CC1CC(C(=O)N2CCC[C@@H](N(C)C(=O)NCc3cc4cc(Cl)ccc4[nH]3)C2)C1] | (R)-3-((5-chloro-1H-indol-2- yl)methyl)-1-methyl-1-(1-(3- methylcyclobutane-1- carbonyl)piperidin-3-yl)urea |
| 118 | 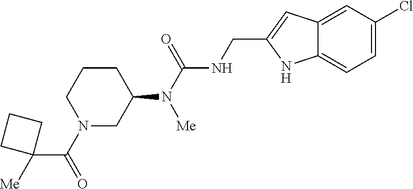 [SMILES: CN(C(=O)NCc1cc2cc(Cl)ccc2[nH]1)[C@@H]1CCCN(C(=O)C2(C)CCC2)C1] | (R)-3-((5-chloro-1H-indol-2- yl)methyl)-1-methyl-1-(1-(1- methylcyclobutane-1- carbonyl)piperidin-3-yl)urea |
| 119 | 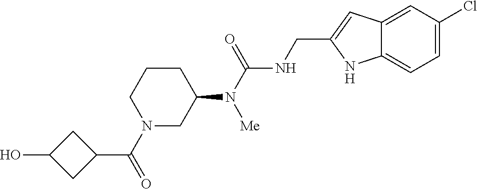 [SMILES: CN(C(=O)NCc1cc2cc(Cl)ccc2[nH]1)[C@@H]1CCCN(C(=O)C2CC(O)C2)C1] | (R)-3-((5-chloro-1H-indol-2- yl)methyl)-1-(1-(3- hydroxycyclobutane-1- carbonyl)piperidin-3-yl)-1- methylurea |
| 120 | 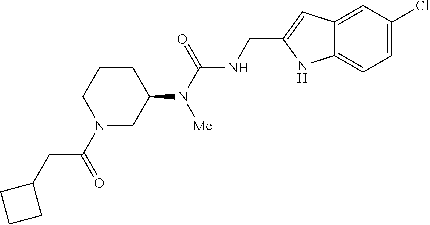 [SMILES: CN(C(=O)NCc1cc2cc(Cl)ccc2[nH]1)[C@@H]1CCCN(C(=O)CC2CCC2)C1] | (R)-3-((5-chloro-1H-indol-2- yl)methyl)-1-(1-(2- cyclobutylacetyl)piperidin-3-yl)-1- methylurea |
| 121 | 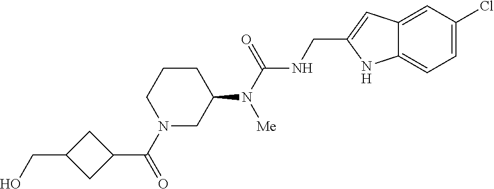 [SMILES: CN(C(=O)NCc1cc2cc(Cl)ccc2[nH]1)[C@@H]1CCCN(C(=O)C2CC(CO)C2)C1] | (R)-3-((5-chloro-1H-indol-2- yl)methyl)-1-(1-(3- (hydroxymethyl)cyclobutane-1- carbonyl)piperidin-3-yl)-1- methylurea |
| 122 | 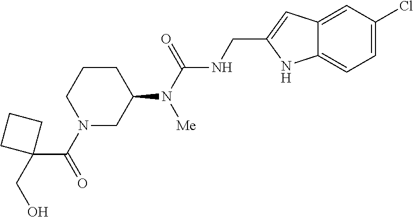 [SMILES: CN(C(=O)NCc1cc2cc(Cl)ccc2[nH]1)[C@@H]1CCCN(C(=O)C2(CO)CCC2)C1] | (R)-3-((5-chloro-1H-indol-2- yl)methyl)-1-(1-(1- (hydroxymethyl)cyclobutane-1- carbonyl)piperidin-3-yl)-1- methylurea |
| 123 | 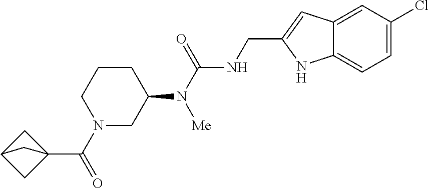 [SMILES: CN(C(=O)NCc1cc2cc(Cl)ccc2[nH]1)[C@@H]1CCCN(C(=O)C23CC(C2)C3)C1] | (R)-1-(1-(bicyclo[1.1.1]pentane-1- carbonyl)piperidin-3-yl)-3-((5- chloro-1H-indol-2-yl)methyl)-1- methylurea |
| 124 | 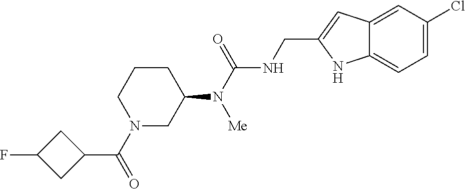 [SMILES: CN(C(=O)NCc1cc2cc(Cl)ccc2[nH]1)[C@@H]1CCCN(C(=O)C2CC(F)C2)C1] | (R)-3-((5-chloro-1H-indol-2- yl)methyl)-1-(1-(3- fluorocyclobutane-1- carbonyl)piperidin-3-yl)-1- methylurea |
| 125 | 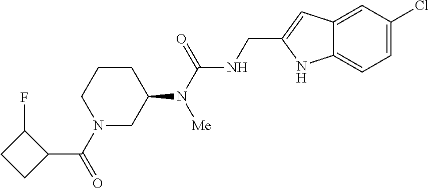 [SMILES: CN(C(=O)NCc1cc2cc(Cl)ccc2[nH]1)[C@@H]1CCCN(C(=O)C2CCC2F)C1] | 3-((5-chloro-1H-indol-2- yl)methyl)-1-((3R)-1-(2- fluorocyclobutane-1- carbonyl)piperidin-3-yl)-1- methylurea |
| 126 | 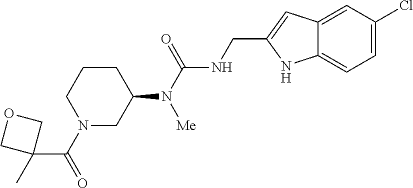 [SMILES: CN(C(=O)NCc1cc2cc(Cl)ccc2[nH]1)[C@@H]1CCCN(C(=O)C2(C)COC2)C1] | (R)-3-((5-chloro-1H-indol-2- yl)methyl)-1-methyl-1-(1-(3- methyloxetane-3- carbonyl)piperidin-3-yl)urea |
| 127 | 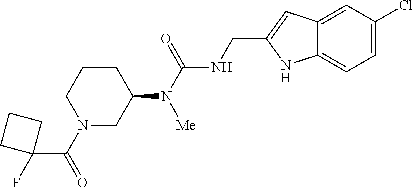 [SMILES: CN(C(=O)NCc1cc2cc(Cl)ccc2[nH]1)[C@@H]1CCCN(C(=O)C2(F)CCC2)C1] | (R)-3-((5-chloro-1H-indol-2- yl)methyl)-1-(1-(1- fluorocyclobutane-1- carbonyl)piperidin-3-yl)-1- methylurea |
| 128 | 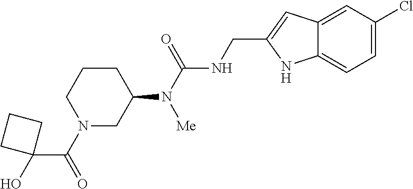 [SMILES: CN(C(=O)NCc1cc2cc(Cl)ccc2[nH]1)[C@@H]1CCCN(C(=O)C2(O)CCC2)C1] | (R)-3-((5-chloro-1H-indol-2- yl)methyl)-1-(1-(1- hydroxycyclobutane-1- carbonyl)piperidin-3-yl)-1- methylurea |
| 129 | 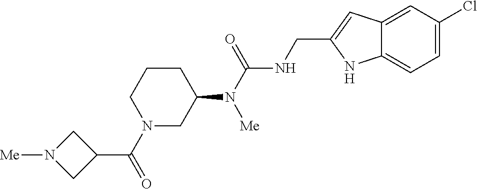 [SMILES: CN1CC(C(=O)N2CCC[C@@H](N(C)C(=O)NCc3cc4cc(Cl)ccc4[nH]3)C2)C1] | (R)-3-((5-chloro-1H-indol-2- yl)methyl)-1-methyl-1-(1-(1- methylazetidine-3- carbonyl)piperidin-3-yl)urea |
| 130 | 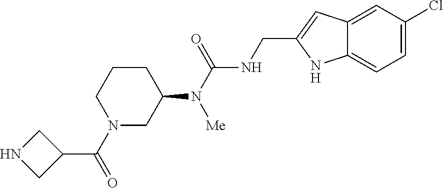 [SMILES: CN(C(=O)NCc1cc2cc(Cl)ccc2[nH]1)[C@@H]1CCCN(C(=O)C2CNC2)C1] | (R)-1-(1-(azetidine-3- carbonyl)piperidin-3-yl)-3-((5- chloro-1H-indol-2-yl)methyl)-1- methylurea |
| 131 | 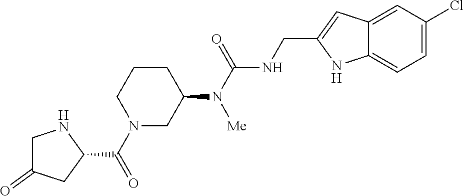 [SMILES: CN(C(=O)NCc1cc2cc(Cl)ccc2[nH]1)[C@@H]1CCCN(C(=O)[C@@H]2CC(=O)CN2)C1] | 3-((5-chloro-1H-indol-2- yl)methyl)-1-methyl-1-((R)-1-((S)- 4-oxopyrrolidine-2- carbonyl)piperidin-3-yl)urea |
| 132 | 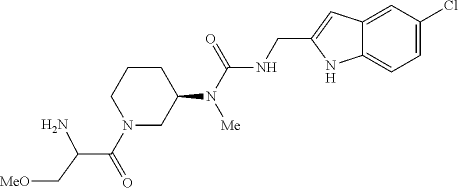 [SMILES: COCC(N)C(=O)N1CCC[C@@H](N(C)C(=O)NCc2cc3cc(Cl)ccc3[nH]2)C1] | 3-((5-chloro-1H-indol-2- yl)methyl)-1-methyl-1-((3R)-1-(O- methylseryl)piperidin-3-yl)urea |
| 133 | 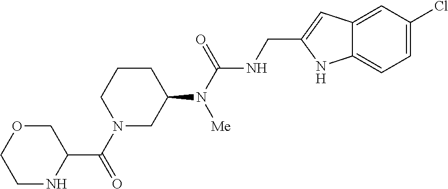 [SMILES: CN(C(=O)NCc1cc2cc(Cl)ccc2[nH]1)[C@@H]1CCCN(C(=O)C2COCCN2)C1] | 3-((5-chloro-1H-indol-2- yl)methyl)-1-methyl-1-((3R)-1- (morpholine-3-carbonyl)piperidin- 3-yl)urea |
| 134 | 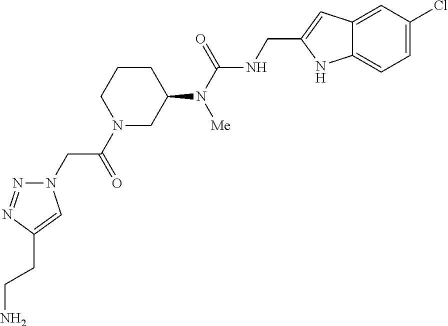 [SMILES: CN(C(=O)NCc1cc2cc(Cl)ccc2[nH]1)[C@@H]1CCCN(C(=O)Cn2cc(CCN)nn2)C1] | (R)-1-(1-(2-(4-(2-aminoethyl)-1H- 1,2,3-triazol-1-yl)acetyl)piperidin- 3-yl)-3-((5-chloro-1H-indol-2- yl)methyl)-1-methylurea |
| 135 | 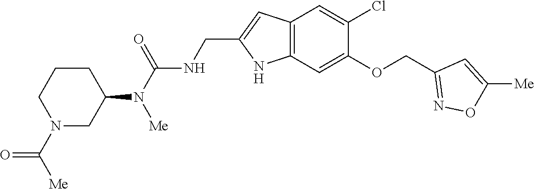 [SMILES: CC(=O)N1CCC[C@@H](N(C)C(=O)NCc2cc3cc(Cl)c(OCc4cc(C)on4)cc3[nH]2)C1] | (R)-1-(1-acetylpiperidin-3-yl)-3- ((5-chloro-6-((5-methylisoxazol-3- yl)methoxy)-1H-indol-2- yl)methyl)-1-methylurea |
| 136 | 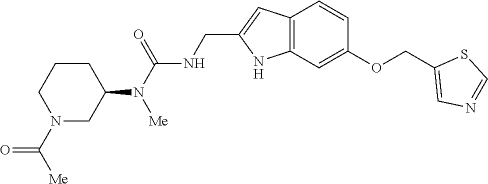 [SMILES: CC(=O)N1CCC[C@@H](N(C)C(=O)NCc2cc3ccc(OCc4cncs4)cc3[nH]2)C1] | (R)-1-(1-acetylpiperidin-3-yl)-1- methyl-3-((6-(thiazol-5- ylmethoxy)-1H-indol-2- yl)methyl)urea |
| 137 | 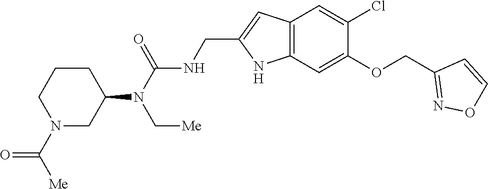 [SMILES: CCN(C(=O)NCc1cc2cc(Cl)c(OCc3ccon3)cc2[nH]1)[C@@H]1CCCN(C(C)=O)C1] | (R)-1-(1-acetylpiperidin-3-yl)-3- ((5-chloro-6-(isoxazol-3- ylmethoxy)-1H-indol-2- yl)methyl)-1-ethylurea |
| 138 | 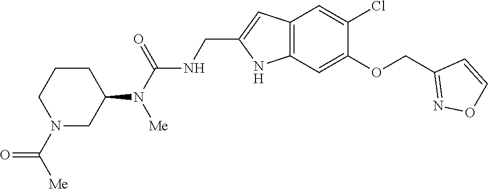 [SMILES: CC(=O)N1CCC[C@@H](N(C)C(=O)NCc2cc3cc(Cl)c(OCc4ccon4)cc3[nH]2)C1] | (R)-1-(1-acetylpiperidin-3-yl)-3- ((5-chloro-6-(isoxazol-3- ylmethoxy)-1H-indol-2- yl)methyl)-1-methylurea |
| 139 | 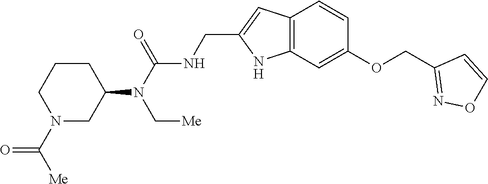 [SMILES: CCN(C(=O)NCc1cc2ccc(OCc3ccon3)cc2[nH]1)[C@@H]1CCCN(C(C)=O)C1] | (R)-1-(1-acetylpiperidin-3-yl)-1- ethyl-3-((6-(isoxazol-3- ylmethoxy)-1H-indol-2- yl)methyl)urea |
| 140 | 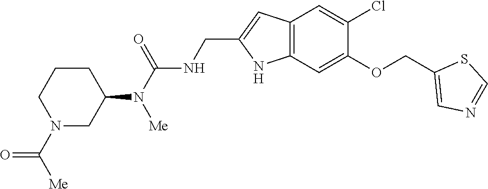 [SMILES: CC(=O)N1CCC[C@@H](N(C)C(=O)NCc2cc3cc(Cl)c(OCc4cncs4)cc3[nH]2)C1] | (R)-1-(1-acetylpiperidin-3-yl)-3- ((5-chloro-6-(thiazol-5- ylmethoxy)-1H-indol-2- yl)methyl)-1-methylurea |
| 141 | 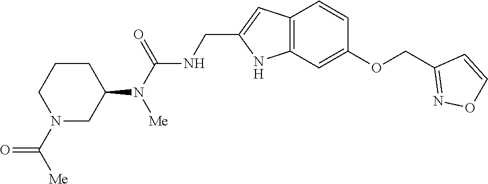 [SMILES: CC(=O)N1CCC[C@@H](N(C)C(=O)NCc2cc3ccc(OCc4ccon4)cc3[nH]2)C1] | (R)-1-(1-acetylpiperidin-3-yl)-3- ((6-(isoxazol-3-ylmethoxy)-1H- indol-2-yl)methyl)-1-methylurea |
| 142 | 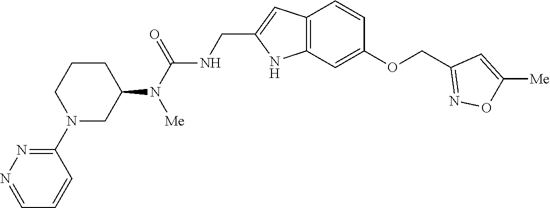 [SMILES: Cc1cc(COc2ccc3cc(CNC(=O)N(C)[C@@H]4CCCN(c5cccnn5)C4)[nH]c3c2)no1] | (R)-1-methyl-3-((6-((5- methylisoxazol-3-yl)methoxy)- 1H-indol-2-yl)methyl)-1-(1- (pyridazin-3-yl)piperidin-3-yl)urea |
| 143 | 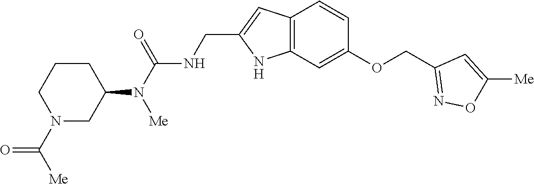 [SMILES: CC(=O)N1CCC[C@@H](N(C)C(=O)NCc2cc3ccc(OCc4cc(C)on4)cc3[nH]2)C1] | (R)-1-(1-acetylpiperidin-3-yl)-1- methyl-3-((6-((5-methylisoxazol- 3-yl)methoxy)-1H-indol-2- yl)methyl)urea |
| 144 | 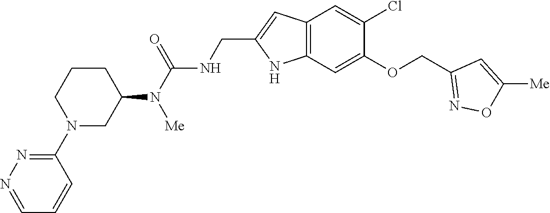 [SMILES: Cc1cc(COc2cc3[nH]c(CNC(=O)N(C)[C@@H]4CCCN(c5cccnn5)C4)cc3cc2Cl)no1] | (R)-3-((5-chloro-6-((5- methylisoxazol-3-yl)methoxy)- 1H-indol-2-yl)methyl)-1-methyl- 1-(1-(pyridazin-3-yl)piperidin-3- yl)urea |
| 145 | 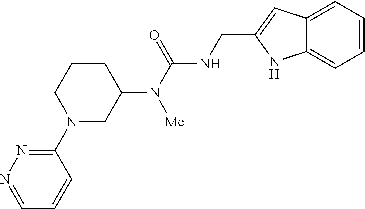 [SMILES: CN(C(=O)NCc1cc2ccccc2[nH]1)C1CCCN(c2cccnn2)C1] | 3-((1H-indol-2-yl)methyl)-1- methyl-1-(1-(pyridazin-3- yl)piperidin-3-yl)urea |
| 146 | 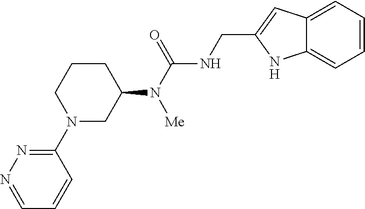 [SMILES: CN(C(=O)NCc1cc2ccccc2[nH]1)[C@@H]1CCCN(c2cccnn2)C1] | (R)-3-((1H-indol-2-yl)methyl)-1- methyl-1-(1-(pyridazin-3- yl)piperidin-3-yl)urea |
| 147 | 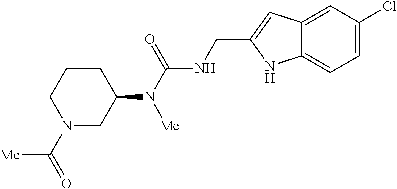 [SMILES: CC(=O)N1CCC[C@@H](N(C)C(=O)NCc2cc3cc(Cl)ccc3[nH]2)C1] | (R)-1-(1-acetylpiperidin-3-yl)-3- ((5-chloro-1H-indol-2-yl)methyl)- 1-methylurea |
| 148 | 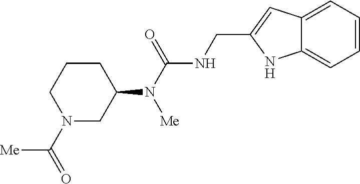 [SMILES: CC(=O)N1CCC[C@@H](N(C)C(=O)NCc2cc3ccccc3[nH]2)C1] | (R)-3-((1H-indol-2-yl)methyl)-1- (1-acetylpiperidin-3-yl)-1- methylurea |
| 149 | 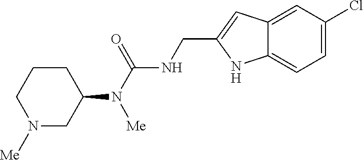 [SMILES: CN1CCC[C@@H](N(C)C(=O)NCc2cc3cc(Cl)ccc3[nH]2)C1] | (R)-3-((5-chloro-1H-indol-2- yl)methyl)-1-methyl-1-(1- methylpiperidin-3-yl)urea |
| 150 | 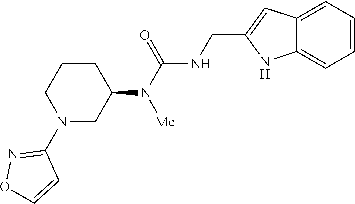 [SMILES: CN(C(=O)NCc1cc2ccccc2[nH]1)[C@@H]1CCCN(c2ccon2)C1] | (R)-3-((1H-indol-2-yl)methyl)-1- (1-(isoxazol-3-yl)piperidin-3-yl)- 1-methylurea |
| 151 | 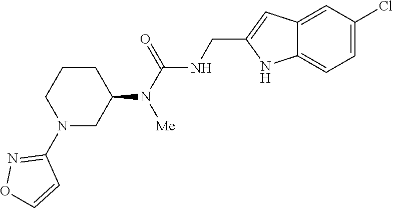 [SMILES: CN(C(=O)NCc1cc2cc(Cl)ccc2[nH]1)[C@@H]1CCCN(c2ccon2)C1] | (R)-3-((5-chloro-1H-indol-2- yl)methyl)-1-(1-(isoxazol-3- yl)piperidin-3-yl)-1-methylurea |
| 152 | 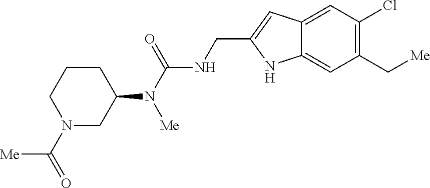 [SMILES: CCc1cc2[nH]c(CNC(=O)N(C)[C@@H]3CCCN(C(C)=O)C3)cc2cc1Cl] | (R)-1-(1-acetylpiperidin-3-yl)-3- ((5-chloro-6-ethyl-1H-indol-2- yl)methyl)-1-methylurea |
| 153 | 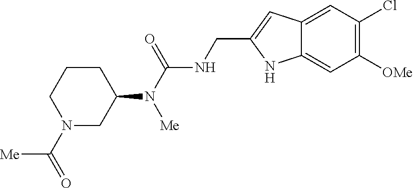 [SMILES: COc1cc2[nH]c(CNC(=O)N(C)[C@@H]3CCCN(C(C)=O)C3)cc2cc1Cl] | (R)-1-(1-acetylpiperidin-3-yl)-3- ((5-chloro-6-methoxy-1H-indol-2- yl)methyl)-1-methylurea |
| 154 | 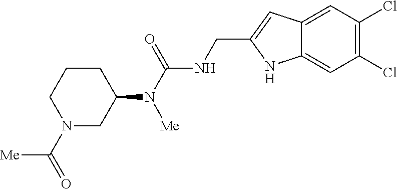 [SMILES: CC(=O)N1CCC[C@@H](N(C)C(=O)NCc2cc3cc(Cl)c(Cl)cc3[nH]2)C1] | (R)-1-(1-acetylpiperidin-3-yl)-3- ((5,6-dichloro-1H-indol-2- yl)methyl)-1-methylurea |
| 155 | 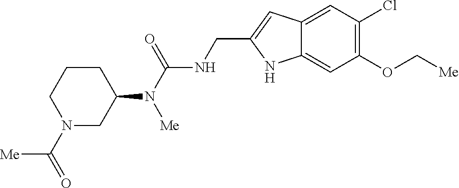 [SMILES: CCOc1cc2[nH]c(CNC(=O)N(C)[C@@H]3CCCN(C(C)=O)C3)cc2cc1Cl] | (R)-1-(1-acetylpiperidin-3-yl)-3- ((5-chloro-6-ethoxy-1H-indol-2- yl)methyl)-1-methylurea |
| 156 | 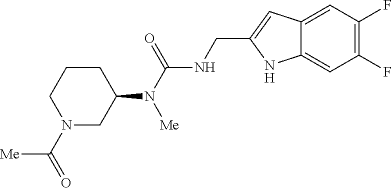 [SMILES: CC(=O)N1CCC[C@@H](N(C)C(=O)NCc2cc3cc(F)c(F)cc3[nH]2)C1] | (R)-1-(1-acetylpiperidin-3-yl)-3- ((5,6-difluoro-1H-indol-2- yl)methyl)-1-methylurea |
| 157 | 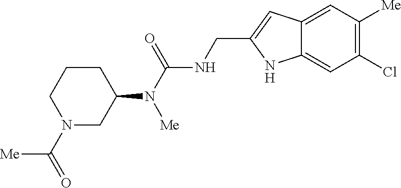 [SMILES: CC(=O)N1CCC[C@@H](N(C)C(=O)NCc2cc3cc(C)c(Cl)cc3[nH]2)C1] | (R)-1-(1-acetylpiperidin-3-yl)-3- ((6-chloro-5-methyl-1H-indol-2- yl)methyl)-1-methylurea |
| 158 | 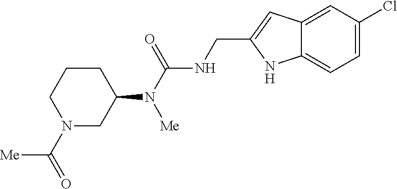 [SMILES: CC(=O)N1CCC[C@@H](N(C)C(=O)NCc2cc3cc(Cl)ccc3[nH]2)C1] | methyl (R)-3-(3-((5-chloro-1H- indol-2-yl)methyl)-1- methylureido)piperidine-1- carboxylate |
| 159 | 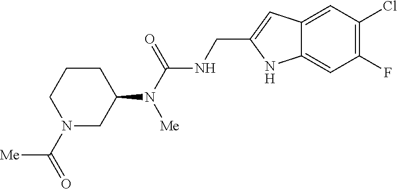 [SMILES: CC(=O)N1CCC[C@@H](N(C)C(=O)NCc2cc3cc(Cl)c(F)cc3[nH]2)C1] | (R)-1-(1-acetylpiperidin-3-yl)-3- ((5-chloro-6-fluoro-1H-indol-2- yl)methyl)-1-methylurea |
| 160 | 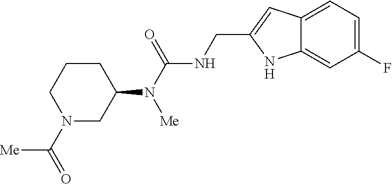 [SMILES: CC(=O)N1CCC[C@@H](N(C)C(=O)NCc2cc3ccc(F)cc3[nH]2)C1] | (R)-1-(1-acetylpiperidin-3-yl)-3- ((6-fluoro-1H-indol-2-yl)methyl)- 1-methylurea |
| 161 | 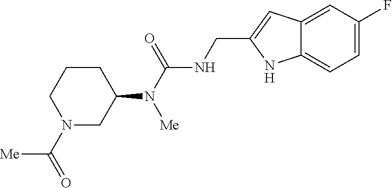 [SMILES: CC(=O)N1CCC[C@@H](N(C)C(=O)NCc2cc3cc(F)ccc3[nH]2)C1] | (R)-1-(1-acetylpiperidin-3-yl)-3- ((5-fluoro-1H-indol-2-yl)methyl)- 1-methylurea |
| 162 | 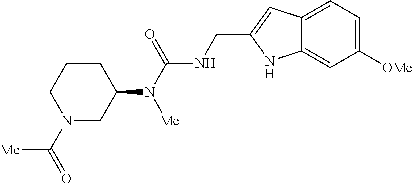 [SMILES: COc1ccc2cc(CNC(=O)N(C)[C@@H]3CCCN(C(C)=O)C3)[nH]c2c1] | (R)-1-(1-acetylpiperidin-3-yl)-3- ((6-methoxy-1H-indol-2- yl)methyl)-1-methylurea |
| 163 | 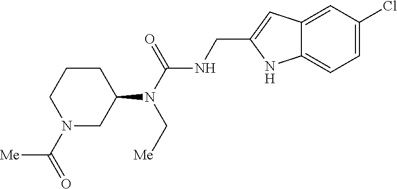 [SMILES: CCN(C(=O)NCc1cc2cc(Cl)ccc2[nH]1)[C@@H]1CCCN(C(C)=O)C1] | (R)-1-(1-acetylpiperidin-3-yl)-3- ((5-chloro-1H-indol-2-yl)methyl)- 1-ethylurea |
| 164 | 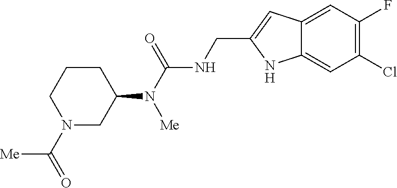 [SMILES: CC(=O)N1CCC[C@@H](N(C)C(=O)NCc2cc3cc(F)c(Cl)cc3[nH]2)C1] | (R)-1-(1-acetylpiperidin-3-yl)-3- ((6-chloro-5-fluoro-1H-indol-2- yl)methyl)-1-methylurea |
| 165 | 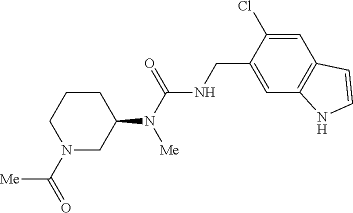 [SMILES: CC(=O)N1CCC[C@@H](N(C)C(=O)NCc2cc3[nH]ccc3cc2Cl)C1] | (R)-1-(1-acetylpiperidin-3-yl)-3- ((5-chloro-1H-indol-6-yl)methyl)- 1-methylurea |
| 166 | 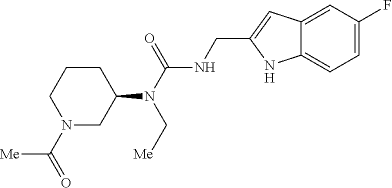 [SMILES: CCN(C(=O)NCc1cc2cc(F)ccc2[nH]1)[C@@H]1CCCN(C(C)=O)C1] | (R)-1-(1-acetylpiperidin-3-yl)-1- ethyl-3-((5-fluoro-1H-indol-2- yl)methyl)urea |
| 167 | 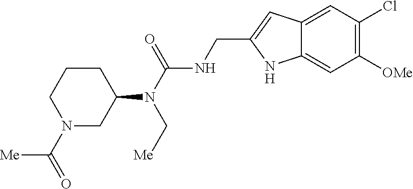 [SMILES: CCN(C(=O)NCc1cc2cc(Cl)c(OC)cc2[nH]1)[C@@H]1CCCN(C(C)=O)C1] | (R)-1-(1-acetylpiperidin-3-yl)-3- ((5-chloro-6-methoxy-1H-indol-2- yl)methyl)-1-ethylurea |
| 168 | 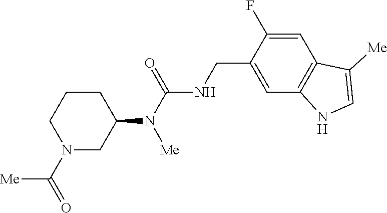 [SMILES: CC(=O)N1CCC[C@@H](N(C)C(=O)NCc2cc3[nH]cc(C)c3cc2F)C1] | (R)-1-(1-acetylpiperidin-3-yl)-3- ((5-fluoro-3-methyl-1H-indol-6- yl)methyl)-1-methylurea |
| 169 | 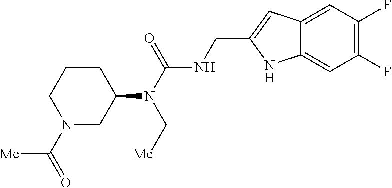 [SMILES: CCN(C(=O)NCc1cc2cc(F)c(F)cc2[nH]1)[C@@H]1CCCN(C(C)=O)C1] | (R)-1-(1-acetylpiperidin-3-yl)-3- ((5,6-difluoro-1H-indol-2- yl)methyl)-1-ethylurea |
| 170 | 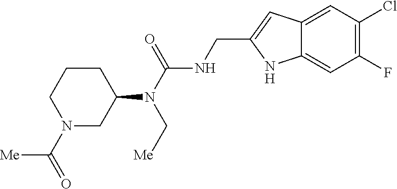 [SMILES: CCN(C(=O)NCc1cc2cc(Cl)c(F)cc2[nH]1)[C@@H]1CCCN(C(C)=O)C1] | (R)-1-(1-acetylpiperidin-3-yl)-3- ((5-chloro-6-fluoro-1H-indol-2- yl)methyl)-1-ethylurea |
| 171 | 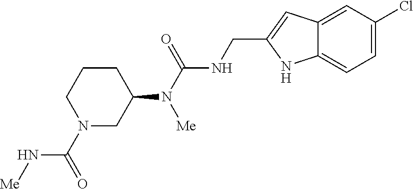 [SMILES: CNC(=O)N1CCC[C@@H](N(C)C(=O)NCc2cc3cc(Cl)ccc3[nH]2)C1] | (R)-3-(3-((5-chloro-1H-indol-2- yl)methyl)-1-methylureido)-N- methylpiperidine-1-carboxamide |
| 172 | 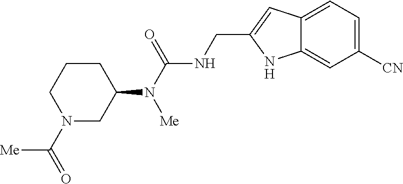 [SMILES: CC(=O)N1CCC[C@@H](N(C)C(=O)NCc2cc3ccc(C#N)cc3[nH]2)C1] | (R)-1-(1-acetylpiperidin-3-yl)-3- ((6-cyano-1H-indol-2-yl)methyl)- 1-methylurea |
| 173 | 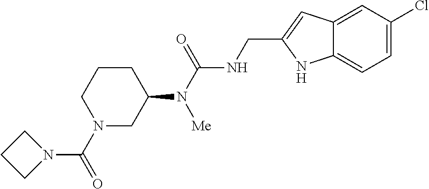 [SMILES: CN(C(=O)NCc1cc2cc(Cl)ccc2[nH]1)[C@@H]1CCCN(C(=O)N2CCC2)C1] | (R)-1-(1-(azetidine-1- carbonyl)piperidin-3-yl)-3-((5- chloro-1H-indol-2-yl)methyl)-1- methylurea |
| 174 | 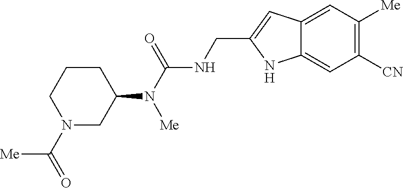 [SMILES: CC(=O)N1CCC[C@@H](N(C)C(=O)NCc2cc3cc(C)c(C#N)cc3[nH]2)C1] | (R)-1-(1-acetylpiperidin-3-yl)-3- ((6-cyano-5-methyl-1H-indol-2- yl)methyl)-1-methylurea |
| 175 | 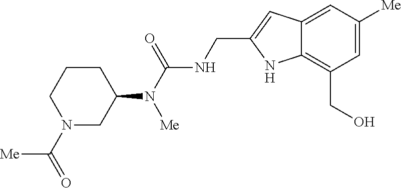 [SMILES: CC(=O)N1CCC[C@@H](N(C)C(=O)NCc2cc3cc(C)cc(CO)c3[nH]2)C1] | (R)-1-(1-acetylpiperidin-3-yl)-3- ((7-(hydroxymethyl)-5-methyl- 1H-indol-2-yl)methyl)-1- methylurea |
| 176 | 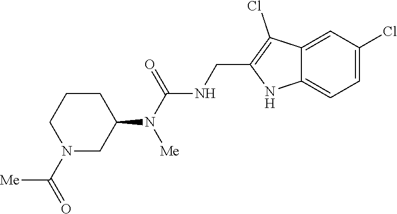 [SMILES: CC(=O)N1CCC[C@@H](N(C)C(=O)NCc2[nH]c3ccc(Cl)cc3c2Cl)C1] | (R)-1-(1-acetylpiperidin-3-yl)-3- ((3,5-dichloro-1H-indol-2- yl)methyl)-1-methylurea |
| 177 | 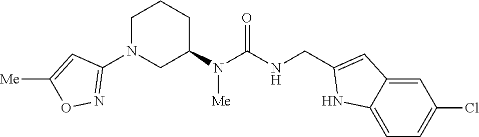 [SMILES: Cc1cc(N2CCC[C@@H](N(C)C(=O)NCc3cc4cc(Cl)ccc4[nH]3)C2)no1] | (R)-3-((5-chloro-1H-indol-2- yl)methyl)-1-methyl-1-(1-(5- methylisoxazol-3-yl)piperidin-3- yl)urea |
| 178 | 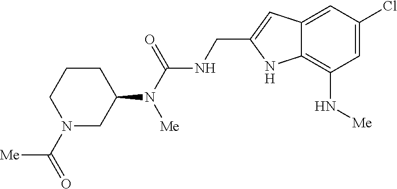 [SMILES: CNc1cc(Cl)cc2cc(CNC(=O)N(C)[C@@H]3CCCN(C(C)=O)C3)[nH]c12] | (R)-1-(1-acetylpiperidin-3-yl)-3- ((5-chloro-7-(methylamino)-1H- indol-2-yl)methyl)-1-methylurea |
| 179 | 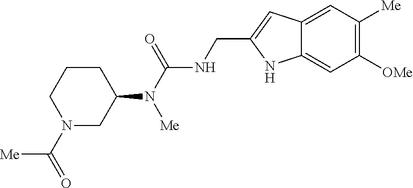 [SMILES: COc1cc2[nH]c(CNC(=O)N(C)[C@@H]3CCCN(C(C)=O)C3)cc2cc1C] | (R)-1-(1-acetylpiperidin-3-yl)-3- ((6-methoxy-5-methyl-1H-indol-2- yl)methyl)-1-methylurea |
| 180 | 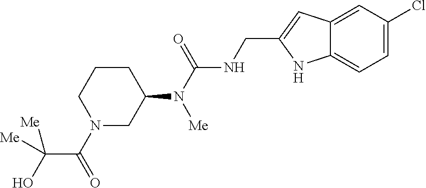 [SMILES: CN(C(=O)NCc1cc2cc(Cl)ccc2[nH]1)[C@@H]1CCCN(C(=O)C(C)(C)O)C1] | (R)-3-((5-chloro-1H-indol-2- yl)methyl)-1-(1-(2-hydroxy-2- methylpropanoyl)piperidin-3-yl)- 1-methylurea |
| 181 | 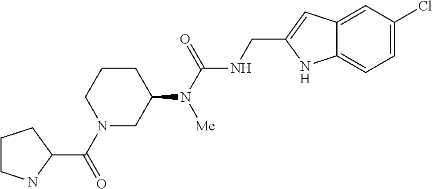 [SMILES: CN(C(=O)NCc1cc2cc(Cl)ccc2[nH]1)[C@@H]1CCCN(C(=O)C2CCCN2)C1] | (R)-3-((5-chloro-1H-indol-2- yl)methyl)-1-methyl-1-(1- (pyrrolidine-1-carbonyl)piperidin- 3-yl)urea |
| 182 | 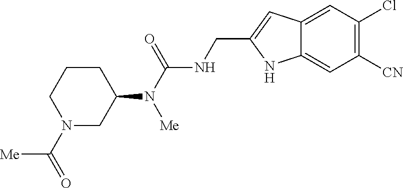 [SMILES: CC(=O)N1CCC[C@@H](N(C)C(=O)NCc2cc3cc(Cl)c(C#N)cc3[nH]2)C1] | (R)-1-(1-acetylpiperidin-3-yl)-3- ((5-chloro-6-cyano-1H-indol-2- yl)methyl)-1-methylurea |
| 183 | 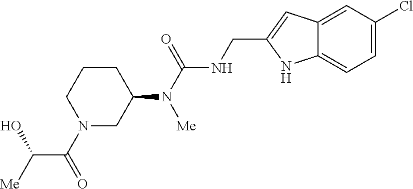 [SMILES: C[C@H](O)C(=O)N1CCC[C@@H](N(C)C(=O)NCc2cc3cc(Cl)ccc3[nH]2)C1] | 3-((5-chloro-1H-indol-2- yl)methyl)-1-((R)-1-((S)-2- hydroxypropanoyl)piperidin-3-yl)- 1-methylurea |
| 184 | 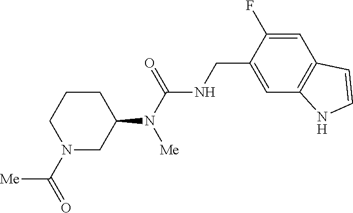 [SMILES: CC(=O)N1CCC[C@@H](N(C)C(=O)NCc2cc3[nH]ccc3cc2F)C1] | (R)-1-(1-acetylpiperidin-3-yl)-3- ((5-fluoro-1H-indol-6-yl)methyl)- 1-methylurea |
| 185 | 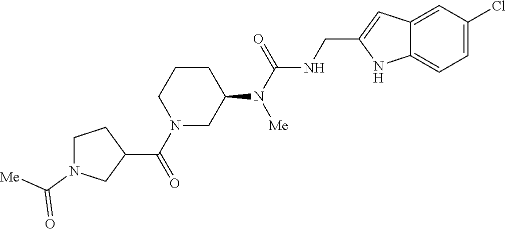 [SMILES: CC(=O)N1CCC(C(=O)N2CCC[C@@H](N(C)C(=O)NCc3cc4cc(Cl)ccc4[nH]3)C2)C1] | 1-((3R)-1-(1-acetylpyrrolidine-3- carbonyl)piperidin-3-yl)-3-((5- chloro-1H-indol-2-yl)methyl)-1- methylurea |
| 186 | 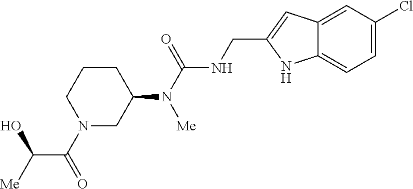 [SMILES: C[C@@H](O)C(=O)N1CCC[C@@H](N(C)C(=O)NCc2cc3cc(Cl)ccc3[nH]2)C1] | 3-((5-chloro-1H-indol-2- yl)methyl)-1-((R)-1-((R)-2- hydroxypropanoyl)piperidin-3-yl)- 1-methylurea |
| 187 | 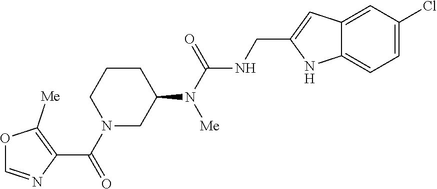 [SMILES: Cc1ocnc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(5- methyloxazole-4- carbonyl)piperidin-3-yl)urea |
| 188 | 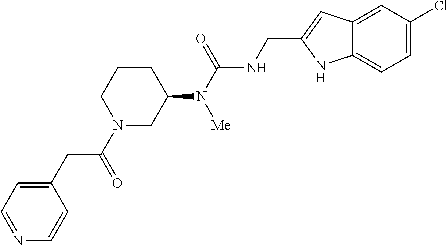 [SMILES: CN(C(=O)NCc1cc2cc(Cl)ccc2[nH]1)[C@@H]1CCCN(C(=O)Cc2ccncc2)C1] | (R)-3-((5-chloro-1H-indol-2- yl)methyl)-1-methyl-1-(1-(2- (pyridin-4-yl)acetyl)piperidin-3- yl)urea |
| 189 | 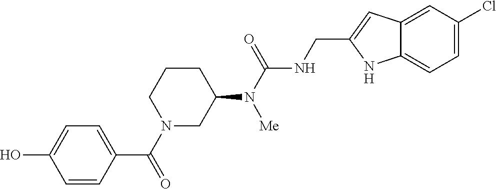 [SMILES: CN(C(=O)NCc1cc2cc(Cl)ccc2[nH]1)[C@@H]1CCCN(C(=O)c2ccc(O)cc2)C1] | (R)-3-((5-chloro-1H-indol-2- yl)methyl)-1-(1-(4- hydroxybenzoyl)piperidin-3-yl)-1- methylurea |
| 190 | 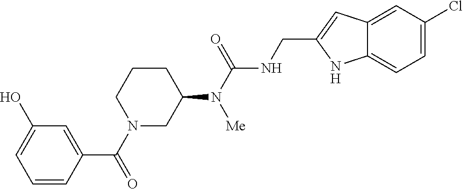 [SMILES: CN(C(=O)NCc1cc2cc(Cl)ccc2[nH]1)[C@@H]1CCCN(C(=O)c2cccc(O)c2)C1] | (R)-3-((5-chloro-1H-indol-2- yl)methyl)-1-(1-(3- hydroxybenzoyl)piperidin-3-yl)-1- methylurea |
| 191 | 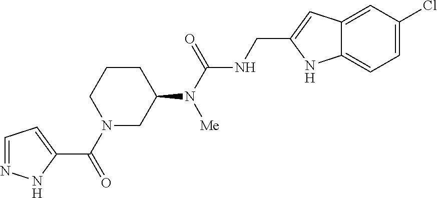 [SMILES: CN(C(=O)NCc1cc2cc(Cl)ccc2[nH]1)[C@@H]1CCCN(C(=O)c2ccn[nH]2)C1] | (R)-1-(1-(1H-pyrazole-5- carbonyl)piperidin-3-yl)-3-((5- chloro-1H-indol-2-yl)methyl)-1- methylurea |
| 192 | 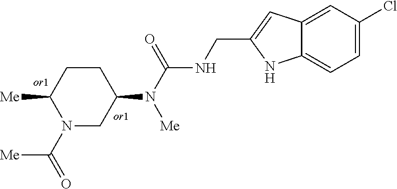 [SMILES: CC(=O)N1C[C@H](N(C)C(=O)NCc2cc3cc(Cl)ccc3[nH]2)CC[C@@H]1C] | 1-((3R,6S or 3S,6R)-1-acetyl-6- methylpiperidin-3-yl)-3-((5- chloro-1H-indol-2-yl)methyl)-1- methylurea |
| 193 | 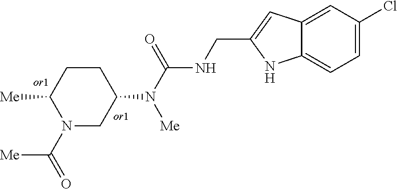 [SMILES: CC(=O)N1C[C@@H](N(C)C(=O)NCc2cc3cc(Cl)ccc3[nH]2)CC[C@H]1C] | 1-((3S,6R or 3R,6S)-1-acetyl-6- methylpiperidin-3-yl)-3-((5- chloro-1H-indol-2-yl)methyl)-1- methylurea |
| 194 | 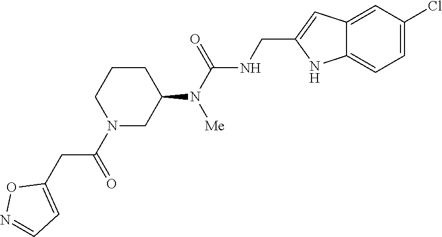 [SMILES: CN(C(=O)NCc1cc2cc(Cl)ccc2[nH]1)[C@@H]1CCCN(C(=O)Cc2ccno2)C1] | (R)-3-((5-chloro-1H-indol-2- yl)methyl)-1-(1-(2-(isoxazol-5- yl)acetyl)piperidin-3-yl)-1- methylurea |
| 195 | 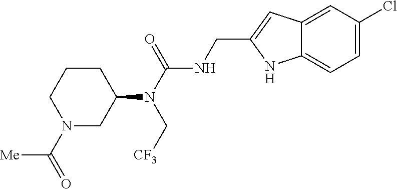 [SMILES: CC(=O)N1CCC[C@@H](N(CC(F)(F)F)C(=O)NCc2cc3cc(Cl)ccc3[nH]2)C1] | (R)-1-(1-acetylpiperidin-3-yl)-3- ((5-chloro-1H-indol-2-yl)methyl)- 1-(2,2,2-trifluoroethyl)urea |
| 196 | 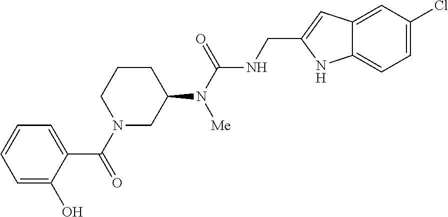 [SMILES: CN(C(=O)NCc1cc2cc(Cl)ccc2[nH]1)[C@@H]1CCCN(C(=O)c2ccccc2O)C1] | (R)-3-((5-chloro-1H-indol-2- yl)methyl)-1-(1-(2- hydroxybenzoyl)piperidin-3-yl)-1- methylurea |
| 197 | 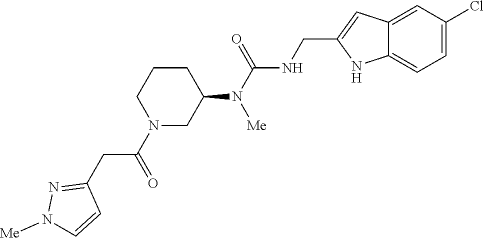 [SMILES: CN(C(=O)NCc1cc2cc(Cl)ccc2[nH]1)[C@@H]1CCCN(C(=O)Cc2ccn(C)n2)C1] | (R)-3-((5-chloro-1H-indol-2- yl)methyl)-1-methyl-1-(1-(2-(1- methyl-1H-pyrazol-3- yl)acetyl)piperidin-3-yl)urea |
| 198 | 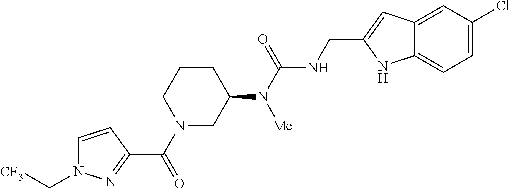 [SMILES: CN(C(=O)NCc1cc2cc(Cl)ccc2[nH]1)[C@@H]1CCCN(C(=O)c2ccn(CC(F)(F)F)n2)C1] | (R)-3-((5-chloro-1H-indol-2- yl)methyl)-1-methyl-1-(1-(1- (2,2,2-trifluoroethyl)-1H-pyrazole- 3-carbonyl)piperidin-3-yl)urea |
| 199 | 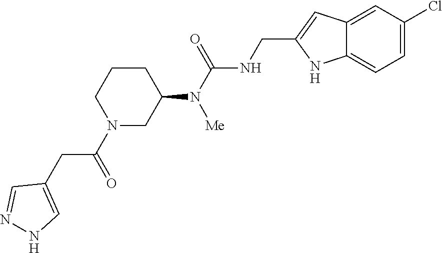 [SMILES: CN(C(=O)NCc1cc2cc(Cl)ccc2[nH]1)[C@@H]1CCCN(C(=O)Cc2cn[nH]c2)C1] | (R)-1-(1-(2-(1H-pyrazol-4- yl)acetyl)piperidin-3-yl)-3-((5- chloro-1H-indol-2-yl)methyl)-1- methylurea |
| 200 | 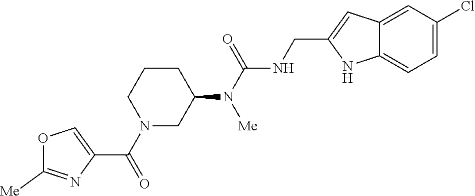 [SMILES: Cc1nc(C(=O)N2CCC[C@@H](N(C)C(=O)NCc3cc4cc(Cl)ccc4[nH]3)C2)co1] | (R)-3-((5-chloro-1H-indol-2- yl)methyl)-1-methyl-1-(1-(2- methyloxazole-4- carbonyl)piperidin-3-yl)urea |
| 201 | 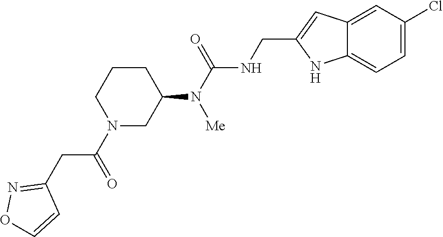 [SMILES: CN(C(=O)NCc1cc2cc(Cl)ccc2[nH]1)[C@@H]1CCCN(C(=O)Cc2ccon2)C1] | (R)-3-((5-chloro-1H-indol-2- yl)methyl)-1-(1-(2-(isoxazol-3- yl)acetyl)piperidin-3-yl)-1- methylurea |
| 202 | 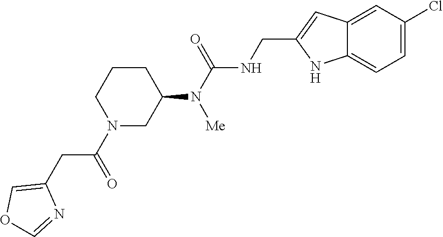 [SMILES: CN(C(=O)NCc1cc2cc(Cl)ccc2[nH]1)[C@@H]1CCCN(C(=O)Cc2cocn2)C1] | (R)-3-((5-chloro-1H-indol-2- yl)methyl)-1-methyl-1-(1-(2- (oxazol-4-yl)acetyl)piperidin-3- yl)urea |
| 203 | 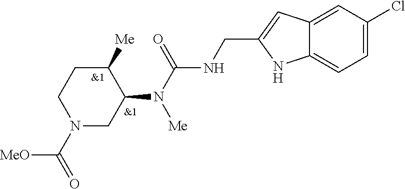 [SMILES: COC(=O)N1CC[C@@H](C)[C@@H](N(C)C(=O)NCc2cc3cc(Cl)ccc3[nH]2)C1] | 1-((3R,4R and 3S,4S)-1-acetyl-4- methylpiperidin-3-yl)-3-((5- chloro-1H-indol-2-yl)methyl)-1- methylurea |
| 204 | 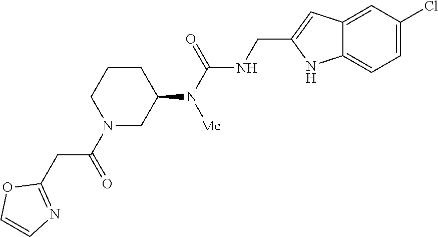 [SMILES: CN(C(=O)NCc1cc2cc(Cl)ccc2[nH]1)[C@@H]1CCCN(C(=O)Cc2ncco2)C1] | (R)-3-((5-chloro-1H-indol-2- yl)methyl)-1-methyl-1-(1-(2- (oxazol-2-yl)acetyl)piperidin-3- yl)urea |
| 205 | 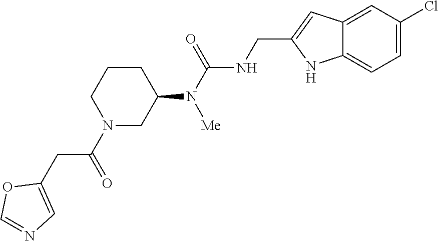 [SMILES: CN(C(=O)NCc1cc2cc(Cl)ccc2[nH]1)[C@@H]1CCCN(C(=O)Cc2cnco2)C1] | (R)-3-((5-chloro-1H-indol-2- yl)methyl)-1-methyl-1-(1-(2- (oxazol-5-yl)acetyl)piperidin-3- yl)urea |
| 206 | 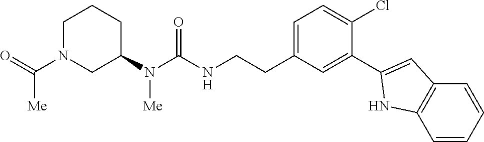 [SMILES: CC(=O)N1CCC[C@@H](N(C)C(=O)NCCc2ccc(Cl)c(-c3cc4ccccc4[nH]3)c2)C1] | (R)-1-(1-acetylpiperidin-3-yl)-3- (4-chloro-3-(1H-indol-2- yl)phenethyl)-1-methylurea |
| 207 | 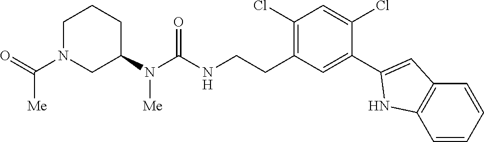 [SMILES: CC(=O)N1CCC[C@@H](N(C)C(=O)NCCc2cc(-c3cc4ccccc4[nH]3)c(Cl)cc2Cl)C1] | (R)-1-(1-acetylpiperidin-3-yl)-3- (2,4-dichloro-5-(1H-indol-2- yl)phenethyl)-1-methylurea |
| 208 | 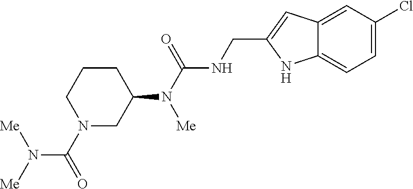 [SMILES: CN(C)C(=O)N1CCC[C@@H](N(C)C(=O)NCc2cc3cc(Cl)ccc3[nH]2)C1] | (R)-3-(3-((5-chloro-1H-indol-2- yl)methyl)-1-methylureido)-N,N- dimethylpiperidine-1-carboxamide |
| 209 | 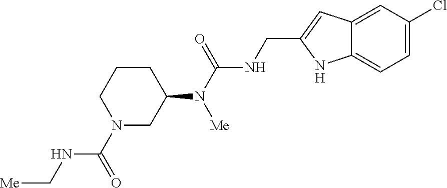 [SMILES: CCNC(=O)N1CCC[C@@H](N(C)C(=O)NCc2cc3cc(Cl)ccc3[nH]2)C1] | (R)-3-(3-((5-chloro-1H-indol-2- yl)methyl)-1-methylureido)-N- ethylpiperidine-1-carboxamide |
| 210 | 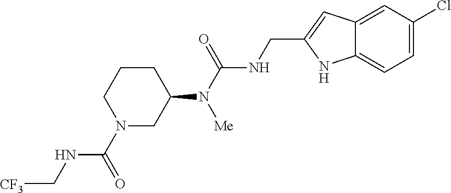 [SMILES: CN(C(=O)NCc1cc2cc(Cl)ccc2[nH]1)[C@@H]1CCCN(C(=O)NCC(F)(F)F)C1] | (R)-3-(3-((5-chloro-1H-indol-2- yl)methyl)-1-methylureido)-N- (2,2,2-trifluoroethyl)piperidine-1- carboxamide |
| 211 | 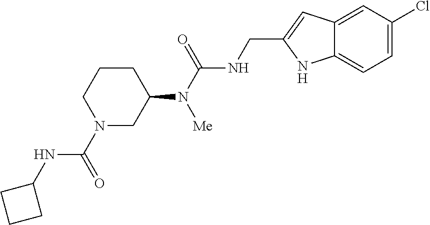 [SMILES: CN(C(=O)NCc1cc2cc(Cl)ccc2[nH]1)[C@@H]1CCCN(C(=O)NC2CCC2)C1] | (R)-3-(3-((5-chloro-1H-indol-2- yl)methyl)-1-methylureido)-N- cyclobutylpiperidine-1- carboxamide |
| 212 | 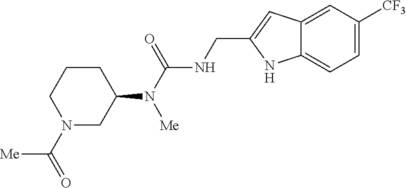 [SMILES: CC(=O)N1CCC[C@@H](N(C)C(=O)NCc2cc3cc(C(F)(F)F)ccc3[nH]2)C1] | (R)-1-(1-acetylpiperidin-3-yl)-1- methyl-3-((5-(trifluoromethyl)- 1H-indol-2-yl)methyl)urea |
| 213 | 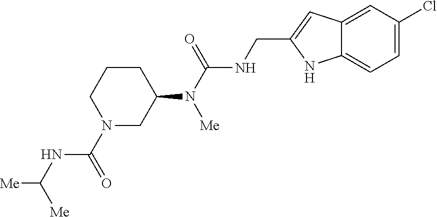 [SMILES: CC(C)NC(=O)N1CCC[C@@H](N(C)C(=O)NCc2cc3cc(Cl)ccc3[nH]2)C1] | (R)-3-(3-((5-chloro-1H-indol-2- yl)methyl)-1-methylureido)-N- isopropylpiperidine-1- carboxamide |
| 214 | 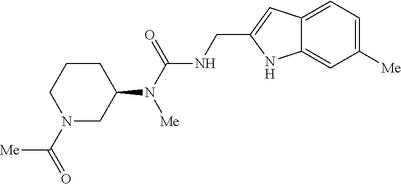 [SMILES: CC(=O)N1CCC[C@@H](N(C)C(=O)NCc2cc3ccc(C)cc3[nH]2)C1] | (R)-1-(1-acetylpiperidin-3-yl)-1- methyl-3-((6-methyl-1H-indol-2- yl)methyl)urea |
| 215 | 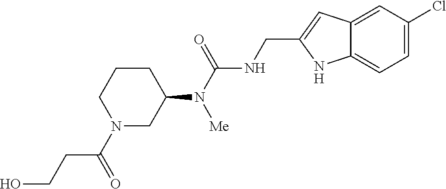 [SMILES: CN(C(=O)NCc1cc2cc(Cl)ccc2[nH]1)[C@@H]1CCCN(C(=O)CCO)C1] | (R)-3-((5-chloro-1H-indol-2- yl)methyl)-1-(1-(3- hydroxypropanoyl)piperidin-3-yl)- 1-methylurea |
| 216 | 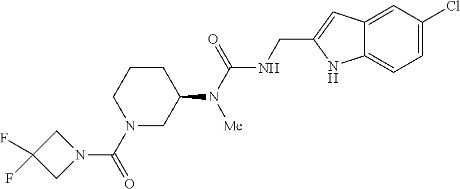 [SMILES: CN(C(=O)NCc1cc2cc(Cl)ccc2[nH]1)[C@@H]1CCCN(C(=O)N2CC(F)(F)C2)C1] | (R)-3-((5-chloro-1H-indol-2- yl)methyl)-1-(1-(3,3- difluoroazetidine-1- carbonyl)piperidin-3-yl)-1- methylurea |
| 217 | 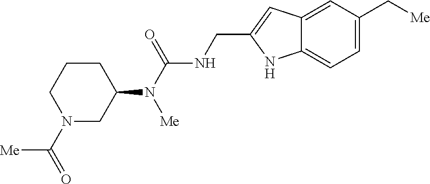 [SMILES: CCc1ccc2[nH]c(CNC(=O)N(C)[C@@H]3CCCN(C(C)=O)C3)cc2c1] | (R)-1-(1-acetylpiperidin-3-yl)-3- ((5-ethyl-1H-indol-2-yl)methyl)-1- methylurea |
| 218 | 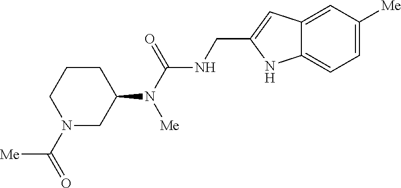 [SMILES: CC(=O)N1CCC[C@@H](N(C)C(=O)NCc2cc3cc(C)ccc3[nH]2)C1] | (R)-1-(1-acetylpiperidin-3-yl)-1- methyl-3-((5-methyl-1H-indol-2- yl)methyl)urea |
| 219 | 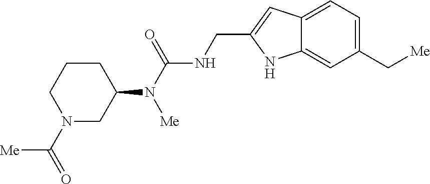 [SMILES: CCc1ccc2cc(CNC(=O)N(C)[C@@H]3CCCN(C(C)=O)C3)[nH]c2c1] | (R)-1-(1-acetylpiperidin-3-yl)-3- ((6-ethyl-1H-indol-2-yl)methyl)-1- methylurea |
| 220 | 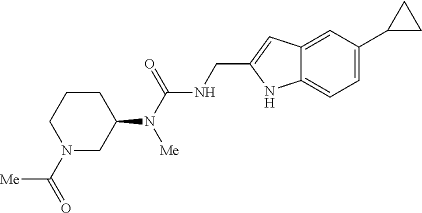 [SMILES: CC(=O)N1CCC[C@@H](N(C)C(=O)NCc2cc3cc(C4CC4)ccc3[nH]2)C1] | (R)-1-(1-acetylpiperidin-3-yl)-3- ((5-cyclopropyl-1H-indol-2- yl)methyl)-1-methylurea |
| 221 | 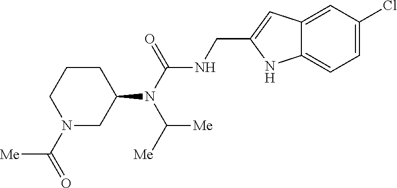 [SMILES: CC(=O)N1CCC[C@@H](N(C(=O)NCc2cc3cc(Cl)ccc3[nH]2)C(C)C)C1] | (R)-1-(1-acetylpiperidin-3-yl)-3- ((5-chloro-1H-indol-2-yl)methyl)- 1-isopropylurea |
| 222 | 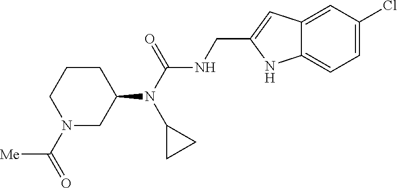 [SMILES: CC(=O)N1CCC[C@@H](N(C(=O)NCc2cc3cc(Cl)ccc3[nH]2)C2CC2)C1] | (R)-1-(1-acetylpiperidin-3-yl)-3- ((5-chloro-1H-indol-2-yl)methyl)- 1-cyclopropylurea |
| 223 | 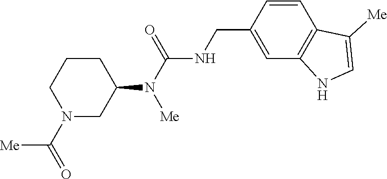 [SMILES: CC(=O)N1CCC[C@@H](N(C)C(=O)NCc2ccc3c(C)c[nH]c3c2)C1] | (R)-1-(1-acetylpiperidin-3-yl)-1- methyl-3-((3-methyl-1H-indol-6- yl)methyl)urea |
| 224 | 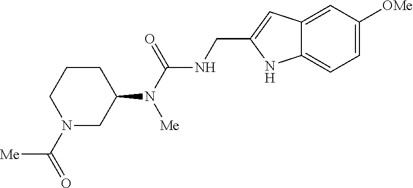 [SMILES: COc1ccc2[nH]c(CNC(=O)N(C)[C@@H]3CCCN(C(C)=O)C3)cc2c1] | (R)-1-(1-acetylpiperidin-3-yl)-3- ((5-methoxy-1H-indol-2- yl)methyl)-1-methylurea |
| 225 | 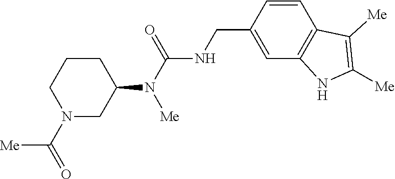 [SMILES: CC(=O)N1CCC[C@@H](N(C)C(=O)NCc2ccc3c(C)c(C)[nH]c3c2)C1] | (R)-1-(1-acetylpiperidin-3-yl)-3- ((2,3-dimethyl-1H-indol-6- yl)methyl)-1-methylurea |
| 226 | 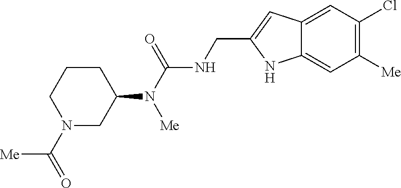 [SMILES: CC(=O)N1CCC[C@@H](N(C)C(=O)NCc2cc3cc(Cl)c(C)cc3[nH]2)C1] | (R)-1-(1-acetylpiperidin-3-yl)-3- ((5-chloro-6-methyl-1H-indol-2- yl)methyl)-1-methylurea |
| 227 | 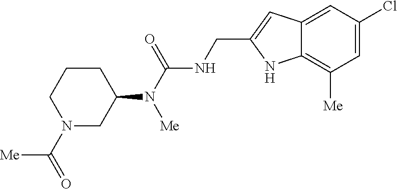 [SMILES: CC(=O)N1CCC[C@@H](N(C)C(=O)NCc2cc3cc(Cl)cc(C)c3[nH]2)C1] | (R)-1-(1-acetylpiperidin-3-yl)-3- ((5-chloro-7-methyl-1H-indol-2- yl)methyl)-1-methylurea |
| 228 | 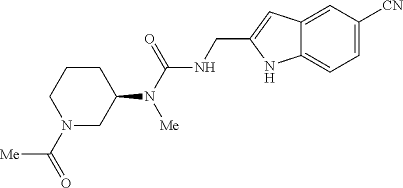 [SMILES: CC(=O)N1CCC[C@@H](N(C)C(=O)NCc2cc3cc(C#N)ccc3[nH]2)C1] | (R)-1-(1-acetylpiperidin-3-yl)-3- ((5-cyano-1H-indol-2-yl)methyl)- 1-methylurea |
| 229 | 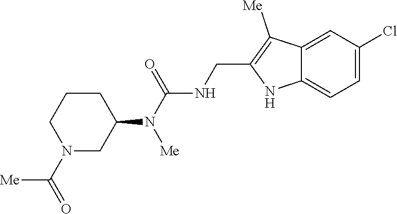 [SMILES: CC(=O)N1CCC[C@@H](N(C)C(=O)NCc2[nH]c3ccc(Cl)cc3c2C)C1] | (R)-1-(1-acetylpiperidin-3-yl)-3- ((5-chloro-3-methyl-1H-indol-2- yl)methyl)-1-methylurea |
| 230 | 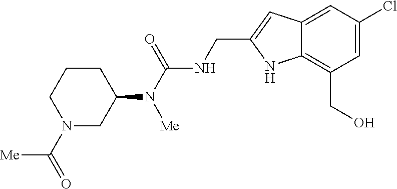 [SMILES: CC(=O)N1CCC[C@@H](N(C)C(=O)NCc2cc3cc(Cl)cc(CO)c3[nH]2)C1] | (R)-1-(1-acetylpiperidin-3-yl)-3- ((5-chloro-7-(hydroxymethyl)-1H- indol-2-yl)methyl)-1-methylurea |
| 231 | 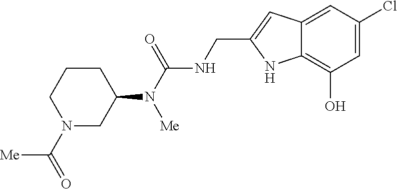 [SMILES: CC(=O)N1CCC[C@@H](N(C)C(=O)NCc2cc3cc(Cl)cc(O)c3[nH]2)C1] | (R)-1-(1-acetylpiperidin-3-yl)-3- ((5-chloro-7-hydroxy-1H-indol-2- yl)methyl)-1-methylurea |
| 232 | 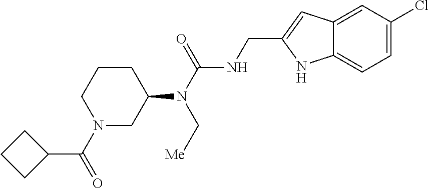 [SMILES: CCN(C(=O)NCc1cc2cc(Cl)ccc2[nH]1)[C@@H]1CCCN(C(=O)C2CCC2)C1] | (R)-3-((5-chloro-1H-indol-2- yl)methyl)-1-(1- (cyclobutanecarbonyl)piperidin-3- yl)-1-ethylurea |
| 233 | 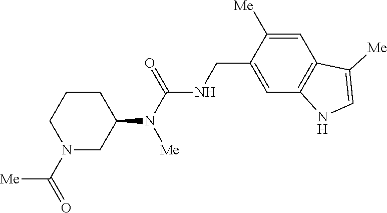 [SMILES: CC(=O)N1CCC[C@@H](N(C)C(=O)NCc2cc3[nH]cc(C)c3cc2C)C1] | (R)-1-(1-acetylpiperidin-3-yl)-3- ((3,5-dimethyl-1H-indol-6- yl)methyl)-1-methylurea |
| 234 | 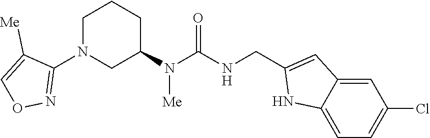 [SMILES: Cc1conc1N1CCC[C@@H](N(C)C(=O)NCc2cc3cc(Cl)ccc3[nH]2)C1] | (R)-3-((5-chloro-1H-indol-2- yl)methyl)-1-methyl-1-(1-(4- methylisoxazol-3-yl)piperidin-3- yl)urea |
| 235 | 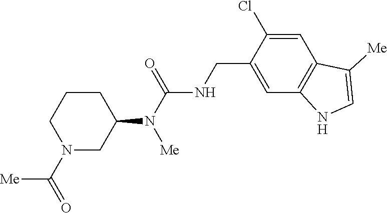 [SMILES: CC(=O)N1CCC[C@@H](N(C)C(=O)NCc2cc3[nH]cc(C)c3cc2Cl)C1] | (R)-1-(1-acetylpiperidin-3-yl)-3- ((5-chloro-3-methyl-1H-indol-6- yl)methyl)-1-methylurea |
| 236 | 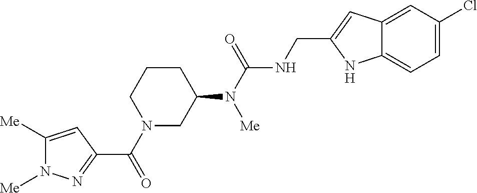 [SMILES: Cc1cc(C(=O)N2CCC[C@@H](N(C)C(=O)NCc3cc4cc(Cl)ccc4[nH]3)C2)nn1C] | (R)-3-((5-chloro-1H-indol-2- yl)methyl)-1-(1-(1,5-dimethyl-1H- pyrazole-3-carbonyl)piperidin-3- yl)-1-methylurea |
| 237 | 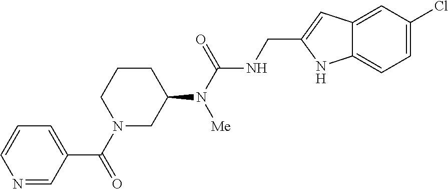 [SMILES: CN(C(=O)NCc1cc2cc(Cl)ccc2[nH]1)[C@@H]1CCCN(C(=O)c2cccnc2)C1] | (R)-3-((5-chloro-1H-indol-2- yl)methyl)-1-methyl-1-(1- nicotinoylpiperidin-3-yl)urea |
| 238 | 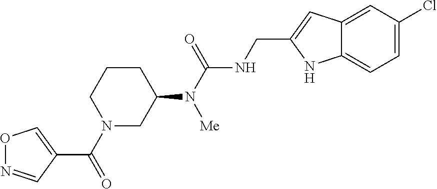 [SMILES: CN(C(=O)NCc1cc2cc(Cl)ccc2[nH]1)[C@@H]1CCCN(C(=O)c2cnoc2)C1] | (R)-3-((5-chloro-1H-indol-2- yl)methyl)-1-(1-(isoxazole-4- carbonyl)piperidin-3-yl)-1- methylurea |
| 239 | 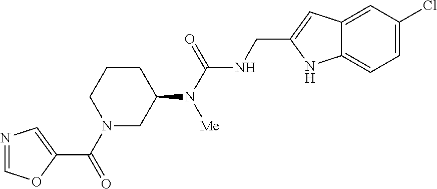 [SMILES: CN(C(=O)NCc1cc2cc(Cl)ccc2[nH]1)[C@@H]1CCCN(C(=O)c2cnco2)C1] | (R)-3-((5-chloro-1H-indol-2- yl)methyl)-1-methyl-1-(1- (oxazole-5-carbonyl)piperidin-3- yl)urea |
| 240 | 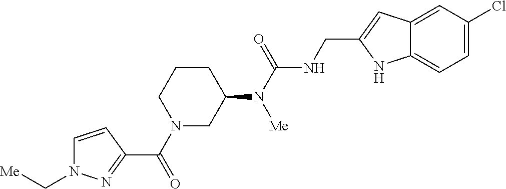 [SMILES: CCn1ccc(C(=O)N2CCC[C@@H](N(C)C(=O)NCc3cc4cc(Cl)ccc4[nH]3)C2)n1] | (R)-3-((5-chloro-1H-indol-2- yl)methyl)-1-(1-(1-ethyl-1H- pyrazole-3-carbonyl)piperidin-3- yl)-1-methylurea |
| 241 | 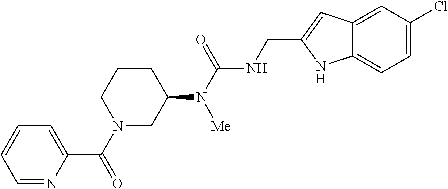 [SMILES: CN(C(=O)NCc1cc2cc(Cl)ccc2[nH]1)[C@@H]1CCCN(C(=O)c2ccccn2)C1] | (R)-3-((5-chloro-1H-indol-2- yl)methyl)-1-methyl-1-(1- picolinoylpiperidin-3-yl)urea |
| 242 | 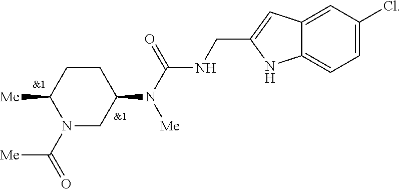 [SMILES: CC(=O)N1C[C@H](N(C)C(=O)NCc2cc3cc(Cl)ccc3[nH]2)CC[C@@H]1C] | 1-((3R,6S and 3S,6R)-1-acetyl-6- methylpiperidin-3-yl)-3-((5- chloro-1H-indol-2-yl)methyl)-1- methylurea |
| 243 | 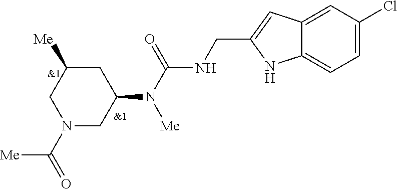 [SMILES: CC(=O)N1C[C@@H](C)C[C@@H](N(C)C(=O)NCc2cc3cc(Cl)ccc3[nH]2)C1] | 1-((3R,5S and 3S,5R)-1-acetyl-5- methylpiperidin-3-yl)-3-((5- chloro-1H-indol-2-yl)methyl)-1- methylurea |
| 244 | 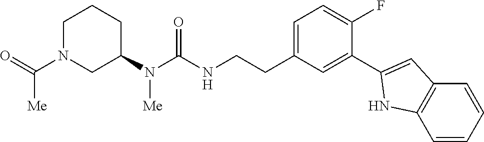 [SMILES: CC(=O)N1CCC[C@@H](N(C)C(=O)NCCc2ccc(F)c(-c3cc4ccccc4[nH]3)c2)C1] | (R)-1-(1-acetylpiperidin-3-yl)-3- (4-fluoro-3-(1H-indol-2- yl)phenethyl)-1-methylurea |
| 245 | 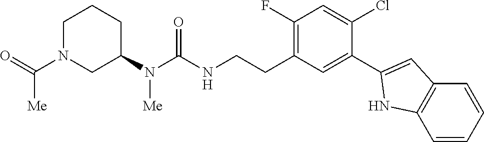 [SMILES: CC(=O)N1CCC[C@@H](N(C)C(=O)NCCc2cc(-c3cc4ccccc4[nH]3)c(Cl)cc2F)C1] | (R)-1-(1-acetylpiperidin-3-yl)-3- (4-chloro-2-fluoro-5-(1H-indol-2- yl)phenethyl)-1-methylurea |
| 246 | 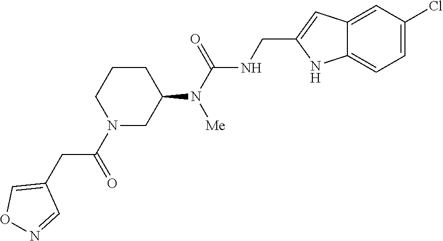 [SMILES: CN(C(=O)NCc1cc2cc(Cl)ccc2[nH]1)[C@@H]1CCCN(C(=O)Cc2cnoc2)C1] | (R)-3-((5-chloro-1H-indol-2- yl)methyl)-1-(1-(2-(isoxazol-4- yl)acetyl)piperidin-3-yl)-1- methylurea |
| 247 | 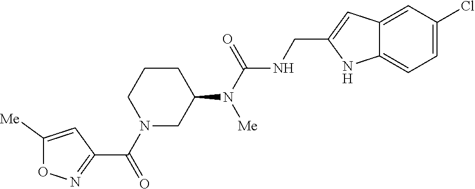 [SMILES: Cc1cc(C(=O)N2CCC[C@@H](N(C)C(=O)NCc3cc4cc(Cl)ccc4[nH]3)C2)no1] | (R)-3-((5-chloro-1H-indol-2- yl)methyl)-1-methyl-1-(1-(5- methylisoxazole-3- carbonyl)piperidin-3-yl)urea |
| 248 | 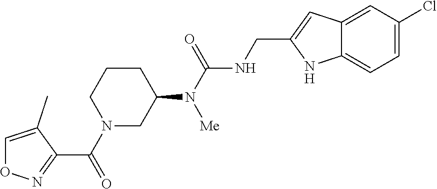 [SMILES: Cc1conc1C(=O)N1CCC[C@@H](N(C)C(=O)NCc2cc3cc(Cl)ccc3[nH]2)C1] | (R)-3-((5-chloro-1H-indol-2- yl)methyl)-1-methyl-1-(1-(4- methylisoxazole-3- carbonyl)piperidin-3-yl)urea |

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
1-(1-acetylpiperidin-3-yl)-3-({5-chloro-6-[(5-methyl-1,2-oxazol-3-yl)methoxy]-1H-indol-2-yl}methyl)-1-methylurea;
3-({5-chloro-6-[(5-methyl-1,2-oxazol-3-yl)methoxy]-1H-indol-2-yl}methyl)-1-methyl-1-[1-(pyridazin-3-yl)piperidin-3-yl]urea;
1-(1-acetylpiperidin-3-yl)-1-methyl-3-({6-[(5-methyl-1,2-oxazol-3-yl)methoxy]-1H-indol-2-yl}methyl)urea;
1-methyl-3-({6-[(5-methyl-1,2-oxazol-3-yl)methoxy]-1H-indol-2-yl}methyl)-1-[1-(pyridazin-3-yl)piperidin-3-yl]urea;
1-(1-acetylpiperidin-3-yl)-1-methyl-3-({6-[(1,2-oxazol-3-yl)methoxy]-1H-indol-2-yl}methyl)urea;
3-[(5-chloro-1H-indol-2-yl)methyl]-1-[1-(2-hydroxyacetyl)piperidin-3-yl]-1-methylurea;
3-[(5-chloro-1H-indol-2-yl)methyl]-1-[1-(1-fluorocyclopropanecarbonyl)piperidin-3-yl]-1-methylurea;
3-[(5-chloro-1H-indol-2-yl)methyl]-1-methyl-1-{1-[2-(2-oxo-1,3-oxazolidin-3-yl)acetyl]piperidin-3-yl}urea;
3-[(5-chloro-1H-indol-2-yl)methyl]-1-methyl-1-[1-(1,3-oxazole-4-carbonyl)piperidin-3-yl]urea;
3-[(5-chloro-1H-indol-2-yl)methyl]-1-methyl-1-[1-(1H-pyrazole-4-carbonyl)piperidin-3-yl]urea;
3-[(5-chloro-1H-indol-2-yl)methyl]-1-methyl-1-[1-(pyrimidine-2-carbonyl)piperidin-3-yl]urea;
1-{1-[2-(carbamoylamino)acetyl]piperidin-3-yl}-3-[(5-chloro-1H-indol-2-yl)methyl]-1-methylurea;
3-[(5-chloro-1H-indol-2-yl)methyl]-1-methyl-1-[1-(3-oxo-3,4-dihydropyrazine-2-carbonyl)piperidin-3-yl]urea;
4-[3-({[(5-chloro-1H-indol-2-yl)methyl]carbamoyl}(methyl)amino)piperidin-1-yl]-4-oxobutanamide;
N-{2-[3-({[(5-chloro-1H-indol-2-yl)methyl]carbamoyl}(methyl)amino)piperidin-1-yl]-2-oxoethyl}-2,2,2-trifluoroacetamide;
3-[(5-chloro-1H-indol-2-yl)methyl]-1-methyl-1-[1-(1,2-oxazole-5-carbonyl)piperidin-3-yl]urea;
3-[(5-chloro-1H-indol-2-yl)methyl]-1-[1-(2-cyanoacetyl)piperidin-3-yl]-1-methylurea;
3-[(5-chloro-1H-indol-2-yl)methyl]-1-methyl-1-(1-propanoylpiperidin-3-yl)urea;
3-[(5-chloro-1H-indol-2-yl)methyl]-1-[1-(2-methoxyacetyl)piperidin-3-yl]-1-methylurea;
3-[(5-chloro-1H-indol-2-yl)methyl]-1-methyl-1-{1-[2-(1H-1,2,3,4-tetrazol-1-yl)acetyl]piperidin-3-yl}urea;
3-[(5-chloro-1H-indol-2-yl)methyl]-1-methyl-1-[1-(1-methyl-1H-1,2,4-triazole-5-carbonyl)piperidin-3-yl]urea;
3-[(5-chloro-1H-indol-2-yl)methyl]-1-methyl-1-[1-(4-methyl-,2-oxazole-5-carbonyl)piperidin-3-yl]urea;
3-[(5-chloro-1H-indol-2-yl)methyl]-1-methyl-1-[1-(1-methylcyclopropanecarbonyl)piperidin-3-yl]urea;
3-[(5-chloro-1H-indol-2-yl)methyl]-1-methyl-1-{1-[2-(3-methyl-1,2,4-oxadiazol-5-yl)acetyl]piperidin-3-yl}urea;
3-[(5-chloro-1H-indol-2-yl)methyl]-1-methyl-1-[1-(1-methyl-1H-pyrazole-4-carbonyl)piperidin-3-yl]urea;
3-[(5-chloro-1H-indol-2-yl)methyl]-1-methyl-1-[1-(1,3-thiazole-2-carbonyl)piperidin-3-yl]urea;
3-[(5-chloro-1H-indol-2-yl)methyl]-1-methyl-1-[1-(1,2-oxazole-3-carbonyl)piperidin-3-yl]urea;
3-[(5-chloro-1H-indol-2-yl)methyl]-1-methyl-1-[1-(4-methyl-1,3-oxazole-5-carbonyl)piperidin-3-yl]urea;
3-[(5-chloro-1H-indol-2-yl)methyl]-1-[1-(2-fluorocyclopropanecarbonyl)piperidin-3-yl]-1-methylurea;
3-[(5-chloro-1H-indol-2-yl)methyl]-1-methyl-1-[1-(pyrimidine-5-carbonyl)piperidin-3-yl]urea;
3-[(5-chloro-1H-indol-2-yl)methyl]-1-methyl-1-[1-(5-methyl-1,3-thiazole-2-carbonyl)piperidin-3-yl]urea;
3-[(5-chloro-1H-indol-2-yl)methyl]-1-[1-(3,3-difluorocyclobutanecarbonyl)piperidin-3-yl]-1-methylurea;
3-[(5-chloro-1H-indol-2-yl)methyl]-1-methyl-1-[1-(1-methyl-1H-pyrazole-3-carbonyl)piperidin-3-yl]urea;
3-[(5-chloro-1H-indol-2-yl)methyl]-1-methyl-1-[1-(3-oxocyclopentanecarbonyl)piperidin-3-yl]urea;
3-[(5-chloro-1H-indol-2-yl)methyl]-1-[1-(3-hydroxycyclobutanecarbonyl)piperidin-3-yl]-1-methylurea;
3-[(5-chloro-1H-indol-2-yl)methyl]-1-[1-(1H-imidazole-4-carbonyl)piperidin-3-yl]-1-methylurea;
3-[(5-chloro-1H-indol-2-yl)methyl]-1-methyl-1-[1-(pyridine-4-carbonyl)piperidin-3-yl]urea;
3-[(5-chloro-1H-indol-2-yl)methyl]-1-[1-(2,2-difluorocyclopropanecarbonyl)piperidin-3-yl]-1-methylurea;
3-[(5-chloro-1H-indol-2-yl)methyl]-1-methyl-1-[1-(3,3,3-trifluoropropanoyl)piperidin-3-yl]urea;
3-[(5-chloro-1H-indol-2-yl)methyl]-1-[1-(2-methoxypropanoyl)piperidin-3-yl]-1-methylurea;
3-[(5-chloro-1H-indol-2-yl)methyl]-1-methyl-1-[1-(1-methyl-1H-1,2,3-triazole-5-carbonyl)piperidin-3-yl]urea;
N-{2-[3-({[(5-chloro-1H-indol-2-yl)methyl]carbamoyl}(methyl)amino)piperidin-1-yl]-2-oxoethyl}acetamide;
3-[(5-chloro-1H-indol-2-yl)methyl]-1-[1-(2-fluoro-4-hydroxybenzoyl)piperidin-3-yl]-1-methylurea;
3-[(5-chloro-1H-indol-2-yl)methyl]-1-(1-cyclopentanecarbonylpiperidin-3-yl)-1-methylurea;
3-[(5-chloro-1H-indol-2-yl)methyl]-1-methyl-1-(1-{[1,2,4]triazolo[4,3-a]pyridine-8-carbonyl}piperidin-3-yl)urea;
3-[(5-chloro-1H-indol-2-yl)methyl]-1-[1-(1H-imidazole-2-carbonyl)piperidin-3-yl]-1-methylurea;
3-[(5-chloro-1H-indol-2-yl)methyl]-1-[1-(2,3-difluorobenzoyl)piperidin-3-yl]-1-methylurea;
3-[(5-chloro-1H-indol-2-yl)methyl]-1-[1-(2-hydroxypropanoyl)piperidin-3-yl]-1-methylurea;
3-[(5-chloro-1H-indol-2-yl)methyl]-1-methyl-1-[1-(5-methyl-2H-1,2,3-triazole-4-carbonyl)piperidin-3-yl]urea;
3-[(5-chloro-1H-indol-2-yl)methyl]-1-methyl-1-f{1-[2-(pyrazin-2-yl)acetyl]piperidin-3-yl}urea;
3-[(5-chloro-1H-indol-2-yl)methyl]-1-methyl-1-[1-(2-methylpropanoyl)piperidin-3-yl]urea;
1-{1-[3-(carbamoylamino)propanoyl]piperidin-3-yl}-3-[(5-chloro-1H-indol-2-yl)methyl]-1-methylurea;
1-(1-benzoylpiperidin-3-yl)-3-[(5-chloro-1H-indol-2-yl)methyl]-1-methylurea;
3-[(5-chloro-1H-indol-2-yl)methyl]-1-methyl-1-(1-{2-[(6-oxo-1,6-dihydropyridazin-3-yl)oxy]acetyl}piperidin-3-yl)urea;
3-[(5-chloro-1H-indol-2-yl)methyl]-1-[1-(5-fluoropyrimidine-2-carbonyl)piperidin-3-yl]-1-methylurea;
3-[(5-chloro-1H-indol-2-yl)methyl]-1-methyl-1-[1-(1-methyl-1H-pyrazole-5-carbonyl)piperidin-3-yl]urea;
3-[(5-chloro-1H-indol-2-yl)methyl]-1-(1-cyclopropanecarbonylpiperidin-3-yl)-1-methylurea;
3-[(5-chloro-1H-indol-2-yl)methyl]-1-[1-(2-methanesulfonylacetyl)piperidin-3-yl]-1-methylurea;
1-[1-(1,3-benzoxazole-5-carbonyl)piperidin-3-yl]-3-[(5-chloro-1H-indol-2-yl)methyl]-1-methylurea;
3-[(5-chloro-1H-indol-2-yl)methyl]-1-[1-(3-methoxy-2-methylpropanoyl)piperidin-3-yl]-1-methylurea;
3-[(5-chloro-1H-indol-2-yl)methyl]-1-methyl-1-[1-(3-methyl-1H-pyrazole-5-carbonyl)piperidin-3-yl]urea;
3-[(5-chloro-1H-indol-2-yl)methyl]-1-methyl-1-[1-(oxolane-2-carbonyl)piperidin-3-yl]urea;
3-[(5-chloro-1H-indol-2-yl)methyl]-1-(1-{2-[(dimethylcarbamoyl)amino]acetyl}piperidin-3-yl)-1-methylurea;
3-[(5-chloro-1H-indol-2-yl)methyl]-1-methyl-1-[1-(5-oxopyrrolidine-2-carbonyl)piperidin-3-yl]urea;
3-[(5-chloro-1H-indol-2-yl)methyl]-1-methyl-1-[1-(3-methyl-,2-oxazole-5-carbonyl)piperidin-3-yl]urea;
3-[(5-chloro-1H-indol-2-yl)methyl]-1-methyl-1-[1-(pyridazine-4-carbonyl)piperidin-3-yl]urea;
3-[(5-chloro-1H-indol-2-yl)methyl]-1-[1-(2-hydroxybutanoyl)piperidin-3-yl]-1-methylurea;
3-[(5-chloro-1H-indol-2-yl)methyl]-1-methyl-1-{1-[2-(2-oxo-1,2-dihydropyridin-1-yl)acetyl]piperidin-3-yl}urea;
3-[(5-chloro-1H-indol-2-yl)methyl]-1-methyl-1-{1-[3-(1H-pyrazol-1-yl)propanoyl]piperidin-3-yl}urea;
3-[(5-chloro-1H-indol-2-yl)methyl]-1-methyl-1-[1-(3,3,3-trifluoro-2-hydroxy-2-methylpropanoyl)piperidin-3-yl]urea;
3-[(5-chloro-1H-indol-2-yl)methyl]-1-[1-(2,3-dihydro-1-benzofuran-7-carbonyl)piperidin-3-yl]-1-methylurea;
3-[(5-chloro-1H-indol-2-yl)methyl]-1-{1-[2-(ethanesulfonyl)acetyl]piperidin-3-yl}-1-methylurea;
3-[(5-chloro-1H-indol-2-yl)methyl]-1-methyl-1-{1-[2-(1H-1,2,4-triazol-1-yl)propanoyl]piperidin-3-yl}urea;
3-[(5-chloro-1H-indol-2-yl)methyl]-1-{1-[1-(difluoromethyl)-1H-pyrazole-3-carbonyl]piperidin-3-yl}-1-methylurea;
3-[(5-chloro-1H-indol-2-yl)methyl]-1-methyl-1-{1-[2-(2H-1,2,3-triazol-2-yl)acetyl]piperidin-3-yl}urea;
3-[(5-chloro-1H-indol-2-yl)methyl]-1-[1-(2-cyclopropylacetyl)piperidin-3-yl]-1-methylurea;
3-[(5-chloro-1H-indol-2-yl)methyl]-1-{1-[5-(methoxymethyl)-1,2-oxazole-4-carbonyl]piperidin-3-yl}-1-methylurea;
3-[(5-chloro-1H-indol-2-yl)methyl]-1-methyl-1-{1-[2-(1H-pyrazol-5-yl)acetyl]piperidin-3-yl}urea;
3-[(5-chloro-1H-indol-2-yl)methyl]-1-[1-(3-hydroxy-3-methylbutanoyl)piperidin-3-yl]-1-methylurea;
3-[(5-chloro-1H-indol-2-yl)methyl]-1-{1-[2-(3-fluoro-4-methoxyphenyl)acetyl]piperidin-3-yl}-1-methylurea;
3-[(5-chloro-1H-indol-2-yl)methyl]-1-(1-cyclobutanecarbonylpiperidin-3-yl)-1-methylurea;
3-[(5-chloro-1H-indol-2-yl)methyl]-1-[1-(2-methoxy-2-methylpropanoyl)piperidin-3-yl]-1-methylurea;
3-[(5-chloro-1H-indol-2-yl)methyl]-1-methyl-1-[1-(2-methyl-1,3-oxazole-5-carbonyl)piperidin-3-yl]urea;
3-[(5-chloro-1H-indol-2-yl)methyl]-1-{1-[2-(1-hydroxycyclobutyl)acetyl]piperidin-3-yl}-1-methylurea;
1-[1-(2,1-benzoxazole-3-carbonyl)piperidin-3-yl]-3-[(5-chloro-1H-indol-2-yl)methyl]-1-methylurea;
3-[(5-chloro-1H-indol-2-yl)methyl]-1-methyl-1-{1-[2-(pyridin-2-yl)acetyl]piperidin-3-yl}urea;
N-{3-[3-({[(5-chloro-1H-indol-2-yl)methyl]carbamoyl}(methyl)amino)piperidin-1-yl]-3-oxopropyl}acetamide;
3-[(5-chloro-1H-indol-2-yl)methyl]-1-methyl-1-[1-(5-methyl-1H-imidazole-4-carbonyl)piperidin-3-yl]urea;
3-[(5-chloro-1H-indol-2-yl)methyl]-1-{1-[5-(hydroxymethyl)-1,2-oxazole-3-carbonyl]piperidin-3-yl}-1-methylurea;
3-[(5-chloro-1H-indol-2-yl)methyl]-1-[1-(1-hydroxycyclopentanecarbonyl)piperidin-3-yl]-1-methylurea;
3-[(5-chloro-1H-indol-2-yl)methyl]-1-[1-(5-methoxypyridine-2-carbonyl)piperidin-3-yl]-1-methylurea;
1-[1-(1-acetylazetidine-3-carbonyl)piperidin-3-yl]-3-[(5-chloro-1H-indol-2-yl)methyl]-1-methylurea;
3-[(5-chloro-1H-indol-2-yl)methyl]-1-methyl-1-{1-[2-(pyridin-3-yl)acetyl]piperidin-3-yl}urea;
3-[(5-chloro-1H-indol-2-yl)methyl]-1-methyl-1-{1-[1-(trifluoromethyl)cyclopropanecarbonyl]piperidin-3-yl}urea;
3-[(5-chloro-1H-indol-2-yl)methyl]-1-[1-(2,3-dihydroxypropanoyl)piperidin-3-yl]-1-methylurea;
N-{1-[3-({[(5-chloro-1H-indol-2-yl)methyl]carbamoyl}(methyl)amino)piperidin-1-yl]-1-oxopropan-2-yl}acetamide;
3-[(5-chloro-1H-indol-2-yl)methyl]-1-methyl-1-{1-[2-(oxan-2-yl)acetyl]piperidin-3-yl}urea;
3-[(5-chloro-1H-indol-2-yl)methyl]-1-[1-(5-fluoro-6-methoxypyridine-3-carbonyl)piperidin-3-yl]-1-methylurea;
3-[(5-chloro-1H-indol-2-yl)methyl]-1-{1-[2-(1-hydroxycyclobutyl)propanoyl]piperidin-3-yl}-1-methylurea;
3-[(5-chloro-1H-indol-2-yl)methyl]-1-[1-(1,3-dimethyl-1H-pyrazole-5-carbonyl)piperidin-3-yl]-1-methylurea;
3-[(5-chloro-1H-indol-2-yl)methyl]-1-methyl-1-{1-[2-(2-oxo-1,2-dihydropyrimidin-1-yl)acetyl]piperidin-3-yl}urea;
3-[(5-chloro-1H-indol-2-yl)methyl]-1-methyl-1-[1-(oxane-4-carbonyl)piperidin-3-yl]urea;
3-[(5-chloro-1H-indol-2-yl)methyl]-1-methyl-1-[1-(morpholine-3-carbonyl)piperidin-3-yl]urea;
3-[(5-chloro-1H-indol-2-yl)methyl]-1-methyl-1-{1-[2-(2-oxopyrrolidin-1-yl)acetyl]piperidin-3-yl}urea;
3-[(5-chloro-1H-indol-2-yl)methyl]-1-methyl-1-{1-[2-(1-methyl-1H-pyrazol-4-yl)acetyl]piperidin-3-yl}urea;
3-[(5-chloro-1H-indol-2-yl)methyl]-1-(1-{imidazo[1,2-a]pyridine-7-carbonyl}piperidin-3-yl)-1-methylurea;
1-[1-(2-amino-3-methoxypropanoyl)piperidin-3-yl]-3-[(5-chloro-1H-indol-2-yl)methyl]-1-methylurea;
N-{1-[3-({[(5-chloro-1H-indol-2-yl)methyl]carbamoyl}(methyl)amino)piperidin-1-yl]-1-oxobutan-2-yl}acetamide;
3-[(5-chloro-1H-indol-2-yl)methyl]-1-methyl-1-[1-(oxane-2-carbonyl)piperidin-3-yl]urea;
3-[(5-chloro-1H-indol-2-yl)methyl]-1-methyl-1-{1-[2-(3-methyl-1H-pyrazol-1-yl)acetyl]piperidin-3-yl}urea;
3-[(5-chloro-1H-indol-2-yl)methyl]-1-methyl-1-[1-(4-oxopyrrolidine-2-carbonyl)piperidin-3-yl]urea;
3-[(5-chloro-1H-indol-2-yl)methyl]-1-(1-{2-[(ethylcarbamoyl)amino]propanoyl}piperidin-3-yl)-1-methylurea;
3-[(5-chloro-1H-indol-2-yl)methyl]-1-[1-(1-methanesulfonylazetidine-3-carbonyl)piperidin-3-yl]-1-methylurea;
3-[(5-chloro-1H-indol-2-yl)methyl]-1-{1-[1-(methoxymethyl)cyclopropanecarbonyl]piperidin-3-yl}-1-methylurea;
3-[(5-chloro-1H-indol-2-yl)methyl]-1-[1-(4-hydroxycyclohexanecarbonyl)piperidin-3-yl]-1-methylurea;
3-[(5-chloro-1H-indol-2-yl)methyl]-1-methyl-1-(1-{6-methylimidazo[1,2-a]pyrimidine-3-carbonyl}piperidin-3-yl)urea;
3-[(5-chloro-1H-indol-2-yl)methyl]-1-[1-(3-hydroxy-3-methylcyclobutanecarbonyl)piperidin-3-yl]-1-methylurea;
1-(1-acetylpiperidin-3-yl)-3-({5-chloro-6-[(1,3-thiazol-5-yl)methoxy]-1H-indol-2-yl}methyl)-1-methylurea;
1-(1-acetylpiperidin-3-yl)-1-methyl-3-({6-[(1,3-thiazol-5-yl)methoxy]-1H-indol-2-yl}methyl)urea;
1-(1-acetylpiperidin-3-yl)-1-ethyl-3-({6-[(1,2-oxazol-3-yl)methoxy]-1H-indol-2-yl}methyl)urea;
1-(1-acetylpiperidin-3-yl)-3-({5-chloro-6-[(1,2-oxazol-3-yl)methoxy]-1H-indol-2-yl}methyl)-1-methylurea;
3-[(5-chloro-1H-indol-2-yl)methyl]-1-methyl-1-[1-(1-methylazetidine-3-carbonyl)piperidin-3-yl]urea;
3-[(5-chloro-1H-indol-2-yl)methyl]-1-[1-(1-hydroxycyclobutanecarbonyl)piperidin-3-yl]-1-methylurea;
1-[1-(azetidine-3-carbonyl)piperidin-3-yl]-3-[(5-chloro-1H-indol-2-yl)methyl]-1-methylurea;
1-(1-acetylpiperidin-3-yl)-3-({5-chloro-6-[(1,2-oxazol-3-yl)methoxy]-1H-indol-2-yl}methyl)-1-ethylurea;
3-[(5-chloro-1H-indol-2-yl)methyl]-1-[1-(1-fluorocyclobutanecarbonyl)piperidin-3-yl]-1-methylurea;
3-[(5-chloro-1H-indol-2-yl)methyl]-1-methyl-1-[1-(3-methyloxetane-3-carbonyl)piperidin-3-yl]urea;
3-[(5-chloro-1H-indol-2-yl)methyl]-1-[1-(2-fluorocyclobutanecarbonyl)piperidin-3-yl]-1-methylurea;
3-[(5-chloro-1H-indol-2-yl)methyl]-1-[1-(3-fluorocyclobutanecarbonyl)piperidin-3-yl]-1-methylurea;
3-[(5-chloro-1H-indol-2-yl)methyl]-1-methyl-1-[1-(oxetane-3-carbonyl)piperidin-3-yl]urea;
1-(1-{bicyclo[1.1.1]pentane-1-carbonyl}piperidin-3-yl)-3-[(5-chloro-1H-indol-2-yl)methyl]-1-methylurea;
3-[(5-chloro-1H-indol-2-yl)methyl]-1-{1-[1-(hydroxymethyl)cyclobutanecarbonyl]piperidin-3-yl}-1-methylurea;
3-[(5-chloro-1H-indol-2-yl)methyl]-1-{1-[3-(hydroxymethyl)cyclobutanecarbonyl]piperidin-3-yl}-1-methylurea;
3-[(5-chloro-1H-indol-2-yl)methyl]-1-[1-(2-cyclobutylacetyl)piperidin-3-yl]-1-methylurea;
3-[(5-chloro-1H-indol-2-yl)methyl]-1-[1-(3-hydroxycyclobutanecarbonyl)piperidin-3-yl]-1-methylurea;
3-((5-chloro-1H-indol-2-yl)methyl)-1-methyl-1-(1-(5-methylisoxazol-3-yl)piperidin-3-yl)urea;
and
3-((5-chloro-1H-indol-2-yl)methyl)-1-methyl-1-(1-(4-methylisoxazol-3-yl)piperidin-3-yl)urea; or a tautomer thereof, or a pharmaceutically acceptable salt of any of the foregoing.

In some embodiments, provided herein is a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from the group consisting of:
(R)-3-((5-chloro-1H-indol-2-yl)methyl)-1-methyl-1-(1-(oxetane-3-carbonyl)piperidin-3-yl)urea;
(R)-3-((5-chloro-1H-indol-2-yl)methyl)-1-methyl-1-(1-(2-(2-oxooxazolidin-3-yl)acetyl)piperidin-3-yl)urea;
(R)-3-((5-chloro-1H-indol-2-yl)methyl)-1-(1-(2-cyanoacetyl)piperidin-3-yl)-1-methylurea;
(R)-3-((5-chloro-1H-indol-2-yl)methyl)-1-(1-(2-hydroxyacetyl)piperidin-3-yl)-1-methylurea;
(R)-3-((5-chloro-1H-indol-2-yl)methyl)-1-(1-(isoxazole-3-carbonyl)piperidin-3-yl)-1-methylurea;
(R)-3-((5-chloro-1H-indol-2-yl)methyl)-1-(1-(isoxazole-5-carbonyl)piperidin-3-yl)-1-methylurea;
(R)-3-((5-chloro-1H-indol-2-yl)methyl)-1-methyl-1-(1-(oxazole-4-carbonyl)piperidin-3-yl)urea;
(R)-3-((5-chloro-1H-indol-2-yl)methyl)-1-methyl-1-(1-(3,3,3-trifluoropropanoyl)piperidin-3-yl)urea;
(R)-3-((5-chloro-1H-indol-2-yl)methyl)-1-methyl-1-(1-(thiazole-2-carbonyl)piperidin-3-yl)urea;
(R)-3-((5-chloro-1H-indol-2-yl)methyl)-1-methyl-1-(1-propionylpiperidin-3-yl)urea;
(R)-1-(1-(1H-pyrazole-4-carbonyl)piperidin-3-yl)-3-((5-chloro-1H-indol-2-yl)methyl)-1-methylurea;
3-((5-chloro-1H-indol-2-yl)methyl)-1-((3R)-1-(2-hydroxypropanoyl)piperidin-3-yl)-1-methylurea;
(R)-1-(1-(1H-imidazole-4-carbonyl)piperidin-3-yl)-3-((5-chloro-1H-indol-2-yl)methyl)-1-methylurea;
3-((5-chloro-1H-indol-2-yl)methyl)-1-((R)-1-((1R,2S)-2-fluorocyclopropane-1-carbonyl)piperidin-3-yl)-1-methylurea;
(R)-3-((5-chloro-1H-indol-2-yl)methyl)-1-(1-(2-methoxyacetyl)piperidin-3-yl)-1-methylurea;
(R)-3-((5-chloro-1H-indol-2-yl)methyl)-1-(1-(1-fluorocyclopropane-1-carbonyl)piperidin-3-yl)-1-methylurea;
(R)-3-((5-chloro-1H-indol-2-yl)methyl)-1-(1-(cyclopropanecarbonyl)piperidin-3-yl)-1-methylurea;
(R)-1-(1-(1H-imidazole-2-carbonyl)piperidin-3-yl)-3-((5-chloro-1H-indol-2-yl)methyl)-1-methylurea;
3-((5-chloro-1H-indol-2-yl)methyl)-1-((3R)-1-(2,2-difluorocyclopropane-1-carbonyl)piperidin-3-yl)-1-methylurea;
(R)-3-((5-chloro-1H-indol-2-yl)methyl)-1-methyl-1-(1-(pyrimidine-5-carbonyl)piperidin-3-yl)urea;
(R)-1-(1-(2-(1H-tetrazol-1-yl)acetyl)piperidin-3-yl)-3-((5-chloro-1H-indol-2-yl)methyl)-1-methylurea;
(R)-3-((5-chloro-1H-indol-2-yl)methyl)-1-methyl-1-(1-(pyrimidine-2-carbonyl)piperidin-3-yl)urea;
(R)-3-((5-chloro-1H-indol-2-yl)methyl)-1-methyl-1-(1-(pyridazine-4-carbonyl)piperidin-3-yl)urea;
(R)-3-((5-chloro-1H-indol-2-yl)methyl)-1-(1-(5-fluoropyrimidine-2-carbonyl)piperidin-3-yl)-1-methylurea;
(R)-3-((5-chloro-1H-indol-2-yl)methyl)-1-methyl-1-(1-(5-methyl-2H-1,2,3-triazole-4-carbonyl)piperidin-3-yl)urea;
(R)-3-((5-chloro-1H-indol-2-yl)methyl)-1-methyl-1-(1-(1-methyl-1H-1,2,3-triazole-5-carbonyl)piperidin-3-yl)urea;
(R)-3-((5-chloro-1H-indol-2-yl)methyl)-1-methyl-1-(1-(4-methyloxazole-5-carbonyl)piperidin-3-yl)urea;
(R)-3-((5-chloro-1H-indol-2-yl)methyl)-1-methyl-1-(1-(1-methyl-1H-1,2,4-triazole-5-carbonyl)piperidin-3-yl)urea;
(R)-1-(1-(carbamoylglycyl)piperidin-3-yl)-3-((5-chloro-1H-indol-2-yl)methyl)-1-methylurea;
(R)-1-(1-(2-(2H-1,2,3-triazol-2-yl)acetyl)piperidin-3-yl)-3-((5-chloro-1H-indol-2-yl)methyl)-1-methylurea;
(R)-3-((5-chloro-1H-indol-2-yl)methyl)-1-methyl-1-(1-(3-methylisoxazole-5-carbonyl)piperidin-3-yl)urea;
(R)-3-((5-chloro-1H-indol-2-yl)methyl)-1-methyl-1-(1-(2-(methylsulfonyl)acetyl)piperidin-3-yl)urea;
(R)-3-((5-chloro-1H-indol-2-yl)methyl)-1-(1-isobutyrylpiperidin-3-yl)-1-methylurea;
(R)-3-((5-chloro-1H-indol-2-yl)methyl)-1-(1-isonicotinoylpiperidin-3-yl)-1-methylurea;
(R)-3-((5-chloro-1H-indol-2-yl)methyl)-1-methyl-1-(1-(5-methylthiazole-2-carbonyl)piperidin-3-yl)urea;
(R)-3-((5-chloro-1H-indol-2-yl)methyl)-1-methyl-1-(1-(4-methylisoxazole-5-carbonyl)piperidin-3-yl)urea;
(R)-3-((5-chloro-1H-indol-2-yl)methyl)-1-methyl-1-(1-(3-oxo-3,4-dihydropyrazine-2-carbonyl)piperidin-3-yl)urea;
(R)-3-((5-chloro-1H-indol-2-yl)methyl)-1-methyl-1-(1-(2-methyloxazole-5-carbonyl)piperidin-3-yl)urea;
(R)-3-((5-chloro-1H-indol-2-yl)methyl)-1-(1-(1-(difluoromethyl)-1H-pyrazole-3-carbonyl)piperidin-3-yl)-1-methylurea;
3-((5-chloro-1H-indol-2-yl)methyl)-1-((3R)-1-(2-hydroxybutanoyl)piperidin-3-yl)-1-methylurea;
(R)-3-((5-chloro-1H-indol-2-yl)methyl)-1-methyl-1-(1-(3-methyl-1H-pyrazole-5-carbonyl)piperidin-3-yl)urea;
(R)-3-((5-chloro-1H-indol-2-yl)methyl)-1-(1-(2,3-difluorobenzoyl)piperidin-3-yl)-1-methylurea;
3-((5-chloro-1H-indol-2-yl)methyl)-1-((3R)-1-(2-methoxypropanoyl)piperidin-3-yl)-1-methylurea;
(R)-3-((5-chloro-1H-indol-2-yl)methyl)-1-(1-(3,3-difluorocyclobutane-1-carbonyl)piperidin-3-yl)-1-methylurea;
(R)-3-((5-chloro-1H-indol-2-yl)methyl)-1-methyl-1-(1-(1-methylcyclopropane-1-carbonyl)piperidin-3-yl)urea;
(R)-4-(3-(3-((5-chloro-1H-indol-2-yl)methyl)-1-methylureido)piperidin-1-yl)-4-oxobutanamide;
(R)-3-((5-chloro-1H-indol-2-yl)methyl)-1-(1-(5-(hydroxymethyl)isoxazole-3-carbonyl)piperidin-3-yl)-1-methylurea;
(R)-3-((5-chloro-1H-indol-2-yl)methyl)-1-(1-(cyclobutanecarbonyl)piperidin-3-yl)-1-methylurea;
(R)-3-((5-chloro-1H-indol-2-yl)methyl)-1-(1-(2-cyclopropylacetyl)piperidin-3-yl)-1-methylurea;
3-((5-chloro-1H-indol-2-yl)methyl)-1-methyl-1-((3R)-1-(3,3,3-trifluoro-2-hydroxy-2-methylpropanoyl)piperidin-3-yl)urea;
(R)-3-((5-chloro-1H-indol-2-yl)methyl)-1-methyl-1-(1-(1-methyl-1H-pyrazole-5-carbonyl)piperidin-3-yl)urea;
(R)-1-(1-benzoylpiperidin-3-yl)-3-((5-chloro-1H-indol-2-yl)methyl)-1-methylurea;
(R)-N-(2-(3-(3-((5-chloro-1H-indol-2-yl)methyl)-1-methylureido)piperidin-1-yl)-2-oxoethyl)acetamide;
(R)-3-((5-chloro-1H-indol-2-yl)methyl)-1-methyl-1-(1-(1-methyl-1H-pyrazole-3-carbonyl)piperidin-3-yl)urea;
(R)-3-((5-chloro-1H-indol-2-yl)methyl)-1-methyl-1-(1-(1-methyl-1H-pyrazole-4-carbonyl)piperidin-3-yl)urea;
(R)-N-(2-(3-(3-((5-chloro-1H-indol-2-yl)methyl)-1-methylureido)piperidin-1-yl)-2-oxoethyl)-2,2,2-trifluoroacetamide;
(R)-3-((5-chloro-1H-indol-2-yl)methyl)-1-methyl-1-(1-(1-(trifluoromethyl)cyclopropane-1-carbonyl)piperidin-3-yl)urea;
(R)-3-((5-chloro-1H-indol-2-yl)methyl)-1-methyl-1-(1-(5-methyl-1H-imidazole-4-carbonyl)piperidin-3-yl)urea;
(R)-1-(1-(2-(1H-pyrazol-5-yl)acetyl)piperidin-3-yl)-3-((5-chloro-1H-indol-2-yl)methyl)-1-methylurea;
3-((5-chloro-1H-indol-2-yl)methyl)-1-methyl-1-((3R)-1-(5-oxopyrrolidine-2-carbonyl)piperidin-3-yl)urea;
3-((5-chloro-1H-indol-2-yl)methyl)-1-methyl-1-((3R)-1-(tetrahydrofuran-2-carbonyl)piperidin-3-yl)urea;
(R)-3-((5-chloro-1H-indol-2-yl)methyl)-1-methyl-1-(1-(2-(pyrazin-2-yl)acetyl)piperidin-3-yl)urea;
(R)-3-((5-chloro-1H-indol-2-yl)methyl)-1-(1-(2-fluoro-4-hydroxybenzoyl)piperidin-3-yl)-1-methylurea;
trans-(R)-3-((5-chloro-1H-indol-2-yl)methyl)-1-(1-(3-hydroxycyclobutane-1-carbonyl)piperidin-3-yl)-1-methylurea;
(R)-3-((5-chloro-1H-indol-2-yl)methyl)-1-methyl-1-(1-(2-(3-methyl-1,2,4-oxadiazol-5-yl)acetyl)piperidin-3-yl)urea;
(R)-3-((5-chloro-1H-indol-2-yl)methyl)-1-methyl-1-(1-(2-(2-oxopyrimidin-1(2H)-yl)acetyl)piperidin-3-yl)urea;
(R)-3-((5-chloro-1H-indol-2-yl)methyl)-1-methyl-1-(1-(2-(pyridin-3-yl)acetyl)piperidin-3-yl)urea;
(R)-3-((5-chloro-1H-indol-2-yl)methyl)-1-methyl-1-(1-(2-(pyridin-2-yl)acetyl)piperidin-3-yl)urea;
(R)-1-(1-(benzo[c]isoxazole-3-carbonyl)piperidin-3-yl)-3-((5-chloro-1H-indol-2-yl)methyl)-1-methylurea;
1-((3R)-1-(2-(1H-1,2,4-triazol-1-yl)propanoyl)piperidin-3-yl)-3-((5-chloro-1H-indol-2-yl)methyl)-1-methylurea;
(R)-3-((5-chloro-1H-indol-2-yl)methyl)-1-(1-(2-(ethylsulfonyl)acetyl)piperidin-3-yl)-1-methylurea;
(R)-1-(1-(benzo[d]oxazole-5-carbonyl)piperidin-3-yl)-3-((5-chloro-1H-indol-2-yl)methyl)-1-methylurea;
(R)-3-((5-chloro-1H-indol-2-yl)methyl)-1-methyl-1-(1-(3-ureidopropanoyl)piperidin-3-yl)urea;
(R)-1-(1-([1,2,4]triazolo[4,3-a]pyridine-8-carbonyl)piperidin-3-yl)-3-((5-chloro-1H-indol-2-yl)methyl)-1-methylurea;
3-((5-chloro-1H-indol-2-yl)methyl)-1-methyl--((3R)-1-(3-oxocyclopentane-1-carbonyl)piperidin-3-yl)urea;
(R)-3-((5-chloro-1H-indol-2-yl)methyl)-1-(1-(imidazo[1,2-a]pyridine-7-carbonyl)piperidin-3-yl)-1-methylurea;
(R)-3-((5-chloro-1H-indol-2-yl)methyl)-1-(1-(1,3-dimethyl-1H-pyrazole-5-carbonyl)piperidin-3-yl)-1-methylurea;
(R)-3-((5-chloro-1H-indol-2-yl)methyl)-1-(1-(5-methoxypicolinoyl)piperidin-3-yl)-1-methylurea;
(R)-3-((5-chloro-1H-indol-2-yl)methyl)-1-(1-(2-methoxy-2-methylpropanoyl)piperidin-3-yl)-1-methylurea;
(R)-3-((5-chloro-1H-indol-2-yl)methyl)-1-(1-(3-hydroxy-3-methylbutanoyl)piperidin-3-yl)-1-methylurea;
(R)-3-((5-chloro-1H-indol-2-yl)methyl)-1-methyl-1-(1-(2-(2-oxopyridin-1(2H)-yl)acetyl)piperidin-3-yl)urea;
3-((5-chloro-1H-indol-2-yl)methyl)-1-((3R)-1-(3-methoxy-2-methylpropanoyl)piperidin-3-yl)-1-methylurea;
(R)-3-((5-chloro-1H-indol-2-yl)methyl)-1-methyl-1-(1-(2-((6-oxo-1,6-dihydropyridazin-3-yl)oxy)acetyl)piperidin-3-yl)urea;
(R)-3-((5-chloro-1H-indol-2-yl)methyl)-1-(1-(cyclopentanecarbonyl)piperidin-3-yl)-1-methylurea;
(R)-3-((5-chloro-1H-indol-2-yl)methyl)-1-methyl-1-(1-(2-(3-methyl-1H-pyrazol-1-yl)acetyl)piperidin-3-yl)urea;
(R)-3-((5-chloro-1H-indol-2-yl)methyl)-1-methyl-1-(1-(2-(1-methyl-1H-pyrazol-4-yl)acetyl)piperidin-3-yl)urea;
(R)-3-((5-chloro-1H-indol-2-yl)methyl)-1-(1-(5-fluoro-6-methoxynicotinoyl)piperidin-3-yl)-1-methylurea;
N-(1-((R)-3-(3-((5-chloro-1H-indol-2-yl)methyl)-1-methylureido)piperidin-1-yl)-1-oxopropan-2-yl)acetamide;
(R)-N-(3-(3-(3-((5-chloro-1H-indol-2-yl)methyl)-1-methylureido)piperidin-1-yl)-3-oxopropyl)acetamide;
(R)-3-((5-chloro-1H-indol-2-yl)methyl)-1-(1-(5-(methoxymethyl)isoxazole-4-carbonyl)piperidin-3-yl)-1-methylurea;
(R)-1-(1-(3-(1H-pyrazol-1-yl)propanoyl)piperidin-3-yl)-3-((5-chloro-1H-indol-2-yl)methyl)-1-methylurea;
(R)-3-((5-chloro-1H-indol-2-yl)methyl)-1-(1-(1-(methoxymethyl)cyclopropane-1-carbonyl)piperidin-3-yl)-1-methylurea;
3-((5-chloro-1H-indol-2-yl)methyl)-1-methyl-1-((3R)-1-(tetrahydro-2H-pyran-2-carbonyl)piperidin-3-yl)urea;
(R)-3-((5-chloro-1H-indol-2-yl)methyl)-1-methyl-1-(1-(2-(2-oxopyrrolidin-1-yl)acetyl)piperidin-3-yl)urea;
(R)-3-((5-chloro-1H-indol-2-yl)methyl)-1-methyl-1-(1-(tetrahydro-2H-pyran-4-carbonyl)piperidin-3-yl)urea;
(R)-1-(1-(1-acetylazetidine-3-carbonyl)piperidin-3-yl)-3-((5-chloro-1H-indol-2-yl)methyl)-1-methylurea;
(R)-3-((5-chloro-1H-indol-2-yl)methyl)-1-(1-(1-hydroxycyclopentane-1-carbonyl)piperidin-3-yl)-1-methylurea;
(R)-3-((5-chloro-1H-indol-2-yl)methyl)-1-(1-(2-(1-hydroxycyclobutyl)acetyl)piperidin-3-yl)-1-methylurea;
(R)-3-((5-chloro-1H-indol-2-yl)methyl)-1-(1-(3-hydroxy-3-methylcyclobutane-1-carbonyl)piperidin-3-yl)-1-methylurea;
(R)-3-((5-chloro-1H-indol-2-yl)methyl)-1-(1-(2,3-dihydrobenzofuran-7-carbonyl)piperidin-3-yl)-1-methylurea;
(R)-3-((5-chloro-1H-indol-2-yl)methyl)-1-(1-((dimethylcarbamoyl)glycyl)piperidin-3-yl)-1-methylurea;
(R)-3-((5-chloro-1H-indol-2-yl)methyl)-1-methyl-1-(1-(6-methylimidazo[1,2-a]pyrimidine-3-carbonyl)piperidin-3-yl)urea;
(R)-3-((5-chloro-1H-indol-2-yl)methyl)-1-methyl-1-(1-(1-(methylsulfonyl)azetidine-3-carbonyl)piperidin-3-yl)urea;
N-(1-((R)-3-(3-((5-chloro-1H-indol-2-yl)methyl)-1-methylureido)piperidin-1-yl)-1-oxobutan-2-yl)acetamide;
3-((5-chloro-1H-indol-2-yl)methyl)-1-((3R)-1-(2-(1-hydroxycyclobutyl)propanoyl)piperidin-3-yl)-1-methylurea;
3-((5-chloro-1H-indol-2-yl)methyl)-1-methyl-1-((3R)-1-(2-(tetrahydro-2H-pyran-2-yl)acetyl)piperidin-3-yl)urea;
(R)-3-((5-chloro-1H-indol-2-yl)methyl)-1-(1-(2-(3-fluoro-4-methoxyphenyl)acetyl)piperidin-3-yl)-1-methylurea;
trans-3-((5-chloro-1H-indol-2-yl)methyl)-1-((R)-1-((4-hydroxycyclohexane-1-carbonyl)piperidin-3-yl)-1-methylurea;
3-((5-chloro-1H-indol-2-yl)methyl)-1-((3R)-1-((ethylcarbamoyl)alanyl)piperidin-3-yl)-1-methylurea;
3-((5-chloro-1H-indol-2-yl)methyl)-1-((3R)-1-(2,3-dihydroxypropanoyl)piperidin-3-yl)-1-methylurea;
(R)-3-((5-chloro-1H-indol-2-yl)methyl)-1-(1-(4-fluoropicolinoyl)piperidin-3-yl)-1-methylurea;
3-((5-chloro-1H-indol-2-yl)methyl)-1-methyl-1-((3R)-1-(tetrahydrofuran-3-carbonyl)piperidin-3-yl)urea;
(R)-3-((5-chloro-1H-indol-2-yl)methyl)-1-(1-(4-fluoro-1-methyl-1H-pyrazole-3-carbonyl)piperidin-3-yl)-1-methylurea;
(R)-3-((5-chloro-1H-indol-2-yl)methyl)-1-(1-(5-fluoropicolinoyl)piperidin-3-yl)-1-methylurea;
(R)-3-((5-chloro-1H-indol-2-yl)methyl)-1-(1-(5-fluoro-1-methyl-1H-pyrazole-3-carbonyl)piperidin-3-yl)-1-methylurea;
3-((5-chloro-1H-indol-2-yl)methyl)-1-methyl-1-((3R)-1-(oxetane-2-carbonyl)piperidin-3-yl)urea;
(R)-3-((5-chloro-1H-indol-2-yl)methyl)-1-methyl-1-(1-(3-methylcyclobutane-1-carbonyl)piperidin-3-yl)urea;
(R)-3-((5-chloro-1H-indol-2-yl)methyl)-1-methyl-1-(1-(1-methylcyclobutane-1-carbonyl)piperidin-3-yl)urea;
(R)-3-((5-chloro-1H-indol-2-yl)methyl)-1-(1-(3-hydroxycyclobutane-1-carbonyl)piperidin-3-yl)-1-methylurea;
(R)-3-((5-chloro-1H-indol-2-yl)methyl)-1-(1-(2-cyclobutylacetyl)piperidin-3-yl)-1-methylurea;
(R)-3-((5-chloro-1H-indol-2-yl)methyl)-1-(1-(3-(hydroxymethyl)cyclobutane-1-carbonyl)piperidin-3-yl)-1-methylurea;
(R)-3-((5-chloro-1H-indol-2-yl)methyl)-1-(1-(1-(hydroxymethyl)cyclobutane-1-carbonyl)piperidin-3-yl)-1-methylurea;
(R)-1-(1-(bicyclo[1.1.1]pentane-1-carbonyl)piperidin-3-yl)-3-((5-chloro-1H-indol-2-yl)methyl)-1-methylurea;
(R)-3-((5-chloro-1H-indol-2-yl)methyl)-1-(1-(3-fluorocyclobutane-1-carbonyl)piperidin-3-yl)-1-methylurea;
3-((5-chloro-1H-indol-2-yl)methyl)-1-((3R)-1-(2-fluorocyclobutane-1-carbonyl)piperidin-3-yl)-1-methylurea;
(R)-3-((5-chloro-1H-indol-2-yl)methyl)-1-methyl-1-(1-(3-methyloxetane-3-carbonyl)piperidin-3-yl)urea;
(R)-3-((5-chloro-1H-indol-2-yl)methyl)-1-(1-(1-fluorocyclobutane-1-carbonyl)piperidin-3-yl)-1-methylurea;
(R)-3-((5-chloro-1H-indol-2-yl)methyl)-1-(1-(1-hydroxycyclobutane-1-carbonyl)piperidin-3-yl)-1-methylurea;
(R)-3-((5-chloro-1H-indol-2-yl)methyl)-1-methyl-1-(1-(1-methylazetidine-3-carbonyl)piperidin-3-yl)urea;
(R)-1-(1-(azetidine-3-carbonyl)piperidin-3-yl)-3-((5-chloro-1H-indol-2-yl)methyl)-1-methylurea;
3-((5-chloro-1H-indol-2-yl)methyl)-1-methyl-1-((R)-1-((S)-4-oxopyrrolidine-2-carbonyl)piperidin-3-yl)urea;
3-((5-chloro-1H-indol-2-yl)methyl)-1-methyl-1-((3R)-1-(O-methylseryl)piperidin-3-yl)urea;
3-((5-chloro-1H-indol-2-yl)methyl)-1-methyl-1-((3R)-1-(morpholine-3-carbonyl)piperidin-3-yl)urea;
(R)-1-(1-(2-(4-(2-aminoethyl)-1H-1,2,3-triazol-1-yl)acetyl)piperidin-3-yl)-3-((5-chloro-1H-indol-2-yl)methyl)-1-methylurea;
(R)-1-(1-acetylpiperidin-3-yl)-3-((5-chloro-6-((5-methylisoxazol-3-yl)methoxy)-1H-indol-2-yl)methyl)-1-methylurea;
(R)-1-(1-acetylpiperidin-3-yl)-1-methyl-3-((6-(thiazol-5-ylmethoxy)-1H-indol-2-yl)methyl)urea;
(R)-1-(1-acetylpiperidin-3-yl)-3-((5-chloro-6-(isoxazol-3-ylmethoxy)-1H-indol-2-yl)methyl)-1-ethylurea;
(R)-1-(1-acetylpiperidin-3-yl)-3-((5-chloro-6-(isoxazol-3-ylmethoxy)-1H-indol-2-yl)methyl)-1-methylurea;
(R)-1-(1-acetylpiperidin-3-yl)-1-ethyl-3-((6-(isoxazol-3-ylmethoxy)-1H-indol-2-yl)methyl)urea;
(R)-1-(1-acetylpiperidin-3-yl)-3-((5-chloro-6-(thiazol-5-ylmethoxy)-1H-indol-2-yl)methyl)-1-methylurea;
(R)-1-(1-acetylpiperidin-3-yl)-3-((6-(isoxazol-3-ylmethoxy)-1H-indol-2-yl)methyl)-1-methylurea;
(R)-1-methyl-3-((6-((5-methylisoxazol-3-yl)methoxy)-1H-indol-2-yl)methyl)-1-(1-(pyridazin-3-yl)piperidin-3-yl)urea;
(R)-1-(1-acetylpiperidin-3-yl)-1-methyl-3-((6-((5-methylisoxazol-3-yl)methoxy)-1H-indol-2-yl)methyl)urea;
(R)-3-((5-chloro-6-((5-methylisoxazol-3-yl)methoxy)-1H-indol-2-yl)methyl)-1-methyl-1-(1-(pyridazin-3-yl)piperidin-3-yl)urea;
3-((1H-indol-2-yl)methyl)-1-methyl-1-(1-(pyridazin-3-yl)piperidin-3-yl)urea;
(R)-3-((1H-indol-2-yl)methyl)-1-methyl-1-(1-(pyridazin-3-yl)piperidin-3-yl)urea;
(R)-1-(1-acetylpiperidin-3-yl)-3-((5-chloro-1H-indol-2-yl)methyl)-1-methylurea;
(R)-3-((1H-indol-2-yl)methyl)-1-(1-acetylpiperidin-3-yl)-1-methylurea;
(R)-3-((5-chloro-1H-indol-2-yl)methyl)-1-methyl-1-(1-methylpiperidin-3-yl)urea;
(R)-3-((1H-indol-2-yl)methyl)-1-(1-(isoxazol-3-yl)piperidin-3-yl)-1-methylurea;
(R)-3-((5-chloro-1H-indol-2-yl)methyl)-1-(1-(isoxazol-3-yl)piperidin-3-yl)-1-methylurea;
(R)-1-(1-acetylpiperidin-3-yl)-3-((5-chloro-6-ethyl-1H-indol-2-yl)methyl)-1-methylurea;
(R)-1-(1-acetylpiperidin-3-yl)-3-((5-chloro-6-methoxy-1H-indol-2-yl)methyl)-1-methylurea;
(R)-1-(1-acetylpiperidin-3-yl)-3-((5,6-dichloro-1H-indol-2-yl)methyl)-1-methylurea;
(R)-1-(1-acetylpiperidin-3-yl)-3-((5-chloro-6-ethoxy-1H-indol-2-yl)methyl)-1-methylurea;
(R)-1-(1-acetylpiperidin-3-yl)-3-((5,6-difluoro-1H-indol-2-yl)methyl)-1-methylurea;
(R)-1-(1-acetylpiperidin-3-yl)-3-((6-chloro-5-methyl-1H-indol-2-yl)methyl)-1-methylurea;
methyl (R)-3-(3-((5-chloro-1H-indol-2-yl)methyl)-1-methylureido)piperidine-1-carboxylate;
(R)-1-(1-acetylpiperidin-3-yl)-3-((5-chloro-6-fluoro-1H-indol-2-yl)methyl)-1-methylurea;
(R)-1-(1-acetylpiperidin-3-yl)-3-((6-fluoro-1H-indol-2-yl)methyl)-1-methylurea;
(R)-1-(1-acetylpiperidin-3-yl)-3-((5-fluoro-1H-indol-2-yl)methyl)-1-methylurea;
(R)-1-(1-acetylpiperidin-3-yl)-3-((6-methoxy-1H-indol-2-yl)methyl)-1-methylurea;
(R)-1-(1-acetylpiperidin-3-yl)-3-((5-chloro-1H-indol-2-yl)methyl)-1-ethylurea;
(R)-1-(1-acetylpiperidin-3-yl)-3-((6-chloro-5-fluoro-1H-indol-2-yl)methyl)-1-methylurea;
(R)-1-(1-acetylpiperidin-3-yl)-3-((5-chloro-1H-indol-6-yl)methyl)-1-methylurea;
(R)-1-(1-acetylpiperidin-3-yl)-1-ethyl-3-((5-fluoro-1H-indol-2-yl)methyl)urea;
(R)-1-(1-acetylpiperidin-3-yl)-3-((5-chloro-6-methoxy-1H-indol-2-yl)methyl)-1-ethylurea;
(R)-1-(1-acetylpiperidin-3-yl)-3-((5-fluoro-3-methyl-1H-indol-6-yl)methyl)-1-methylurea;
(R)-1-(1-acetylpiperidin-3-yl)-3-((5,6-difluoro-1H-indol-2-yl)methyl)-1-ethylurea;
(R)-1-(1-acetylpiperidin-3-yl)-3-((5-chloro-6-fluoro-1H-indol-2-yl)methyl)-1-ethylurea;
(R)-3-(3-((5-chloro-1H-indol-2-yl)methyl)-1-methylureido)-N-methylpiperidine-1-carboxamide;
(R)-1-(1-acetylpiperidin-3-yl)-3-((6-cyano-1H-indol-2-yl)methyl)-1-methylurea;
(R)-1-(1-(azetidine-1-carbonyl)piperidin-3-yl)-3-((5-chloro-1H-indol-2-yl)methyl)-1-methylurea;
(R)-1-(1-acetylpiperidin-3-yl)-3-((6-cyano-5-methyl-1H-indol-2-yl)methyl)-1-methylurea;
(R)-1-(1-acetylpiperidin-3-yl)-3-((7-(hydroxymethyl)-5-methyl-1H-indol-2-yl)methyl)-1-methylurea;
(R)-1-(1-acetylpiperidin-3-yl)-3-((3,5-dichloro-1H-indol-2-yl)methyl)-1-methylurea;
(R)-3-((5-chloro-1H-indol-2-yl)methyl)-1-methyl-1-(1-(5-methylisoxazole-3-carbonyl)piperidin-3-yl)urea;
(R)-1-(1-acetylpiperidin-3-yl)-3-((5-chloro-7-(methylamino)-1H-indol-2-yl)methyl)-1-methylurea;
(R)-1-(1-acetylpiperidin-3-yl)-3-((6-methoxy-5-methyl-1H-indol-2-yl)methyl)-1-methylurea;
(R)-3-((5-chloro-1H-indol-2-yl)methyl)-1-(1-(2-hydroxy-2-methylpropanoyl)piperidin-3-yl)-1-methylurea;
(R)-3-((5-chloro-1H-indol-2-yl)methyl)-1-methyl-1-(1-(pyrrolidine-1-carbonyl)piperidin-3-yl)urea;
(R)-1-(1-acetylpiperidin-3-yl)-3-((5-chloro-6-cyano-1H-indol-2-yl)methyl)-1-methylurea;
3-((5-chloro-1H-indol-2-yl)methyl)-1-((R)-1-((S)-2-hydroxypropanoyl)piperidin-3-yl)-1-methylurea;
(R)-1-(1-acetylpiperidin-3-yl)-3-((5-fluoro-1H-indol-6-yl)methyl)-1-methylurea;
1-((3R)-1-(1-acetylpyrrolidine-3-carbonyl)piperidin-3-yl)-3-((5-chloro-1H-indol-2-yl)methyl)-1-methylurea;
3-((5-chloro-1H-indol-2-yl)methyl)-1-((R)-1-((R)-2-hydroxypropanoyl)piperidin-3-yl)-1-methylurea;
(R)-3-((5-chloro-1H-indol-2-yl)methyl)-1-methyl-1-(1-(5-methyloxazole-4-carbonyl)piperidin-3-yl)urea;
(R)-3-((5-chloro-1H-indol-2-yl)methyl)-1-methyl-1-(1-(2-(pyridin-4-yl)acetyl)piperidin-3-yl)urea;
(R)-3-((5-chloro-1H-indol-2-yl)methyl)-1-(1-(4-hydroxybenzoyl)piperidin-3-yl)-1-methylurea;
(R)-3-((5-chloro-1H-indol-2-yl)methyl)-1-(1-(3-hydroxybenzoyl)piperidin-3-yl)-1-methylurea;
(R)-1-(1-(1H-pyrazole-5-carbonyl)piperidin-3-yl)-3-((5-chloro-1H-indol-2-yl)methyl)-1-methylurea;
1-((3R,6S or 3S,6R)-1-acetyl-6-methylpiperidin-3-yl)-3-((5-chloro-1H-indol-2-yl)methyl)-1-methylurea;
1-((3R,6S)-1-acetyl-6-methylpiperidin-3-yl)-3-((5-chloro-1H-indol-2-yl)methyl)-1-methylurea;
1-((3S,6R or 3R,6S)-1-acetyl-6-methylpiperidin-3-yl)-3-((5-chloro-1H-indol-2-yl)methyl)-1-methylurea;
1-((3S,6R)-1-acetyl-6-methylpiperidin-3-yl)-3-((5-chloro-1H-indol-2-yl)methyl)-1-methylurea;
(R)-3-((5-chloro-1H-indol-2-yl)methyl)-1-(1-(2-(isoxazol-5-yl)acetyl)piperidin-3-yl)-1-methylurea;
(R)-1-(1-acetylpiperidin-3-yl)-3-((5-chloro-1H-indol-2-yl)methyl)-1-(2,2,2-trifluoroethyl)urea;
(R)-3-((5-chloro-1H-indol-2-yl)methyl)-1-(1-(2-hydroxybenzoyl)piperidin-3-yl)-1-methylurea;
(R)-3-((5-chloro-1H-indol-2-yl)methyl)-1-methyl-1-(1-(2-(1-methyl-1H-pyrazol-3-yl)acetyl)piperidin-3-yl)urea;
(R)-3-((5-chloro-1H-indol-2-yl)methyl)-1-methyl-1-(1-(1-(2,2,2-trifluoroethyl)-1H-pyrazole-3-carbonyl)piperidin-3-yl)urea;
(R)-1-(1-(2-(1H-pyrazol-4-yl)acetyl)piperidin-3-yl)-3-((5-chloro-1H-indol-2-yl)methyl)-1-methylurea;
(R)-3-((5-chloro-1H-indol-2-yl)methyl)-1-methyl-1-(1-(2-methyloxazole-4-carbonyl)piperidin-3-yl)urea;
(R)-3-((5-chloro-1H-indol-2-yl)methyl)-1-(1-(2-(isoxazol-3-yl)acetyl)piperidin-3-yl)-1-methylurea;
(R)-3-((5-chloro-1H-indol-2-yl)methyl)-1-methyl-1-(1-(2-(oxazol-4-yl)acetyl)piperidin-3-yl)urea;
1-((3R,4R and 3S,4S)-1-acetyl-4-methylpiperidin-3-yl)-3-((5-chloro-1H-indol-2-yl)methyl)-1-methylurea;
(R)-3-((5-chloro-1H-indol-2-yl)methyl)-1-methyl-1-(1-(2-(oxazol-2-yl)acetyl)piperidin-3-yl)urea;
(R)-3-((5-chloro-1H-indol-2-yl)methyl)-1-methyl-1-(1-(2-(oxazol-5-yl)acetyl)piperidin-3-yl)urea;
(R)-1-(1-acetylpiperidin-3-yl)-3-(4-chloro-3-(1H-indol-2-yl)phenethyl)-1-methylurea;
(R)-1-(1-acetylpiperidin-3-yl)-3-(2,4-dichloro-5-(1H-indol-2-yl)phenethyl)-1-methylurea;
(R)-3-(3-((5-chloro-1H-indol-2-yl)methyl)-1-methylureido)-N,N-dimethylpiperidine-1-carboxamide;
(R)-3-(3-((5-chloro-1H-indol-2-yl)methyl)-1-methylureido)-N-ethylpiperidine-1-carboxamide;
(R)-3-(3-((5-chloro-1H-indol-2-yl)methyl)-1-methylureido)-N-(2,2,2-trifluoroethyl)piperidine-1-carboxamide;
(R)-3-(3-((5-chloro-1H-indol-2-yl)methyl)-1-methylureido)-N-cyclobutylpiperidine-1-carboxamide;
(R)-1-(1-acetylpiperidin-3-yl)-1-methyl-3-((5-(trifluoromethyl)-1H-indol-2-yl)methyl)urea;
(R)-3-(3-((5-chloro-1H-indol-2-yl)methyl)-1-methylureido)-N-isopropylpiperidine-1-carboxamide;
(R)-1-(1-acetylpiperidin-3-yl)-1-methyl-3-((6-methyl-1H-indol-2-yl)methyl)urea;
(R)-3-((5-chloro-1H-indol-2-yl)methyl)-1-(1-(3-hydroxypropanoyl)piperidin-3-yl)-1-methylurea;
(R)-3-((5-chloro-1H-indol-2-yl)methyl)-1-(1-(3,3-difluoroazetidine-1-carbonyl)piperidin-3-yl)-1-methylurea;
(R)-1-(1-acetylpiperidin-3-yl)-3-((5-ethyl-1H-indol-2-yl)methyl)-1-methylurea;
(R)-1-(1-acetylpiperidin-3-yl)-1-methyl-3-((5-methyl-1H-indol-2-yl)methyl)urea;
(R)-1-(1-acetylpiperidin-3-yl)-3-((6-ethyl-1H-indol-2-yl)methyl)-1-methylurea;
(R)-1-(1-acetylpiperidin-3-yl)-3-((5-cyclopropyl-1H-indol-2-yl)methyl)-1-methylurea;
(R)-1-(1-acetylpiperidin-3-yl)-3-((5-chloro-1H-indol-2-yl)methyl)-1-isopropylurea;
(R)-1-(1-acetylpiperidin-3-yl)-3-((5-chloro-1H-indol-2-yl)methyl)-1-cyclopropylurea;
(R)-1-(1-acetylpiperidin-3-yl)-1-methyl-3-((3-methyl-1H-indol-6-yl)methyl)urea;
(R)-1-(1-acetylpiperidin-3-yl)-3-((5-methoxy-1H-indol-2-yl)methyl)-1-methylurea;
(R)-1-(1-acetylpiperidin-3-yl)-3-((2,3-dimethyl-1H-indol-6-yl)methyl)-1-methylurea;
(R)-1-(1-acetylpiperidin-3-yl)-3-((5-chloro-6-methyl-1H-indol-2-yl)methyl)-1-methylurea;
(R)-1-(1-acetylpiperidin-3-yl)-3-((5-chloro-7-methyl-1H-indol-2-yl)methyl)-1-methylurea;
(R)-1-(1-acetylpiperidin-3-yl)-3-((5-cyano-1H-indol-2-yl)methyl)-1-methylurea;
(R)-1-(1-acetylpiperidin-3-yl)-3-((5-chloro-3-methyl-1H-indol-2-yl)methyl)-1-methylurea;
(R)-1-(1-acetylpiperidin-3-yl)-3-((5-chloro-7-(hydroxymethyl)-1H-indol-2-yl)methyl)-1-methylurea;
(R)-1-(1-acetylpiperidin-3-yl)-3-((5-chloro-7-hydroxy-1H-indol-2-yl)methyl)-1-methylurea;
(R)-3-((5-chloro-1H-indol-2-yl)methyl)-1-(1-(cyclobutanecarbonyl)piperidin-3-yl)-1-ethylurea;
(R)-1-(1-acetylpiperidin-3-yl)-3-((3,5-dimethyl-1H-indol-6-yl)methyl)-1-methylurea;
(R)-3-((5-chloro-1H-indol-2-yl)methyl)-1-methyl-1-(1-(4-methylisoxazole-3-carbonyl)piperidin-3-yl)urea;
(R)-1-(1-acetylpiperidin-3-yl)-3-((5-chloro-3-methyl-1H-indol-6-yl)methyl)-1-methylurea;
(R)-3-((5-chloro-1H-indol-2-yl)methyl)-1-(1-(1, 5-dimethyl-1H-pyrazole-3-carbonyl)piperidin-3-yl)-1-methylurea;
(R)-3-((5-chloro-1H-indol-2-yl)methyl)-1-methyl-1-(1-nicotinoylpiperidin-3-yl)urea;
(R)-3-((5-chloro-1H-indol-2-yl)methyl)-1-(1-(isoxazole-4-carbonyl)piperidin-3-yl)-1-methylurea;
(R)-3-((5-chloro-1H-indol-2-yl)methyl)-1-methyl-1-(1-(oxazole-5-carbonyl)piperidin-3-yl)urea;
(R)-3-((5-chloro-1H-indol-2-yl)methyl)-1-(1-(1-ethyl-1H-pyrazole-3-carbonyl)piperidin-3-yl)-1-methylurea;
(R)-3-((5-chloro-1H-indol-2-yl)methyl)-1-methyl-1-(1-picolinoylpiperidin-3-yl)urea;
1-((3R,6S and 3S,6R)-1-acetyl-6-methylpiperidin-3-yl)-3-((5-chloro-1H-indol-2-yl)methyl)-1-methylurea;
1-((3R,6S)-1-acetyl-6-methylpiperidin-3-yl)-3-((5-chloro-1H-indol-2-yl)methyl)-1-methylurea;
1-((3S,6R)-1-acetyl-6-methylpiperidin-3-yl)-3-((5-chloro-1H-indol-2-yl)methyl)-1-methylurea;
1-((3R,5S and 3S,5R)-1-acetyl-5-methylpiperidin-3-yl)-3-((5-chloro-1H-indol-2-yl)methyl)-1-methylurea;
1-((3R,5S)-1-acetyl-5-methylpiperidin-3-yl)-3-((5-chloro-1H-indol-2-yl)methyl)-1-methylurea;
1-((3S,5R)-1-acetyl-5-methylpiperidin-3-yl)-3-((5-chloro-1H-indol-2-yl)methyl)-1-methylurea;
(R)-1-(1-acetylpiperidin-3-yl)-3-(4-fluoro-3-(1H-indol-2-yl)phenethyl)-1-methylurea;
(R)-1-(1-acetylpiperidin-3-yl)-3-(4-chloro-2-fluoro-5-(1H-indol-2-yl)phenethyl)-1-methylurea;
(R)-3-((5-chloro-1H-indol-2-yl)methyl)-1-(1-(2-(isoxazol-4-yl)acetyl)piperidin-3-yl)-1-methylurea;
(R)-3-((5-chloro-1H-indol-2-yl)methyl)-1-methyl-1-(1-(5-methylisoxazol-3-yl)piperidin-3-yl)urea; and
(R)-3-((5-chloro-1H-indol-2-yl)methyl)-1-methyl-1-(1-(4-methylisoxazol-3-yl)piperidin-3-yl)urea;
   or a stereoisomer or tautomer thereof, or a pharmaceutically acceptable salt of any of the foregoing.

### Methods of Treatment

Provided herein is a method of modulating SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of inhibiting SLC6A19 in a cell, comprising exposing the cell to (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of reducing systemic amino acid levels in an individual in need thereof, comprising administering to the individual (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the amino acid is phenylalanine, tyrosine, glutamine, or glycine. In some embodiments, the systemic phenylalanine, tyrosine, glutamine, or glycine levels in the individual is reduced upon treatment. In some embodiments, the phenylalanine, tyrosine, glutamine, or glycine level is reduced at least 10%, at least 20%, at least 30% or at least 50% upon administration of the compound.

Provided herein is a method of treating a SLC6A19-mediated disease, disorder, or condition in an individual in need thereof, comprising administering to the individual (i) an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the SLC6A19-mediated disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders. In some embodiments, the SLC6A19-mediated disease, disorder, or condition is phenylketonuria (PKU), chronic kidney disease (CKD), methabolic syndrome, or metabolic diseases. In some embodiments, the SLC6A19-mediated disease, disorder, or condition is phenylketonuria (PKU). In some embodiments, the SLC6A19-mediated disease, disorder, or condition is chronic kidney disease (CKD). In some embodiments, the SLC6A19-mediated disease, disorder, or condition is a metabolic disease. In some embodiments, the SLC6A19-mediated disease, disorder, or condition is methabolic syndrome. In some embodiments, the SLC6A19-mediated disease, disorder, or condition is associated with abnormal levels of amino acids. In some embodiments, the SLC6A19-mediated disease, disorder, or condition is associated with a genetic defect in phenylalanine hydroxylase.

In some embodiments, the compounds provided herein increase the lifespan of the individual. In some embodiments, the lifespan is increased at least 5, at least 10, or at least 20 years upon treatment.

In some embodiments, the compounds provided herein inhibit SLC6A19 at a concentration of less than 10 μM, less than 1 μM, less than 0.5 μM, less than 0.1 μM, less that 0.010 μM, or less that 0.001 μM. In some embodiments, the compounds provided herein inhibit SLC6A19 at a concentration of 1-10 μM, 0.01 to 1 μM, or 0.01 to 10 μM.

In some embodiments, the compounds have an IC₅₀ of less than 10 nM, less than 10 μM, less than 1 μM, less than 0.5 μM, or less than 0.1 μM. In some embodiments, the compounds provided herein have an IC₅₀ of 1 to 10 nM, 1 to 10 μM, 0.01 to 1 μM, 0.01 to 10 μM, 0.001 to 0.01 μM or 0.001 to 0.010 μM.

In some embodiments, the individual receiving treatment is a juvenile human or an infant. In some embodiments, the individual is less than 10 years old, less than 9 years old, less than 8 years old, less than 7 years old, less than 6 years old, less than 5 years old, less than 4 years old, less than 3 years old, less than 2 years old, or less than one year old.

In some embodiments, the individual has abnormal levels of amino acids. In some embodiments, the SLC6A19-mediated disease, disorder, or condition is associated with a genetic defect in phenylalanine hydroxylase.

In some embodiments of the foregoing, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) an effective amount of a compound of formula (I), or any variation or embodiment thereof, or a stereoisomer or tautomer thereof, or a pharmaceutically acceptable salt of any of the foregoing, and (ii) instructions for use in treating an SLC6A19-mediated disease, disorder, or condition in an individual in need thereof. Also provided herein are kits, comprising (i) a pharmaceutical composition comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients; and (ii) instructions for use in treating an SLC6A19-mediated disease, disorder, or condition in an individual in need thereof.

Articles of manufacture are also provided, wherein the article of manufacture comprises a compound of formula (I), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising (i) an effective amount of a compound of formula (I), or any variation or embodiment thereof, or a stereoisomer or tautomer thereof, or a pharmaceutically acceptable salt of any of the foregoing, or (ii) a pharmaceutical composition comprising a compound of formula (I), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Preparing

The present disclosure further provides processes for preparing the compounds of present invention. In some aspects, provided herein are processes of preparing a compound of formula (I), (I-A), (I-A1), (I-A2), (I-A3), (I-A4), (I-A5), (I-A6), (I-B), (I-B1), (I-B32), (I-B33), (I-B34), (I-C), (I-C1), (I-C2), (I-D), (I-D1), (I-D2), (I-D3), (I-D4), (I-D5), (I-D6), or a stereoisomer or tautomer thereof, or a pharmaceutically acceptable salt of any of the foregoing.

In some embodiments, a process for preparing a compound of formula (I), or a stereoisomer or tautomer thereof, or a pharmaceutically acceptable salt of any of the foregoing, comprises:
reacting a compound of formula (I-1):

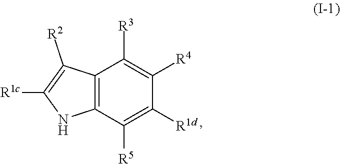

or a stereoisomer or tautomer thereof, or a pharmaceutically acceptable salt of any of the foregoing, wherein
one of R^{1c} and R^{1d} is

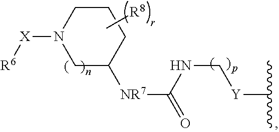

and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} bis optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Y is absent or

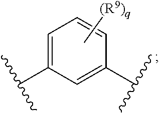

n is an integer from 1 to 2;
p is an integer from 1 to 4;
q is an integer from 0 to 4; and
r is an integer from 0 to 11;
with a compound of formula (I-2):

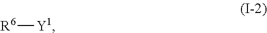

or a stereoisomer or tautomer thereof, or a pharmaceutically acceptable salt of any of the foregoing, wherein:
Y¹ is —C(O)OH or —C(O)-halo;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂; and
R^{z} is independently at each occurrence, C₁₋₆alkyl;
in the presence of one or more coupling reagents, to provide a compound of formula (I).

In some embodiments, the compound of formula (I-2) is a carboxylic acid or an acid halide (—C(O)-halo). In some embodiments, the compound of formula (I-2) is a carboxylic acid.

In some embodiments, the compound of formula (I-2) is an acid halide (—C(O)-halo). In some embodiments, the one or more coupling reagents comprises a base. In some embodiments, the base is a tertiary amine. In some embodiments the base is DIEA. In some embodiments, the one or more coupling reagents comprises EDCI, HOBt and/or a tertiary amine base such as DIEA.

In some embodiments, a process for preparing a compound of formula (I), or a stereoisomer or tautomer thereof, or a pharmaceutically acceptable salt of any of the foregoing, comprises:
reacting a compound of formula (II-1):

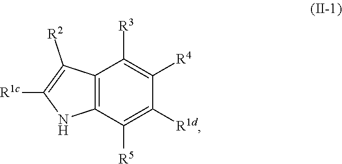

or a stereoisomer or tautomer thereof, or a pharmaceutically acceptable salt of any of the foregoing, wherein:
one of R^{1c} and R^{id} is

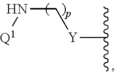

and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z},
wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Q¹H or —C(O)-LG;
LG is a leaving group;
Y is absent or

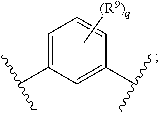

p is an integer from 1 to 4;
q is an integer from 0 to 4; and
with a compound of formula (I-2):

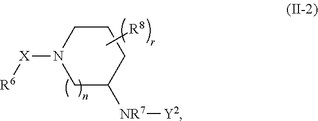

or a stereoisomer or tautomer thereof, or a pharmaceutically acceptable salt of any of the foregoing, wherein:
Y² is —H;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
n is an integer from 1 to 2;
r is an integer from 0 to 11.
each R^{a} is independently —OH, halo, —CN, C₁₋₆alkyl, C₁₋₆alkoxy, —N(R^{w1})(R^{w2}) —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl; and
X is absent or —C(O)—;
in the presence of one or more coupling reagents, to provide a compound of formula (I).

In some embodiments, the compound of formula (II-1) is an amine and the compound of formula (II-2) is a carbamate. In some embodiments, the compound of formula (II-1) is an amine and the compound of formula (II-2) is an amide. In some embodiments, the one or more coupling reagents comprises a base. In some embodiments, the base is a tertiary amine base such as DIEA.

In some embodiments, the leaving group is an alkoxy group. In some embodiments, the alkoxy group is substituted with one or more electronegative atoms. In some embodiments the alkoxy group is optionally substituted with one or more halo. In some embodiments, LG is —OCH₂CCl₃.

In some embodiments, the compound of formula (II-1) is an amine and the compound of formula (II-2) is an amine. In some embodiments, the one or more coupling reagents comprises a carbonate ester. In some embodiments, the carbonate ester is triphosgene. In some embodiments, the the one or more coupling reagents further comprises a base. In some embodiments, the base is a tertiary amine base such as DIEA.

### Enumerated Embodiments

Enumerated Embodiment 1. A compound of formula (I):

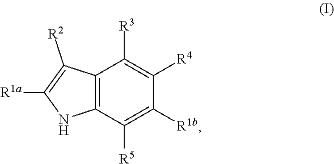

or a stereoisomer or tautomer thereof, or a pharmaceutically acceptable salt of any of the foregoing, wherein
one of R^{1a} and R^{1b} is

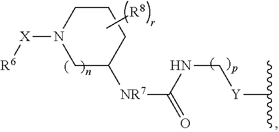

and
the other of R^{1a} and R^{1b} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein the C₁₋₆alkyl of R³ and R⁵ is optionally substituted with one or more —OH;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
   the C₁₋₆alkyl, C₆₋₂₀aryl, and 5-20 membered heteroaryl of R⁶ are each independently optionally substituted with one or more R^{a}, and
   the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
X is absent or —C(O)—;
Y is absent or

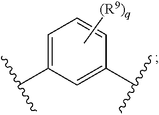

n is an integer from 1 to 2;
p is an integer from 1 to 4;
q is an integer from 0 to 4; and
r is an integer from 0 to 11.

Enumerated Embodiment 2. The compound of Enumerated Embodiment 1, wherein the compound is a compound of formula (I-A):

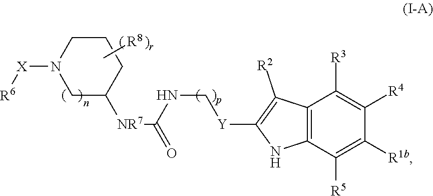

or a stereoisomer or tautomer thereof, or a pharmaceutically acceptable salt of any of the foregoing.

Enumerated Embodiment 3. The compound of Enumerated Embodiment 1, wherein the compound is a compound of formula (I-B):

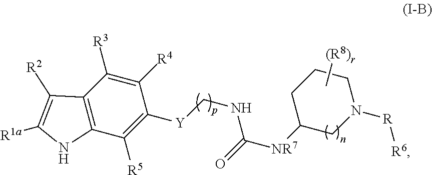

or a stereoisomer or tautomer thereof, or a pharmaceutically acceptable salt of any of the foregoing.

Enumerated Embodiment 4. The compound of Enumerated Embodiment 1 or Enumerated Embodiment 2, wherein the compound is a compound of formula (I-C):

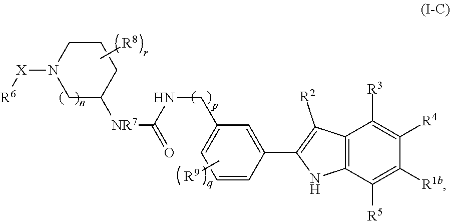

or a stereoisomer or tautomer thereof, or a pharmaceutically acceptable salt of any of the foregoing.

Enumerated Embodiment 5. The compound of Enumerated Embodiment 1 or Enumerated Embodiment 2, wherein the compound is a compound of formula (I-D):

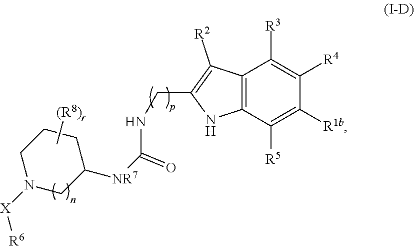

or a stereoisomer or tautomer thereof, or a pharmaceutically acceptable salt of any of the foregoing.

Enumerated Embodiment 6. The compound of any one of Enumerated Embodiments 1-5, or a stereoisomer or tautomer thereof, or a pharmaceutically acceptable salt of any of the foregoing, wherein X is —C(O)—.

Enumerated Embodiment 7. The compound of any one of Enumerated Embodiments 1-6, or a stereoisomer or tautomer thereof, or a pharmaceutically acceptable salt of any of the foregoing, wherein n is 1.

Enumerated Embodiment 8. The compound of any one of Enumerated Embodiments 1-7, or a stereoisomer or tautomer thereof, or a pharmaceutically acceptable salt of any of the foregoing, wherein n is 2.

Enumerated Embodiment 9. The compound of any one of Enumerated Embodiments 1-8, or a stereoisomer or tautomer thereof, or a pharmaceutically acceptable salt of any of the foregoing, wherein p is an integer from 1 to 3.

Enumerated Embodiment 10. The compound of any one of Enumerated Embodiments 1-9, or a stereoisomer or tautomer thereof, or a pharmaceutically acceptable salt of any of the foregoing, wherein p is 1 or 2.

Enumerated Embodiment 11. The compound of any one of Enumerated Embodiments 1-10, or a stereoisomer or tautomer thereof, or a pharmaceutically acceptable salt of any of the foregoing, wherein p is 1.

Enumerated Embodiment 12. The compound of any one of Enumerated Embodiments 1-11, or a stereoisomer or tautomer thereof, or a pharmaceutically acceptable salt of any of the foregoing, wherein R⁷ is C₁₋₃alkyl, C₁₋₃cycloalkyl, or C₁₋₃haloalkyl.

Enumerated Embodiment 13. The compound of any one of Enumerated Embodiments 1-12, or a stereoisomer or tautomer thereof, or a pharmaceutically acceptable salt of any of the foregoing, wherein R⁷ is C₁₋₃alkyl.

Enumerated Embodiment 14. The compound of any one of Enumerated Embodiments 1-13, or a stereoisomer or tautomer thereof, or a pharmaceutically acceptable salt of any of the foregoing, wherein R⁴ is H, halo, —CN, C₁₋₆alkyl, C₁₋₆cycloalkyl, C₁₋₆haloalkyl.

Enumerated Embodiment 15. The compound of any one of Enumerated Embodiments 1-14, or a stereoisomer or tautomer thereof, or a pharmaceutically acceptable salt of any of the foregoing, wherein R⁴ is H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}.

Enumerated Embodiment 16. The compound of Enumerated Embodiment 2, or a stereoisomer or tautomer thereof, or a pharmaceutically acceptable salt of any of the foregoing, wherein R^{1b} is H, halo, —CN, H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}.

Enumerated Embodiment 17. The compound of Enumerated Embodiment 3, or a stereoisomer or tautomer thereof, or a pharmaceutically acceptable salt of any of the foregoing, wherein R^{1a} is H, halo, —CN, H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}.

Enumerated Embodiment 18. The compound of any one of Enumerated Embodiments 1-17, or a stereoisomer or tautomer thereof, or a pharmaceutically acceptable salt of any of the foregoing, wherein R⁶ is C₁₋₃alkyl, C₁₋₃haloalkyl, C₁₋₃alkoxy, C₃₋₆cycloalkyl, —N(R^{w1})(R^{w2}), C₆₋₁₀ aryl, 3-6 membered heterocyclyl, or 5-10 membered heteroaryl, wherein
the C₁₋₃alkyl, C₆₋₁₀aryl, and 5-10 membered heteroaryl of R⁶ are each independently optionally substituted with one or more R^{a}, and
the C₃₋₆cycloalkyl, and 3-6 membered heterocyclyl of R⁶ are each independently optionally substituted with one or more R^{b}.

Enumerated Embodiment 19. The compound of any one of Enumerated Embodiments 1-18, or a stereoisomer or tautomer thereof, or a pharmaceutically acceptable salt of any of the foregoing, wherein R⁶ is 5-20 membered heteroaryl optionally substituted with one or more R^{a}.

Enumerated Embodiment 20. The compound of Enumerated Embodiment 19, or a stereoisomer or tautomer thereof, or a pharmaceutically acceptable salt of any of the foregoing, wherein R⁶ is selected from the group consisting of

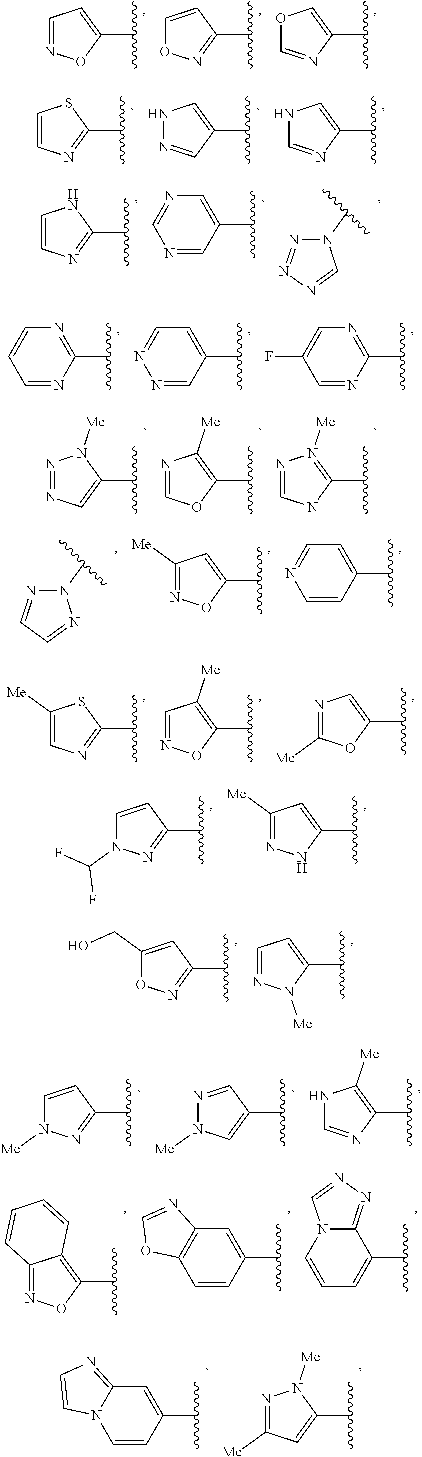

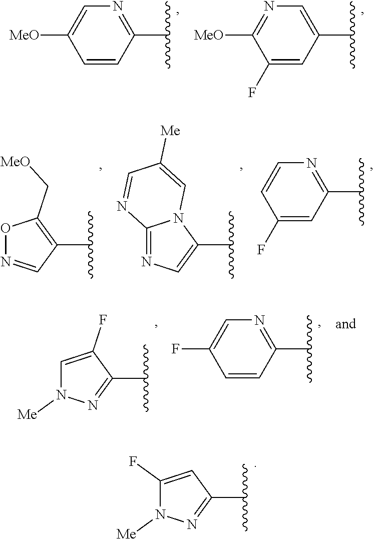

Enumerated Embodiment 21. The compound of any one of Enumerated Embodiments 1-18, or a stereoisomer or tautomer thereof, or a pharmaceutically acceptable salt of any of the foregoing, wherein R⁶ is 3-10 membered heterocyclyl optionally substituted with one or more R^{b}.

Enumerated Embodiment 22. The compound of Enumerated Embodiment 21, or a stereoisomer or tautomer thereof, or a pharmaceutically acceptable salt of any of the foregoing, wherein R⁶ is selected from the group consisting of

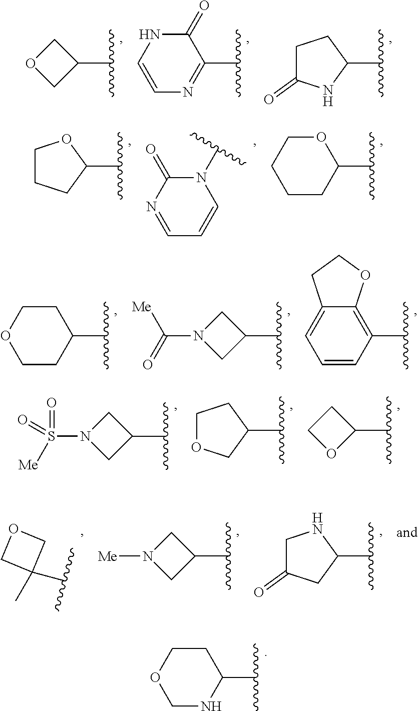

Enumerated Embodiment 23. The compound of any one of Enumerated Embodiments 1-18, or a stereoisomer or tautomer thereof, or a pharmaceutically acceptable salt of any of the foregoing, wherein R⁶ is C₆₋₂₀aryl optionally substituted with one or more R^{a}.

Enumerated Embodiment 24. The compound of Enumerated Embodiment 23, or a stereoisomer or tautomer thereof, or a pharmaceutically acceptable salt of any of the foregoing, wherein R⁶ is selected from the group consisting of

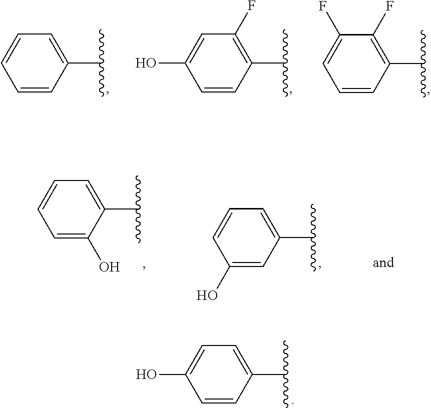

Enumerated Embodiment 25. The compound of any one of Enumerated Embodiments 1-18, or a stereoisomer or tautomer thereof, or a pharmaceutically acceptable salt of any of the foregoing, wherein R⁶ is C₃₋₁₀cycloalkyl optionally substituted with one or more R^{b}.

Enumerated Embodiment 26. The compound of Enumerated Embodiment 25, or a stereoisomer or tautomer thereof, or a pharmaceutically acceptable salt of any of the foregoing, wherein R⁶ is selected from the group consisting of

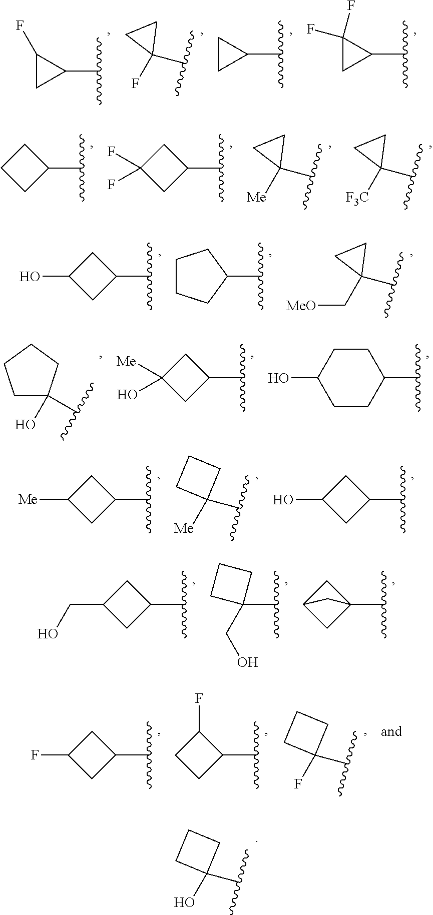

Enumerated Embodiment 27. The compound of any one of Enumerated Embodiments 1-18, or a stereoisomer or tautomer thereof, or a pharmaceutically acceptable salt of any of the foregoing, wherein R⁶ is C₁₋₆alkyl optionally substituted with one or more R^{a}.

Enumerated Embodiment 28. The compound of any one of Enumerated Embodiments 1-27, or a stereoisomer or tautomer thereof, or a pharmaceutically acceptable salt of any of the foregoing, wherein R² is H, halo, —CN, C₁₋₃alkyl, C₁₋₄cycloalkyl, C₁₋₃haloalkyl.

Enumerated Embodiment 29. The compound of any one of Enumerated Embodiments 1-28, or a stereoisomer or tautomer thereof, or a pharmaceutically acceptable salt of any of the foregoing, wherein R³ and R⁵ are each independently H, —OH, halo, —N(R^{w1})(R^{w2}), or C₁₋₄alkyl, wherein the C₁₋₄alkyl is optionally substituted with one or more —OH.

Enumerated Embodiment 30. The compound of any one of Enumerated Embodiments 1-29, or a stereoisomer or tautomer thereof, or a pharmaceutically acceptable salt of any of the foregoing, wherein R^{a} is independently C₁₋₆alkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c}.

Enumerated Embodiment 31. The compound of any one of Enumerated Embodiments 1-30, or a stereoisomer or tautomer thereof, or a pharmaceutically acceptable salt of any of the foregoing, wherein R^{b} is independently C₁₋₆alkyl optionally substituted with one or more R^{c}.

Enumerated Embodiment 32. The compound of any one of Enumerated Embodiments 1-31, or a stereoisomer or tautomer thereof, or a pharmaceutically acceptable salt of any of the foregoing, wherein R^{c} is independently —OH, halo, —N(R^{w1})(R^{w2}), C₁₋₃alkyl, —C₁₋₃alkylN(R^{w1})(R^{w2}), or C₁₋₃alkoxy.

Enumerated Embodiment 33. The compound of any one of Enumerated Embodiments 1-32, or a stereoisomer or tautomer thereof, or a pharmaceutically acceptable salt of any of the foregoing, wherein R^{w1} and R^{w2} are independently at each occurrence H, C₁₋₆alkyl, C₁₋₆haloalkyl, or C₃₋₁₀cycloalkyl.

Enumerated Embodiment 34. The compound of Enumerated Embodiment 1, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from Table 1.

Enumerated Embodiment 35. A process for preparing a compound of Enumerated Embodiment 1, or a stereoisomer or tautomer thereof, or a pharmaceutically acceptable salt of any of the foregoing, wherein the process comprises:
reacting a compound of formula (I-1):

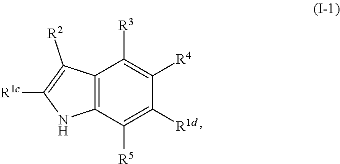

or a stereoisomer or tautomer thereof, or a pharmaceutically acceptable salt of any of the foregoing, wherein
one of R^{1c} and R^{1d} is

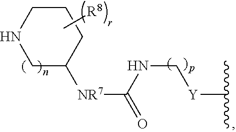

and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Y is absent or

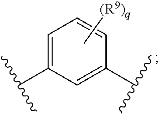

n is an integer from 1 to 2;
p is an integer from 1 to 4;
q is an integer from 0 to 4; and
r is an integer from 0 to 11;
with a compound of formula (I-2):

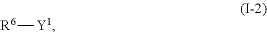

or a stereoisomer or tautomer thereof, or a pharmaceutically acceptable salt of any of the foregoing, wherein:
Y¹ is —C(O)OH or —C(O)-halo;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂; and
R^{z} is independently at each occurrence, C₁₋₆alkyl;
in the presence of one or more coupling reagents.

Enumerated Embodiment 36. The process of Enumerated Embodiment 35, wherein the one or more coupling reagents comprises EDCI, HOBt and/or DIEA.

Enumerated Embodiment 37. A process for preparing a compound of Enumerated Embodiment 1, or a stereoisomer or tautomer thereof, or a pharmaceutically acceptable salt of any of the foregoing, wherein the process comprises:
reacting a compound of formula (II-1):

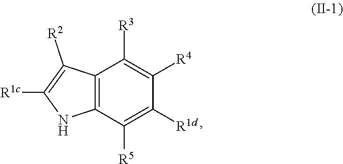

or a stereoisomer or tautomer thereof, or a pharmaceutically acceptable salt of any of the foregoing, wherein:
one of R^{1c} and R^{1d} is

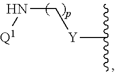

and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Q¹H or —C(O)-LG;
LG is a leaving group;
Y is absent or

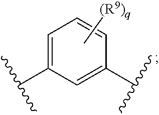

p is an integer from 1 to 4;
q is an integer from 0 to 4; and
with a compound of formula (I-2):

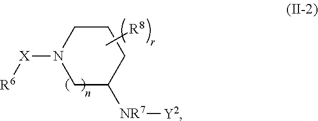

or a stereoisomer or tautomer thereof, or a pharmaceutically acceptable salt of any of the foregoing, wherein:
Y² is —H;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
n is an integer from 1 to 2;
r is an integer from 0 to 11.
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
   the C₁₋₆alkyl, C₃₋₁₀cycloalkyl, and 3-10 membered heterocyclyl of R^{a} are each optionally substituted with one or more R^{b}, and
   the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
   the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl; and
X is absent or —C(O)—;
in the presence of one or more coupling reagents.

Enumerated Embodiment 38. The process of Enumerated Embodiment 37, wherein the one or more coupling reagents comprises a base.

Enumerated Embodiment 39. The process of Enumerated Embodiment 37, wherein the one or more coupling reagents comprises a carbonate ester.

Enumerated Embodiment 40. The process of Enumerated Embodiment 39, wherein the carbonate ester is triphosgene.

Enumerated Embodiment 41. The process of any one of Enumerated Embodiments 37-40, wherein LG is an alkoxy group optionally substituted with one or more halo.

Enumerated Embodiment 42. A pharmaceutical composition comprising (i) a compound of any one of Enumerated Embodiments 1-34, or a stereoisomer or tautomer thereof, or a pharmaceutically acceptable salt of any of the foregoing, and (ii) one or more pharmaceutically acceptable excipients.

Enumerated Embodiment 43. A method of modulating SLC6A19 in a cell, comprising exposing the cell to an effective amount of a compound of any one of Enumerated Embodiments 1-34, or a stereoisomer or tautomer thereof, or a pharmaceutically acceptable salt of any of the foregoing, or a pharmaceutical composition of Enumerated Embodiment 42.

Enumerated Embodiment 44. A method of inhibiting SLC6A19 in a cell, comprising exposing the cell to an effective amount of a compound of any one of Enumerated Embodiments 1-34, or a stereoisomer or tautomer thereof, or a pharmaceutically acceptable salt of any of the foregoing, or a pharmaceutical composition of Enumerated Embodiment 42.

Enumerated Embodiment 45. A method of reducing systemic phenylalanine, tyrosine, glutamine, or glycine levels in an individual in need thereof, comprising administering to the individual an effective amount of a compound of any one of Enumerated Embodiments 1-34, or a stereoisomer or tautomer thereof, or a pharmaceutically acceptable salt of any of the foregoing, or a pharmaceutical composition of Enumerated Embodiment 42.

Enumerated Embodiment 46. A method of treating a SLC6A19-mediated disease, disorder, or condition in an individual in need thereof, comprising administering to the individual an effective amount of a compound of any one of Enumerated Embodiments 1-34, or a stereoisomer or tautomer thereof, or a pharmaceutically acceptable salt of any of the foregoing, or a pharmaceutical composition of Enumerated Embodiment 42.

Enumerated Embodiment 47. The method of Enumerated Embodiment 46, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Enumerated Embodiment 48. The method of Enumerated Embodiment 46 or Enumerated Embodiment 47, wherein the disease, disorder, or condition is selected from the group consisting of phenylketonuria (PKU), chronic kidney disease (CKD), methabolic syndrome, and metabolic diseases.

Enumerated Embodiment 49. The method of Enumerated Embodiment 46, wherein the disease, disorder, or condition is associated with abnormal levels of amino acids.

Enumerated Embodiment 50. The method of Enumerated Embodiment 46, or Enumerated Embodiment 49, wherein the disease, disorder, or condition is associated with a genetic defect in phenylalanine hydroxylase.

Enumerated Embodiment 51. A kit, comprising (i) a compound of any one of Enumerated Embodiments 1-34, or a stereoisomer or tautomer thereof, or a pharmaceutically acceptable salt of any of the foregoing, or a pharmaceutical composition of Enumerated Embodiment 43, and (ii) instructions for use in treating an SLC6A19-mediated disease, disorder, or condition in an individual in need thereof.

Enumerated Embodiment 52. The kit of Enumerated Embodiment 51, wherein the disease, disorder, or condition is associated with abnormal levels of amino acids.

Enumerated Embodiment 53. The kit of Enumerated Embodiment 51, or Enumerated Embodiment 52, wherein the disease, disorder, or condition is associated with a genetic defect in phenylalanine hydroxylase.

Enumerated Embodiment 54. The kit of Enumerated Embodiment 51, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

Enumerated Embodiment 55. The kit of Enumerated Embodiment 51, wherein the individual has a genetic defect in phenylalanine hydroxylase.

### General Synthetic Schemes

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### SYNTHETIC EXAMPLES

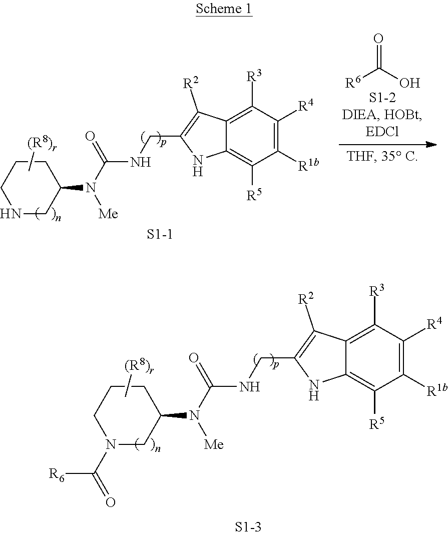

Compounds of formula S1-3 can be prepared according to Scheme 1. Reaction of amine S1-1 with carboxylic acid S1-2 in the presence of a coupling agent such as EDCI, a catalyst such as HOBt, and a tertiary amine base such as DIEA in an aprotic solvent such as THF gives compounds of formula S1-3, which may require further deprotection or functional group manipulations using standard conditions to arrive at the final compound.

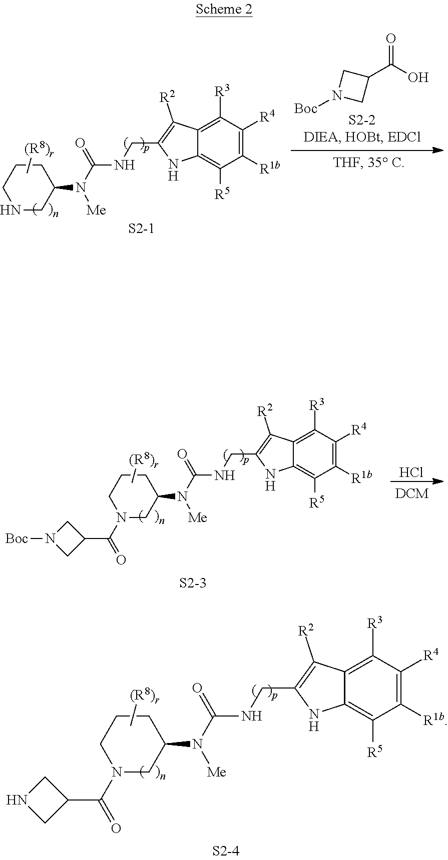

Compounds of formula S2-4 can be prepared according to Scheme 2. Amide bond formation between amine S2-1 and carboxylic acid S2-2 under the conditions described in Scheme 1 generates S2-3. Cleavage of the N-Boc protecting group with a protic acid such as HCl in an aprotic solvent such as DCM gives compounds of formula S2-4. If desired, compounds of formula S2-4 may be further elaborated.

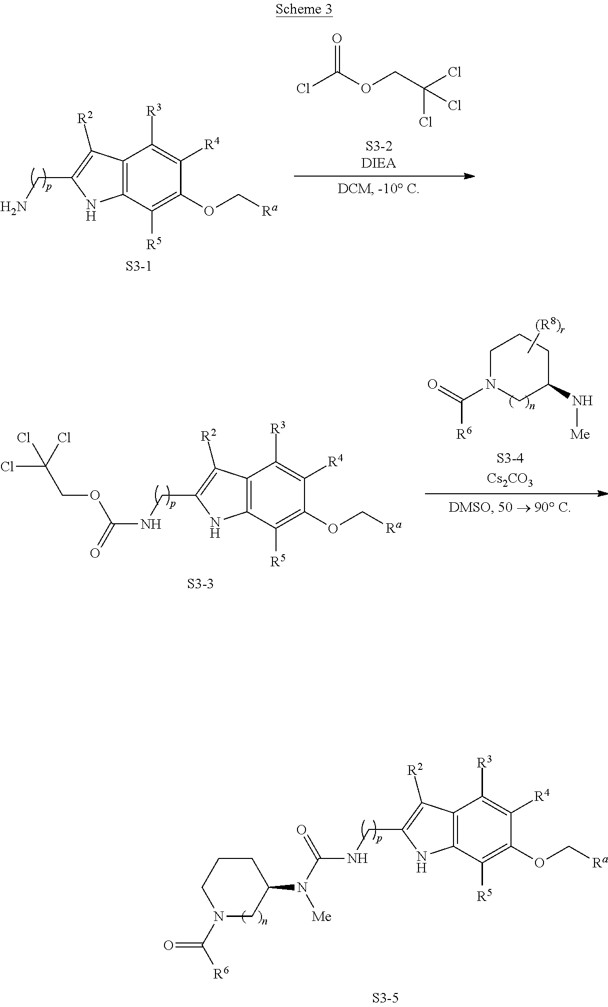

Compounds of formula S3-5 can be prepared according to Scheme 3. Treatment of amine S3-1 with a chloroformate derivative such as S3-2 in the presence of a tertiary amine base such as DIEA gives carbamate S3-3. Heating carbamate S3-3 with amine S3-4 and cesium carbonate in DMSO gives compounds of formula S3-5.

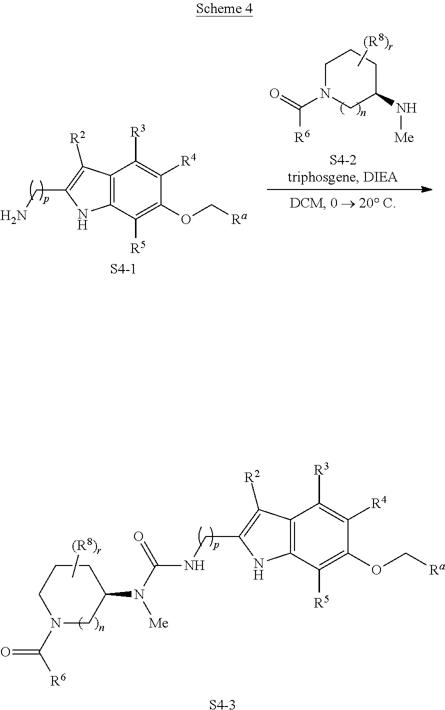

Compounds of formula S4-3 can be prepared according to Scheme 4. Urea formation between amines S4-1 and S4-2 occurs in the presence of triphosgene and DIEA in an aprotic solvent such as DCM to give compounds of formula S4-3, which may require further deprotection or functional group manipulations using standard conditions to arrive at the final compound.

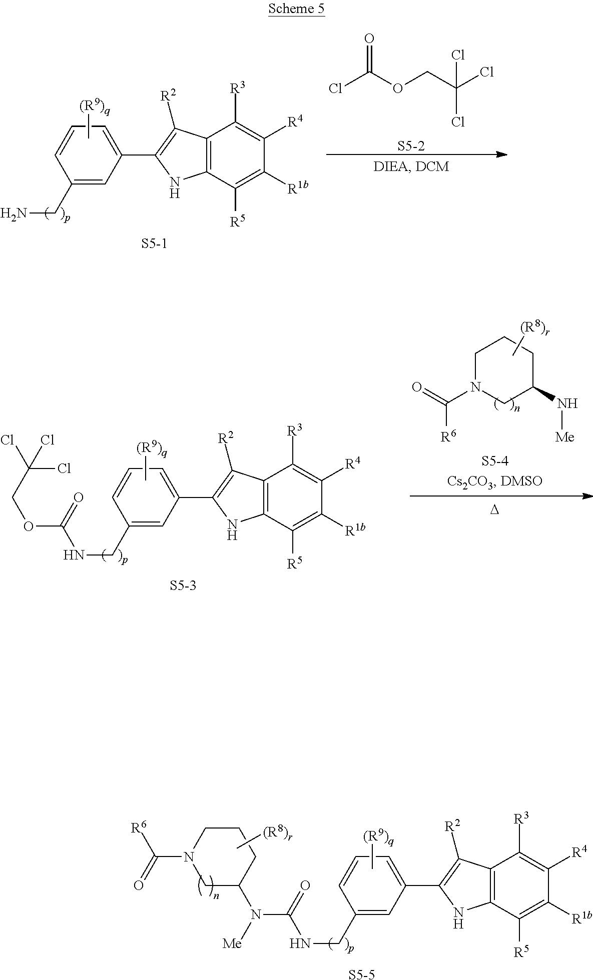

Compounds of formula S5-5 can be prepared according to Scheme 5. Treatment of amine S5-1 with a chloroformate derivative such as S5-2 in the presence of a tertiary amine base such as DIEA gives carbamate S5-3. Heating carbamate S5-3 with amine S5-4 and cesium carbonate in DMSO gives compounds of formula S5-5.

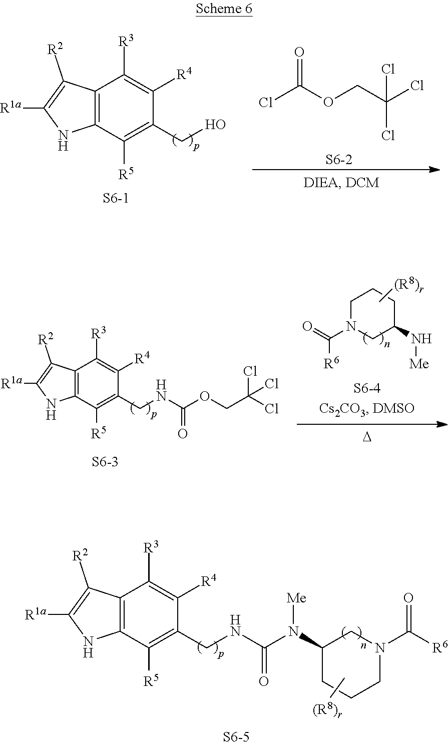

Compounds of formula S6-5 can be prepared according to Scheme 6. Treatment of amine S6-1 with a chloroformate derivative such as S6-2 in the presence of a tertiary amine base such as DIEA gives carbamate S6-3. Heating carbamate S6-3 with amine S6-4 and cesium carbonate in DMSO gives compounds of formula S6-5.

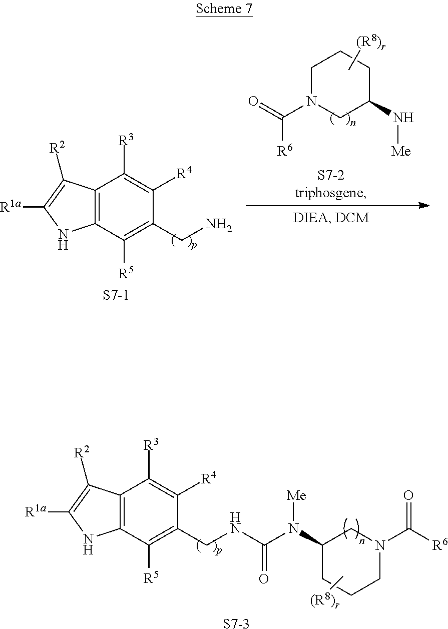

Compounds of formula S7-3 can be prepared according to Scheme 7. Urea formation between amines S7-1 and S7-2 occurs in the presence of triphosgene and DIEA in an aprotic solvent such as DCM to give compounds of formula S7-3, which may require further deprotection or functional group manipulations using standard conditions to arrive at the final compound.

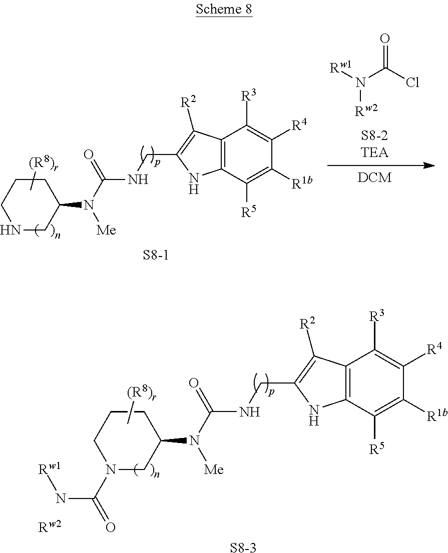

Compounds of formula S8-3 can be prepared according to Scheme 8. Urea formation between amine S8-1 and a carbamoyl chloride such as S8-2 in the presence of an amine base such as TEA in an aprotic solvent such as DCM gives urea S8-3.

### EXAMPLES

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | |
|---|---|
| ° C. | degrees Celsius |
| μL | microliter |
| [M + XX]⁺ | observed mass |
| AC₅₀ | half-maximal activity concentration |
| ACN | acetonitrile |
| app | apparent (NMR) |
| Boc₂O | Di-tert-butyl dicarbonate |
| BH₃•THF | borane-tetrahydrofuran complex |
| BBr₃ | boron tribromide |
| Calc'd | calculated |
| cataCXium | Chloro[(di(1-adamantyl)- |
| A Pd G2 | N-butylphosphine)-2-(2-aminobiphenyl)]palladium(II) |
| Cbz-Cl | benzyl chloroformate |
| CO₂ | carbon dioxide |
| Cs₂CO₃ | cesium carbonate |
| d | deuterated (NMR solvents) |
| d | doublet (NMR) |
| dd | doublet of doublets (NMR) |
| DABCO | 1,4-diazobicyclo[2.2.2]octane |
| DCM | dichloromethane |
| DIAD | diisopropyl azodicarboxylate |
| DMAP | 4-Dimethylaminopyridine |
| DPPA | Diphenyl phosphoryl azide |
| DMF | N,N-dimethylformamide |
| EC₅₀ | half-maximal effective concentration |
| EDCI | 1-Ethyl-3-(3-dimethylaminopropyl)carbodiimide |
| ESI | electrospray ionization |
| EtOAc | ethyl acetate |
| EtOH | ethanol |
| eq | equivalents |
| g | grams |
| h | hours |
| H | hydrogen |
| HATU | 1-[Bis(dimethylamino)methylene]-1H-1,2,3- |
| | triazolo[4,5-b]pyridinium 3-oxid hexafluorophosphate |
| HCl | hydrochloric acid |
| HPLC | high-performance liquid chromatography |
| IC₅₀ | half-maximal inhibitory concentration |
| In vacuo | in a vacuum |
| IUPAC | International Union of Pure and Applied Chemistry |
| MHz | megahertz |
| J | J-coupling value (NMR) |
| K₂CO₃ | potassium carbonate |
| LDA | lithium diisopropylamide |
| LiHMDS | lithium bis(trimethylsilyl)amide |
| MeOH | Methanol |
| MeCN | acetonitrile |
| m | multiplet (NMR) |
| mg | milligrams |
| min | minutes |
| mL | milliliter |
| mmol | millimole |
| mM | millimolar |
| M | molarity or molar |
| MS | mass spectrometry |
| MsCl | methanesulfonyl chloride |
| Ms₂O | methanesulfonic anhydride |
| MTBE | methyl tert-butyl ether |
| n/a | not applicable |
| NBS | N-bromosuccinimide |
| NCS | N-chlorosuccinimide |
| NIS | N-iodosuccinimide |
| NfF | nonafluorobutanesulfonyl fluoride |
| NH₄ | ammonium |
| NH₄OH | ammonium hydroxide |
| NH₄HCO₃ | ammonium bicarbonate |
| Na₂SO₄ | sodium sulfate |
| NaBH₃CN | sodium cyanoborohydride |
| NMI | N-methylimidazole |
| NMM | N-methylmorpholine |
| NMR | nuclear magnetic resonance |
| NaOH | sodium hydroxide |
| PCC | Pyridinium chlorochromate |
| PCy₃ | Tricyclohexylphosphine |
| Pd₂(dba)₃ | Tris(dibenzylideneacetone)dipalladium(0) |
| PdCl₂(dppf) | [1,1′- |
| | Bis(diphenylphosphino)ferrocene]dichloropalladium(II) |
| pH | potential of hydrogen |
| PPh₃ | triphenyl phosphine |
| s | singlet (NMR) |
| SFC | super fluid chromatography |
| t | triplet (NMR) |
| T3P | Propanephosphonic acid anhydride |
| TBAB | tetrabutylammonium bromide |
| TBAF | tetrabutylammonium fluoride |
| TEA | triethylamine |
| TFA | trifluoroacetic acid |
| TFCH | N,N,N′,N′-tetramethylchloroformamidinium |
| | hexafluorophosphate |
| THF | Tetrahydrofuran |
| TMAD | Tetramethylazodicarboxamide |
| TMSCl | trimethylsilyl chloride |
| TsCl | p-toluenesulfonyl chloride |
| wt. % | weight percent |
| XPhos | Dicyclohexyl[2′,4′,6′-tris(propan-2-yl)[1,1′- |
| | biphenyl]-2-yl]phosphane |
| Xantphos | (9,9-Dimethyl-9H-xanthene-4,5- |
| | diyl)bis(diphenylphosphane) |

### Intermediate A-1: (R)-3-((5-chloro-1H-indol-2-yl)methyl)-1-methyl-1-(piperidin-3-yl)urea

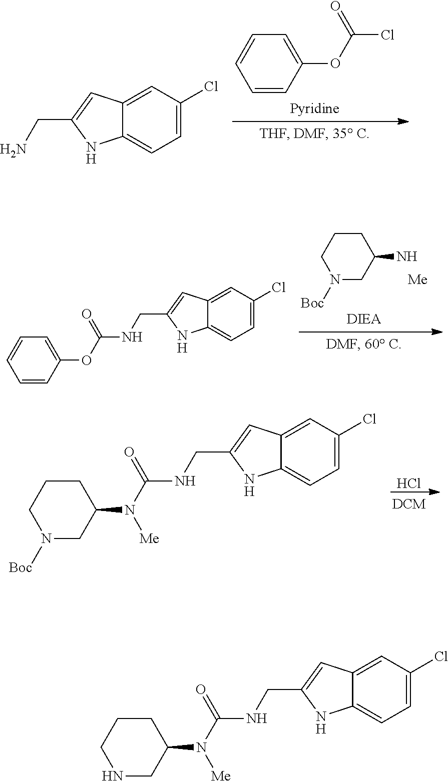

Step 1. To a solution of (5-chloro-1H-indol-2-yl)methanamine (3.15 g, 17.4 mmol, 1 eq) in THE (87 mL, 0.2 M) and DMF (5 mL, 3.5 M) at 0° C. was added pyridine (2.1 mL, 26.2 mmol, 1.5 eq) followed by drop-wise addition of phenyl chloroformate (2.406 mL, 19.2 mmol, 1.1 eq). The resulting mixture was stirred at 0° C. for 0.5 h and then warmed to 35° C. After 16 h the reaction mixture was cooled to room temperature, diluted with ethyl acetate (3×100 mL) then washed sequentially with 5% aq. LiCl (75 mL), water (50 mL) and brine (30 mL). The organic layer was dried over anhydrous Na₂SO₄, filtered and concentrated under reduced pressure. The resulting crude material was purified by column chromatography to give phenyl ((5-chloro-1H-indol-2-yl)methyl)carbamate. LC-MS (ESI) m/z: [M+H]⁺ calcd for C₁₆H₁₃ClN₂O₂: 301.07; found 301.1.

Step 2. To a solution of phenyl ((5-chloro-1H-indol-2-yl)methyl)carbamate (3.48 g, 11.6 mmol, 1 eq) and DIEA (6.05 mL, 34.7 mmol, 3 eq) in DMF (39 mL, 0.3 M) was added tert-butyl (3R)-3-(methylamino)piperidine-1-carboxylate (3.72 g, 17.4 mmol, 1.5 eq) and the resulting mixture was heated to 60° C. After 16 h the reaction was cooled to room temperature. The reaction mixture was diluted with DCM (3×75 mL) then washed sequentially with water (50 mL), 5% aq. LiCl (50 mL) and brine (30 mL). The organic layer was dried over anhydrous Na₂SO₄, filtered and concentrated under reduced pressure. The resulting crude material was purified by column chromatography to give tert-butyl (3R)-3-({[(5-chloro-1H-indol-2-yl)methyl]carbamoyl}(methyl)amino)piperidine-1-carboxylate. LC-MS (ESI) m/z: [M+H]⁺ calcd for C₂₁H₂₉ClN₄O₃: 421.20; found 320.9 [M−Boc+H]⁺.

Step 3. To a solution of tert-butyl (3R)-3-({[(5-chloro-1H-indol-2-yl)methyl]carbamoyl}(methyl)amino)piperidine-1-carboxylate (1.65 g, 3.92 mmol, 1 eq) in DCM (13 mL, 0.3 M) was added HCl (20 mL, 4 M in dioxane, 78.4 mmol, 20 eq). The resulting mixture was heated to 30° C. After 2 h, the reaction was cooled to room temperature and concentrated under reduced pressure. The residue was partitioned between DCM (75 mL) and 1 N NaOH (50 mL). The aqueous phase was washed with DCM (2×75 mL). The combined organic layers were washed sequentially with 5% aq. LiCl (50 mL) and brine (50 mL), dried over anhydrous Na₂SO₄, filtered and concentrated under reduced pressure to give (R)-3-((5-chloro-1H-indol-2-yl)methyl)-1-methyl-1-(piperidin-3-yl)urea which was used without further purification. LC-MS (ESI) m/z: [M+H]⁺ calcd for C₁₆H₂₁ClN₄O: 321.15; found 321.5.

### Intermediate A-2: (R)-N-methyl-1-(pyridazin-3-yl)piperidin-3-amine

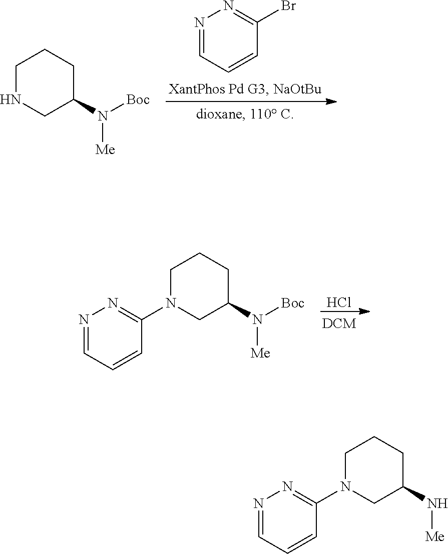 [SMILES: CN(C(=O)OC(C)(C)C)[C@@H]1CCCNC1 ; Brc1cccnn1 ; CN(C(=O)OC(C)(C)C)[C@@H]1CCCN(c2cccnn2)C1 ; Cl ; CN[C@@H]1CCCN(c2cccnn2)C1]

Step 1. To tert-butyl (R)-methyl(piperidin-3-yl)carbamate (1.51 g, 7.07 mmol, 1.0 eq) was added to a solution of 3-bromopyridazine (0.750 g, 4.71 mmol), sodium tert-butoxide (1.36 g, 14.1 mmol, 2.0 eq) and XantPhos Pd G3 (0.671 g, 0.708 mmol, 0.10 eq) in dioxane (13.4 mL). The resulting mixture heated to 110° C. After 16 h the reaction was cooled to room temperature then was diluted with EtOAc (100 mL) and washed with water (2×100 mL). The organic layer was dried over Na₂SO₄, filtered, and concentrated under reduced pressure. The residue was purified by column chromatography to tert-butyl (R)-methyl(1-(pyridazin-3-yl)piperidin-3-yl)carbamate. LC-MS (ESI) m/z: [M+H]⁺ calcd for C₁₅H₂₄N₄O₂: 292.19; found 292.9.

Step 2. To a solution tert-butyl (R)-methyl(1-(pyridazin-3-yl)piperidin-3-yl)carbamate (0.578 g, 1.97 mmol, 1.0 eq) in dichloromethane (3.95 mL) at 0° C. was added hydrogen chloride (4.94 mL, 4 M in dioxane, 19.7 mmol, 10.0 eq). The mixture was warmed to room temperature. After 2 h the reaction was concentrated under reduced pressure to give (R)-N-methyl-1-(pyridazin-3-yl)piperidin-3-amine which was used without further purification. LC-MS (ESI) m/z: [M+H]⁺ calcd for C₁₀H₁₆N₄: 192.14; found 193.2.

### Intermediate A-3: 5-chloro-6-methoxy-1H-indole-2-carboxylic acid

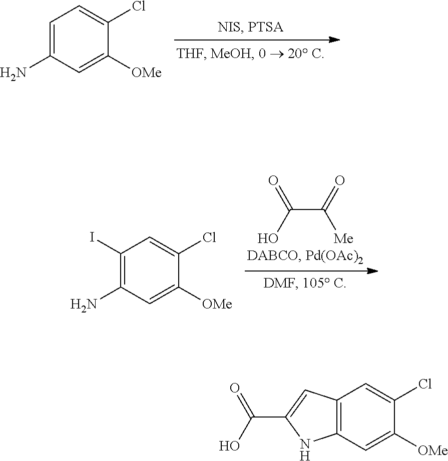 [SMILES: COc1cc(N)ccc1Cl ; COc1cc(N)c(I)cc1Cl ; CC(=O)C(=O)O ; COc1cc2[nH]c(C(=O)O)cc2cc1Cl]

Step 1: 3 reactions were carried out in parallel as described below: To a solution of 4-chloro-3-methoxyaniline (33.0 g, 209 mmol, 1.00 eq) in THE (330 mL) and MeOH (330 mL) were added N-iodosuccinimide (37.6 g, 167 mmol, 0.80 eq) and p-toluene sulfonic acid (36.0 g, 209 mmol, 1.00 eq) portion-wise at 0° C. The reaction mixture was then allowed to warm to 20° C. and stirred for 1 h, at which point the 3 reactions were combined for work up. The combined reaction mixture was poured into ice-water (500 mL) and stirred for 10 min. The aqueous phase was extracted with ethyl acetate (3×300 mL). The combined organic layers were washed with brine (500 mL), dried over anhydrous Na₂SO₄, filtered and concentrated under vacuum to give a residue. The residue was purified by column chromatography to give 4-chloro-2-iodo-5-methoxyaniline. LC-MS (ESI) m/z: [M+H]⁺ calcd for C₇H₇ClINO: 282.93; found: 284.0.

Step 2: To a mixture of compound 4-chloro-2-iodo-5-methoxyaniline (40.0 g, 141 mmol, 1.00 eq), 2-oxopropanoic acid (37.2 g, 423 mmol, 3.00 eq) and DABCO (47.0 g, 423 mmol, 3.00 eq) in DMF (400 mL) was degassed and purged with N₂ 3 times, then Pd(OAc)₂ (3.17 g, 14.1 mmol, 0.10 eq) was added to the mixture in one portion at 25° C. under a N₂ atmosphere. The reaction mixture was then heated to 105° C. and stirred for 3 h. After cooling the mixture to room temperature, the reaction was poured into ice-water (500 mL) and stirred for 10 min. The aqueous phase was extracted with ethyl acetate (3×500 mL). The combined organic layers were washed with brine (500 mL), dried over anhydrous Na₂SO₄, filtered and concentrated in vacuo to give a residue. The residue was triturated with MTBE at 20° C. for 30 min and filtered. The filter cake was collected to give 5-chloro-6-methoxy-1H-indole-2-carboxylic acid. LC-MS (ESI) m/z: [M+H]⁺ calcd for C₁₀H₈ClNO₃: 225.02; found: 226.0.

### Intermediate A-4: tert-butyl ((5-chloro-6-hydroxy-1H-indol-2-yl)methyl)carbamate

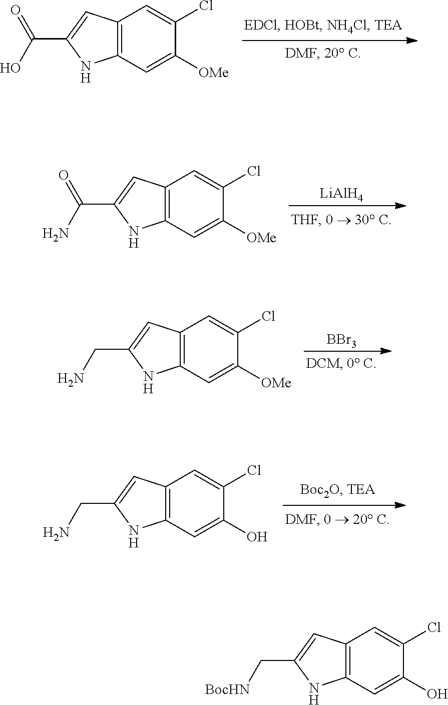

Step 1: To a solution of 5-chloro-6-methoxy-1H-indole-2-carboxylic acid (15.8 g, 70 mmol, 1.00 eq) in DMF (160 mL) were added EDCI (16.1 g, 84 mmol, 1.20 eq) and HOBt (11.0 g, 84 mmol, 1.20 eq). The mixture was stirred at 20° C. for 0.5 h, then NH₄C₁ (5.62 g, 105 mmol, 1.50 eq) and triethylamine (105 mmol, 14.6 mL, 1.50 eq) were added portion-wise. The reaction mixture was then stirred at 20° C. for 16 h, then was quenched with water (300 mL) and stirred for 10 min. The aqueous phase was extracted with ethyl acetate (3×100 mL). The combined organic layers were washed with brine (200 mL), dried over anhydrous Na₂SO₄, filtered and concentrated in vacuo to give a residue. The residue was triturated with methyl tert-butyl ether (200 mL) at 20° C. for 30 min and filtered. The filter cake was collected to give 5-chloro-6-methoxy-1H-indole-2-carboxamide. LC-MS (ESI) m/z: [M+H]⁺ calcd for C₁₀H₉ClN₂O₂: 224.04; found: 224.9.

Step 2: 2 reactions were carried out in parallel as described below: To a solution of 5-chloro-6-methoxy-1H-indole-2-carboxamide (23.0 g, 102 mmol) in THE (700 mL) was added LiAlH₄ (31.1 g, 819 mmol, 8.00 eq) portion-wise and carefully at 0° C. under N₂. After the addition was complete, the mixture was allowed to warm to 30° C. and stirred for 16 h. After cooling, 2 reactions were combined for work up. The combined reaction mixture was cooled to 0° C. and quenched by dropwise addition of water (31 mL), followed by slow addition of 15% aq. NaOH (31 mL) and water (93 mL). The mixture was then allowed to warm to 20° C. and stir 0.5 h, then filtered to remove the precipitate. The filtrate was concentrated in vacuo, then was dissolved in water (30 mL) and ethyl acetate (60 mL). The pH was adjusted to 2 by addition of 6M HCl, and extracted with ethyl acetate (3×60 mL). The aqueous phase was then adjusted to pH 11 by addition of 4M NaOH, and extracted with ethyl acetate (3×60 mL). The combined organic layers were washed with brine (100 mL), dried over Na₂SO₄, filtered and concentrated under reduced pressure to give (5-chloro-6-methoxy-1H-indol-2-yl) methanamine. LC-MS (ESI) m/z: [M+H]⁺+calcd for C₁₀H₁₁ClN₂O: 210.06; found: 211.1.

Step 3: To a solution of (5-chloro-6-methoxy-1H-indol-2-yl) methanamine (2.50 g, 11.9 mmol, 1.00 eq) in dichloromethane (50 mL) was added dropwise BBr₃ (47.5 mmol, 4.6 mL, 4.00 eq) at 0° C., and the reaction mixture was stirred at 0° C. for 2 h. The mixture was poured into water (15 mL) at 0° C. and adjusted to pH=7 by addition of saturated aqueous NaHCO₃, then extracted with ethyl acetate (3×20 mL). The combined organic phases were washed with brine (20 mL), dried over Na₂SO₄, filtered and concentrated under reduced pressure to give 2-(aminomethyl)-5-chloro-1H-indol-6-ol, which was used without further purification. LC-MS (ESI) m/z: [M+H]⁺ calcd for C₉H₉ClN₂O: 196.04; found: 197.1.

Step 4: To a solution of 2-(aminomethyl)-5-chloro-1H-indol-6-ol (2.30 g, 9.36 mmol, 80% purity) in DMF (20 mL) were added Boc₂O (7.49 mmol, 1.72 mL, 0.80 eq) and TEA (28.1 mmol, 3.90 mL, 3.00 eq) at 0° C. under N₂. The reaction mixture was then stirred at 20° C. for 1 h. The mixture was poured into water (20 mL) and stirred for 5 min. The aqueous phase was extracted with ethyl acetate (2×20 mL). The combined organic phases were washed with brine (2×20 mL), dried over anhydrous Na₂SO₄, filtered and concentrated in vacuo. The residue was purified by column chromatography to give tert-butyl ((5-chloro-6-hydroxy-1H-indol-2-yl) methyl) carbamate, Intermediate A-15. LC-MS (ESI) m/z: [M+H]⁺ calcd for C₁₄H₁₇ClN₂O3: 296.09; found: 297.1.

The following compounds in Table B-4 were synthesized using procedures similar to Intermediate A-4 using the appropriate starting materials.

TABLE B-4

| | | | Exact | LCMS, |
|---|---|---|---|---|
| Intermediate | | | mass | found |
| No. | Structure | IUPAC name | (g/mol) | [M + H]⁺ |
| B-4-1 | 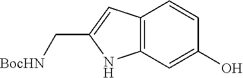 [SMILES: CC(C)(C)OC(=O)NCc1cc2ccc(O)cc2[nH]1] | tert-butyl ((6-hydroxy- 1H-indol-2- yl)methyl)carbamate | 262.13 | 263.1 |

### Intermediate A-5: (5-chloro-6-((5-methylisoxazol-3-yl)methoxy)-1H-indol-2-yl)methanamine

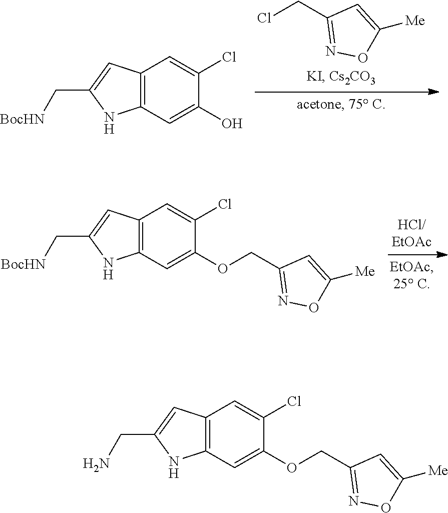 [SMILES: CC(C)(C)OC(=O)NCc1cc2cc(Cl)c(O)cc2[nH]1 ; Cc1cc(CCl)no1 ; Cc1cc(COc2cc3[nH]c(CNC(=O)OC(C)(C)C)cc3cc2Cl)no1 ; Cc1cc(COc2cc3[nH]c(CN)cc3cc2Cl)no1]

Step 1: A mixture of tert-butyl ((5-chloro-6-hydroxy-1H-indol-2-yl)methyl)carbamate (3.00 g, 10.1 mmol, 1.0 eq), 3-(chloromethyl)-5-methyl-isoxazole (1.20 g, 9.10 mmol, 0.90 eq), Cs₂CO₃ (3.29 g, 10.1 mmol, 1.0 eq) and KI (168 mg, 1.01 mmol, 0.10 eq) in acetone (30 mL) was degassed and purged with N₂ 3 times, then was stirred at 75° C. for 16 h under N₂ atmosphere. The mixture was allowed to cool to RT, then was poured into water (50 mL) and extracted with EtOAc (2×50 mL). The organic layer was washed with brine (100 mL) and dried over Na₂SO₄, and concentrated in vacuo. The resultant residue was washed with MTBE (30 mL) then isolated by filtration to give tert-butyl ((5-chloro-6-((5-methylisoxazol-3-yl)methoxy)-1H-indol-2-yl)methyl)carbamate, which was used without further purification. LC-MS (ESI) m/z: [M+H]⁺ calcd for C₁₉H₂₂ClN304: 391.13; found: 392.3.

Step 2: To a solution of tert-butyl ((5-chloro-6-((5-methylisoxazol-3-yl)methoxy)-1H-indol-2-yl)methyl)carbamate (2.78 g, 7.09 mmol, 1.0 eq) in EtOAc (20 mL) was added 4 M HCl in EtOAc (120 mmol, 30 mL, 16.9 eq) and the mixture was stirred at 20° C. for 1 h. The reaction mixture was filtered and the resulting solid was washed with MTBE (30 mL) then isolated by filtration to give (5-chloro-6-((5-methylisoxazol-3-yl)methoxy)-1H-indol-2-yl)methanamine hydrochloride. LC-MS (ESI) m/z: [M+H]⁺ calcd for C₁₄H₁₄ClN₃O₂: 291.08; found: 275.1 [M −NH₂+H]⁺.

The following compounds in Table B-5 were synthesized using procedures similar to Intermediate A-5 using the appropriate starting materials.

TABLE B-5

| | | | Exact | LCMS |
|---|---|---|---|---|
| Intermediate | | | mass | found |
| No. | Structure | IUPAC name | (g/mol) | [M + H]⁺ |
| B-5-1 | 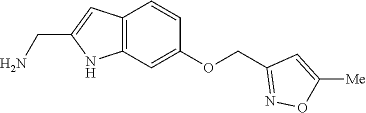 [SMILES: Cc1cc(COc2ccc3cc(CN)[nH]c3c2)no1] | (6-((5- methylisoxazol- 3-yl)methoxy)- 1H-indol-2- yl)methanamine | 257.12 | 241.1 [M − NH₂]⁻ |
| B-5-2 | 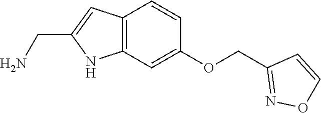 [SMILES: NCc1cc2ccc(OCc3ccon3)cc2[nH]1] | (6-(isoxazol-3- ylmethoxy)-1H- indol-2- yl)methanamine | 243.10 | 244.1 |
| B-5-3 | 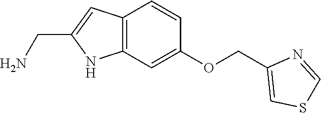 [SMILES: NCc1cc2ccc(OCc3cscn3)cc2[nH]1] | (6-(thiazol-4- ylmethoxy)-1H- indol-2- yl)methanamine | 259.08 | 260.1 |
| B-5-4 | 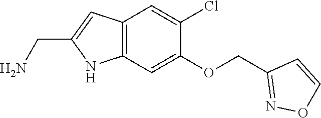 [SMILES: NCc1cc2cc(Cl)c(OCc3ccon3)cc2[nH]1] | (5-chloro-6- (isoxazol-3- ylmethoxy)-1H- indol-2- yl)methanamine | 277.06 | 261.1 [][M − NH₂]⁺ |
| B-5-5 | 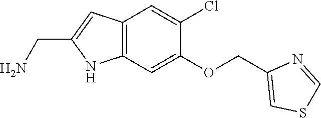 [SMILES: NCc1cc2cc(Cl)c(OCc3cscn3)cc2[nH]1] | (5-chloro-6- (thiazol-4- ylmethoxy)-1H- indol-2- yl)methanamine | 293.04 | 277.0 [M − NH₂]⁺ |

### Intermediate A-6: (R)-1-(isoxazol-3-yl)-N-methylpiperidin-3-amine

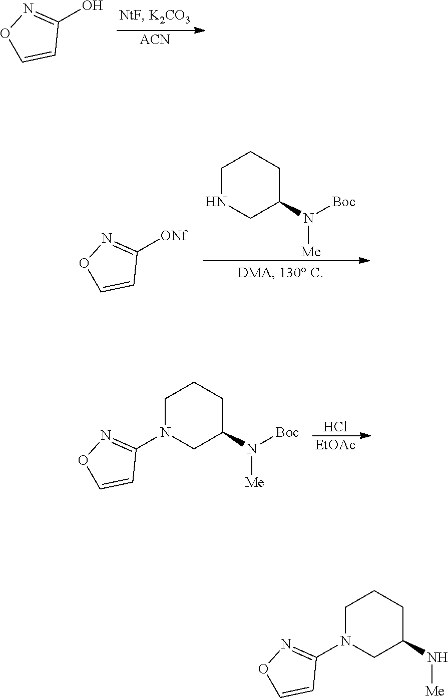

Step 1: To a solution of isoxazol-3-ol (200 mg, 2.35 mmol, 1.00 eq) in ACN (4 mL) were added K₂CO₃ (650 mg, 4.70 mmol, 2 eq) and 1,1,2,2,3,3,4,4,4-nonafluorobutane-1-sulfonyl fluoride (852 mg, 2.82 mmol, 1.20 eq) in one portion, then the reaction mixture was stirred at 20° C. for 12 h. The reaction mixture was poured into H₂O (15 mL) and stirred for 3 mins. The aqueous phase was extracted with ethyl acetate (3×10 mL). The combined organic phase was washed with brine (20 mL), dried over anhydrous Na₂SO₄, filtered and concentrated under reduced pressure. The resulting residue was purified by prep-TLC to give isoxazol-3-yl 1,1,2,2,3,3,4,4,4-nonafluorobutane-1-sulfonate. LC-MS (ESI) m/z: [M+H]⁺ calcd for C₇H₂F₉NO₄S: 366.96; found 368.0.

Step 2: A mixture of isoxazol-3-yl 1,1,2,2,3,3,4,4,4-nonafluorobutane-1-sulfonate (600 mg, 1.63 mmol, 1.20 eq) and tert-butyl (R)-methyl(piperidin-3-yl)carbamate (300 mg, 1.40 mmol, 1.03 eq) in DMA (4 mL) was heated to 130° C. for 12 h then cooled to room temperature. The reaction mixture was poured into H₂O (20 mL) and stirred for 3 mins, the aqueous phase was extracted with ethyl acetate (3×10 mL). The combined organic phase was washed with brine (20 mL), dried over anhydrous Na₂SO₄, filtered and concentrated under reduced pressure. The resulting residue was purified by prep-TLC to give tert-butyl (R)-(1-(isoxazol-3-yl)piperidin-3-yl)(methyl)carbamate. LC-MS (ESI) m/z: [M+H]⁺ calcd for C₁₄H₂₃N₃O₃: 281.17; found 181.8 [M−Boc+H]⁺.

Step 3: A solution of tert-butyl (R)-(1-(isoxazol-3-yl)piperidin-3-yl)(methyl)carbamate (140 mg, 497 μmol, 1.00 eq) in HCl (3 mL, 4 M in EtOAc) was stirred at 20° C. for 1 h. The reaction mixture was concentrated to vacuo to give (R)-1-(isoxazol-3-yl)-N-methylpiperidin-3-amine hydrochloride, which was used without further purification. LC-MS (ESI) m/z: [M+H]⁺ calcd for C₉H₁₅N₃O: 181.12; found 182.2.

The following compounds in Table B-6 were synthesized using procedures similar to Intermediate A-6 using the appropriate starting materials.

TABLE B-6

| | | | Exact | LCMS |
|---|---|---|---|---|
| Intermediate | | | mass | found |
| No. | Structure | IUPAC name | (g/mol) | [M + H]⁺ |
| B-6-1 | 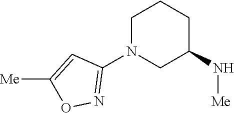 [SMILES: CN[C@@H]1CCCN(c2cc(C)on2)C1] | (R)-N-methyl-1- (5- methylisoxazol- 3-yl)piperidin-3- amine | 195.14 | 196.2 |

### Intermediate A-7: (R)-1-(3-((2,2,2-trifluoroethyl)amino)piperidin-1-yl)ethan-1-one

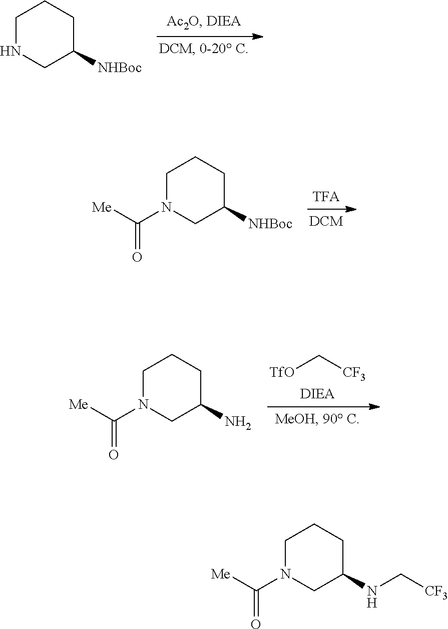 [SMILES: CC(C)(C)OC(=O)N[C@@H]1CCCNC1 ; CC(=O)N1CCC[C@@H](NC(=O)OC(C)(C)C)C1 ; CC(=O)N1CCC[C@@H](N)C1 ; O=S(=O)(OCC(F)(F)F)C(F)(F)F ; CC(=O)N1CCC[C@@H](NCC(F)(F)F)C1]

Step 1: tert-butyl (R)-(1-acetylpiperidin-3-yl)carbamate. To a mixture of tert-butyl (R)-piperidin-3-ylcarbamate (10.0 g, 49.9 mmol, 1.00 eq) and DIEA (25.8 g, 199 mmol, 4.00 eq) in DCM (100 mL) at 0° C. was added dropwise Ac₂O (5.35 g, 52.4 mmol, 1.05 eq). The reaction mixture was warmed to 20° C. and stirred for 1 h. The reaction mixture was diluted with H₂O (50 mL) and extracted with ethyl acetate (2×100 mL). The combined organic layers were dried over Na₂SO₄, filtered and concentrated under reduced pressure. The resulting residue was triturated with petroleum ether (30 mL) for 10 mins then filtered and the filter cake was collected to give tert-butyl (R)-(1-acetylpiperidin-3-yl)carbamate. LC-MS (ESI) m/z: [M+H]⁺ calcd for C₁₂H₂₂N₂O₃: 242.16; found 243.2.

Step 2: (R)-1-(3-aminopiperidin-1-yl)ethan-1-one. To a solution of tert-butyl (R)-(1-acetylpiperidin-3-yl)carbamate (3.00 g, 12.3 mmol, 1.00 eq) in DCM (20 mL) was added dropwise TFA (10 mL). The reaction mixture was stirred at 20° C. for 1 h then concentrated directly. The resulting residue was dissolved in DCM (10 mL), adjusted to pH 7 with Amberlyst 21 (2 g), filtered, and concentrated under reduced pressure to give (R)-1-(3-aminopiperidin-1-yl)ethan-1-one which was used without of further purification. LC-MS (ESI) m/z: [M+H]⁺ calcd for C₇H₁₄N₂O: 142.11; found 143.3.

Step 3: (R)-1-(3-((2,2,2-trifluoroethyl)amino)piperidin-1-yl)ethan-1-one. A mixture of (R)-1-(3-aminopiperidin-1-yl)ethan-1-one (972 mg, 6.84 mmol, 1.00 eq), 2,2,2-trifluoroethyl trifluoromethanesulfonate (3.17 g, 13.6 mmol, 2.00 eq) and DIEA (1.77 g, 13.6 mmol, 2.00 eq) in MeOH (1 mL) was heated to 90° C. After 4 h the reaction mixture was cooled to room temperature, diluted with H₂O (10 mL) and extracted into ethyl acetate (2×10 mL). The organic layers were combined, dried over Na₂SO₄, filtered and concentrated under reducted pressure. The resulting residue was purified by prep-HPLC to give (R)-1-(3-((2,2,2-trifluoroethyl)amino)piperidin-1-yl)ethan-1-one. LC-MS (ESI) m/z: [M+H]⁺ calcd for C₉H₁₅F₃N₂O: 224.11; found 225.1.

### Intermediate A-8: 1-((3R,4R and 3S,4S)-4-methyl-3-(methylamino)piperidin-1-yl)ethan-1-one

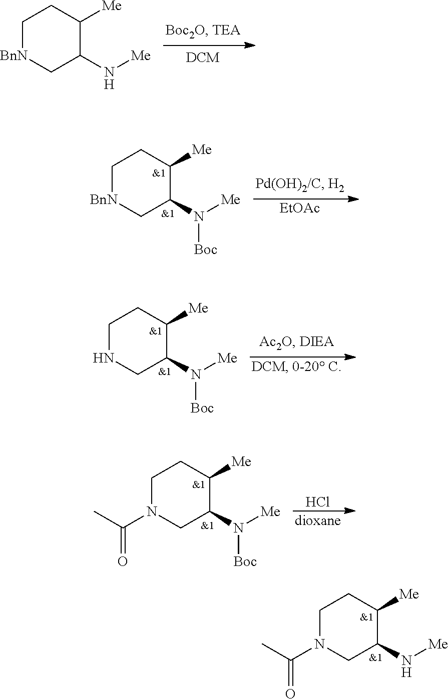

Step 1: tert-butyl ((3R,4R and 3S,4S)-1-benzyl-4-methylpiperidin-3-yl)(methyl)carbamate. To a suspension of 1-benzyl-N,4-dimethylpiperidin-3-amine hydrochloride (1.00 g, 3.43 mmol, 1.00 eq) in DCM (20 mL) were added portion wise TEA (1.74 g, 17.1 mmol, 5.00 eq) and Boc₂O (899 mg, 4.12 mmol, 1.20 eq). The reaction was stirred for 12 h then quenched by addition of H₂O (10 mL) and extracted into EtOAc (3×15 mL). The combined organic layers were washed with brine (10 mL), dried over Na₂SO₄, filtered and concentrated under reduced pressure to give tert-butyl ((3R,4R and 3S,4S)-1-benzyl-4-methylpiperidin-3-yl)(methyl)carbamate, which was used without of further purification. LC-MS (ESI) m/z: [M+H]⁺ calcd for C₁₉H₃₀N₂O₂: 318.23; found 319.2.

Step 2: tert-butyl methyl((3R,4R and 3S,4S)-4-methylpiperidin-3-yl)carbamate. A solution of tert-butyl ((3R,4R and 3S,4S)-1-benzyl-4-methylpiperidin-3-yl)(methyl)carbamate (800 mg, 2.51 mmol, 1.00 eq) in EtOAc (10 mL) was degassed then charged with nitrogen three times. Pd(OH)₂/C (1.76 g, 2.51 mmol, 20%, 1.00 eq) was added in one portion, then the reaction system was degassed and charged with H₂ (15 psi) three times. The reaction mixture was stirred at 25° C. for 12 h under H₂ (15 psi) atmosphere. The reaction mixture was filtered through Celite, the filter cake was washed with MeOH (2×10 mL) and the filtrate was concentrate under reduced pressure to give tert-butyl methyl((3R,4R and 3S,4S)-4-methylpiperidin-3-yl)carbamate, which was used without of further purification. LC-MS (ESI) m/z: [M+H]⁺ calcd for C₁₂H₂₄N₂O₂: 228.18; found 229.2.

Step 3: tert-butyl ((3R,4R and 3S,4S)-1-acetyl-4-methylpiperidin-3-yl)(methyl)carbamate. To a solution of tert-butyl methyl((3R,4R and 3S,4S)-4-methylpiperidin-3-yl)carbamate (420 mg, 1.84 mmol, 1.00 eq) in DCM (5 mL) at 0° C. were added dropwise DIEA (713 mg, 5.52 mmol, 3.00 eq) and Ac₂O (206 mg, 2.02 mmol, 1.10 eq). The reaction mixture was warmed to 20° C. After 2 h, the reaction mixture was cooled to 0° C. and quenched by addition of H₂O (10 mL). The mixture was extracted into EtOAc (3×5 mL) and the combined organic phase was washed with brine (10 mL), dried over anhydrous Na₂SO₄, filtered and concentrated under reduced pressure to give tert-butyl ((3R,4R and 3S,4S)-1-acetyl-4-methylpiperidin-3-yl)(methyl)carbamate, which was used without of further purification. LC-MS (ESI) m/z: [M+H]⁺ calcd for C₁₄H₂₆N₂O₃: 270.19; found 271.2.

Step 4: 1-((3R,4R and 3S,4S)-4-methyl-3-(methylamino)piperidin-1-yl)ethan-1-one. To a solution of tert-butyl ((3R,4R and 3S,4S)-1-acetyl-4-methylpiperidin-3-yl)(methyl)carbamate (440 mg, 1.63 mmol, 1.00 eq) in dioxane (0.5 mL) was added dropwise HCl (4 M in dioxane, 5 mL). The reaction mixture was stirred at 20° C. for 1 h. The reaction mixture was concentrated under reduced pressure directly to give 1-((3R,4R and 3S,4S)-4-methyl-3-(methylamino)piperidin-1-yl)ethan-1-one, which was used without of further purification. LC-MS (ESI) m/z: [M+H]⁺ calcd for C₉H₁₈N₂O: 170.14; found 171.2.

### Intermediate A-9: 2-(4-chloro-3-(1H-indol-2-yl)phenyl)ethan-1-amine

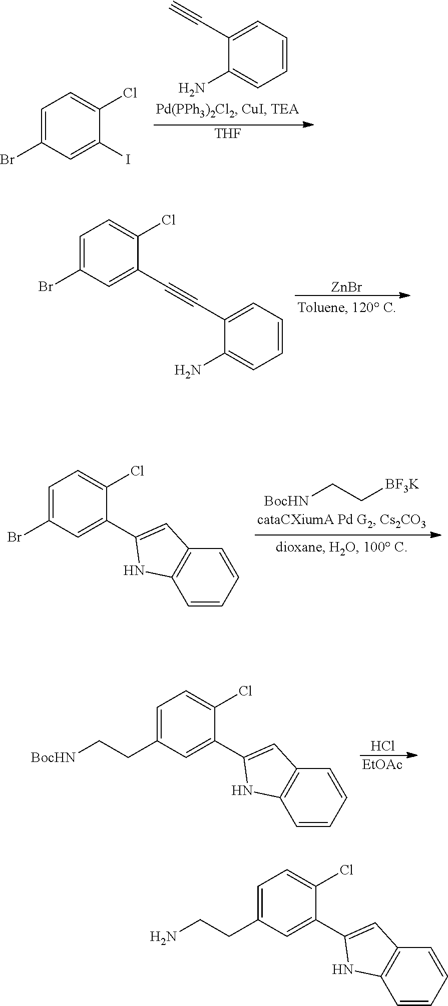

Step 1: 2-((5-bromo-2-chlorophenyl)ethynyl)aniline. A mixture of 4-bromo-1-chloro-2-10 dobenzene (4.00 g, 12.6 mmol, 1.00 eq), 2-ethynylaniline (1.48 g, 12.6 mmol, 1.00 eq), CuI (480 mg, 2.52 mmol, 0.20 eq) and TEA (6.38 g, 63.0 mmol, 5.00 eq) in THE (10 mL) was degassed and purged with N₂ 3 times. Pd(PPh₃)₂C₁₂ was added (884 mg, 1.26 mmol, 0.10 eq) and the reaction mixture was stirred at 20° C. under N₂ atmosphere for 12 h. The reaction mixture was quenched with H₂O (50 mL) then extracted with EtOAc (2×50 mL). The combined organic layers were washed with brine (2×40 mL), dried over Na₂SO₄, filtered and concentrated under reduced pressure. The resulting residue was purified by column chromatography to give 2-((5-bromo-2-chlorophenyl)ethynyl)aniline. LC-MS (ESI) m/z: [M+H]⁺ calcd for C₁₄H₉BrClN: 304.96; found 306.0.

Step 2: 2-(5-bromo-2-chlorophenyl)-1H-indole. To a solution of 2-((5-bromo-2-chlorophenyl)ethynyl)aniline (1.40 g, 4.57 mmol, 1.00 eq) in toluene (60 mL) was added ZnBr₂ (2.57 g, 11.4 mmol, 2.50 eq) and the resulting mixture was heated to 120° C. After 12 h the reaction was cooled to room temperature, quenched with H₂O (60 mL), and extracted into EtOAc (2×50 mL). The combined organic layers were washed with brine (2×30 mL), dried over Na₂SO₄, filtered and concentrated under reduced pressure. The resulting residue was purified by column chromatography to give 2-(5-bromo-2-chlorophenyl)-1H-indole. LC-MS (ESI) m/z: [M+H]⁺ calcd for C₁₄H₉BrClN: 304.96; found 306.0.

Step 3: tert-butyl (4-chloro-3-(1H-indol-2-yl)phenethyl)carbamate. A mixture of 2-(5-bromo-2-chlorophenyl)-1H-indole (600 mg, 1.96 mmol, 1.00 eq), potassium (2-((tert-butoxycarbonyl)amino)ethyl)trifluoroborate (737 mg, 2.94 mmol, 1.50 eq), and Cs₂CO₃ (1.28 g, 3.91 mmol, 2.00 eq) in dioxane (4 mL) and H₂O (1 mL) was degassed and purged with N₂ 3 times. Chloro[(di(1-adamantyl)-N-butylphosphine)-2-(2-aminobiphenyl)]palladium(II) (130 mg, 195 μmol, 0.10 eq) was added and the reaction mixture was heated to 100° C. under N₂ atmosphere. After 16 h, the reaction was cooled to room temperature and quenched with H₂O (60 mL) then extracted into EtOAc (2×50 mL). The combined organic layers were washed with H₂O (2×30 mL), dried over Na₂SO₄, filtered and concentrated under reduced pressure. The resulting residue was purified by column chromatography to give tert-butyl (4-chloro-3-(1H-indol-2-yl)phenethyl)carbamate. LC-MS (ESI) m/z: [M+H]⁺ calcd for C₂₁H₂₃ClN₂O₂: 370.14; found 371.2.

Step 4: 2-(4-chloro-3-(1H-indol-2-yl)phenyl)ethan-1-amine. To a solution of tert-butyl (4-chloro-3-(1H-indol-2-yl)phenethyl)carbamate (400 mg, 1.08 mmol, 1.00 eq) in EtOAc (0.5 mL) was added HCl (4M in EtOAc, 5 mL). The reaction mixture was stirred at 20° C. After 2 h the reaction mixture was filtered and concentrated under reduced pressure to give 2-(4-chloro-3-(1H-indol-2-yl)phenyl)ethan-1-amine hydrochloride, which was used without of further purification. LC-MS (ESI) m/z: [M+H]⁺ calcd for C₁₆H₁₅ClN₂: 270.09; found 271.1.

The following compounds in Table B-9 were synthesized using procedures similar to Intermediate A-9 using the appropriate starting materials.

TABLE B-9

| | | | Exact | LCMS |
|---|---|---|---|---|
| Intermediate | | | mass | found |
| No. | Structure | IUPAC name | (g/mol) | [M + H]⁺ |
| B-9-1 | 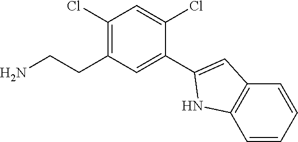 [SMILES: NCCc1cc(-c2cc3ccccc3[nH]2)c(Cl)cc1Cl] | 2-(2,4-dichloro- 5-(1H-indol-2- yl)phenyl)ethan- 1-amine | 304.05 | 305.5 |
| B-9-2 | 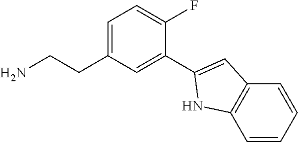 [SMILES: NCCc1ccc(F)c(-c2cc3ccccc3[nH]2)c1] | 2-(4-fluoro-3- (1H-indol-2- yl)phenyl)ethan -1-amine | 254.12 | 255.2 |

### Intermediate A-10: (6-methyl-1H-indol-2-yl)methanamine

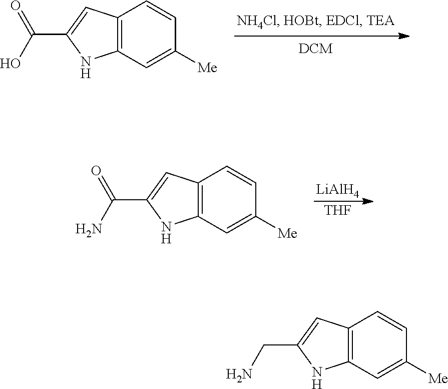 [SMILES: Cc1ccc2cc(C(=O)O)[nH]c2c1 ; Cc1ccc2cc(C(N)=O)[nH]c2c1 ; "LiAlH4" ; Cc1ccc2cc(CN)[nH]c2c1 ; "THF"]

Step 1: 6-methyl-1H-indole-2-carboxamide. To a solution of 6-methyl-1H-indole-2-carboxylic acid (400 mg, 2.28 mmol, 1.00 eq) in DCM at 20° C. (4 mL) under N₂ atmosphere was added EDCI (525 mg, 2.74 mmol, 1.20 eq) and HOBt (370 mg, 2.74 mmol, 1.20 eq). After 10 mins, triethylamine (3.42 mmol, 477 μL, 1.50 eq) and NH₄C₁ (183 mg, 3.42 mmol, 1.50 eq) were added and the reaction was stirred at 20° C. After 2 h the reaction mixture was poured into water (10 mL) and stirred for 5 min then extracted into ethyl acetate (2×10 mL). The combined organic phases were washed with brine (2×10 mL), dried over anhydrous Na₂SO₄, filtered and concentrated under reduced pressure. The resulting residue was purified by silica gel chromatography to give 6-methyl-1H-indole-2-carboxamide. LC-MS (ESI) m/z: [M+H]⁺ calcd for C₁₀H₁₀N₂O: 174.08; found 175.1.

Step 2: (6-methyl-1H-indol-2-yl)methanamine. To a solution of 6-methyl-1H-indole-2-carboxamide (330 mg, 1.90 mmol, 1.00 eq) in THE (15 mL) at 0° C. under N₂ atmosphere was added LiAlH₄ (577 mg, 15.2 mmol, 8.00 eq) portion wise. The reaction mixture was stirred at 25° C. for 30 mins and then heated to 80° C. and stirred for an additional 2 h. The reaction mixture was cooled to 0° C. then quenched by dropwise addition of water (2 mL) followed by 15% aq. NaOH (0.55 mL). The resulting mixture was filtered then extracted into ethyl acetate (2×10 mL) and the combined organic phase was concentrated. The resulting residue was diluted with H₂O (5 mL) and adjusted to pH 4 with 1M HCl. The aqueous phase was extracted with ethyl acetate (20 mL) then the aqueous phase adjusted to pH 8 with 5 M NaOH and extracted into ethyl acetate (2×10 mL). The combined organic phase was washed with brine (2×10 mL), dried over anhydrous Na₂SO₄, filtered and concentrated in vacuum to give (6-methyl-1H-indol-2-yl)methanamine which was used without further purification. LC-MS (ESI) m/z: [M+H]⁺ calcd for C₁₀H₁₂N₂: 160.10; found 161.1.

The following compounds in Table B-10 were synthesized using procedures similar to Intermediate A-10 using the appropriate starting materials.

TABLE B-10

| | | | Exact | LCMS |
|---|---|---|---|---|
| Intermediate | | | mass | found |
| No. | Structure | IUPAC name | (g/mol) | [M + H]⁺ |
| B-10-1 | 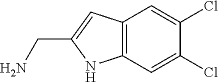 [SMILES: NCc1cc2cc(Cl)c(Cl)cc2[nH]1] | (5,6-dichloro-1H-indol- 2-yl)methanamine | 214.01 | 215.0 |
| B-10-2 | 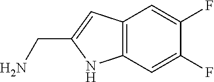 [SMILES: NCc1cc2cc(F)c(F)cc2[nH]1] | (5,6-difluoro-1H-indol-2- yl)methanamine | 182.07 | 183.1 |
| B-10-3 | 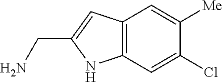 [SMILES: Cc1cc2cc(CN)[nH]c2cc1Cl] | (6-chloro-5-methyl-1H- indol-2-yl)methanamine | 194.06 | 195.1 |
| B-10-4 | 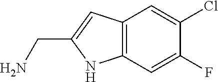 [SMILES: NCc1cc2cc(Cl)c(F)cc2[nH]1] | (5-chloro-6-fluoro-1H- indol-2-yl)methanamine | 198.04 | 199.1 |
| B-10-5 | 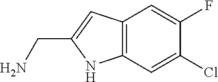 [SMILES: NCc1cc2cc(F)c(Cl)cc2[nH]1] | (6-chloro-5-fluoro-1H- indol-2-yl)methanamine | 198.01 | 199.1 |
| B-10-6 | 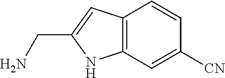 [SMILES: N#Cc1ccc2cc(CN)[nH]c2c1] | 2-(aminomethyl)-1H- indole-6-carbonitrile | 171.08 | 172.1 |
| B-10-7 | 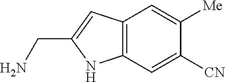 [SMILES: Cc1cc2cc(CN)[nH]c2cc1C#N] | 2-(aminomethyl)-5- methyl-1H-indole-6- carbonitrile | 185.10 | 186.1 |
| B-10-8 | 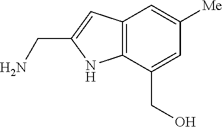 [SMILES: Cc1cc(CO)c2[nH]c(CN)cc2c1] | (2-(aminomethyl)-5- methyl-1H-indol-7- yl)methanol | 190.11 | 191.3 |
| B-10-9 | 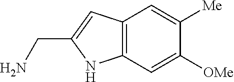 [SMILES: COc1cc2[nH]c(CN)cc2cc1C] | (6-methoxy-5-methyl- 1H-indol-2- yl)methanamine | 191.11 | 191.1 |
| B-10-10 | 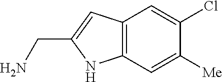 [SMILES: Cc1cc2[nH]c(CN)cc2cc1Cl] | (5-chloro-6-methyl-1H- indol-2-yl)methanamine | 194.06 | 195.1 |
| B-10-11 | 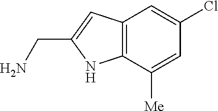 [SMILES: Cc1cc(Cl)cc2cc(CN)[nH]c12] | (5-chloro-7-methyl-1H- indol-2-yl)methanamine | 194.06 | 178.2 [M − NH₂ + H]⁺ |
| B-10-12 | 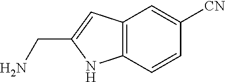 [SMILES: N#Cc1ccc2[nH]c(CN)cc2c1] | 2-(aminomethyl)-1H- indole-5-carbonitrile | 171.08 | 172.1 |
| B-10-13 | 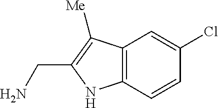 [SMILES: Cc1c(CN)[nH]c2ccc(Cl)cc12] | (5-chloro-3-methyl-1H- indol-2-yl)methanamine | 194.06 | 178.1 [M − NH₂ + H]⁺ |
| B-10-14 | 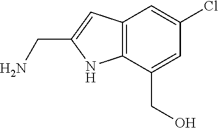 [SMILES: NCc1cc2cc(Cl)cc(CO)c2[nH]1] | (2-(aminomethyl)-5- chloro-1H-indol-7- yl)methanol | 210.06 | 211.1 |
| B-10-15 | 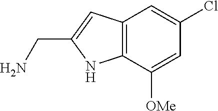 [SMILES: COc1cc(Cl)cc2cc(CN)[nH]c12] | (5-chloro-7-methoxy-1H- indol-2-yl)methanamine | 210.06 | 211.0 |
| B-10-16 | 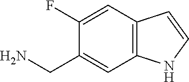 [SMILES: NCc1cc2[nH]ccc2cc1F] | (5-fluoro-1H-indol-6- yl)methanamine | 164.07 | 148.1 [[M − NH₂ + H]⁺ |
| B-10-17 | 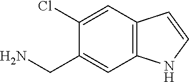 [SMILES: NCc1cc2[nH]ccc2cc1Cl] | (5-chloro-1H-indol-6- yl)methanamine | 180.05 | 164.1 [ [M − NH₂ + H]⁺ |
| B-10-18 | 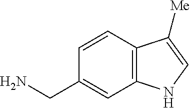 [SMILES: Cc1c[nH]c2cc(CN)ccc12] | (3-methyl-1H-indol-6- yl)methanamine | 160.10 | 144.1 [ [M − NH₂ + H]⁺ |
| B-10-19 | 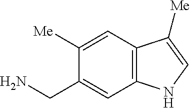 [SMILES: Cc1cc2c(C)c[nH]c2cc1CN] | (3,5-dimethyl-1H-indol- 6-yl)methanamine | 174.12 | 158.2 [ [M − NH₂ + H]⁺ |
| B-10-20 | 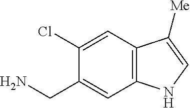 [SMILES: Cc1c[nH]c2cc(CN)c(Cl)cc12] | (5-chloro-3-methyl-1H- indol-6-yl)methanamine | 194.06 | 177.9 [ [M − NH₂ + H]⁺ |
| B-10-21 | 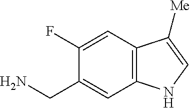 [SMILES: Cc1c[nH]c2cc(CN)c(F)cc12] | (5-fluoro-3-methyl-1H- indol-6-yl)methanamine | 178.09 | 162.1 [ [M − NH₂ + H]⁺ |
| B-10-22 | 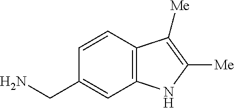 [SMILES: Cc1[nH]c2cc(CN)ccc2c1C] | (2,3-dimethyl-1H-indol- 6-yl)methanamine | 174.12 | 158.1 [ [M − NH₂ + H]⁺ |

### Intermediate A-11: (R)-1-(3-(isopropylamino)piperidin-1-yl)ethan-1-one

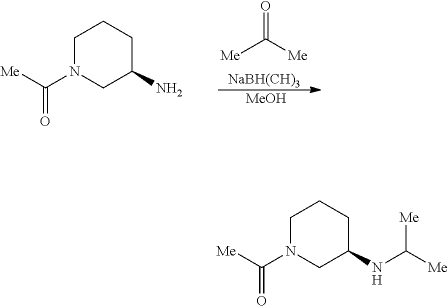

To a solution of (R)-1-(3-aminopiperidin-1-yl)ethan-1-one (350 mg, 2.46 mmol, 1.00 eq) in MeOH (2 mL) under N₂ atmosphere was added acetone (157 mg, 2.71 mmol, 1.10 eq) followed by NaBH₃CN (309 mg, 4.92 mmol, 2.00 eq) in portions. The resulting mixture was stirred at 20° C. for 2 h. The reaction was quenched with H₂O (10 mL) and extracted into ethyl acetate (3×5 mL). The combined organic layers were dried over anhydrous Na₂SO₄, filtered and concentrated under reduced pressure to give (R)-1-(3-(isopropylamino)piperidin-1-yl)ethan-1-one, which was used without of further purification. LC-MS (ESI) m/z: [M+H]⁺ calcd for C₁₀H₂₀N₂O: 184.16; found 185.3.

### Intermediate A-12: (R)-1-(3-(cyclopropylamino)piperidin-1-yl)ethan-1-one

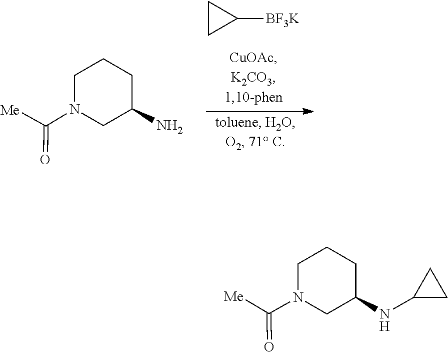

2 reactions were carried out in parallel.

To a solution of (R)-1-(3-aminopiperidin-1-yl)ethan-1-one (200 mg, 1.41 mmol, 1.00 eq) in toluene (0.65 mL) and H₂O (0.2 mL) were added potassium cyclopropyltrifluoroborate (229 mg, 1.55 mmol, 1.10 eq), CuOAc (70 mg, 351 μmol, 0.25 eq), K₂CO₃ (389 mg, 2.81 mmol, 2.00 eq) and 1,10-phenanthroline (30.0 mg, 169 μmol, 0.12 eq), and the reaction mixture was degassed and purged with O₂ 3 times. The reaction mixture was heated to 70° C. under 02 atmosphere. After 45 mins the reaction was cooled to 20° C. and two parallel reactions were combined for workup. The reaction mixture was quenched with H₂O (15 mL) and extracted into ethyl acetate (3×10 mL). The combined organic phases were washed with brine (10 mL), dried over Na₂SO₄, filtered and concentrated under reduced pressure to give (R)-1-(3-(cyclopropylamino)piperidin-1-yl)ethan-1-one which was used without of further purification.

LC-MS (ESI) m/z: [M+H]⁺ calcd for C₁₀H₁₈N₂O: 182.14; found 183.3.

### Intermediate A-13: (R)-N-methyl-1-(4-methylisoxazol-3-yl)piperidin-3-amine

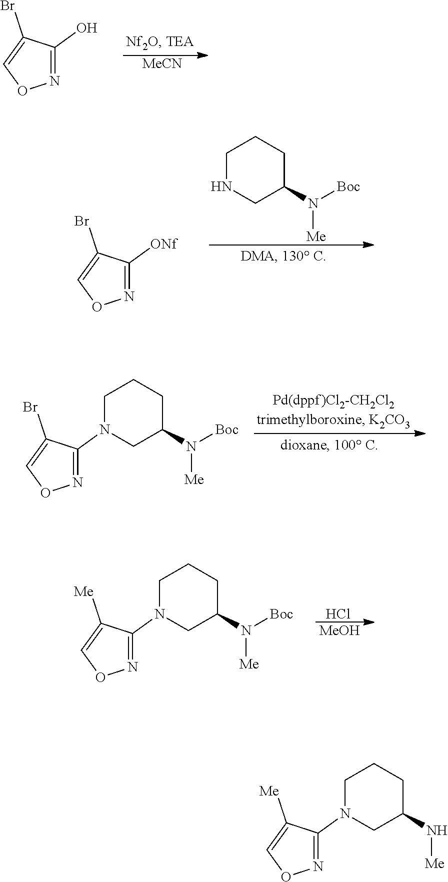

Step 1: 4-bromoisoxazol-3-yl 1,1,2,2,3,3,4,4,4-nonafluorobutane-1-sulfonate. To a solution of 4-bromoisoxazol-3-ol (420 mg, 2.56 mmol, 1.00 eq) in MeCN (10 mL) were added portion wise TEA (648 mg, 6.40 mmol, 2.50 eq) and Nf₂O (1.64 g, 2.82 mmol, 1.10 eq) and the resulting mixture was stirred at 20° C. After 16 h the reaction was poured into H₂O (20 mL) and extracted into ethyl acetate (3×20 mL). The combined organic layers were washed with brine (30 mL), dried over Na₂SO₄, filtered and concentrated under reduced pressure. The resulting residue was purified by silica gel chromatography to give 4-bromoisoxazol-3-yl 1,1,2,2,3,3,4,4,4-nonafluorobutane-1-sulfonate.

Step 2: tert-butyl (R)-(1-(4-bromoisoxazol-3-yl)piperidin-3-yl)(methyl)carbamate. To a solution of 4-bromoisoxazol-3-yl 1,1,2,2,3,3,4,4,4-nonafluorobutane-1-sulfonate (1.30 g, 80% purity) in DMA (30 mL) was added tert-butyl (R)-methyl(piperidin-3-yl)carbamate (437 mg, 2.04 mmol, 1.00 eq) and the resulting mixture was heated to 130° C. After 16 h the reaction was cooled to room temperature, poured into H₂O (30 mL), and the aqueous phase was extracted into ethyl acetate (3×40 mL). The combined organic layers were washed with brine (50 mL), dried over Na₂SO₄, filtered and concentrated under reduced pressure. The resulting residue was purified by silica gel chromatography to give tert-butyl (R)-(1-(4-bromoisoxazol-3-yl)piperidin-3-yl)(methyl)carbamate.

Step 3: tert-butyl (R)-methyl(1-(4-methylisoxazol-3-yl)piperidin-3-yl)carbamate. A mixture of tert-butyl (R)-(1-(4-bromoisoxazol-3-yl)piperidin-3-yl)(methyl)carbamate (200 mg, 555 μmol, 1.00 eq), trimethylboroxine (1 M in THF, 34.8 mg, 0.50 eq) and K₂CO₃ (230 mg, 1.67 mmol, 3.00 eq) in dioxane (5 mL) was degassed and purged with N₂ 3 times, then Pd(dppf)Cl₂·CH₂Cl₂ (45.0 mg, 55.5 μmol, 0.10 eq) was added. The reaction mixture was heated to 100° C. under N₂ atmosphere. After 16 h, the reaction was cooled to room temperature, poured into H₂O (15 mL) and extracted into ethyl acetate (3×15 mL). The combined organic layers were washed with brine (20 mL), dried over Na₂SO₄, filtered and concentrated under reduced pressure. The resulting residue was purified by silica gel chromatography to give tert-butyl (R)-methyl(1-(4-methylisoxazol-3-yl)piperidin-3-yl)carbamate. LC-MS (ESI) m/z: [M+H]⁺ calcd for C₁₅H₂₅N₃O₃: 295.19; found 296.3.

Step 4: (R)-N-methyl-1-(4-methylisoxazol-3-yl)piperidin-3-amine. To a solution of tert-butyl (R)-methyl(1-(4-methylisoxazol-3-yl)piperidin-3-yl)carbamate (60.0 mg, 213 μmol, 1.00 eq) in MeOH (1 mL) was added dropwise HCl (4 M in MeOH, 5 mL) and the resulting mixture was stirred at 20° C. After 2 h the reaction was concentrated under reduced pressure to give (R)-N-methyl-1-(4-methylisoxazol-3-yl)piperidin-3-amine hydrochloride, which was used without of further purification. LC-MS (ESI) m/z: [M+H]⁺ calcd for C₁₀H₁₇N₃O: 195.14; found 196.0.

### Intermediate A-14: 1-(2-methyl-5-(methylamino)piperidin-1-yl)ethan-1-one

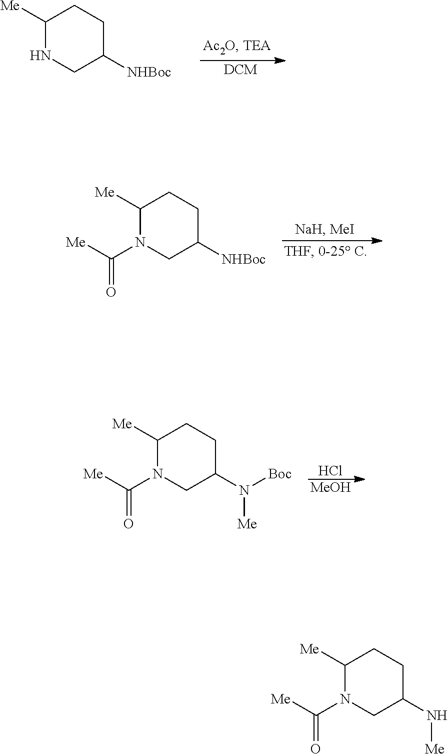

Step 1: tert-butyl (1-acetyl-6-methylpiperidin-3-yl)carbamate. To a solution of tert-butyl (6-methylpiperidin-3-yl)carbamate (2.00 g, 9.33 mmol, 1.00 eq) in DCM (40 mL) were added portion wise TEA (944 mg, 9.33 mmol, 1.00 eq) and Ac₂O (953 mg, 9.33 mmol, 1.00 eq) and the resulting mixture was stirred at 20° C. After 2 h the reaction was quenched with H₂O (30 mL) and extracted into DCM (3×20 mL). The combined organic layers were washed with brine (50 mL), dried over Na₂SO₄, filtered and concentrated under reduced pressure to give tert-butyl (1-acetyl-6-methylpiperidin-3-yl)carbamate, which was used without further purification.

Step 2: tert-butyl (1-acetyl-6-methylpiperidin-3-yl)(methyl)carbamate. To a solution of tert-butyl (1-acetyl-6-methylpiperidin-3-yl)carbamate (1.80 g, 7.02 mmol, 1.00 eq) in THE (15 mL) at 0° C. under N₂ atmosphere was added portion wise NaH (562 mg, 14.0 mmol, 60%, 2.00 eq) then the mixture was warmed to 25° C. After 1 h the reaction was cooled to 0° C., Mel (1.99 g, 14.0 mmol, 2.00 eq) was added portion wise, and the reaction was warmed to 25° C. After 16 h the reaction was quenched with sat. aq. NH₄C₁ (30 mL) and extracted into EtOAc (2×20 mL). The combined organic layers were washed with brine (20 mL), dried over Na₂SO₄, filtered and concentrated under reduced pressure. The resulting residue was purified by column chromatography to give tert-butyl (1-acetyl-6-methylpiperidin-3-yl)(methyl)carbamate. LC-MS (ESI) m/z: [M+H]⁺ calcd for C₁₄H₂₆N₂O₃: 270.19; found 271.1.

Step 3: 1-(2-methyl-5-(methylamino)piperidin-1-yl)ethan-1-one. To a solution of tert-butyl (1-acetyl-6-methylpiperidin-3-yl)(methyl)carbamate (1.28 g, 4.73 mmol, 1.00 eq) in MeOH (10 mL) was added portion wise HCl (4 M in MeOH, 30 mL) and then the reaction mixture was stirred at 20° C. After 2 h the reaction was concentrated under reduced pressure to give 1-(2-methyl-5-(methylamino)piperidin-1-yl)ethan-1-one which was used without further purification. LC-MS (ESI) m/z: [M+H]⁺ calcd for C₉H₁₈N₂O: 170.14; found 171.0.

The following compounds in Table B-14 were synthesized using procedures similar to

### Intermediate A-14 Using the Appropriate Starting Materials

TABLE B-14

| | | | Exact | LCMS |
|---|---|---|---|---|
| Intermediate | | | mass | found |
| No. | Structure | IUPAC name | (g/mol) | [M + H]⁺ |
| B-14-1 | 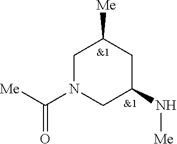 [SMILES: CN[C@@H]1C[C@H](C)CN(C(C)=O)C1] | 1-((3R,5S and 3S,5R)-3- methyl-5-(methylamino) piperidin-1-yl)ethan-1-one | 170.14 | 171.2 |

### Intermediate A-15: 2-(4-chloro-2-fluoro-5-(1H-indol-2-yl)phenyl)ethan-1-amine

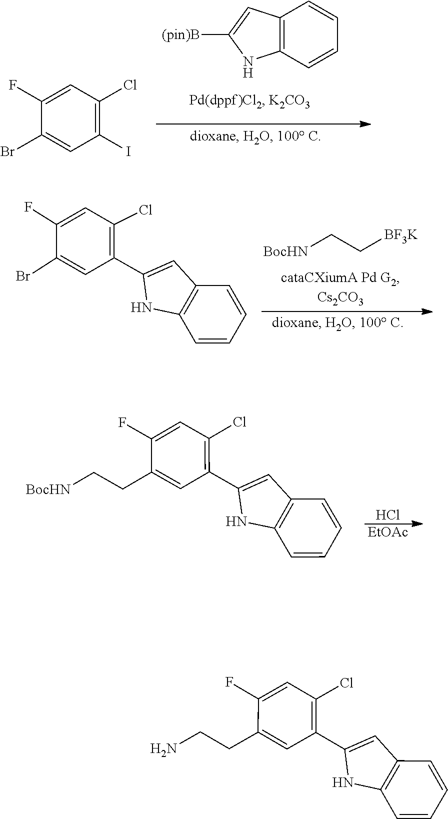

Step 1: 2-(5-bromo-2-chloro-4-fluorophenyl)-1H-indole. A mixture of 1-bromo-4-chloro-2-fluoro-5-iodobenzene (4.48 g, 13.0 mmol, 1.00 eq), 2-(4,4,5,5-tetramethyl-1,3,2-dioxaborolan-2-yl)-1H-indole (2.61 g, 10.7 mmol, 0.8 eq) and K₂CO₃ (3.70 g, 26.0 mmol, 2.00 eq) in dioxane (34 mL) and H₂O (2 mL) was degassed and purged with N₂ 3 times, then Pd(dppf)C₁₂ (1.10 g, 1.34 mmol, 0.10 eq) was added. The reaction mixture was heated to 100° C. under N₂ atmosphere. After 2 h, the reaction was cooled to room temperature, quenched with H₂O (20 mL), and extracted with ethyl acetate (3×20 mL). The combined organic layers were washed with brine (20 mL), dried over Na₂SO₄, filtered and concentrated under reduced pressure. The resulting residue was purified by column chromatography to give 2-(5-bromo-2-chloro-4-fluorophenyl)-1H-indole.

Step 2: tert-butyl (4-chloro-2-fluoro-5-(1H-indol-2-yl)phenethyl)carbamate. To a mixture of 2-(5-bromo-2-chloro-4-fluorophenyl)-1H-indole (1.00 g, 3.08 mmol, 1.00 eq) and potassium (2-((tert-butoxycarbonyl)amino)ethyl)trifluoroborate (773 mg, 3.08 mmol, 1.00 eq) in dioxane (20 mL) and H₂O (4 mL) were added Cs₂CO₃ (2.01 g, 6.16 mmol, 2.00 eq) and chloro[(di(1-adamantyl)-N-butylphosphine)-2-(2-aminobiphenyl)]palladium(II) (206 mg, 308 μmol, 0.10 eq) under N₂. The reaction mixture was heated to 100° C. After 16 h the reaction was cooled to room temperature, quenched with H₂O (20 mL) and extracted with ethyl acetate (3×20 mL). The combined organic layers were washed with brine (20 mL), dried over Na₂SO₄, filtered and concentrated under reduced pressure. The resulting residue was purified by column chromatography to give tert-butyl (4-chloro-2-fluoro-5-(1H-indol-2-yl)phenethyl)carbamate.

LC-MS (ESI) m/z: [M+H]⁺ calcd for C₂₁H₂₂ClFN₂O₂: 388.14; found 389.2.

Step 3: 2-(4-chloro-2-fluoro-5-(1H-indol-2-yl)phenyl)ethan-1-amine. To a solution of tert-butyl (4-chloro-2-fluoro-5-(1H-indol-2-yl)phenethyl)carbamate (900 mg, 2.31 mmol, 1.00 eq) in ethyl acetate (2 mL) was added HCl (4 M in EtOAc, 11 mL). After 2 h the reaction mixture was concentrated to give 2-(4-chloro-2-fluoro-5-(1H-indol-2-yl)phenyl)ethan-1-amine hydrochloride, which was used without further purification.

### Example 1: (R)-3-((5-chloro-1H-indol-2-yl)methyl)-1-methyl-1-(1-(oxetane-3-carbonyl)piperidin-3-yl)urea (Compound 1)

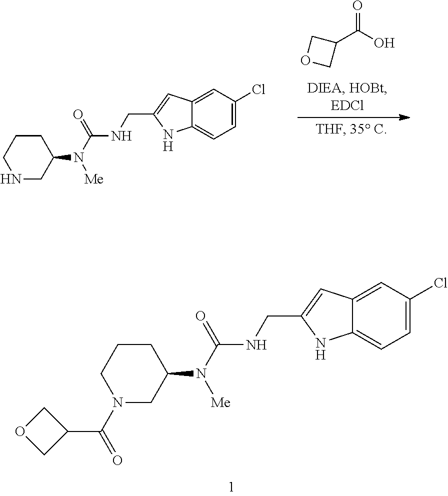 [SMILES: O=C(O)C1COC1 ; CN(C(=O)NCc1cc2cc(Cl)ccc2[nH]1)[C@@H]1CCCNC1 ; CN(C(=O)NCc1cc2cc(Cl)ccc2[nH]1)[C@@H]1CCCN(C(=O)C2COC2)C1]

To a solution of oxetane-3-carboxylic acid (0.069 g, 0.68 mmol, 1.2 eq) in THE (2.8 mL, 0.2M) were added HOBt (0.134 g, 86 w/w %, 0.85 mmol, 1.5 eq) and EDC hydrochloride (0.163 g, 0.85 mmol, 1.5 eq) followed by DIEA (0.30 mL, 1.702 mmol, 3 eq). The resulting mixture was stirred for 5 mins then (R)-3-((5-chloro-1H-indol-2-yl)methyl)-1-methyl-1-(piperidin-3-yl)urea (0.182 g, 0.57 mmol, 1 eq) was added and the reaction was heated to 35° C. After 16 h the reaction was cooled to room temperature and concentrated under reduced pressure. The residue was purified by column chromatography to give (R)-3-((5-chloro-1H-indol-2-yl)methyl)-1-methyl-1-(1-(oxetane-3-carbonyl)piperidin-3-yl)urea. LC-MS (ESI) m/z: [M+H]⁺ calcd for C₂₀H₂₅ClN₄O₃: 405.17; found 405.3. ¹H NMR (500 MHz, MeOD) δ 7.42 (dd, J=4.8, 2.0 Hz, 1H), 7.27 (d, J=8.6 Hz, 1H), 7.00 (ddd, J=8.7, 4.8, 2.0 Hz, 1H), 6.26 (d, J=3.8 Hz, 1H), 4.84-4.75 (m, 3H), 4.53-4.41 (m, 3H), 4.25-4.10 (m, 1H), 4.07-3.94 (m, 1H), 3.44-3.31 (m, 1H), 3.08-2.88 (m, 1H), 2.88-2.80 (m, 3H), 2.79-2.48 (m, 1H), 2.13-1.96 (m, 1H), 1.88-1.72 (m, 3H), 1.57-1.44 (m, 1H).

The following compounds in Table T-1 were synthesized using procedures similar to Compound 1 using the appropriate starting materials.

TABLE T-1

| | | | Exact | LCMS, |
|---|---|---|---|---|
| | | | mass | found |
| Cmpd No. | Structure | IUPAC name | (g/mol) | [M + H]⁺ |
| 2 | 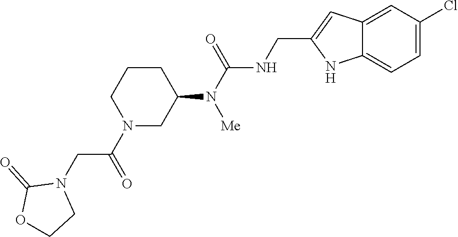 [SMILES: CN(C(=O)NCc1cc2cc(Cl)ccc2[nH]1)[C@@H]1CCCN(C(=O)CN2CCOC2=O)C1] | (R)-3-((5-chloro-1H- indol-2-yl)methyl)-1- methyl-1-(1-(2-(2- oxooxazolidin-3- yl)acetyl)piperidin-3- yl)urea | 447.17 | 448.1 |
| 3 | 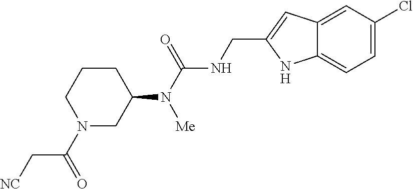 [SMILES: CN(C(=O)NCc1cc2cc(Cl)ccc2[nH]1)[C@@H]1CCCN(C(=O)CC#N)C1] | (R)-3-((5-chloro-1H- indol-2-yl)methyl)-1-(1- (2-cyanoacetyl)piperidin- 3-yl)-1-methylurea | 387.15 | 388.0 |
| 4 | 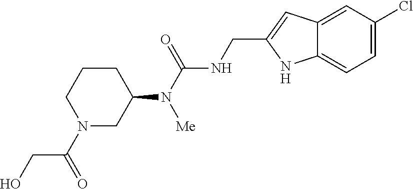 [SMILES: CN(C(=O)NCc1cc2cc(Cl)ccc2[nH]1)[C@@H]1CCCN(C(=O)CO)C1] | (R)-3-((5-chloro-1H- indol-2-yl)methyl)-1-(1- (2- hydroxyacetyl)piperidin- 3-yl)-1-methylurea | 378.15 | 379.0 |
| 5 | 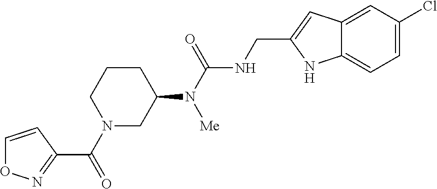 [SMILES: CN(C(=O)NCc1cc2cc(Cl)ccc2[nH]1)[C@@H]1CCCN(C(=O)c2ccon2)C1] | (R)-3-((5-chloro-1H- indol-2-yl)methyl)-1-(1- (isoxazole-3- carbonyl)piperidin-3-yl)- 1-methylurea | 415.14 | 416.0 |
| 6 | 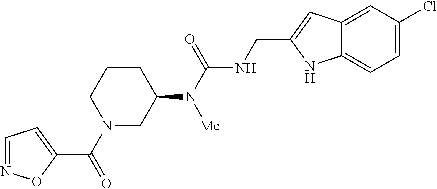 [SMILES: CN(C(=O)NCc1cc2cc(Cl)ccc2[nH]1)[C@@H]1CCCN(C(=O)c2ccno2)C1] | (R)-3-((5-chloro-1H- indol-2-yl)methyl)-1-(1- (isoxazole-5- carbonyl)piperidin-3-yl)- 1-methylurea | 415.14 | 416.0 |
| 7 | 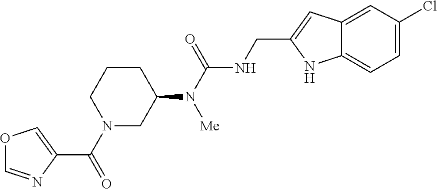 [SMILES: CN(C(=O)NCc1cc2cc(Cl)ccc2[nH]1)[C@@H]1CCCN(C(=O)c2cocn2)C1] | (R)-3-((5-chloro-1H- indol-2-yl)methyl)-1- methyl-1-(1-(oxazole-4- carbonyl)piperidin-3- yl)urea | 415.14 | 416.0 |
| 8 | 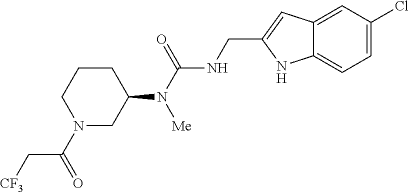 [SMILES: CN(C(=O)NCc1cc2cc(Cl)ccc2[nH]1)[C@@H]1CCCN(C(=O)CC(F)(F)F)C1] | (R)-3-((5-chloro-1H- indol-2-yl)methyl)-1- methyl-1-(1-(3,3,3- trifluoropropanoyl)piperidin- 3-yl)urea | 430.14 | 431.0 |
| 9 | 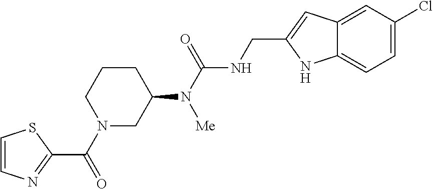 [SMILES: CN(C(=O)NCc1cc2cc(Cl)ccc2[nH]1)[C@@H]1CCCN(C(=O)c2nccs2)C1] | (R)-3-((5-chloro-1H- indol-2-yl)methyl)-1- methyl-1-(1-(thiazole-2- carbonyl)piperidin-3- yl)urea | 431.12 | 429.1 |
| 10 | 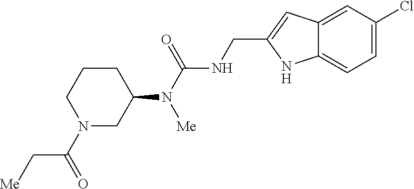 [SMILES: CCC(=O)N1CCC[C@@H](N(C)C(=O)NCc2cc3cc(Cl)ccc3[nH]2)C1] | (R)-3-((5-chloro-1H- indol-2-yl)methyl)-1- methyl-1-(1- propionylpiperidin-3- yl)urea | 376.17 | 377.1 |
| 11 | 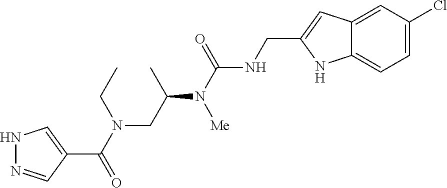 [SMILES: CCN(C[C@@H](C)N(C)C(=O)NCc1cc2cc(Cl)ccc2[nH]1)C(=O)c1cn[nH]c1] | (R)-1-(1-(1H-pyrazole-4- carbonyl)piperidin-3-yl)- 3-((5-chloro-1H-indol-2- yl)methyl)-1-methylurea | 414.16 | 415.0 |
| 12 | 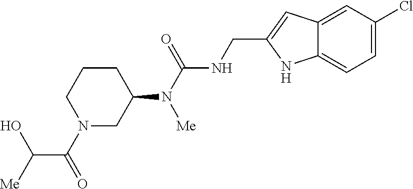 [SMILES: CC(O)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(2- hydroxypropanoyl)piperidin- 3-yl)-1-methylurea | 430.18 | 393.0 |
| 13 | 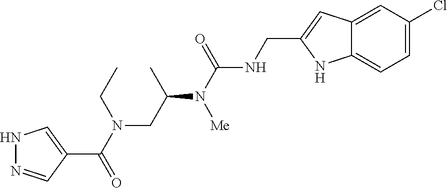 [SMILES: CCN(C[C@@H](C)N(C)C(=O)NCc1cc2cc(Cl)ccc2[nH]1)C(=O)c1cn[nH]c1] | (R)-1-(1-(1H-imidazole-4- carbonyl)piperidin-3-yl)- 3-((5-chloro-1H-indol-2- yl)methyl)-1-methylurea | 414.16 | 415.1 |
| 14 | 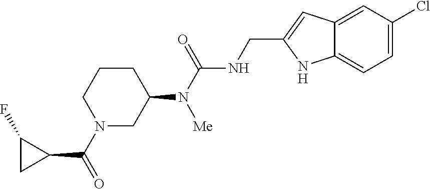 [SMILES: CN(C(=O)NCc1cc2cc(Cl)ccc2[nH]1)[C@@H]1CCCN(C(=O)[C@H]2C[C@@H]2F)C1] | 3-((5-chloro-1H-indol-2- yl)methyl)-1-((R)-1- ((1R,2S)-2- fluorocyclopropane-1- carbonyl)piperidin-3-yl)- 1-methylurea | 406.16 | 407.0 |
| 15 | 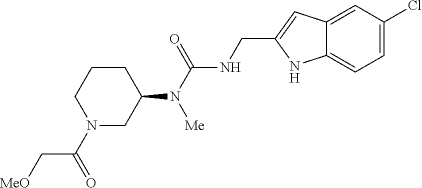 [SMILES: COCC(=O)N1CCC[C@@H](N(C)C(=O)NCc2cc3cc(Cl)ccc3[nH]2)C1] | (R)-3-((5-chloro-1H- indol-2-yl)methyl)-1-(1- (2- methoxyacetyl)piperidin- 3-yl)-1-methylurea | 392.16 | 393.0 |
| 16 | 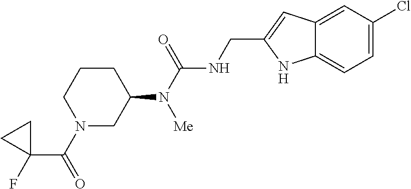 [SMILES: CN(C(=O)NCc1cc2cc(Cl)ccc2[nH]1)[C@@H]1CCCN(C(=O)C2(F)CC2)C1] | (R)-3-((5-chloro-1H- indol-2-yl)methyl)-1-(1- (1-fluorocyclopropane-1- carbonyl)piperidin-3-yl)- 1-methylurea | 406.16 | 407.0 |
| 17 | 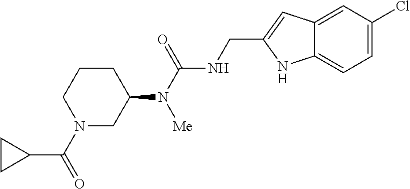 [SMILES: CN(C(=O)NCc1cc2cc(Cl)ccc2[nH]1)[C@@H]1CCCN(C(=O)C2CC2)C1] | (R)-3-((5-chloro-1H- indol-2-yl)methyl)-1-(1- (cyclopropanecarbonyl)pi- peridin-3-yl)-1- methylurea | 388.17 | 389.0 |
| 18 | 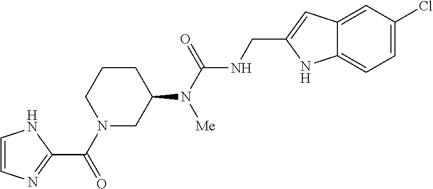 [SMILES: CN(C(=O)NCc1cc2cc(Cl)ccc2[nH]1)[C@@H]1CCCN(C(=O)c2ncc[nH]2)C1] | (R)-1-(1-(1H-imidazole-2- carbonyl)piperidin-3-yl)- 3-((5-chloro-1H-indol-2- yl)methyl)-1-methylurea | 414.16 | 415.1 |
| 19 | 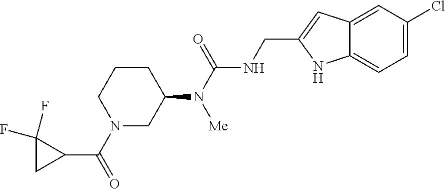 [SMILES: CN(C(=O)NCc1cc2cc(Cl)ccc2[nH]1)[C@@H]1CCCN(C(=O)C2CC2(F)F)C1] | 3-((5-chloro-1H-indol-2- yl)methyl)-1-((3R)-1-(2,2- difluorocyclopropane-1- carbonyl)piperidin-3-yl)- 1-methylurea | 424.15 | 425.0 |
| 20 | 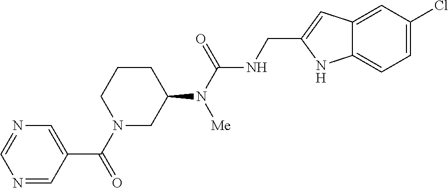 [SMILES: CN(C(=O)NCc1cc2cc(Cl)ccc2[nH]1)[C@@H]1CCCN(C(=O)c2cncnc2)C1] | (R)-3-((5-chloro-1H- indol-2-y1)methyl)-1- methyl-1-(1-(pyrimidine- 5-carbonyl)piperidin-3- yl)urea | 426.16 | 427.0 |
| 21 | 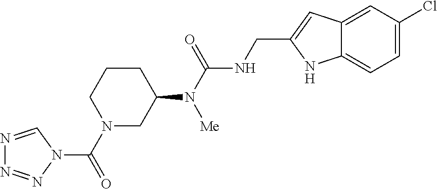 [SMILES: CN(C(=O)NCc1cc2cc(Cl)ccc2[nH]1)[C@@H]1CCCN(C(=O)n2cnnn2)C1] | (R)-1-(1-(2-(1H-tetrazol- 1-yl)acetyl)piperidin-3- yl)-3-((5-chloro-1H-indol- 2-yl)methyl)-1-methylurea | 416.15 | 431.1 |
| 22 | 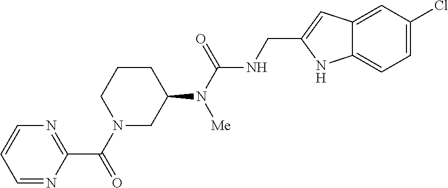 [SMILES: CN(C(=O)NCc1cc2cc(Cl)ccc2[nH]1)[C@@H]1CCCN(C(=O)c2ncccn2)C1] | (R)-3-((5-chloro-1H- indol-2-yl)methyl)-1- methyl-1-(1-(pyrimidine- 2-carbonyl)piperidin-3- yl)urea | 426.16 | 427.1 |
| 23 | 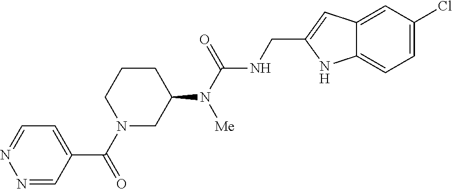 [SMILES: CN(C(=O)NCc1cc2cc(Cl)ccc2[nH]1)[C@@H]1CCCN(C(=O)c2ccnnc2)C1] | (R)-3-((5-chloro-1H- indol-2-yl)methyl)-1- methyl-1-(1-(pyridazine- 4-carbonyl)piperidin-3- yl)urea | 426.16 | 427.0 |
| 24 | 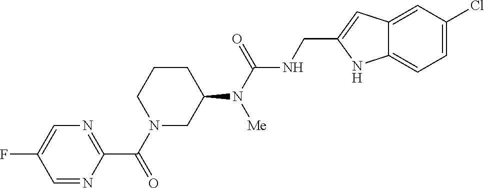 [SMILES: CN(C(=O)NCc1cc2cc(Cl)ccc2[nH]1)[C@@H]1CCCN(C(=O)c2ncc(F)cn2)C1] | (R)-3-((5-chloro-1H- indol-2-yl)methyl)-1-(1- (5-fluoropyrimidine-2- carbonyl)piperidin-3-yl)- 1-methylurea | 444.15 | 445.0 |
| 25 | 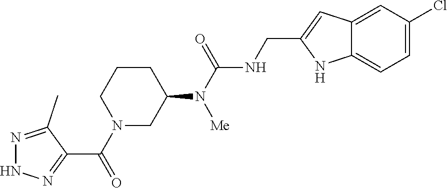 [SMILES: Cc1n[nH]nc1C(=O)N1CCC[C@@H](N(C)C(=O)NCc2cc3cc(Cl)ccc3[nH]2)C1] | (R)-3-((5-chloro-1H- indol-2-yl)methyl)-1- methyl-1-(1-(5-methyl- 2H-1,2,3-triazole-4- carbonyl)piperidin-3- yl)urea | 429.17 | 430.1 |
| 26 | 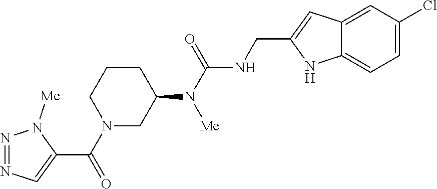 [SMILES: CN(C(=O)NCc1cc2cc(Cl)ccc2[nH]1)[C@@H]1CCCN(C(=O)c2cnnn2C)C1] | (R)-3-((5-chloro-1H- indol-2-yl)methyl)-1- methyl-1-(1-(1-methyl- 1H-1,2,3-triazole-5- carbonyl)piperidin-3- yl)urea | 429.17 | 430.0 |
| 27 | 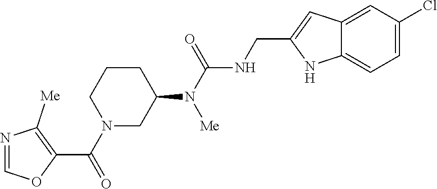 [SMILES: Cc1ncoc1C(=O)N1CCC[C@@H](N(C)C(=O)NCc2cc3cc(Cl)ccc3[nH]2)C1] | (R)-3-((5-chloro-1H- indol-2-y1)methyl)-1- methyl-1-(1-(4- methyloxazole-5- carbonyl)piperidin-3- yl)urea | 429.16 | 430.1 |
| 28 | 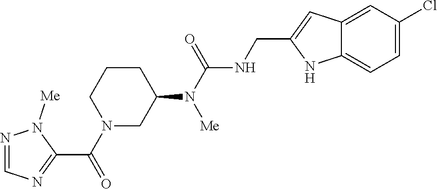 [SMILES: CN(C(=O)NCc1cc2cc(Cl)ccc2[nH]1)[C@@H]1CCCN(C(=O)c2ncnn2C)C1] | (R)-3-((5-chloro-1H- indol-2-yl)methyl)-1- methyl-1-(1-(1-methyl- 1H-1,2,4-triazole-5- carbonyl)piperidin-3- yl)urea | 429.17 | 430.0 |
| 29 | 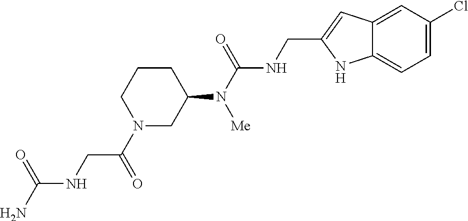 [SMILES: CN(C(=O)NCc1cc2cc(Cl)ccc2[nH]1)[C@@H]1CCCN(C(=O)CNC(N)=O)C1] | (R)-1-(1- (carbamoylglycyl)piperidin- 3-yl)-3-((5-chloro-1H- indol-2-yl)methyl)-1- methylurea | 420.17 | 421.1 |
| 30 | 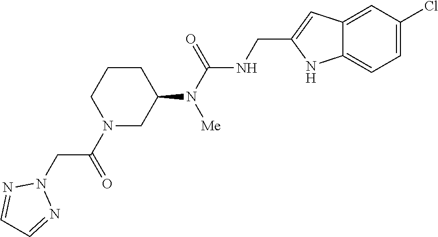 [SMILES: CN(C(=O)NCc1cc2cc(Cl)ccc2[nH]1)[C@@H]1CCCN(C(=O)Cn2nccn2)C1] | (R)-1-(1-(2-(2H-1,2,3- triazol-2- yl)acetyl)piperidin-3-yl)- 3-((5-chloro-1H-indol-2- yl)methyl)-1-methylurea | 429.17 | 430.2 |
| 31 | 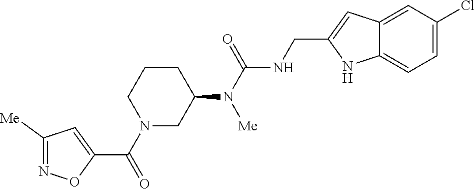 [SMILES: Cc1cc(C(=O)N2CCC[C@@H](N(C)C(=O)NCc3cc4cc(Cl)ccc4[nH]3)C2)on1] | (R)-3-((5-chloro-1H- indol-2-yl)methyl)-1- methyl-1-(1-(3- methylisoxazole-5- carbonyl)piperidin-3- yl)urea | 429.16 | 430.1 |
| 32 | 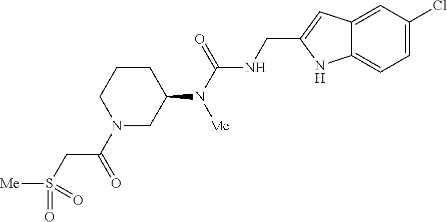 [SMILES: CN(C(=O)NCc1cc2cc(Cl)ccc2[nH]1)[C@@H]1CCCN(C(=O)CS(C)(=O)=O)C1] | (R)-3-((5-chloro-1H- indol-2-yl)methyl)-1- methyl-1-(1-(2- (methylsulfonyl)acetyl)piper- din-3-yl)urea | 440.13 | 441.0 |
| 33 | 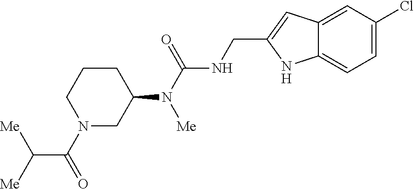 [SMILES: CC(C)C(=O)N1CCC[C@@H](N(C)C(=O)NCc2cc3cc(Cl)ccc3[nH]2)C1] | (R)-3-((5-chloro-1H- indol-2-yl)methyl)-1-(1- isobutyrylpiperidin-3-yl)- 1-methylurea | 391.18 | 391.1 |
| 34 | 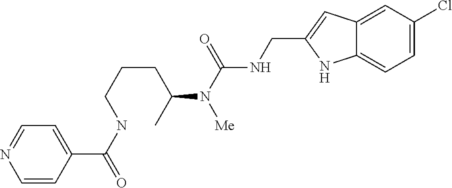 [SMILES: C[C@@H](CCCNC(=O)c1ccncc1)N(C)C(=O)NCc1cc2cc(Cl)ccc2[nH]1] | (R)-3-((5-chloro-1H- indol-2-yl)methyl)-1-(1- isonicotinoylpiperidin-3- yl)-1-methylurea | 425.16 | 426.1 |
| 35 | 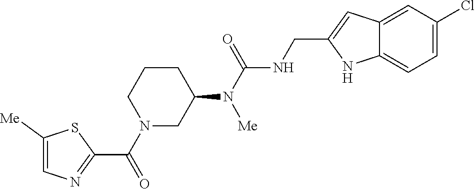 [SMILES: Cc1cnc(C(=O)N2CCC[C@@H](N(C)C(=O)NCc3cc4cc(Cl)ccc4[nH]3)C2)s1] | (R)-3-((5-chloro-1H- indol-2-yl)methyl)-1- methyl-1-(1-(5- methylthiazole-2- carbonyl)piperidin-3- yl)urea | 445.13 | 446.1 |
| 36 | 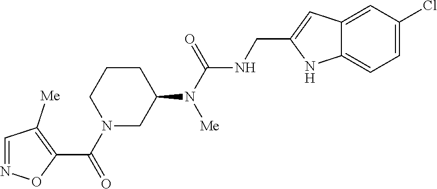 [SMILES: Cc1cnoc1C(=O)N1CCC[C@@H](N(C)C(=O)NCc2cc3cc(Cl)ccc3[nH]2)C1] | (R)-3-((5-chloro-1H- indol-2-yl)methyl)-1- methyl-1-(1-(4- methylisoxazole-5- carbonyl)piperidin-3- yl)urea | 429.16 | 430.0 |
| 37 | 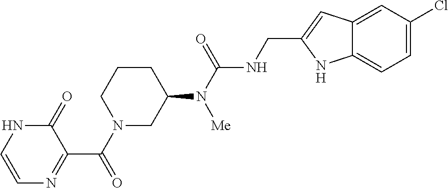 [SMILES: CN(C(=O)NCc1cc2cc(Cl)ccc2[nH]1)[C@@H]1CCCN(C(=O)c2ncc[nH]c2=O)C1] | (R)-3-((5-chloro-1H- indol-2-yl)methyl)-1- methyl-1-(1-(3-oxo-3,4- dihydropyrazine-2- carbonyl)piperidin-3- yl)urea | 442.15 | 443.0 |
| 38 | 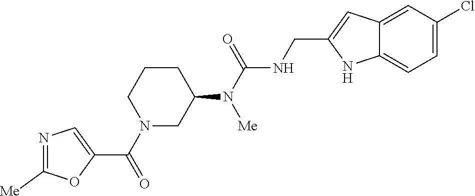 [SMILES: Cc1ncc(C(=O)N2CCC[C@@H](N(C)C(=O)NCc3cc4cc(Cl)ccc4[nH]3)C2)o1] | (R)-3-((5-chloro-1H- indol-2-yl)methyl)-1- methyl-1-(1-(2- methyloxazole-5- carbonyl)piperidin-3- yl)urea | 429.16 | 430.1 |
| 39 | 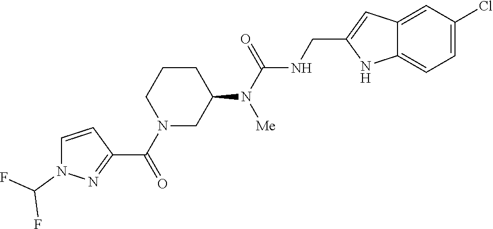 [SMILES: CN(C(=O)NCc1cc2cc(Cl)ccc2[nH]1)[C@@H]1CCCN(C(=O)c2ccn(C(F)F)n2)C1] | (R)-3-((5-chloro-1H- indol-2-yl)methyl)-1-(1- (1-(difluoromethyl)-1H- pyrazole-3- carbonyl)piperidin-3-yl)- 1-methylurea | 464.15 | 465.0 |
| 40 | 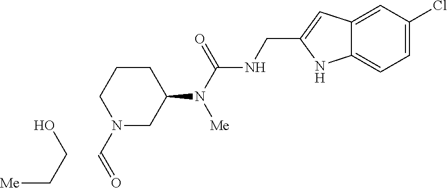 | 3-((5-chloro-1H-indol-2- yl)methyl)-1-((3R)-1-(2- hydroxybutanoyl)piperidin- 3-yl)-1-methylurea | 406.18 | 407.1 |
| 41 | 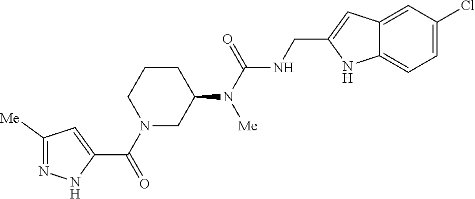 [SMILES: Cc1cc(C(=O)N2CCC[C@@H](N(C)C(=O)NCc3cc4cc(Cl)ccc4[nH]3)C2)[nH]n1] | (R)-3-((5-chloro-1H- indol-2-yl)methyl)-1- methyl-1-(1-(3-methyl- 1H-pyrazole-5- carbonyl)piperidin-3- yl)urea | 428.17 | 429.1 |
| 42 | 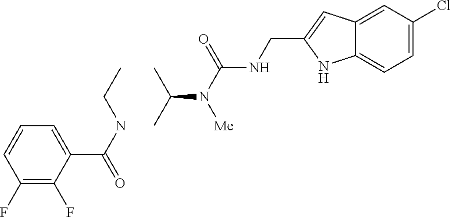 | (R)-3-((5-chloro-1H- indol-2-yl)methyl)-1-(1- (2,3- difluorobenzoyl)piperidin- 3-yl)-1-methylurea | 460.15 | 461.0 |
| 43 | 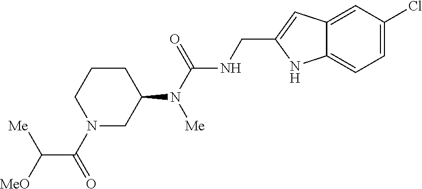 [SMILES: COC(C)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(2- methoxypropanoyl)piperidin- 3-yl)-1-methylurea | 406.18 | 407.0 |
| 44 | 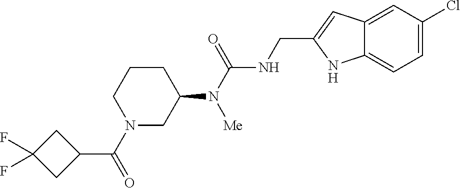 [SMILES: CN(C(=O)NCc1cc2cc(Cl)ccc2[nH]1)[C@@H]1CCCN(C(=O)C2CC(F)(F)C2)C1] | (R)-3-((5-chloro-1H- indol-2-yl)methyl)-1-(1- (3,3-difluorocyclobutane- 1-carbonyl)piperidin-3- yl)-1-methylurea | 438.16 | 439.1 |
| 45 | 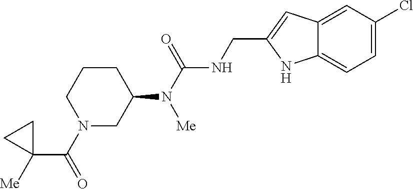 [SMILES: CN(C(=O)NCc1cc2cc(Cl)ccc2[nH]1)[C@@H]1CCCN(C(=O)C2(C)CC2)C1] | (R)-3-((5-chloro-1H- indol-2-yl)methyl)-1- methyl-1-(1-(1- methylcyclopropane-1- carbonyl)piperidin-3- yl)urea | 402.18 | 403.1 |
| 46 | 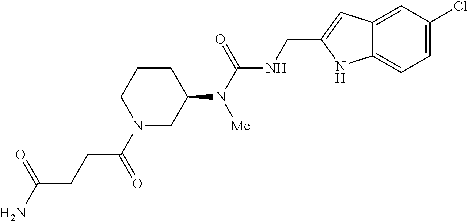 [SMILES: CN(C(=O)NCc1cc2cc(Cl)ccc2[nH]1)[C@@H]1CCCN(C(=O)CCC(N)=O)C1] | (R)-4-(3-(3-((5-chloro-1H- indol-2-y1)methyl)-1- methylureido)piperidin-1- yl)-4-oxobutanamide | 419.17 | 420.1 |
| 47 | 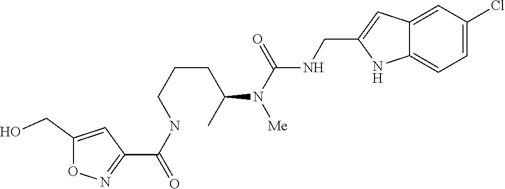 [SMILES: C[C@@H](CCCNC(=O)c1cc(CO)on1)N(C)C(=O)NCc1cc2cc(Cl)ccc2[nH]1] | (R)-3-((5-chloro-1H- indol-2-yl)methyl)-1-(1- (5- (hydroxymethyl)isoxazole- 3-carbonyl)piperidin-3- yl)-1-methylurea | 445.15 | 446.1 |
| 48 | 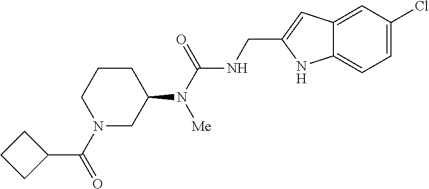 [SMILES: CN(C(=O)NCc1cc2cc(Cl)ccc2[nH]1)[C@@H]1CCCN(C(=O)C2CCC2)C1] | (R)-3-((5-chloro-1H- indol-2-yl)methyl)-1-(1- (cyclobutanecarbonyl)piper- idin-3-yl)-1-methylurea | 402.18 | 403.1 |
| 49 | 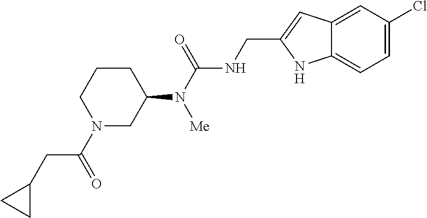 [SMILES: CN(C(=O)NCc1cc2cc(Cl)ccc2[nH]1)[C@@H]1CCCN(C(=O)CC2CC2)C1] | (R)-3-((5-chloro-1H- indol-2-yl)methyl)-1-(1- (2- cyclopropylacetyl)piperidin- 3-yl)-1-methylurea | 402.18 | 403.1 |
| 50 | 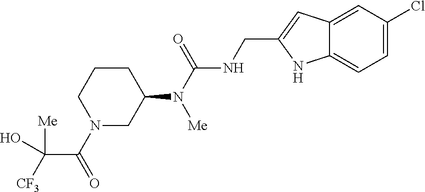 [SMILES: CN(C(=O)NCc1cc2cc(Cl)ccc2[nH]1)[C@@H]1CCCN(C(=O)C(C)(O)C(F)(F)F)C1] | 3-((5-chloro-1H-indol-2- yl)methyl)-1-methyl-1- ((3R)-1-(3,3,3-trifluoro-2- hydroxy-2- methylpropanoyl)piperidin- 3-yl)urea | 460.15 | 461.0 |
| 51 | 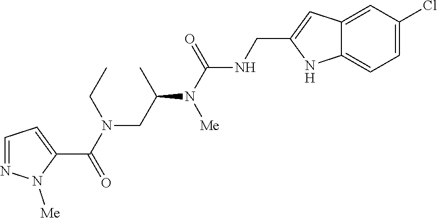 [SMILES: CCN(C[C@@H](C)N(C)C(=O)NCc1cc2cc(Cl)ccc2[nH]1)C(=O)c1ccnn1C] | (R)-3-((5-chloro-1H- indol-2-yl)methyl)-1- methyl-1-(1-(1-methyl- 1H-pyrazole-5- carbonyl)piperidin-3- yl)urea | 428.17 | 429.1 |
| 52 | 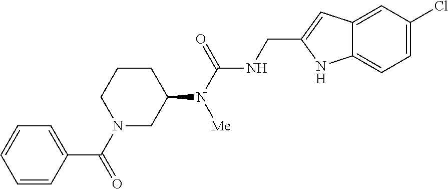 [SMILES: CN(C(=O)NCc1cc2cc(Cl)ccc2[nH]1)[C@@H]1CCCN(C(=O)c2ccccc2)C1] | (R)-1-(1-benzoylpiperidin- 3-yl)-3-((5-chloro-1H- indol-2-yl)methyl)-1- methylurea | 424.17 | 425.1 |
| 53 | 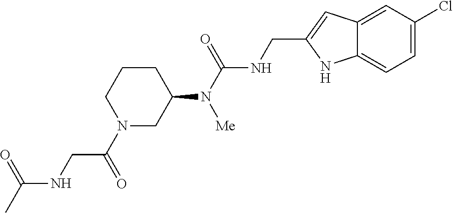 [SMILES: CC(=O)NCC(=O)N1CCC[C@@H](N(C)C(=O)NCc2cc3cc(Cl)ccc3[nH]2)C1] | (R)-N-(2-(3-(3-((5-chloro- 1H-indol-2-yl)methyl)-1- methylureido)piperidin-1- yl)-2-oxoethyl)acetamide | 419.17 | 420.0 |
| 54 | 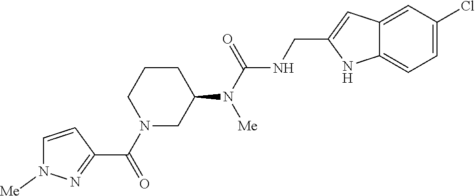 [SMILES: CN(C(=O)NCc1cc2cc(Cl)ccc2[nH]1)[C@@H]1CCCN(C(=O)c2ccn(C)n2)C1] | (R)-3-((5-chloro-1H- indol-2-yl)methyl)-1- methyl-1-(1-(1-methyl- 1H-pyrazole-3- carbonyl)piperidin-3- yl)urea | 428.17 | 429.2 |
| 55 | 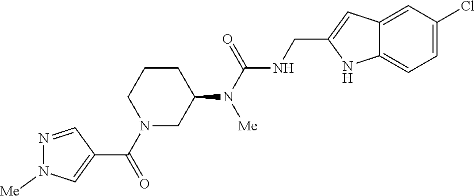 [SMILES: CN(C(=O)NCc1cc2cc(Cl)ccc2[nH]1)[C@@H]1CCCN(C(=O)c2cnn(C)c2)C1] | (R)-3-((5-chloro-1H- indol-2-yl)methyl)-1- methyl-1-(1-(1-methyl- 1H-pyrazole-4- carbonyl)piperidin-3- yl)urea | 428.17 | 429.1 |
| 56 | 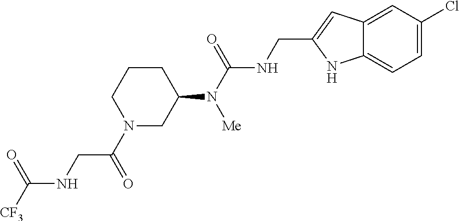 [SMILES: CN(C(=O)NCc1cc2cc(Cl)ccc2[nH]1)[C@@H]1CCCN(C(=O)CNC(=O)C(F)(F)F)C1] | (R)-N-(2-(3-(3-((5-chloro- 1H-indol-2-yl)methyl)-1- methylureido)piperidin-1- yl)-2-oxoethyl)-2,2,2- trifluoroacetamide | 473.14 | 474.1 |
| 57 | 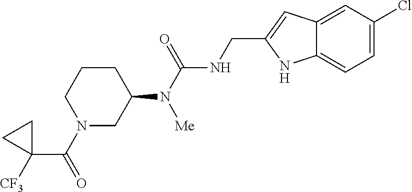 [SMILES: CN(C(=O)NCc1cc2cc(Cl)ccc2[nH]1)[C@@H]1CCCN(C(=O)C2(C(F)(F)F)CC2)C1] | (R)-3-((5-chloro-1H- indol-2-yl)methyl)-1- methyl-1-(1-(1- (trifluoromethyl)cyclopro- pane-1- carbonyl)piperidin-3- yl)urea | 465.15 | 457.1 |
| 58 | 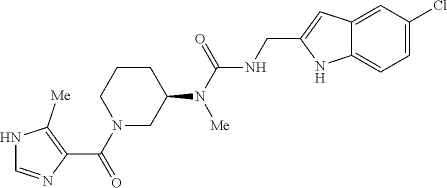 [SMILES: Cc1[nH]cnc1C(=O)N1CCC[C@@H](N(C)C(=O)NCc2cc3cc(Cl)ccc3[nH]2)C1] | (R)-3-((5-chloro-1H- indol-2-yl)methyl)-1- methyl-1-(1-(5-methyl- 1H-imidazole-4- carbonyl)piperidin-3- yl)urea | 428.17 | 429.1 |
| 59 | 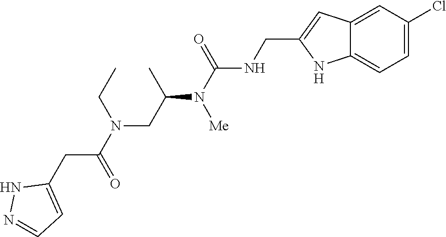 [SMILES: CCN(C[C@@H](C)N(C)C(=O)NCc1cc2cc(Cl)ccc2[nH]1)C(=O)Cc1ccn[nH]1] | (R)-1-(1-(2-(1H-pyrazol- 5-yl)acetyl)piperidin-3- yl)-3-((5-chloro-1H-indol- 2-yl)methyl)-1-methylurea | 428.17 | 429.0 |
| 60 | 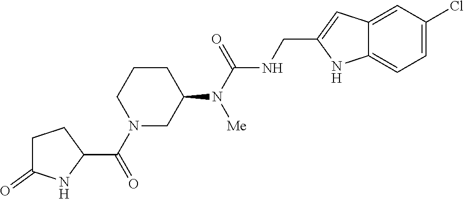 [SMILES: CN(C(=O)NCc1cc2cc(Cl)ccc2[nH]1)[C@@H]1CCCN(C(=O)C2CCC(=O)N2)C1] | 3-((5-chloro-1H-indol-2- yl)methyl)-1-methyl-1- ((3R)-1-(5-oxopyrrolidine- 2-carbonyl)piperidin-3- yl)urea | 431.17 | 432.1 |
| 61 | 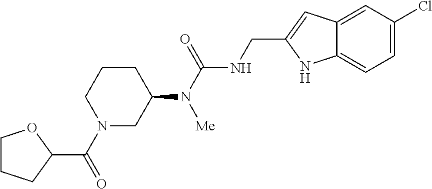 [SMILES: CN(C(=O)NCc1cc2cc(Cl)ccc2[nH]1)[C@@H]1CCCN(C(=O)C2CCCO2)C1] | 3-((5-chloro-1H-indol-2- yl)methyl)-1-methyl-1- ((3R)-1-(tetrahydrofuran- 2-carbonyl)piperidin-3- yl)urea | 418.18 | 419.1 |
| 62 | 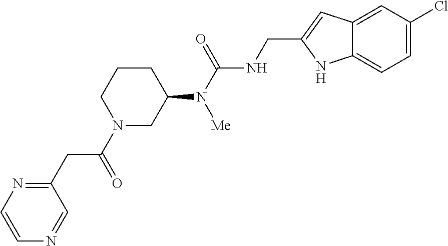 [SMILES: CN(C(=O)NCc1cc2cc(Cl)ccc2[nH]1)[C@@H]1CCCN(C(=O)Cc2cnccn2)C1] | (R)-3-((5-chloro-1H- indol-2-yl)methyl)-1- methyl-1-(1-(2-(pyrazin- 2-yl)acetyl)piperidin-3- yl)urea | 440.17 | 441.0 |
| 63 | 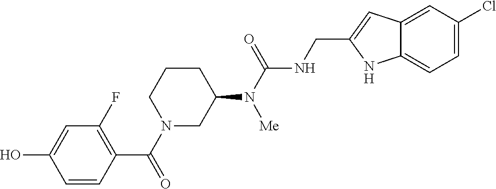 [SMILES: CN(C(=O)NCc1cc2cc(Cl)ccc2[nH]1)[C@@H]1CCCN(C(=O)c2ccc(O)cc2F)C1] | (R)-3-((5-chloro-1H- indol-2-yl)methyl)-1-(1- (2-fluoro-4- hydroxybenzoyl)piperidin- 3-yl)-1-methylurea | 458.15 | 459.1 |
| 64 | 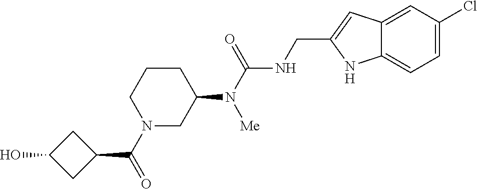 [SMILES: CN(C(=O)NCc1cc2cc(Cl)ccc2[nH]1)[C@@H]1CCCN(C(=O)[C@H]2C[C@H](O)C2)C1] | trans-(R)-3-((5-chloro-1H- indol-2-yl)methyl)-1-(1- (3-hydroxycyclobutane-1- carbonyl)piperidin-3-yl)- 1-methylurea | 418.18 | 419.2 |
| 65 | 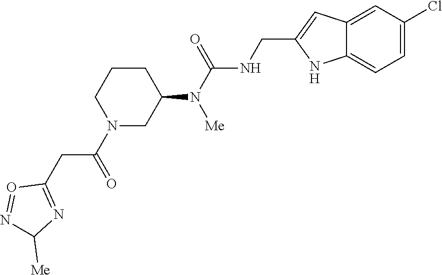 | (R)-3-((5-chloro-1H- indol-2-yl)methyl)-1- methyl-1-(1-(2-(3-methyl- 1,2,4-oxadiazol-5- yl)acetyl)piperidin-3- yl)urea | 444.17 | 445.2 |
| 66 | 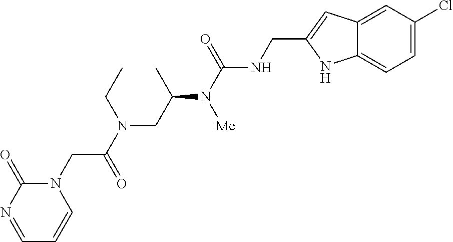 [SMILES: CCN(C[C@@H](C)N(C)C(=O)NCc1cc2cc(Cl)ccc2[nH]1)C(=O)Cn1cccnc1=O] | (R)-3-((5-chloro-1H- indol-2-yl)methyl)-1- methyl-1-(1-(2-(2- oxopyrimidin-1(2H)- yl)acetyl)piperidin-3- yl)urea | 456.17 | 457.2 |
| 67 | 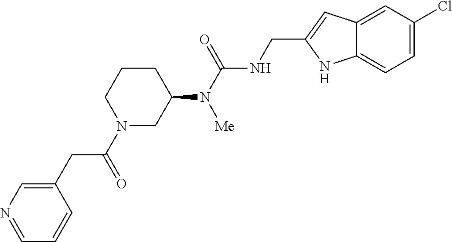 [SMILES: CN(C(=O)NCc1cc2cc(Cl)ccc2[nH]1)[C@@H]1CCCN(C(=O)Cc2cccnc2)C1] | (R)-3-((5-chloro-1H- indol-2-y1)methyl)-1- methyl-1-(1-(2-(pyridin-3- yl)acetyl)piperidin-3- yl)urea | 439.18 | 440.2 |
| 68 | 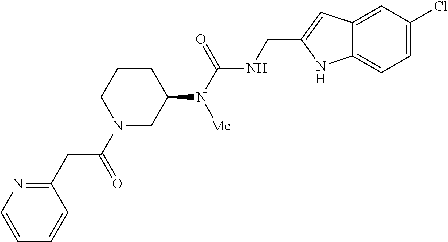 [SMILES: CN(C(=O)NCc1cc2cc(Cl)ccc2[nH]1)[C@@H]1CCCN(C(=O)Cc2ccccn2)C1] | (R)-3-((5-chloro-1H- indol-2-yl)methyl)-1- methyl-1-(1-(2-(pyridin-2- yl)acetyl)piperidin-3- yl)urea | 439.18 | 440.1 |
| 69 | 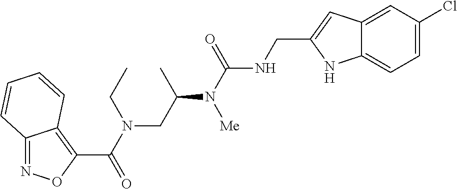 [SMILES: CCN(C[C@@H](C)N(C)C(=O)NCc1cc2cc(Cl)ccc2[nH]1)C(=O)c1onc2ccccc12] | (R)-1-(1- (benzo[c]isoxazole-3- carbonyl)piperidin-3-yl)- 3-((5-chloro-1H-indol-2- yl)methyl)-1-methylurea | 465.16 | 466.1 |
| 70 | 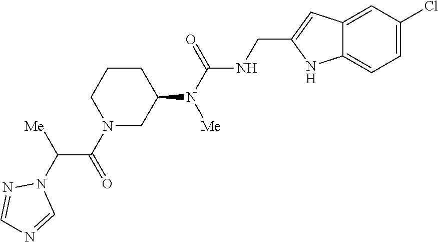 [SMILES: CC(C(=O)N1CCC[C@@H](N(C)C(=O)NCc2cc3cc(Cl)ccc3[nH]2)C1)n1cncn1] | 1-((3R)-1-(2-(1H-1,2,4- triazol-1- yl)propanoyl)piperidin-3- yl)-3-((5-chloro-1H-indol- 2-yl)methyl)-1-methylurea | 443.18 | 444.1 |
| 71 | 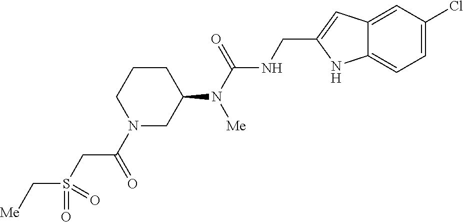 [SMILES: CCS(=O)(=O)CC(=O)N1CCC[C@@H](N(C)C(=O)NCc2cc3cc(Cl)ccc3[nH]2)C1] | (R)-3-((5-chloro-1H- indol-2-yl)methyl)-1-(1- (2- (ethylsulfonyl)acetyl)piper- idin-3-yl)-1-methylurea | 454.14 | 455.1 |
| 72 | 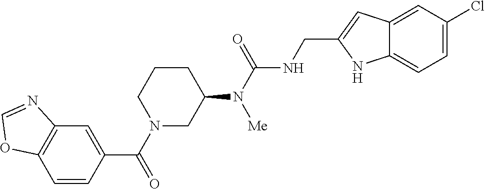 [SMILES: CN(C(=O)NCc1cc2cc(Cl)ccc2[nH]1)[C@@H]1CCCN(C(=O)c2ccc3ocnc3c2)C1] | (R)-1-(1- (benzo[d]oxazole-5- carbonyl)piperidin-3-yl)- 3-((5-chloro-1H-indol-2- yl)methyl)-1-methylurea | 465.16 | 466.1 |
| 73 | 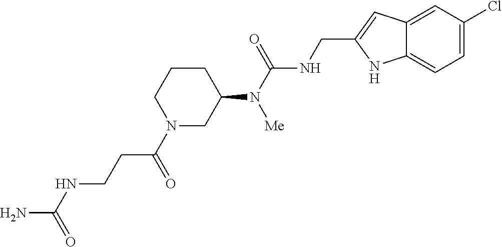 [SMILES: CN(C(=O)NCc1cc2cc(Cl)ccc2[nH]1)[C@@H]1CCCN(C(=O)CCNC(N)=O)C1] | (R)-3-((5-chloro-1H- indol-2-yl)methyl)-1- methyl-1-(1-(3- ureidopropanoyl)piperidin- 3-yl)urea | 434.18 | 435.1 |
| 74 | 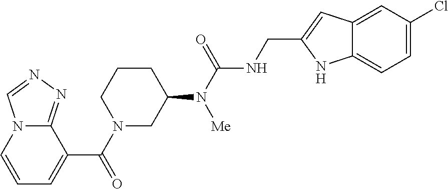 [SMILES: CN(C(=O)NCc1cc2cc(Cl)ccc2[nH]1)[C@@H]1CCCN(C(=O)c2cccn3cnnc23)C1] | (R)-1-(1- ([1,2,4]triazolo[4,3- a]pyridine-8- carbonyl)piperidin-3-yl)- 3-((5-chloro-1H-indol-2- yl)methyl)-1-methylurea | 465.17 | 460.0 |
| 75 | 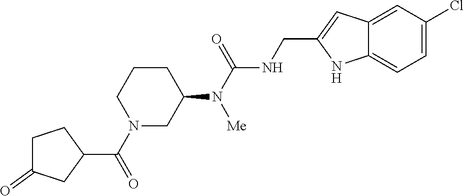 [SMILES: CN(C(=O)NCc1cc2cc(Cl)ccc2[nH]1)[C@@H]1CCCN(C(=O)C2CCC(=O)C2)C1] | 3-((5-chloro-1H-indol-2- yl)methyl)-1-methyl-1- ((3R)-1-(3- oxocyclopentane-1- carbonyl)piperidin-3- yl)urea | 430.18 | 431.1 |
| 76 | 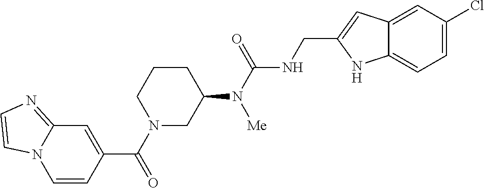 [SMILES: CN(C(=O)NCc1cc2cc(Cl)ccc2[nH]1)[C@@H]1CCCN(C(=O)c2ccn3ccnc3c2)C1] | (R)-3-((5-chloro-1H- indol-2-y1)methyl)-1-(1- (imidazo[1,2-a]pyridine- 7-carbonyl)piperidin-3- yl)-1-methylurea | 464.17 | 465.0 |
| 77 | 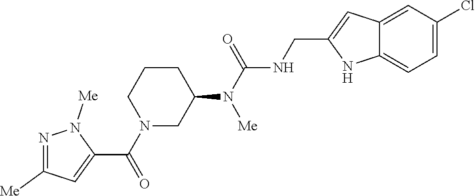 [SMILES: Cc1cc(C(=O)N2CCC[C@@H](N(C)C(=O)NCc3cc4cc(Cl)ccc4[nH]3)C2)n(C)n1] | (R)-3-((5-chloro-1H- indol-2-yl)methyl)-1-(1- (1,3-dimethyl-1H- pyrazole-5- carbonyl)piperidin-3-yl)- 1-methylurea | 442.19 | 443.1 |
| 78 | 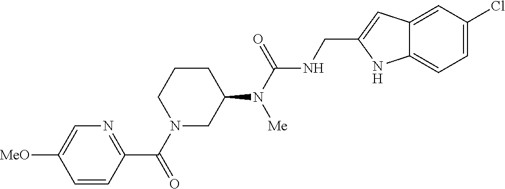 [SMILES: COc1ccc(C(=O)N2CCC[C@@H](N(C)C(=O)NCc3cc4cc(Cl)ccc4[nH]3)C2)nc1] | (R)-3-((5-chloro-1H- indol-2-yl)methyl)-1-(1- (5- methoxypicolinoyl)piperidin- 3-yl)-1-methylurea | 455.17 | 456.1 |
| 79 | 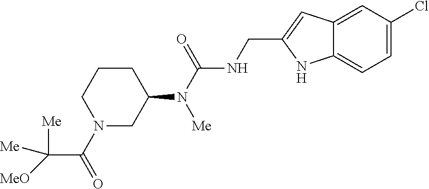 [SMILES: COC(C)(C)C(=O)N1CCC[C@@H](N(C)C(=O)NCc2cc3cc(Cl)ccc3[nH]2)C1] | (R)-3-((5-chloro-1H- indol-2-yl)methyl)-1-(1- (2-methoxy-2- methylpropanoyl)piperidin- 3-yl)-1-methylurea | 420.19 | 421.1 |
| 80 | 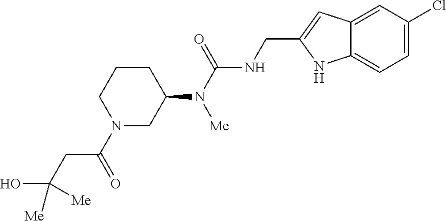 [SMILES: CN(C(=O)NCc1cc2cc(Cl)ccc2[nH]1)[C@@H]1CCCN(C(=O)CC(C)(C)O)C1] | (R)-3-((5-chloro-1H- indol-2-yl)methyl)-1-(1- (3-hydroxy-3- methylbutanoyl)piperidin- 3-yl)-1-methylurea | 420.19 | 421.1 |
| 81 | 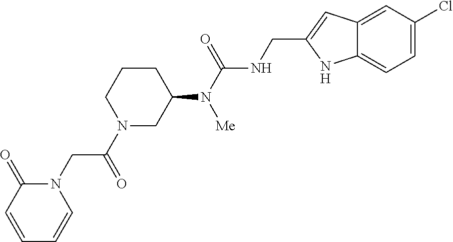 [SMILES: CN(C(=O)NCc1cc2cc(Cl)ccc2[nH]1)[C@@H]1CCCN(C(=O)Cn2ccccc2=O)C1] | (R)-3-((5-chloro-1H- indol-2-yl)methyl)-1- methyl-1-(1-(2-(2- oxopyridin-1(2H)- yl)acetyl)piperidin-3- yl)urea | 455.17 | 456.2 |
| 82 | 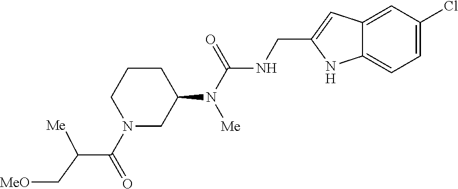 [SMILES: COCC(C)C(=O)N1CCC[C@@H](N(C)C(=O)NCc2cc3cc(Cl)ccc3[nH]2)C1] | 3-((5-chloro-1H-indol-2- yl)methyl)-1-((3R)-1-(3- methoxy-2- methylpropanoyl)piperidin- 3-yl)-1-methylurea | 420.19 | 421.1 |
| 83 | 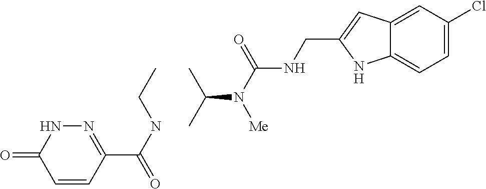 | (R)-3-((5-chloro-1H- indol-2-yl)methyl)-1- methyl-1-(1-(2-((6-oxo- 1,6-dihydropyridazin-3- yl)oxy)acetyl)piperidin-3- yl)urea | 472.16 | 473.0 |
| 84 | 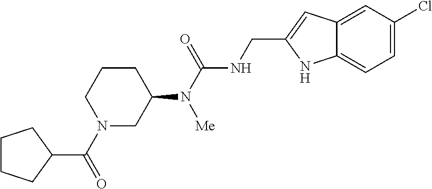 [SMILES: CN(C(=O)NCc1cc2cc(Cl)ccc2[nH]1)[C@@H]1CCCN(C(=O)C2CCCC2)C1] | (R)-3-((5-chloro-1H- indol-2-yl)methyl)-1-(1- (cyclopentanecarbonyl)piper- idin-3-yl)-1-methylurea | 416.20 | 417.2 |
| 85 | 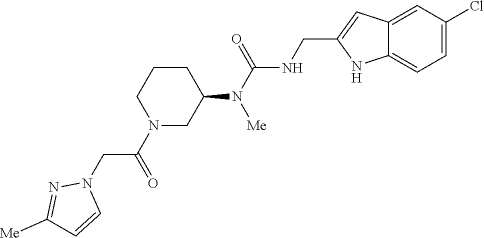 [SMILES: Cc1ccn(CC(=O)N2CCC[C@@H](N(C)C(=O)NCc3cc4cc(Cl)ccc4[nH]3)C2)n1] | (R)-3-((5-chloro-1H- indol-2-yl)methyl)-1- methyl-1-(1-(2-(3-methyl- 1H-pyrazol-1- yl)acetyl)piperidin-3- yl)urea | 442.19 | 443.1 |
| 86 | 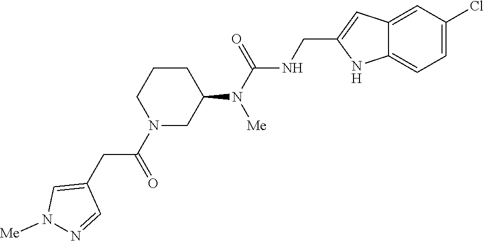 [SMILES: CN(C(=O)NCc1cc2cc(Cl)ccc2[nH]1)[C@@H]1CCCN(C(=O)Cc2cnn(C)c2)C1] | (R)-3-((5-chloro-1H- indol-2-yl)methyl)-1- methyl-1-(1-(2-(1-methyl- 1H-pyrazol-4- yl)acetyl)piperidin-3- yl)urea | 442.19 | 443.1 |
| 87 | 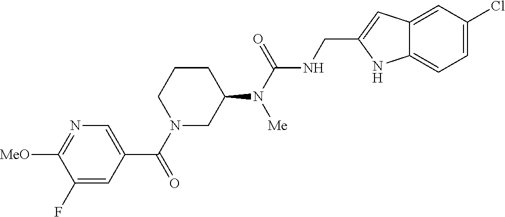 [SMILES: COc1ncc(C(=O)N2CCC[C@@H](N(C)C(=O)NCc3cc4cc(Cl)ccc4[nH]3)C2)cc1F] | (R)-3-((5-chloro-1H- indol-2-yl)methyl)-1-(1- (5-fluoro-6- methoxynicotinoyl)piperidin- 3-yl)-1-methylurea | 473.16 | 474.0 |
| 88 | 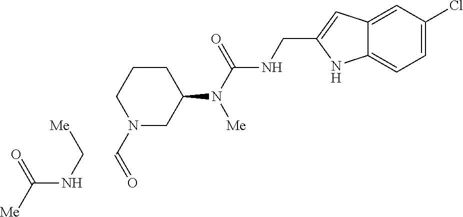 | N-(1-((R)-3-(3-((5-chloro- 1H-indol-2-yl)methyl)-1- methylureido)piperidin-1- yl)-1-oxopropan-2- yl)acetamide | 433.19 | 434.1 |
| 89 | 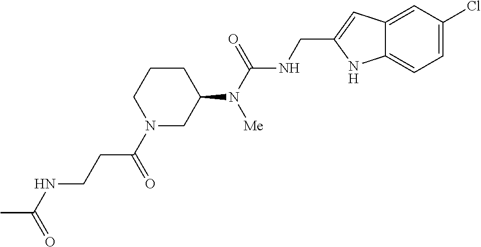 [SMILES: CC(=O)NCCC(=O)N1CCC[C@@H](N(C)C(=O)NCc2cc3cc(Cl)ccc3[nH]2)C1] | (R)-N-(3-(3-(3-((5-chloro- 1H-indol-2-yl)methyl)-1- methylureido)piperidin-1- yl)-3- oxopropyl)acetamide | 433.19 | 434.1 |
| 90 | 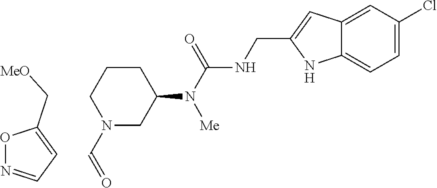 | (R)-3-((5-chloro-1H- indol-2-yl)methyl)-1-(1- (5- (methoxymethyl)isoxazole- 4-carbonyl)piperidin-3- yl)-1-methylurea | 459.17 | 460.0 |
| 91 | 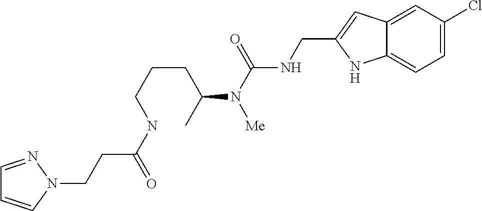 [SMILES: C[C@@H](CCCNC(=O)CCn1cccn1)N(C)C(=O)NCc1cc2cc(Cl)ccc2[nH]1] | (R)-1-(1-(3-(1H-pyrazol- 1-yl)propanoyl)piperidin- 3-yl)-3-((5-chloro-1H- indol-2-yl)methyl)-1- methylurea | 442.19 | 443.1 |
| 92 | 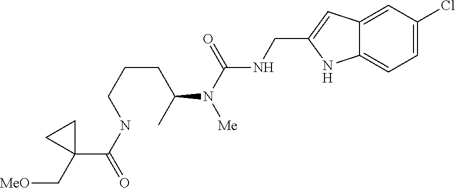 [SMILES: COCC1(C(=O)NCCC[C@H](C)N(C)C(=O)NCc2cc3cc(Cl)ccc3[nH]2)CC1] | (R)-3-((5-chloro-1H- indol-2-yl)methyl)-1-(1- (1- (methoxymethyl)cyclopro- pane-1- carbonyl)piperidin-3-yl)- 1-methylurea | 432.19 | 433.1 |
| 93 | 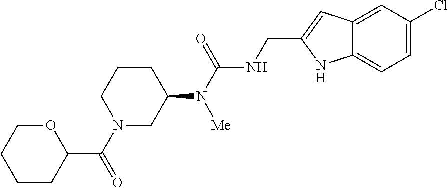 [SMILES: CN(C(=O)NCc1cc2cc(Cl)ccc2[nH]1)[C@@H]1CCCN(C(=O)C2CCCCO2)C1] | 3-((5-chloro-1H-indol-2- yl)methyl)-1-methyl-1- ((3R)-1-(tetrahydro-2H- pyran-2- carbonyl)piperidin-3- yl)urea | 432.19 | 433.0 |
| 94 | 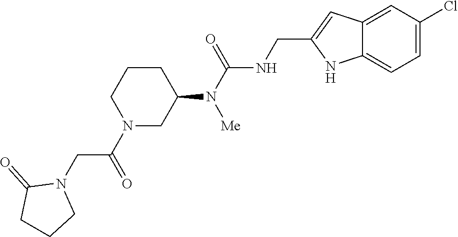 [SMILES: CN(C(=O)NCc1cc2cc(Cl)ccc2[nH]1)[C@@H]1CCCN(C(=O)CN2CCCC2=O)C1] | (R)-3-((5-chloro-1H- indol-2-yl)methyl)-1- methyl-1-(1-(2-(2- oxopyrrolidin-1- yl)acetyl)piperidin-3- yl)urea | 445.19 | 446.1 |
| 95 | 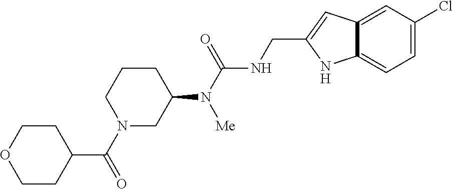 | (R)-3-((5-chloro-1H- indol-2-yl)methyl)-1- methyl-1-(1-(tetrahydro- 2H-pyran-4- carbonyl)piperidin-3- yl)urea | 432.19 | 433.1 |
| 96 | 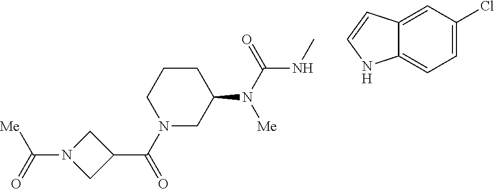 | (R)-1-(1-(1- acetylazetidine-3- carbonyl)piperidin-3-yl)- 3-((5-chloro-1H-indol-2- yl)methyl)-1-methylurea | 445.19 | 446.1 |
| 97 | 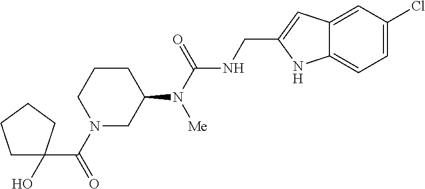 [SMILES: CN(C(=O)NCc1cc2cc(Cl)ccc2[nH]1)[C@@H]1CCCN(C(=O)C2(O)CCCC2)C1] | (R)-3-((5-chloro-1H- indol-2-yl)methyl)-1-(1- (1-hydroxycyclopentane- 1-carbonyl)piperidin-3- yl)-1-methylurea | 432.19 | 433.1 |
| 98 | 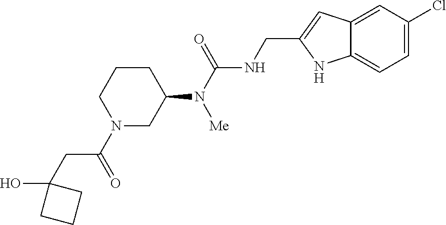 [SMILES: CN(C(=O)NCc1cc2cc(Cl)ccc2[nH]1)[C@@H]1CCCN(C(=O)CC2(O)CCC2)C1] | (R)-3-((5-chloro-1H- indol-2-yl)methyl)-1-(1- (2-(1- hydroxycyclobutyl)acetyl) piperidin-3-yl)-1- methylurea | 432.19 | 433.1 |
| 99 | 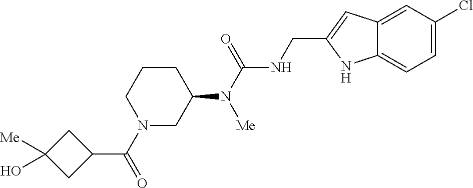 [SMILES: CN(C(=O)NCc1cc2cc(Cl)ccc2[nH]1)[C@@H]1CCCN(C(=O)C2CC(C)(O)C2)C1] | (R)-3-((5-chloro-1H- indol-2-yl)methyl)-1-(1- (3-hydroxy-3- methylcyclobutane-1- carbonyl)piperidin-3-yl)- 1-methylurea | 432.19 | 433.1 |
| 100 | 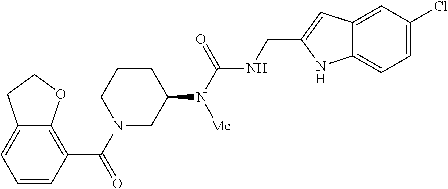 [SMILES: CN(C(=O)NCc1cc2cc(Cl)ccc2[nH]1)[C@@H]1CCCN(C(=O)c2cccc3c2OCC3)C1] | (R)-3-((5-chloro-1H- indol-2-yl)methyl)-1-(1- (2,3-dihydrobenzofuran-7- carbonyl)piperidin-3-yl)- 1-methylurea | 466.18 | 467.1 |
| 101 | 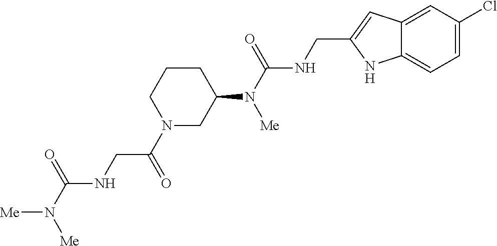 [SMILES: CN(C)C(=O)NCC(=O)N1CCC[C@@H](N(C)C(=O)NCc2cc3cc(Cl)ccc3[nH]2)C1] | (R)-3-((5-chloro-1H- indol-2-y1)methyl)-1-(1- ((dimethylcarbamoyl)glycyl) piperidin-3-yl)-1- methylurea | 448.20 | 449.1 |
| 102 | 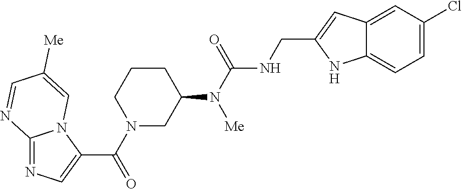 [SMILES: Cc1cnc2ncc(C(=O)N3CCC[C@@H](N(C)C(=O)NCc4cc5cc(Cl)ccc5[nH]4)C3)n2c1] | (R)-3-((5-chloro-1H- indol-2-yl)methyl)-1- methyl-1-(1-(6- methylimidazo[1,2- a]pyrimidine-3- carbonyl)piperidin-3- yl)urea | 479.18 | 480.1 |
| 103 | 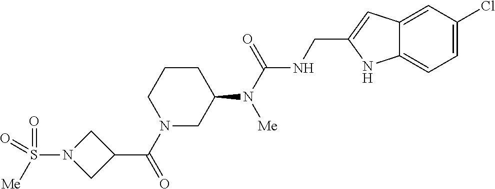 [SMILES: CN(C(=O)NCc1cc2cc(Cl)ccc2[nH]1)[C@@H]1CCCN(C(=O)C2CN(S(C)(=O)=O)C2)C1] | (R)-3-((5-chloro-1H- indol-2-yl)methyl)-1- methyl-1-(1-(1- (methylsulfonyl)azetidine- 3-carbonyl)piperidin-3- yl)urea | 481.16 | 482.0 |
| 104 | 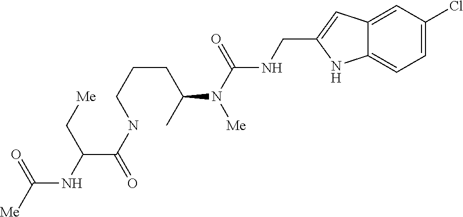 [SMILES: CCC(NC(C)=O)C(=O)NCCC[C@H](C)N(C)C(=O)NCc1cc2cc(Cl)ccc2[nH]1] | N-(1-((R)-3-(3-((5-chloro- 1H-indol-2-yl)methyl)-1- methylureido)piperidin-1- yl)-1-oxobutan-2- yl)acetamide | 447.20 | 448.1 |
| 105 | 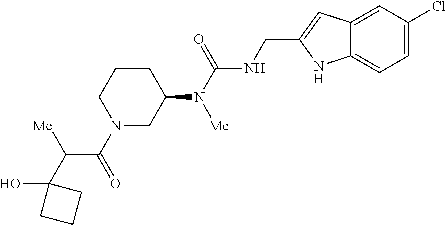 [SMILES: CC(C(=O)N1CCC[C@@H](N(C)C(=O)NCc2cc3cc(Cl)ccc3[nH]2)C1)C1(O)CCC1] | 3-((5-chloro-1H-indol-2- yl)methyl)-1-((3R)-1-(2- (1- hydroxycyclobutyl)propan- oyl)piperidin-3-yl)-1- methylurea | 446.21 | 447.1 |
| 106 | 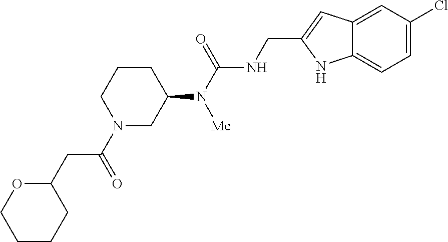 [SMILES: CN(C(=O)NCc1cc2cc(Cl)ccc2[nH]1)[C@@H]1CCCN(C(=O)CC2CCCCO2)C1] | 3-((5-chloro-1H-indol-2- yl)methyl)-1-methyl-1- ((3R)-1-(2-(tetrahydro- 2H-pyran-2- yl)acetyl)piperidin-3- yl)urea | 446.21 | 447.1 |
| 107 | 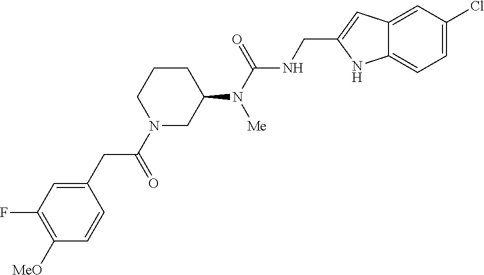 [SMILES: COc1ccc(CC(=O)N2CCC[C@@H](N(C)C(=O)NCc3cc4cc(Cl)ccc4[nH]3)C2)cc1F] | (R)-3-((5-chloro-1H- indol-2-yl)methyl)-1-(1- (2-(3-fluoro-4- methoxyphenyl)acetyl)piper- idin-3-yl)-1-methylurea | 486.18 | 487.2 |
| 108 | 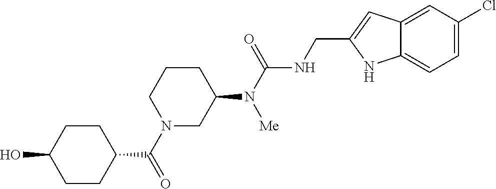 [SMILES: CN(C(=O)NCc1cc2cc(Cl)ccc2[nH]1)[C@@H]1CCCN(C(=O)[C@H]2CC[C@H](O)CC2)C1] | trans-3-((5-chloro-1H- indol-2-yl)methyl)-1-((R)- 1-((4- hydroxycyclohexane-1- carbonyl)piperidin-3-yl)- 1-methylurea | 446.21 | 447.1 |
| 109 | 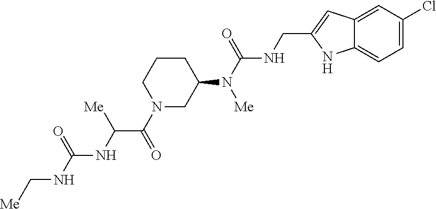 [SMILES: CCNC(=O)NC(C)C(=O)N1CCC[C@@H](N(C)C(=O)NCc2cc3cc(Cl)ccc3[nH]2)C1] | 3-((5-chloro-1H-indol-2- yl)methyl)-1-((3R)-1- ((ethylcarbamoyl)alanyl)pi- peridin-3-yl)-1- methylurea | 462.21 | 463.2 |
| 110 | 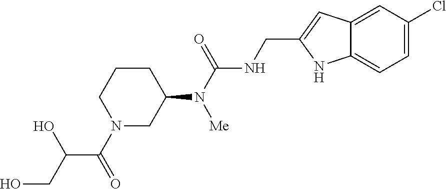 [SMILES: CN(C(=O)NCc1cc2cc(Cl)ccc2[nH]1)[C@@H]1CCCN(C(=O)C(O)CO)C1] | 3-((5-chloro-1H-indol-2- yl)methyl)-1-((3R)-1-(2,3- dihydroxypropanoyl)piper- idin-3-yl)-1-methylurea | 408.16 | 409.0 |
| 111 | 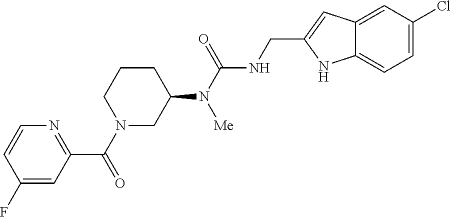 [SMILES: CN(C(=O)NCc1cc2cc(Cl)ccc2[nH]1)[C@@H]1CCCN(C(=O)c2cc(F)ccn2)C1] | (R)-3-((5-chloro-1H- indol-2-yl)methyl)-1-(1- (4- fluoropicolinoyl)piperidin- 3-yl)-1-methylurea | 443.15 | 444.18 |
| 112 | 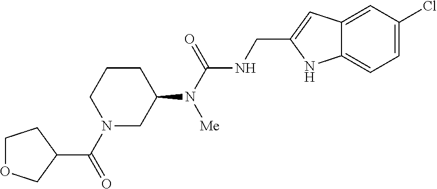 [SMILES: CN(C(=O)NCc1cc2cc(Cl)ccc2[nH]1)[C@@H]1CCCN(C(=O)C2CCOC2)C1] | 3-((5-chloro-1H-indol-2- yl)methyl)-1-methyl-1- ((3R)-1-(tetrahydrofuran- 3-carbonyl)piperidin-3- yl)urea | 418.18 | 419.26 |
| 113 | 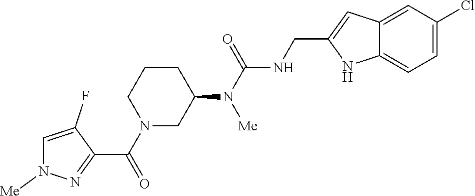 [SMILES: CN(C(=O)NCc1cc2cc(Cl)ccc2[nH]1)[C@@H]1CCCN(C(=O)c2nn(C)cc2F)C1] | (R)-3-((5-chloro-1H- indol-2-yl)methyl)-1-(1- (4-fluoro-1-methyl-1H- pyrazole-3- carbonyl)piperidin-3-yl)- 1-methylurea | 446.16 | 447.19 |
| 114 | 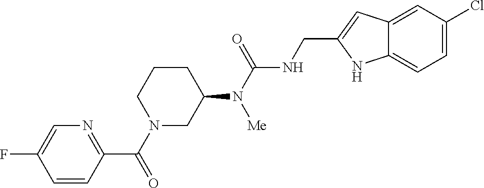 [SMILES: CN(C(=O)NCc1cc2cc(Cl)ccc2[nH]1)[C@@H]1CCCN(C(=O)c2ccc(F)cn2)C1] | (R)-3-((5-chloro-1H- indol-2-yl)methyl)-1-(1- (5- fluoropicolinoyl)piperidin- 3-yl)-1-methylurea | 443.15 | 444.06 |
| 115 | 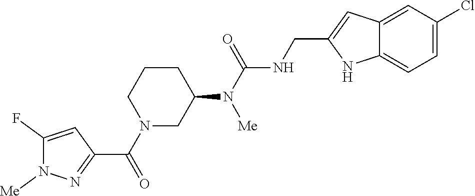 [SMILES: CN(C(=O)NCc1cc2cc(Cl)ccc2[nH]1)[C@@H]1CCCN(C(=O)c2cc(F)n(C)n2)C1] | (R)-3-((5-chloro-1H- indol-2-yl)methyl)-1-(1- (5-fluoro-1-methyl-1H- pyrazole-3- carbonyl)piperidin-3-yl)- 1-methylurea | 446.16 | 447.06 |
| 116 | 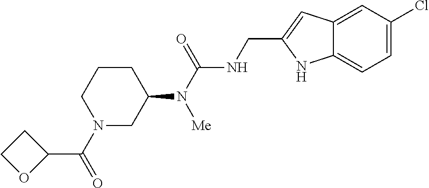 [SMILES: CN(C(=O)NCc1cc2cc(Cl)ccc2[nH]1)[C@@H]1CCCN(C(=O)C2CCO2)C1] | 3-((5-chloro-1H-indol-2- yl)methyl)-1-methyl-1- ((3R)-1-(oxetane-2- carbonyl)piperidin-3- yl)urea | 404.16 | 404.81 |
| 117 | 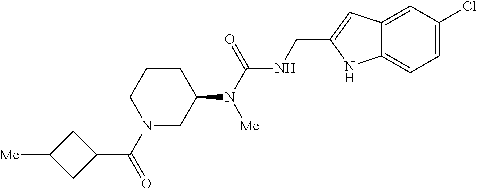 [SMILES: CC1CC(C(=O)N2CCC[C@@H](N(C)C(=O)NCc3cc4cc(Cl)ccc4[nH]3)C2)C1] | (R)-3-((5-chloro-1H- indol-2-yl)methyl)-1- methyl-1-(1-(3- methylcyclobutane-1- carbonyl)piperidin-3- yl)urea | 416.20 | 416.99 |
| 118 | 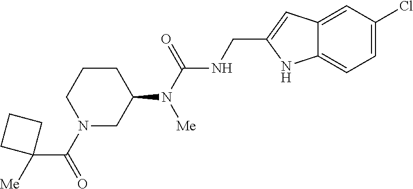 [SMILES: CN(C(=O)NCc1cc2cc(Cl)ccc2[nH]1)[C@@H]1CCCN(C(=O)C2(C)CCC2)C1] | (R)-3-((5-chloro-1H- indol-2-yl)methyl)-1- methyl-1-(1-(1- methylcyclobutane-1- carbonyl)piperidin-3- yl)urea | 416.20 | 416.91 |
| 119 | 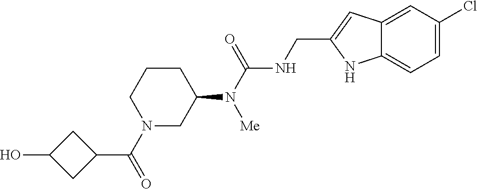 [SMILES: CN(C(=O)NCc1cc2cc(Cl)ccc2[nH]1)[C@@H]1CCCN(C(=O)C2CC(O)C2)C1] | (R)-3-((5-chloro-1H- indol-2-yl)methyl)-1-(1- (3-hydroxycyclobutane-1- carbonyl)piperidin-3-yl)- 1-methylurea | 418.18 | 418.83 |
| 120 | 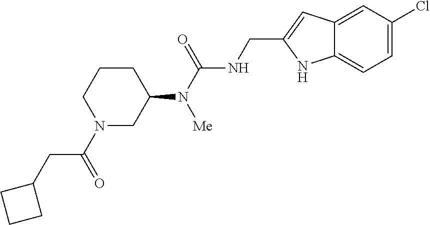 [SMILES: CN(C(=O)NCc1cc2cc(Cl)ccc2[nH]1)[C@@H]1CCCN(C(=O)CC2CCC2)C1] | (R)-3-((5-chloro-1H- indol-2-yl)methyl)-1-(1- (2- cyclobutylacetyl)piperidin- 3-yl)-1-methylurea | 416.20 | 416.97 |
| 121 | 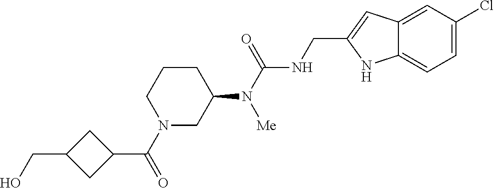 [SMILES: CN(C(=O)NCc1cc2cc(Cl)ccc2[nH]1)[C@@H]1CCCN(C(=O)C2CC(CO)C2)C1] | (R)-3-((5-chloro-1H- indol-2-yl)methyl)-1-(1- (3- (hydroxymethyl)cyclobutane- 1-carbonyl)piperidin-3- yl)-1-methylurea | 432.19 | 433.21 |
| 122 | 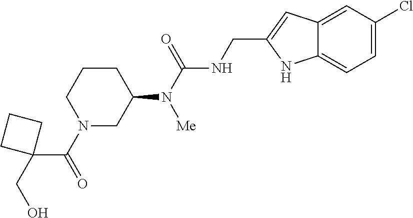 [SMILES: CN(C(=O)NCc1cc2cc(Cl)ccc2[nH]1)[C@@H]1CCCN(C(=O)C2(CO)CCC2)C1] | (R)-3-((5-chloro-1H- indol-2-yl)methyl)-1-(1- (1- (hydroxymethyl)cyclobutane- 1-carbonyl)piperidin-3- yl)-1-methylurea | 432.19 | 433.00 |
| 123 | 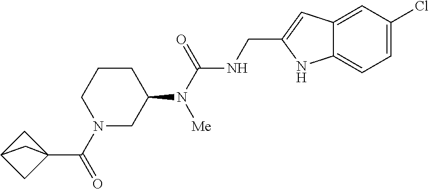 [SMILES: CN(C(=O)NCc1cc2cc(Cl)ccc2[nH]1)[C@@H]1CCCN(C(=O)C23CC(C2)C3)C1] | (R)-1-(1- (bicyclo[1.1.1 ]pentane-1- carbonyl)piperidin-3-yl)- 3-((5-chloro-1H-indol-2- yl)methyl)-1-methylurea | 414.18 | 415.27 |
| 124 | 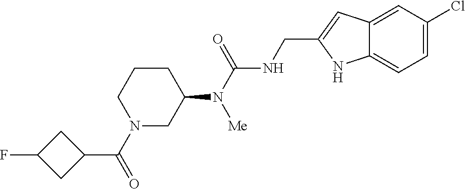 [SMILES: CN(C(=O)NCc1cc2cc(Cl)ccc2[nH]1)[C@@H]1CCCN(C(=O)C2CC(F)C2)C1] | (R)-3-((5-chloro-1H- indol-2-yl)methyl)-1-(1- (3-fluorocyclobutane-1- carbonyl)piperidin-3-yl)- 1-methylurea | 420.17 | 421.29 |
| 125 | 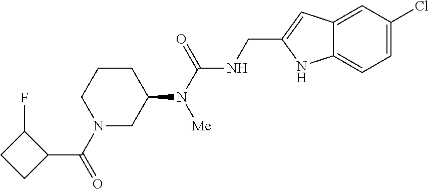 [SMILES: CN(C(=O)NCc1cc2cc(Cl)ccc2[nH]1)[C@@H]1CCCN(C(=O)C2CCC2F)C1] | 3-((5-chloro-1H-indol-2- yl)methyl)-1-((3R)-1-(2- fluorocyclobutane-1- carbonyl)piperidin-3-yl)- 1-methylurea | 420.17 | 420.85 |
| 126 | 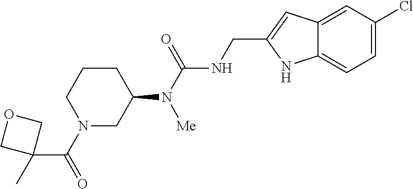 [SMILES: CN(C(=O)NCc1cc2cc(Cl)ccc2[nH]1)[C@@H]1CCCN(C(=O)C2(C)COC2)C1] | (R)-3-((5-chloro-1H- indol-2-yl)methyl)-1- methyl-1-(1-(3- methyloxetane-3- carbonyl)piperidin-3- yl)urea | 418.18 | 418.87 |
| 127 | 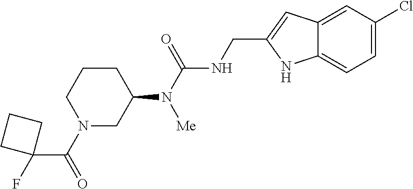 [SMILES: CN(C(=O)NCc1cc2cc(Cl)ccc2[nH]1)[C@@H]1CCCN(C(=O)C2(F)CCC2)C1] | (R)-3-((5-chloro-1H- indol-2-yl)methyl)-1-(1- (1-fluorocyclobutane-1- carbonyl)piperidin-3-yl)- 1-methylurea | 420.17 | 420.91 |
| 128 | 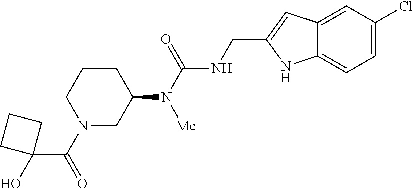 [SMILES: CN(C(=O)NCc1cc2cc(Cl)ccc2[nH]1)[C@@H]1CCCN(C(=O)C2(O)CCC2)C1] | (R)-3-((5-chloro-1H- indol-2-yl)methyl)-1-(1- (1-hydroxycyclobutane-1- carbonyl)piperidin-3-yl)- 1-methylurea | 418.18 | 418.88 |
| 129 | 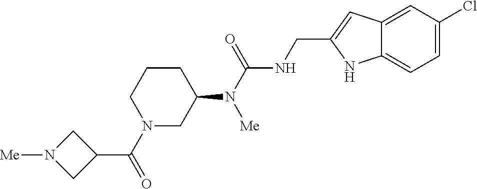 [SMILES: CN1CC(C(=O)N2CCC[C@@H](N(C)C(=O)NCc3cc4cc(Cl)ccc4[nH]3)C2)C1] | (R)-3-((5-chloro-1H- indol-2-yl)methyl)-1- methyl-1-(1-(1- methylazetidine-3- carbonyl)piperidin-3- yl)urea | 417.19 | 418.11 |
| 180 | 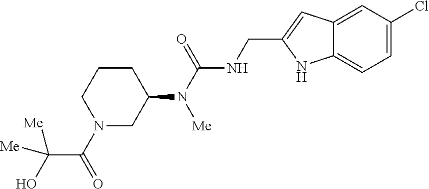 [SMILES: CN(C(=O)NCc1cc2cc(Cl)ccc2[nH]1)[C@@H]1CCCN(C(=O)C(C)(C)O)C1] | (R)-3-((5-chloro-1H- indol-2-yl)methyl)-1-(1- (2-hydroxy-2- methylpropanoyl)piperidin- 3-yl)-1-methylurea | 406.18 | 406.8 |
| 183 | 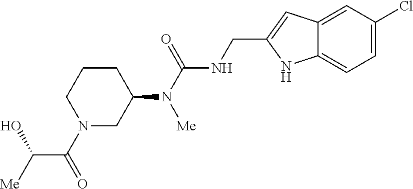 [SMILES: C[C@H](O)C(=O)N1CCC[C@@H](N(C)C(=O)NCc2cc3cc(Cl)ccc3[nH]2)C1] | 3-((5-chloro-1H-indol-2- yl)methyl)-1-((R)-1-((S)- 2- hydroxypropanoyl)piperidin- 3-yl)-1-methylurea | 392.16 | 393.2 |
| 185 | 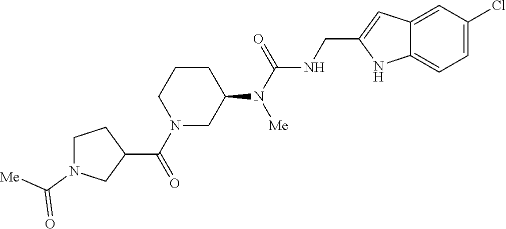 [SMILES: CC(=O)N1CCC(C(=O)N2CCC[C@@H](N(C)C(=O)NCc3cc4cc(Cl)ccc4[nH]3)C2)C1] | 1-((3R)-1-(1- acetylpyrrolidine-3- carbonyl)piperidin-3-yl)- 3-((5-chloro-1H-indol-2- yl)methyl)-1-methylurea | 459.20 | 459.9 |
| 186 | 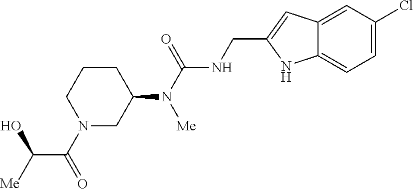 [SMILES: C[C@@H](O)C(=O)N1CCC[C@@H](N(C)C(=O)NCc2cc3cc(Cl)ccc3[nH]2)C1] | 3-((5-chloro-1H-indol-2- yl)methyl)-1-((R)-1-((R)- 2- hydroxypropanoyl)piperidin- 3-yl)-1-methylurea | 392.16 | 393.2 |
| 187 | 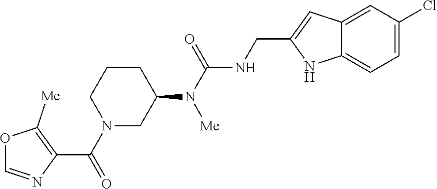 [SMILES: Cc1ocnc1C(=O)N1CCC[C@@H](N(C)C(=O)NCc2cc3cc(Cl)ccc3[nH]2)C1] | (R)-3-((5-chloro-1H- indol-2-yl)methyl)-1- methyl-1-(1-(5- methyloxazole-4- carbonyl)piperidin-3- yl)urea | 429.16 | 430.1 |
| 188 | 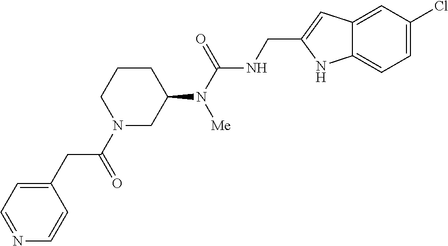 [SMILES: CN(C(=O)NCc1cc2cc(Cl)ccc2[nH]1)[C@@H]1CCCN(C(=O)Cc2ccncc2)C1] | (R)-3-((5-chloro-1H- indol-2-yl)methyl)-1- methyl-1-(1-(2-(pyridin-4- yl)acetyl)piperidin-3- yl)urea | 439.18 | 440.0 |
| 189 | 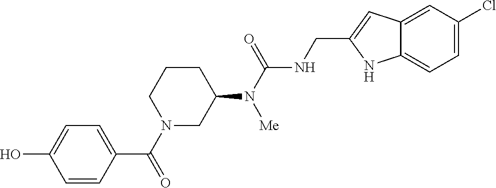 [SMILES: CN(C(=O)NCc1cc2cc(Cl)ccc2[nH]1)[C@@H]1CCCN(C(=O)c2ccc(O)cc2)C1] | (R)-3-((5-chloro-1H- indol-2-yl)methyl)-1-(1- (4- hydroxybenzoyl)piperidin- 3-yl)-1-methylurea | 440.16 | 441.2 |
| 190 | 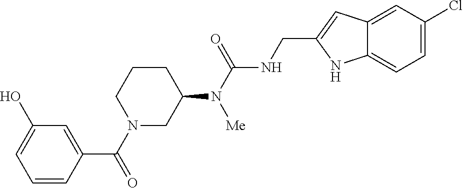 [SMILES: CN(C(=O)NCc1cc2cc(Cl)ccc2[nH]1)[C@@H]1CCCN(C(=O)c2cccc(O)c2)C1] | (R)-3-((5-chloro-1H- indol-2-yl)methyl)-1-(1- (3- hydroxybenzoyl)piperidin- 3-yl)-1-methylurea | 440.16 | 441.0 |
| 191 | 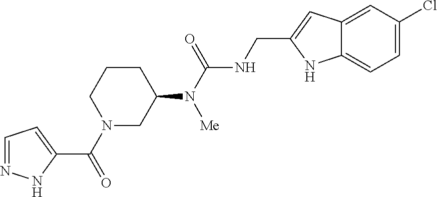 [SMILES: CN(C(=O)NCc1cc2cc(Cl)ccc2[nH]1)[C@@H]1CCCN(C(=O)c2ccn[nH]2)C1] | (R)-1-(1-(1H-pyrazole-5- carbonyl)piperidin-3-yl)- 3-((5-chloro-1H-indol-2- yl)methyl)-1-methylurea | 414.16 | 415.2 |
| 194 | 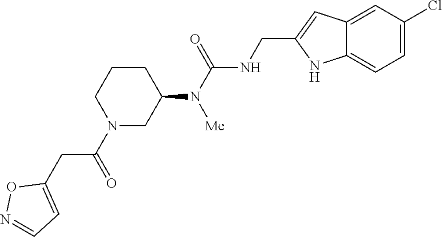 [SMILES: CN(C(=O)NCc1cc2cc(Cl)ccc2[nH]1)[C@@H]1CCCN(C(=O)Cc2ccno2)C1] | (R)-3-((5-chloro-1H- indol-2-yl)methyl)-1-(1- (2-(isoxazol-5- yl)acetyl)piperidin-3-yl)- 1-methylurea | 429.16 | 430.1 |
| 196 | 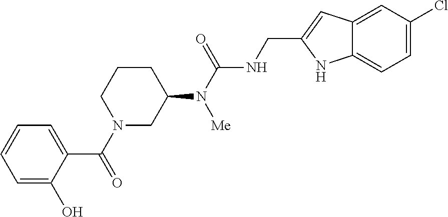 [SMILES: CN(C(=O)NCc1cc2cc(Cl)ccc2[nH]1)[C@@H]1CCCN(C(=O)c2ccccc2O)C1] | (R)-3-((5-chloro-1H- indol-2-yl)methyl)-1-(1- (2- hydroxybenzoyl)piperidin- 3-yl)-1-methylurea | 440.16 | 441.2 |
| 197 | 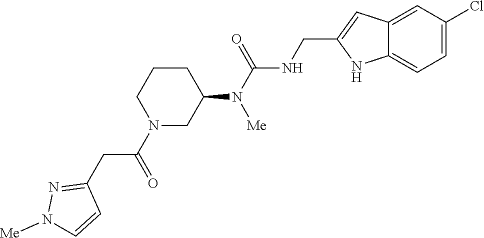 [SMILES: CN(C(=O)NCc1cc2cc(Cl)ccc2[nH]1)[C@@H]1CCCN(C(=O)Cc2ccn(C)n2)C1] | (R)-3-((5-chloro-1H- indol-2-yl)methyl)-1- methyl-1-(1-(2-(1-methyl- 1H-pyrazol-3- yl)acetyl)piperidin-3- yl)urea | 442.19 | 443.1 |
| 198 | 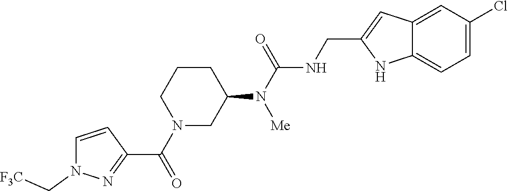 [SMILES: CN(C(=O)NCc1cc2cc(Cl)ccc2[nH]1)[C@@H]1CCCN(C(=O)c2ccn(CC(F)(F)F)n2)C1] | (R)-3-((5-chloro-1H- indol-2-yl)methyl)-1- methyl-1-(1-(1-(2,2,2- trifluoroethyl)-1H- pyrazole-3- carbonyl)piperidin-3- yl)urea | 496.16 | 497.1 |
| 200 | 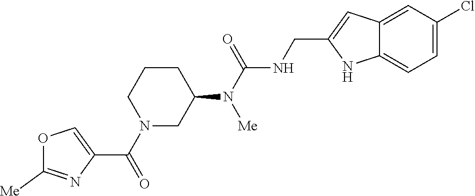 [SMILES: Cc1nc(C(=O)N2CCC[C@@H](N(C)C(=O)NCc3cc4cc(Cl)ccc4[nH]3)C2)co1] | (R)-3-((5-chloro-1H- indol-2-yl)methyl)-1- methyl-1-(1-(2- methyloxazole-4- carbonyl)piperidin-3- yl)urea | 429.16 | 430.0 |
| 201 | 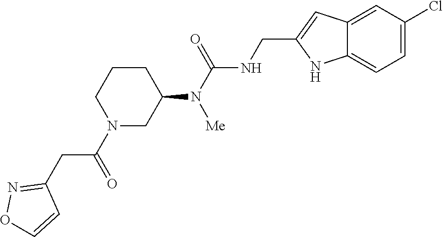 [SMILES: CN(C(=O)NCc1cc2cc(Cl)ccc2[nH]1)[C@@H]1CCCN(C(=O)Cc2ccon2)C1] | (R)-3-((5-chloro-1H- indol-2-yl)methyl)-1-(1- (2-(isoxazol-3- yl)acetyl)piperidin-3-yl)- 1-methylurea | 429.16 | 430.1 |
| 202 | 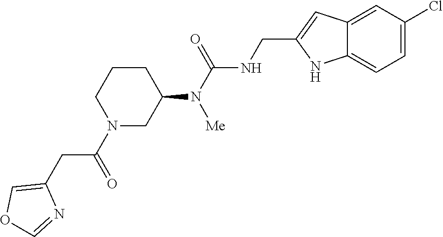 [SMILES: CN(C(=O)NCc1cc2cc(Cl)ccc2[nH]1)[C@@H]1CCCN(C(=O)Cc2cocn2)C1] | (R)-3-((5-chloro-1H- indol-2-yl)methyl)-1- methyl-1-(1-(2-(oxazol-4- yl)acetyl)piperidin-3- yl)urea | 429.16 | 430.1 |
| 204 | 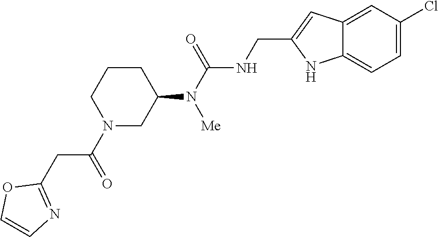 [SMILES: CN(C(=O)NCc1cc2cc(Cl)ccc2[nH]1)[C@@H]1CCCN(C(=O)Cc2ncco2)C1] | (R)-3-((5-chloro-1H- indol-2-yl)methyl)-1- methyl-1-(1-(2-(oxazol-2- yl)acetyl)piperidin-3- yl)urea | 429.16 | 430.2 |
| 205 | 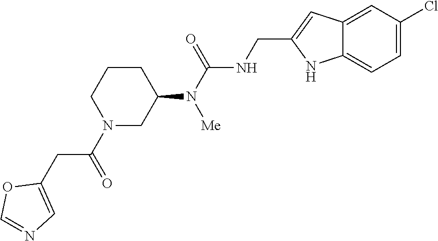 [SMILES: CN(C(=O)NCc1cc2cc(Cl)ccc2[nH]1)[C@@H]1CCCN(C(=O)Cc2cnco2)C1] | (R)-3-((5-chloro-1H- indol-2-yl)methyl)-1- methyl-1-(1-(2-(oxazol-5- yl)acetyl)piperidin-3- yl)urea | 429.16 | 430.1 |
| 215 | 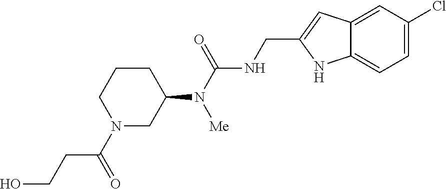 [SMILES: CN(C(=O)NCc1cc2cc(Cl)ccc2[nH]1)[C@@H]1CCCN(C(=O)CCO)C1] | (R)-3-((5-chloro-1H- indol-2-yl)methyl)-1-(1- (3- hydroxypropanoyl)piperidin- 3-yl)-1-methylurea | 392.16 | 393.2 |
| 232 | 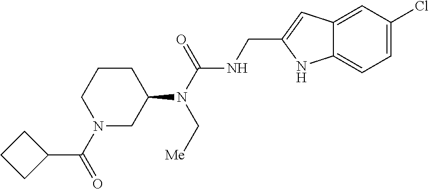 [SMILES: CCN(C(=O)NCc1cc2cc(Cl)ccc2[nH]1)[C@@H]1CCCN(C(=O)C2CCC2)C1] | (R)-3-((5-chloro-1H- indol-2-yl)methyl)-1-(1- (cyclobutanecarbonyl)piper- idin-3-yl)-1-ethylurea | 416.20 | 417.2 |
| 236 | 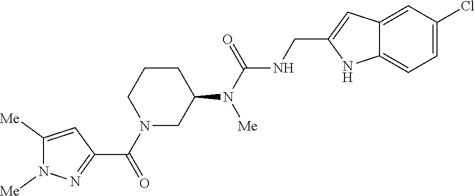 [SMILES: Cc1cc(C(=O)N2CCC[C@@H](N(C)C(=O)NCc3cc4cc(Cl)ccc4[nH]3)C2)nn1C] | (R)-3-((5-chloro-1H- indol-2-yl)methyl)-1-(1- (1,5-dimethyl-1H- pyrazole-3- carbonyl)piperidin-3-yl)- 1-methylurea | 442.19 | 443.0 |
| 237 | 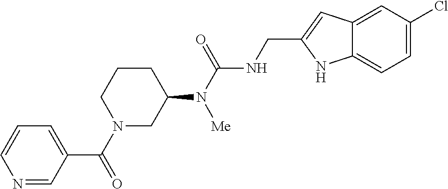 [SMILES: CN(C(=O)NCc1cc2cc(Cl)ccc2[nH]1)[C@@H]1CCCN(C(=O)c2cccnc2)C1] | (R)-3-((5-chloro-1H- indol-2-yl)methyl)-1- methyl-1-(1- nicotinoylpiperidin-3- yl)urea | 425.16 | 426.0 |
| 238 | 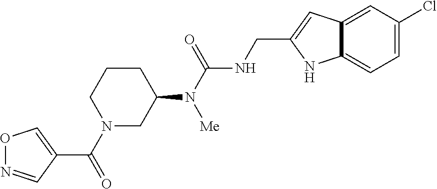 | (R)-3-((5-chloro-1H- indol-2-yl)methyl)-1-(1- (isoxazole-4- carbonyl)piperidin-3-yl)- 1-methylurea | 415.14 | 416.0 |
| 239 | 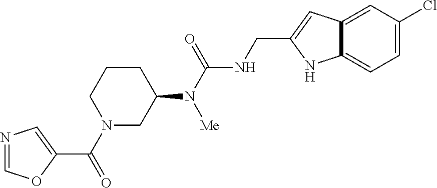 | (R)-3-((5-chloro-1H- indol-2-yl)methyl)-1- methyl-1-(1-(oxazole-5- carbonyl)piperidin-3- yl)urea | 415.14 | 416.0 |
| 240 | 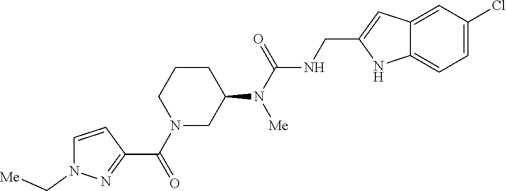 [SMILES: CCn1ccc(C(=O)N2CCC[C@@H](N(C)C(=O)NCc3cc4cc(Cl)ccc4[nH]3)C2)n1] | (R)-3-((5-chloro-1H- indol-2-yl)methyl)-1-(1- (1-ethyl-1H-pyrazole-3- carbonyl)piperidin-3-yl)- 1-methylurea | 442.19 | 443.1 |
| 241 | 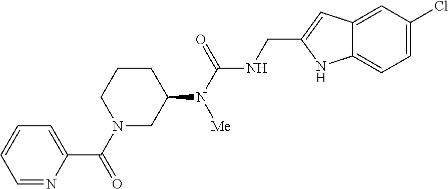 [SMILES: CN(C(=O)NCc1cc2cc(Cl)ccc2[nH]1)[C@@H]1CCCN(C(=O)c2ccccn2)C1] | (R)-3-((5-chloro-1H- indol-2-yl)methyl)-1- methyl-1-(1- picolinoylpiperidin-3- yl)urea | 425.16 | 426.0 |
| 242 | 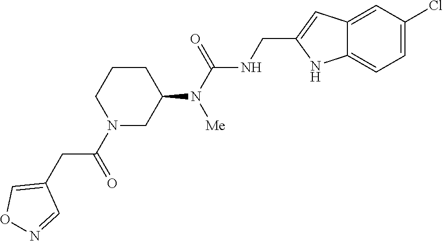 [SMILES: CN(C(=O)NCc1cc2cc(Cl)ccc2[nH]1)[C@@H]1CCCN(C(=O)Cc2cnoc2)C1] | (R)-3-((5-chloro-1H- indol-2-yl)methyl)-1-(1- (2-(isoxazol-4- yl)acetyl)piperidin-3-yl)- 1-methylurea | 429.16 | 430.3 |

### Example 2: (R)-1-(1-(azetidine-3-carbonyl)piperidin-3-yl)-3-((5-chloro-1H-indol-2-yl)methyl)-1-methylurea (Compound 130)

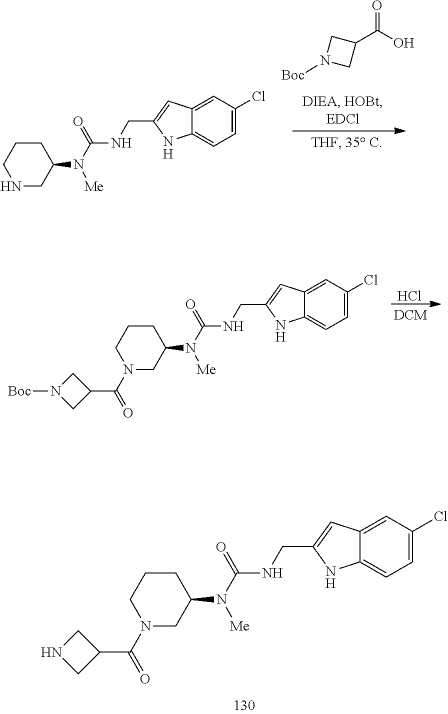 [SMILES: CC(C)(C)OC(=O)N1CC(C(=O)O)C1 ; CN(C(=O)NCc1cc2cc(Cl)ccc2[nH]1)[C@@H]1CCCNC1 ; CN(C(=O)NCc1cc2cc(Cl)ccc2[nH]1)[C@@H]1CCCN(C(=O)C2CN(C(=O)OC(C)(C)C)C2)C1 ; Cl ; CN(C(=O)NCc1cc2cc(Cl)ccc2[nH]1)[C@@H]1CCCN(C(=O)C2CNC2)C1]

Step 1. To a stirred solution of 1-(tert-butoxycarbonyl)azetidine-3-carboxylic acid (0.108 g, 0.535 mmol, 1.2 eq) in THE (2.2 mL, 0.2 M) were added HOBt (0.105 g, 86 w/w⁰%, 0.669 mmol, 1.5 eq) and EDC hydrochloride (0.128 g, 0.669 mmol, 1.5 eq) followed by DIEA (0.233 mL, 1.34 mmol, 3 eq). The resulting mixture was stirred for 5 mins then (R)-3-((5-chloro-1H-indol-2-yl)methyl)-1-methyl-1-(piperidin-3-yl)urea (0.143 g, 0.446 mmol, 1 eq) was added and the reaction was heated to 35° C. After 16 h the reaction was cooled to room temperature and concentrated under reduced pressure. The resulting residue was purified by column chromatography to give tert-butyl (R)-3-(3-(3-((5-chloro-1H-indol-2-yl)methyl)-1-methylureido)piperidine-1-carbonyl)azetidine-1-carboxylate. LC-MS (ESI) m/z: [M+H]⁺ calcd for C₂₅H₃₄ClN₅O₄: 503.24; found 403.8 [M−Boc+H]⁺.

Step 2. To a solution of tert-butyl (R)-3-(3-(3-((5-chloro-1H-indol-2-yl)methyl)-1-methylureido)piperidine-1-carbonyl)azetidine-1-carboxylate (0.155 g, 0.308 mmol, 1 eq) in DCM (1.02 mL, 0.3M) was added hydrogen chloride solution (1.54 mL, 4 M in dioxane, 6.15 mmol, 20 eq) and the resulting mixture was heated to 30° C. After 2 h the reaction was cooled to room temperature and concentrated under reduced pressure. The resulting residue was purified by column chromatography to give (R)-1-(1-(azetidine-3-carbonyl)piperidin-3-yl)-3-((5-chloro-1H-indol-2-yl)methyl)-1-methylurea. LC-MS (ESI) m/z: [M+H]⁺ calcd for C₂₀H₂₆ClN₅O₂: 404.19; found 403.9. ¹H NMR (500 MHz, MeOD) δ 8.54 (s, 1H), 7.42 (t, J=2.4 Hz, 1H), 7.27 (dd, J=8.6, 2.2 Hz, 1H), 7.04-6.97 (m, 1H), 6.25 (s, 1H), 4.53-4.40 (m, 3H), 4.29-4.11 (m, 4H), 4.09-3.97 (m, 2H), 3.56-3.40 (m, 1H), 3.12-2.92 (m, 1H), 2.86 (d, J=3.9 Hz, 3H), 2.80-2.53 (m, 1H), 1.90-1.76 (m, 3H), 1.59-1.46 (m, 1H).

The following compounds in Table T-2 were synthesized using procedures similar to Compound 130 using the appropriate starting materials.

TABLE T-2

| | | | Exact | LCMS, |
|---|---|---|---|---|
| Compound | | | mass | found |
| No. | Structure | IUPAC name | (g/mol) | [M + H]⁺ |
| 131 | 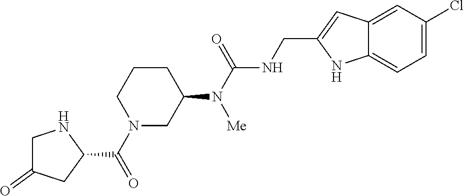 [SMILES: CN(C(=O)NCc1cc2cc(Cl)ccc2[nH]1)[C@@H]1CCCN(C(=O)[C@@H]2CC(=O)CN2)C1] | 3-((5-chloro-1H- indol-2-yl)methyl)-1- methyl-1-((R)-1-((S)- 4-oxopyrrolidine-2- carbonyl)piperidin-3- yl)urea | 431.17 | 432.2 |
| 132 | 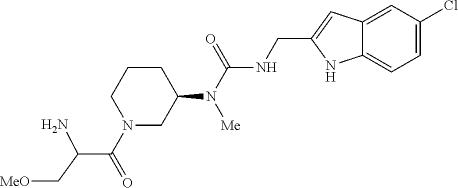 [SMILES: COCC(N)C(=O)N1CCC[C@@H](N(C)C(=O)NCc2cc3cc(Cl)ccc3[nH]2)C1] | 3-((5-chloro-1H- indol-2-yl)methyl)-1- methyl-1-((3R)-1-(O- methylseryl)piperidin- 3-yl)urea | 421.19 | 422.2 |
| 133 | 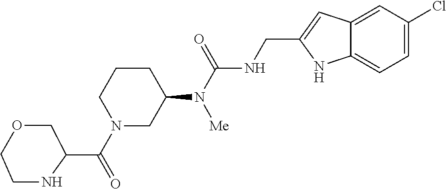 [SMILES: CN(C(=O)NCc1cc2cc(Cl)ccc2[nH]1)[C@@H]1CCCN(C(=O)C2COCCN2)C1] | 3-((5-chloro-1H- indol-2-yl)methyl)-1- methyl-1-((3R)-1- (morpholine-3- carbonyl)piperidin-3- yl)urea | 433.19 | 434.2 |
| 134 | 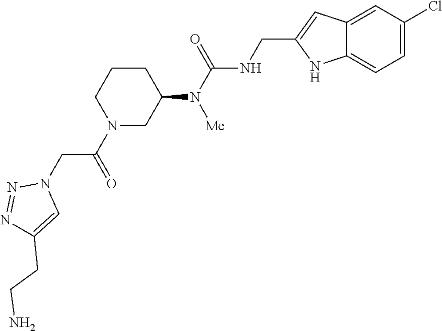 [SMILES: CN(C(=O)NCc1cc2cc(Cl)ccc2[nH]1)[C@@H]1CCCN(C(=O)Cn2cc(CCN)nn2)C1] | (R)-1-(1-(2-(4-(2- aminoethyl)-1H-1,2,3- triazol-1- yl)acetyl)piperidin-3- yl)-3-((5-chloro-1H- indol-2-yl)methyl)-1- methylurea | 472.21 | 473.24 |
| 199 | 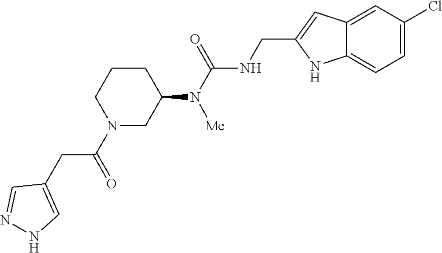 [SMILES: CN(C(=O)NCc1cc2cc(Cl)ccc2[nH]1)[C@@H]1CCCN(C(=O)Cc2cn[nH]c2)C1] | (R)-1-(1-(2-(1H- pyrazol-4- yl)acetyl)piperidin-3- yl)-3-((5-chloro-1H- indol-2-yl)methyl)-1- methylurea | 428.17 | 429.2 |

### Example 3: (R)-1-(1-acetylpiperidin-3-yl)-3-((5-chloro-6-((5-methylisoxazol-3-yl)methoxy)-1H-indol-2-yl)methyl)-1-methylurea (Compound 135)

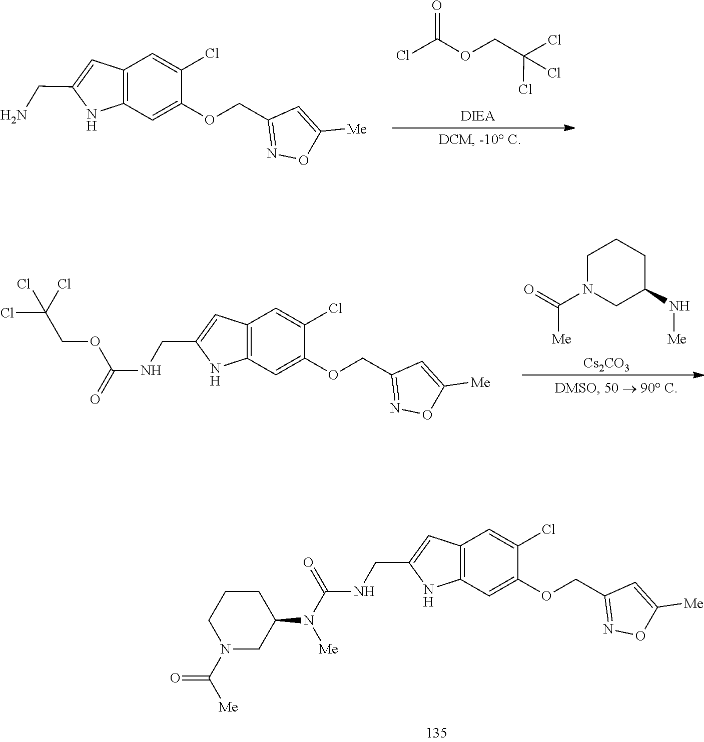

Step 1: To a solution of (5-chloro-6-((5-methylisoxazol-3-yl)methoxy)-1H-indol-2-yl)methanamine (1.30 g, 3.96 mmol, 1.0 eq) in DCM (13 mL) was added DIEA (11.8 mmol, 2.10 mL, 3.0 eq). The mixture cooled to −10° C., then a solution of 2,2,2-trichloroethyl chloroformate (530 mL, 3.96 mmol, 1.00 eq) in DCM (2 mL) was added. The mixture was stirred at −10° C. for 1 h, then was filtered. The filtrate was extracted with EtOAc (2×50 mL). The organic phase was then washed with brine (50 mL), dried over Na₂SO₄, and concentrated in vacuo to give 2,2,2-trichloroethyl ((5-chloro-6-((5-methylisoxazol-3-yl)methoxy)-1H-indol-2-yl)methyl)carbamate, which was used without further purification. LC-MS (ESI) m/z: [M+H]⁺ calcd for C₁₇H₁₅Cl₄N₃O₄: 464.98; found 465.9.

Step 2: To a solution of (R)-1-(3-(methylamino)piperidin-1-yl)ethan-1-one (618 mg, 3.96 mmol, 1.0 eq) in DMSO (10 mL) was added Cs₂CO₃ (1.55 g, 4.75 mmol, 1.2 eq). The resulting mixture was heated to 50° C. and stirred for 0.5 h. A solution of 2,2,2-trichloroethyl ((5-chloro-6-((5-methylisoxazol-3-yl)methoxy)-1H-indol-2-yl)methyl)carbamate (1.85 g, 3.96 mmol, 1.00 eq) in DMSO (5 mL) was added and the mixture was stirred at 90° C. for 15.5 h. The reaction solution was cooled to room temperature, filtered and the filtrate was extracted with EtOAc (2×10 mL). The organic layer was washed with brine (10 mL), dried over Na₂SO₄, and concentrated in vacuo. The residue was purified by column chromatography to give (R)-1-(1-acetylpiperidin-3-yl)-3-((5-chloro-6-((5-methylisoxazol-3-yl)methoxy)-1H-indol-2-yl)methyl)-1-methylurea.

LC-MS (ESI) m/z: [M+H]⁺ calcd for C₂₃H₂₈ClN₅O₄: 473.18; found 474.1. ¹H NMR (400 MHz, DMSO-d₆) δ 10.80 (s, 1H), 7.48 (s, 1H), 7.17 (d, J=4.4 Hz, 1H), 6.91-6.79 (m, 1H), 6.32 (s, 1H), 6.13 (s, 1H), 5.18 (s, 2H), 4.35-4.26 (m, 3H), 3.91-3.90 (m, 2H), 3.05-2.88 (m, 1H), 2.76-2.73 (m, 3H), 2.55-2.41 (m, 4H), 1.98 (d, J=10.4 Hz, 3H), 1.70-1.67 (m, 3H), 1.65-1.32 (in, 1H).

The following compounds in Table T-2.1 were synthesized using procedures similar to Compound 135 using the appropriate starting materials.

TABLE T-2.1

| | | | Exact | LCMS, |
|---|---|---|---|---|
| Compound | | | mass | found |
| No. | Structure | IUPAC name | (g/mol) | [M + H]⁺ |
| 192 | 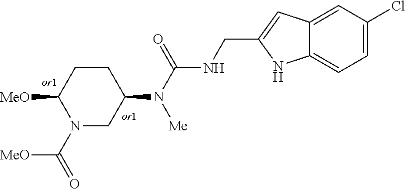 [SMILES: COC(=O)N1C[C@H](N(C)C(=O)NCc2cc3cc(Cl)ccc3[nH]2)CC[C@@H]1OC] | 1-((3R,6S or 3S,6R)- 1-acetyl-6- methylpiperidin-3- yl)-3-((5-chloro-1H- indol-2-yl)methyl)- 1-methylurea (SFC column: Chiralpak IH-3, second eluting isomer) | 376.17 | 377.2 |
| 193 | 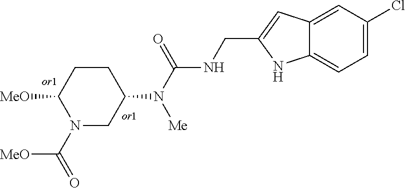 [SMILES: COC(=O)N1C[C@@H](N(C)C(=O)NCc2cc3cc(Cl)ccc3[nH]2)CC[C@H]1OC] | 1-((3S,6R or 3R,6S)- 1-acetyl-6- methylpiperidin-3- yl)-3-((5-chloro-1H- indol-2-yl)methyl)- 1-methylurea (SFC column: Chiralpak IH-3, second eluting isomer) | 376.17 | 377.2 |
| 203 | 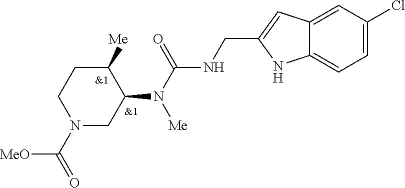 [SMILES: COC(=O)N1CC[C@@H](C)[C@@H](N(C)C(=O)NCc2cc3cc(Cl)ccc3[nH]2)C1] | 1-((3R,4R and 3S,4S)-1-acetyl-4- methylpiperidin-3- yl)-3-((5-chloro-1H- indol-2-yl)methyl)- 1-methylurea | 376.17 | 377.1 |
| 206 | 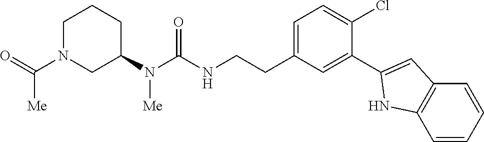 [SMILES: CC(=O)N1CCC[C@@H](N(C)C(=O)NCCc2ccc(Cl)c(-c3cc4ccccc4[nH]3)c2)C1] | (R)-1-(1- acetylpiperidin-3- yl)-3-(4-chloro-3- (1H-indol-2- yl)phenethyl)-1- methylurea | 452.20 | 453.4 |
| 207 | 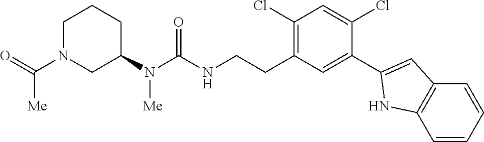 [SMILES: CC(=O)N1CCC[C@@H](N(C)C(=O)NCCc2cc(-c3cc4ccccc4[nH]3)c(Cl)cc2Cl)C1] | (R)-1-(1- acetylpiperidin-3- yl)-3-(2,4-dichloro- 5-(1H-indol-2- yl)phenethyl)-1- methylurea | 486.16 | 487.3 |
| 234 | 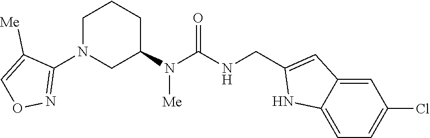 [SMILES: Cc1conc1N1CCC[C@@H](N(C)C(=O)NCc2cc3cc(Cl)ccc3[nH]2)C1] | (R)-3-((5-chloro-1H- indol-2-yl)methyl)- 1-methyl-1-(1-(4- methylisoxazol-3- yl)piperidin-3- yl)urea | 401.16 | 402.2 |
| 242 | 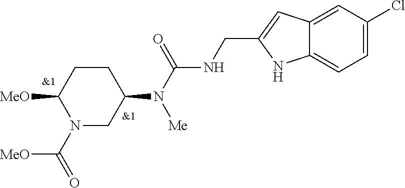 [SMILES: COC(=O)N1C[C@H](N(C)C(=O)NCc2cc3cc(Cl)ccc3[nH]2)CC[C@@H]1OC] | 1-((3R,6S and 3S,6R)-1-acetyl-6- methylpiperidin-3- yl)-3-((5-chloro-1H- indol-2-yl)methyl)- 1-methylurea | 376.17 | 377.3 |
| 243 | 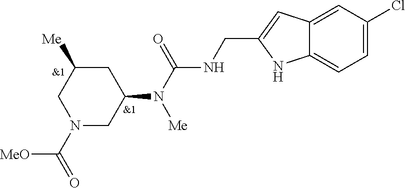 [SMILES: COC(=O)N1C[C@@H](C)C[C@@H](N(C)C(=O)NCc2cc3cc(Cl)ccc3[nH]2)C1] | 1-((3R,5S and 3S,5R)-1-acetyl-5- methylpiperidin-3- yl)-3-((5-chloro-1H- indol-2-yl)methyl)- 1-methylurea | 376.17 | 377.1 |
| 244 | 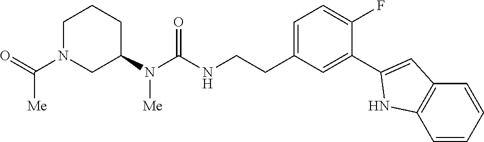 [SMILES: CC(=O)N1CCC[C@@H](N(C)C(=O)NCCc2ccc(F)c(-c3cc4ccccc4[nH]3)c2)C1] | (R)-1-(1- acetylpiperidin-3- yl)-3-(4-fluoro-3- (1H-indol-2- yl)phenethyl)-1- methylurea | 436.23 | 437.3 |
| 245 | 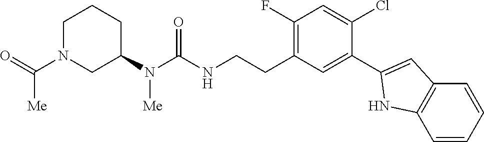 [SMILES: CC(=O)N1CCC[C@@H](N(C)C(=O)NCCc2cc(-c3cc4ccccc4[nH]3)c(Cl)cc2F)C1] | (R)-1-(1- acetylpiperidin-3- yl)-3-(4-chloro-2- fluoro-5-(1H-indol- 2-yl)phenethyl)-1- methylurea | 470.19 | 471.3 |

### Example 4: (R)-1-(1-acetylpiperidin-3-yl)-1-methyl-3-((6-(thiazol-5-ylmethoxy)-1H-indol-2-yl)methyl)urea (Compound 136)

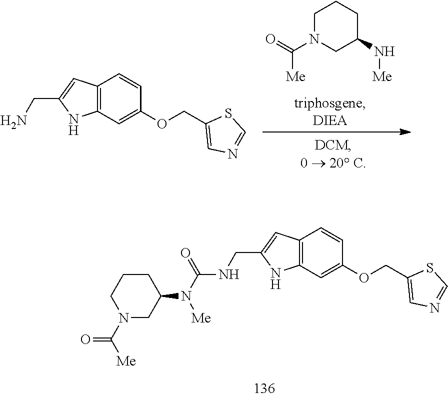 [SMILES: NCc1cc2ccc(OCc3cncs3)cc2[nH]1 ; CN[C@@H]1CCCN(C(C)=O)C1 ; CC(=O)N1CCC[C@@H](N(C)C(=O)NCc2cc3ccc(OCc4cncs4)cc3[nH]2)C1]

To a mixture of (6-(thiazol-5-ylmethoxy)-1H-indol-2-yl)methanamine, HCl salt (150 mg, 578 μmol, 1.00 eq) and (R)-1-(3-(methylamino)piperidin-1-yl)ethan-1-one (90.3 mg, 578 μmol, 1.00 eq) in DCM (2 mL) at 0° C. were added DIEA (255 mL, 1.45 mmol, 2.50 eq) and triphosgene (86.0 mg, 289 μmol, 0.50 eq). The mixture was allowed to warm to RT then was stirred for 2 h. The mixture was quenched by addition of H₂O (5 mL) and extracted with DCM (3×5 mL). The combined organic layers were washed with brine (5 mL), then concentrated in vacuo. Purification by column chromatography gave (R)-1-(1-acetylpiperidin-3-yl)-1-methyl-3-((6-(thiazol-5-ylmethoxy)-1H-indol-2-yl)methyl)urea. LC-MS (ESI) m/z: [M+H]⁺ calcd for C₂₂H₂₇N₅₀₃S: 441.18; found 442.2. ¹H NMR (400 MHz, DMSO-d₆) δ 9.80 (d, J=11.6 Hz, 1H), 8.31 (d, J=5.6 Hz, 1H), 7.38 (d, J=8.4 Hz, 1H), 7.01 (s, 1H), 6.76 (d, J=8.4 Hz, 1H), 5.46 (s, 2H), 4.86-4.45 (m, 3H), 4.11-4.02 (m, 1H), 4.02-3.88 (m, 1H), 3.20-3.03 (m, 1H), 2.87 (d, J=7.2 Hz, 3H), 2.76-2.52 (m, 1H), 2.13 (d, J=4.4 Hz, 3H), 1.86-1.84 (m, 3H), 1.62-1.52 (m, 1H).

The following compounds in Table T-3 were synthesized using procedures similar to Compound 136 using the appropriate starting materials.

TABLE T-3

| | | | Exact | LCMS, |
|---|---|---|---|---|
| Compound | | | mass | found |
| No. | Structure | IUPAC name | (g/mol) | [M + H]⁺ |
| 137 | 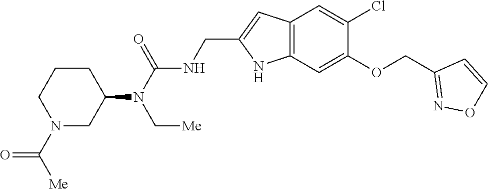 [SMILES: CCN(C(=O)NCc1cc2cc(Cl)c(OCc3ccon3)cc2[nH]1)[C@@H]1CCCN(C(C)=O)C1] | (R)-1-(1- acetylpiperidin-3-yl)- 3-((5-chloro-6- (isoxazol-3- ylmethoxy)-1H-indol- 2-yl)methyl)-1- ethylurea | 473.18 | 474.2 |
| 138 | 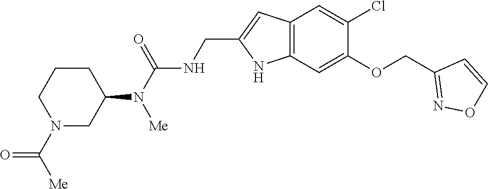 [SMILES: CC(=O)N1CCC[C@@H](N(C)C(=O)NCc2cc3cc(Cl)c(OCc4ccon4)cc3[nH]2)C1] | (R)-1-(1- acetylpiperidin-3-yl)- 3-((5-chloro-6- (isoxazol-3- ylmethoxy)-1H-indol- 2-yl)methyl)-1- methylurea | 459.17 | 460.1 |
| 139 | 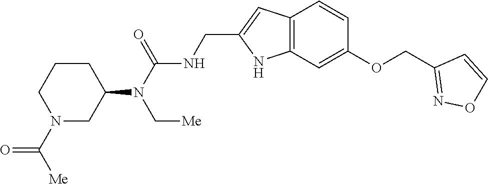 [SMILES: CCN(C(=O)NCc1cc2ccc(OCc3ccon3)cc2[nH]1)[C@@H]1CCCN(C(C)=O)C1] | (R)-1-(1- acetylpiperidin-3-yl)- 1-ethyl-3-((6- (isoxazol-3- ylmethoxy)-1H-indol- 2-yl)methyl)urea | 439.22 | 440.3 |
| 140 | 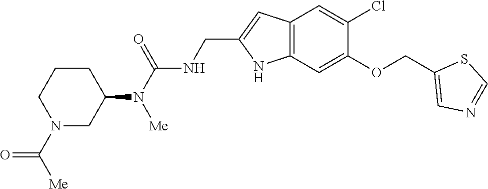 [SMILES: CC(=O)N1CCC[C@@H](N(C)C(=O)NCc2cc3cc(Cl)c(OCc4cncs4)cc3[nH]2)C1] | (R)-1-(1- acetylpiperidin-3-yl)- 3-((5-chloro-6- (thiazol-5-ylmethoxy)- 1H-indol-2- yl)methyl)-1- methylurea | 475.14 | 476.1 |
| 141 | 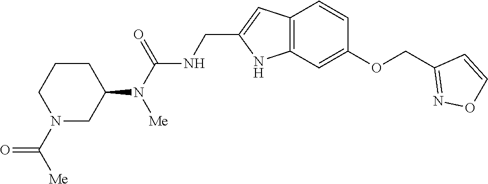 [SMILES: CC(=O)N1CCC[C@@H](N(C)C(=O)NCc2cc3ccc(OCc4ccon4)cc3[nH]2)C1] | (R)-1-(1- acetylpiperidin-3-yl)- 3-((6-(isoxazol-3- ylmethoxy)-1H-indol- 2-yl)methyl)-1- methylurea | 425.21 | 426.2 |
| 142 | 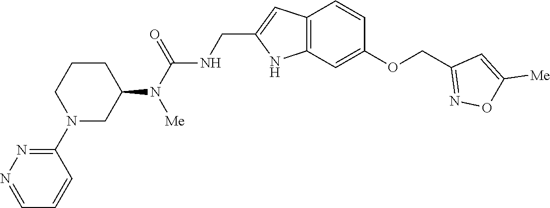 [SMILES: Cc1cc(COc2ccc3cc(CNC(=O)N(C)[C@@H]4CCCN(c5cccnn5)C4)[nH]c3c2)no1] | (R)-1-methyl-3-((6- ((5-methylisoxazol-3- yl)methoxy)-1H-indol- 2-yl)methyl)-1-(1- (pyridazin-3- yl)piperidin-3-yl)urea | 475.23 | 476.3 |
| 143 | 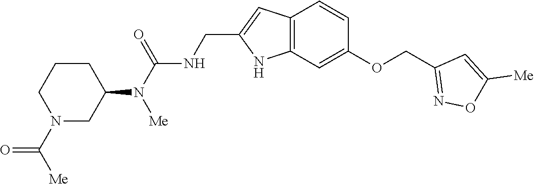 [SMILES: CC(=O)N1CCC[C@@H](N(C)C(=O)NCc2cc3ccc(OCc4cc(C)on4)cc3[nH]2)C1] | (R)-1-(1- acetylpiperidin-3-yl)- 1-methyl-3-((6-((5- methylisoxazol-3- yl)methoxy)-1H-indol- 2-yl)methyl)urea | 439.22 | 440.3 |
| 144 | 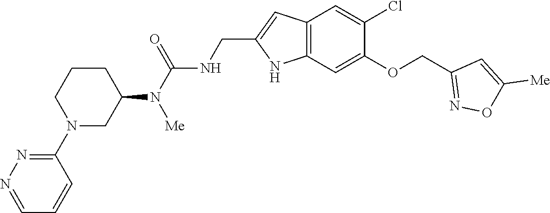 [SMILES: Cc1cc(COc2cc3[nH]c(CNC(=O)N(C)[C@@H]4CCCN(c5cccnn5)C4)cc3cc2Cl)no1] | (R)-3-((5-chloro-6-((5- methylisoxazol-3- yl)methoxy)-1H-indol- 2-yl)methyl)-1- methyl-1-(1- (pyridazin-3- yl)piperidin-3-yl)urea | 509.19 | 510.1 |
| 145 | 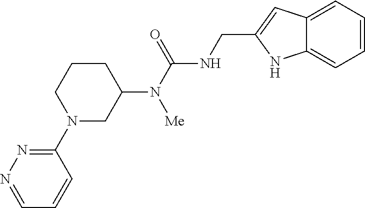 [SMILES: CN(C(=O)NCc1cc2ccccc2[nH]1)C1CCCN(c2cccnn2)C1] | 3-((1H-indol-2- yl)methyl)-1-methyl- 1-(1 -(pyridazin-3- yl)piperidin-3-yl)urea | 364.20 | 365.3 |
| 146 | 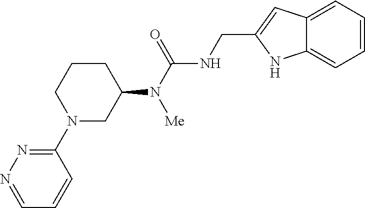 [SMILES: CN(C(=O)NCc1cc2ccccc2[nH]1)[C@@H]1CCCN(c2cccnn2)C1] | (R)-3-((1H-indol-2- yl)methyl)-1-methyl- 1-(1-(pyridazin-3- yl)piperidin-3-yl)urea | 364.20 | 365.3 |
| 150 | 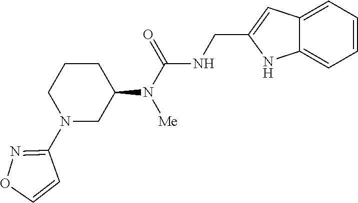 [SMILES: CN(C(=O)NCc1cc2ccccc2[nH]1)[C@@H]1CCCN(c2ccon2)C1] | (R)-3-((1H-indol-2- yl)methyl)-1-(1- (isoxazol-3- yl)piperidin-3-yl)-1- methylurea | 353.18 | 354.2 |
| 151 | 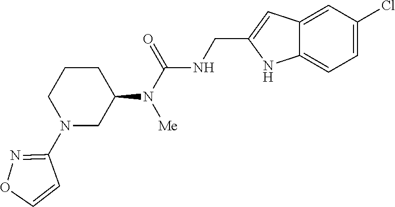 [SMILES: CN(C(=O)NCc1cc2cc(Cl)ccc2[nH]1)[C@@H]1CCCN(c2ccon2)C1] | (R)-3-((5-chloro-1H- indol-2-yl)methyl)-1- (1-(isoxazol-3- yl)piperidin-3-yl)-1- methylurea | 387.15 | 388.2 |
| 152 | 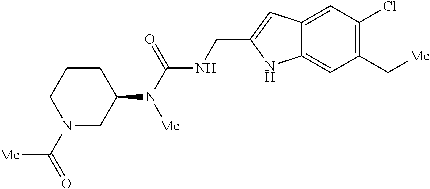 [SMILES: CCc1cc2[nH]c(CNC(=O)N(C)[C@@H]3CCCN(C(C)=O)C3)cc2cc1Cl] | (R)-1-(1- acetylpiperidin-3-yl)- 3-((5-chloro-6-ethyl- 1H-indol-2- yl)methyl)-1- methylurea | 390.18 | 391.1 |
| 153 | 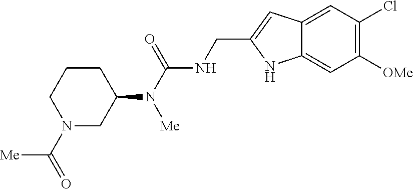 [SMILES: COc1cc2[nH]c(CNC(=O)N(C)[C@@H]3CCCN(C(C)=O)C3)cc2cc1Cl] | (R)-1-(1- acetylpiperidin-3-yl)- 3-((5-chloro-6- methoxy-1H-indol-2- yl)methyl)-1- methylurea | 392.16 | 393.1 |
| 154 | 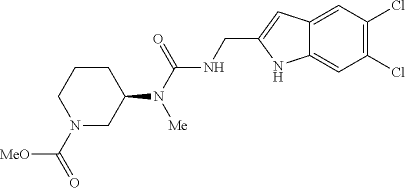 [SMILES: COC(=O)N1CCC[C@@H](N(C)C(=O)NCc2cc3cc(Cl)c(Cl)cc3[nH]2)C1] | (R)-1-(1- acetylpiperidin-3-yl)- 3-((5,6-dichloro-1H- indol-2-yl)methyl)-1- methylurea | 396.11 | 397.0 |
| 155 | 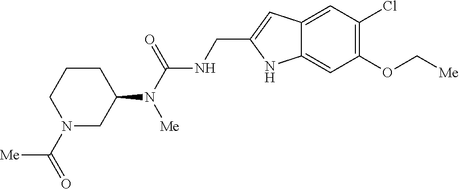 [SMILES: CCOc1cc2[nH]c(CNC(=O)N(C)[C@@H]3CCCN(C(C)=O)C3)cc2cc1Cl] | (R)-1-(1- acetylpiperidin-3-yl)- 3-((5-chloro-6-ethoxy- 1H-indol-2- yl)methyl)-1- methylurea | 406.18 | 407.1 |
| 156 | 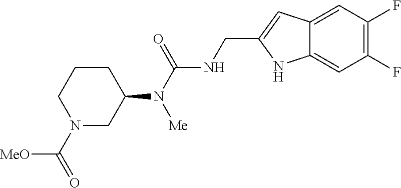 [SMILES: COC(=O)N1CCC[C@@H](N(C)C(=O)NCc2cc3cc(F)c(F)cc3[nH]2)C1] | (R)-1-(1- acetylpiperidin-3-yl)- 3-((5,6-difluoro-1H- indol-2-yl)methyl)-1- methylurea | 364.17 | 365.0 |
| 157 | 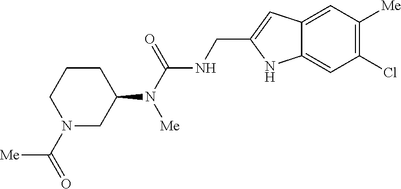 [SMILES: CC(=O)N1CCC[C@@H](N(C)C(=O)NCc2cc3cc(C)c(Cl)cc3[nH]2)C1] | (R)-1-(1- acetylpiperidin-3-yl)- 3-((6-chloro-5-methyl- 1H-indol-2- yl)methyl)-1- methylurea | 376.17 | 377.0 |
| 159 | 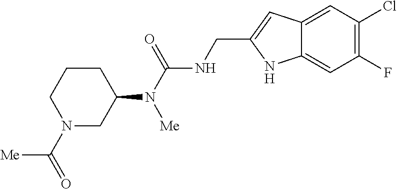 [SMILES: CC(=O)N1CCC[C@@H](N(C)C(=O)NCc2cc3cc(Cl)c(F)cc3[nH]2)C1] | (R)-1-(1- acetylpiperidin-3-yl)- 3-((5-chloro-6-fluoro- 1H-indol-2- yl)methyl)-1- methylurea | 380.14 | 381.1 |
| 160 | 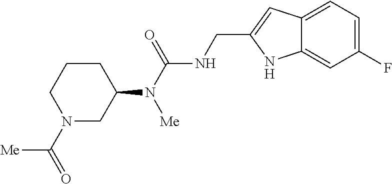 [SMILES: CC(=O)N1CCC[C@@H](N(C)C(=O)NCc2cc3ccc(F)cc3[nH]2)C1] | (R)-1-(1- acetylpiperidin-3-yl)- 3-((6-fluoro-1H-indol- 2-yl)methyl)-1- methylurea | 346.18 | 347.2 |
| 161 | 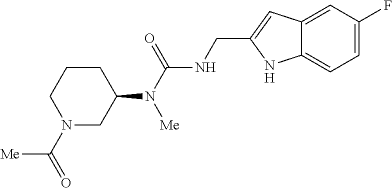 [SMILES: CC(=O)N1CCC[C@@H](N(C)C(=O)NCc2cc3cc(F)ccc3[nH]2)C1] | (R)-1-(1- acetylpiperidin-3-yl)- 3-((5-fluoro-1H-indol- 2-yl)methyl)-1- methylurea | 346.18 | 347.2 |
| 162 | 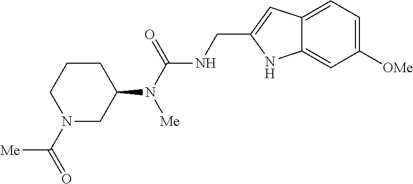 [SMILES: COc1ccc2cc(CNC(=O)N(C)[C@@H]3CCCN(C(C)=O)C3)[nH]c2c1] | (R)-1-(1- acetylpiperidin-3-yl)- 3-((6-methoxy-1H- indol-2-yl)methyl)-1- methylurea | 358.20 | 359.1 |
| 163 | 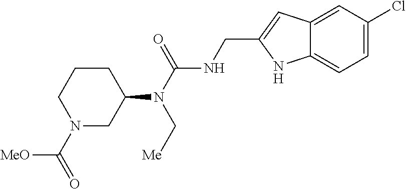 [SMILES: CCN(C(=O)NCc1cc2cc(Cl)ccc2[nH]1)[C@@H]1CCCN(C(=O)OC)C1] | (R)-1-(1- acetylpiperidin-3-yl)- 3-((5-chloro-1H-indol- 2-yl)methyl)-1- ethylurea | 376.17 | 377.1 |
| 164 | 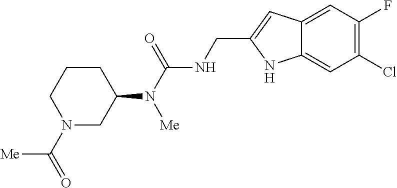 [SMILES: CC(=O)N1CCC[C@@H](N(C)C(=O)NCc2cc3cc(F)c(Cl)cc3[nH]2)C1] | (R)-1-(1- acetylpiperidin-3-yl)- 3-((6-chloro-5-fluoro- 1H-indol-2- yl)methyl)-1- methylurea | 380.14 | 381.2 |
| 165 | 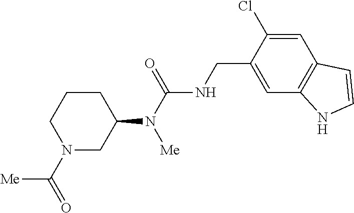 [SMILES: CC(=O)N1CCC[C@@H](N(C)C(=O)NCc2cc3[nH]ccc3cc2Cl)C1] | (R)-1-(1- acetylpiperidin-3-yl)- 3-((5-chloro-1H-indol- 6-yl)methyl)-1- methylurea | 362.15 | 363.0 |
| 166 | 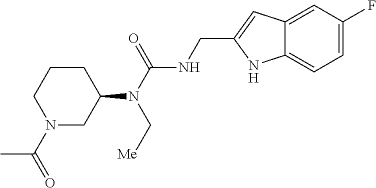 [SMILES: CCN(C(=O)NCc1cc2cc(F)ccc2[nH]1)[C@@H]1CCCN(C(C)=O)C1] | (R)-1-(1- acetylpiperidin-3-yl)- 1-ethyl-3-((5-fluoro- 1H-indol-2- yl)methyl)urea | 360.20 | 361.2 |
| 167 | 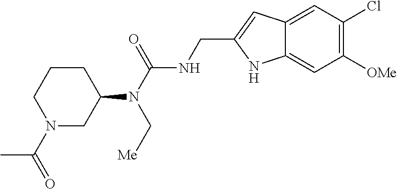 [SMILES: CCN(C(=O)NCc1cc2cc(Cl)c(OC)cc2[nH]1)[C@@H]1CCCN(C(C)=O)C1] | (R)-1-(1- acetylpiperidin-3-yl)- 3-((5-chloro-6- methoxy-1H-indol-2- yl)methyl)-1-ethylurea | 406.18 | 407.1 |
| 168 | 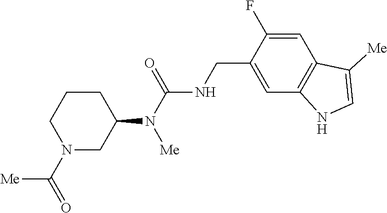 [SMILES: CC(=O)N1CCC[C@@H](N(C)C(=O)NCc2cc3[nH]cc(C)c3cc2F)C1] | (R)-1-(1- acetylpiperidin-3-yl)- 3-((5-fluoro-3-methyl- 1H-indol-6- yl)methyl)-1- methylurea | 360.20 | 361.1 |
| 169 | 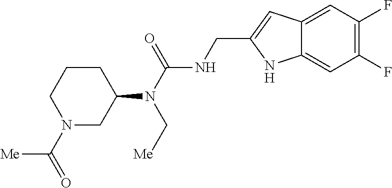 [SMILES: CCN(C(=O)NCc1cc2cc(F)c(F)cc2[nH]1)[C@@H]1CCCN(C(C)=O)C1] | (R)-1-(1- acetylpiperidin-3-yl)- 3-((5,6-difluoro-1H- indol-2-yl)methyl)-1- ethylurea | 378.19 | 379.2 |
| 170 | 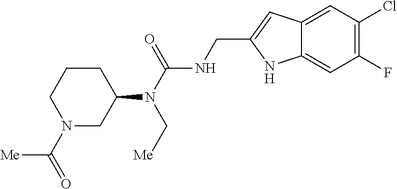 [SMILES: CCN(C(=O)NCc1cc2cc(Cl)c(F)cc2[nH]1)[C@@H]1CCCN(C(C)=O)C1] | (R)-1-(1- acetylpiperidin-3-yl)- 3-((5-chloro-6-fluoro- 1H-indol-2- yl)methyl)-1-ethylurea | 394.16 | 395.1 |
| 172 | 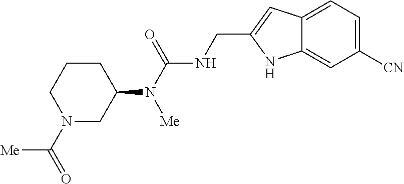 [SMILES: CC(=O)N1CCC[C@@H](N(C)C(=O)NCc2cc3ccc(C#N)cc3[nH]2)C1] | (R)-1-(1- acetylpiperidin-3-yl)- 3-((6-cyano-1H-indol- 2-yl)methyl)-1- methylurea | 353.19 | 354.2 |
| 174 | 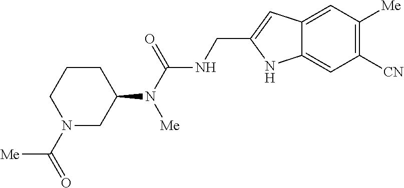 [SMILES: CC(=O)N1CCC[C@@H](N(C)C(=O)NCc2cc3cc(C)c(C#N)cc3[nH]2)C1] | (R)-1-(1- acetylpiperidin-3-yl)- 3-((6-cyano-5-methyl- 1H-indol-2- yl)methyl)-1- methylurea | 367.20 | 368.2 |
| 175 | 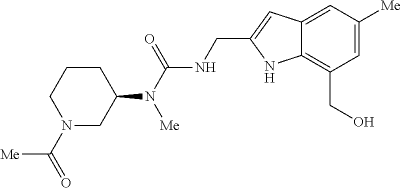 [SMILES: CC(=O)N1CCC[C@@H](N(C)C(=O)NCc2cc3cc(C)cc(CO)c3[nH]2)C1] | (R)-1-(1- acetylpiperidin-3-yl)- 3-((7- (hydroxymethyl)-5- methyl-1H-indol-2- yl)methyl)-1- methylurea | 372.22 | 371.2 [M − H]⁻ |
| 176 | 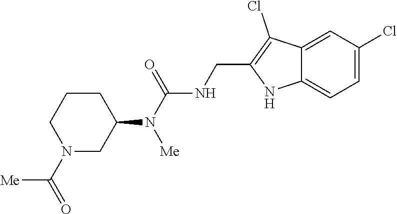 [SMILES: CC(=O)N1CCC[C@@H](N(C)C(=O)NCc2[nH]c3ccc(Cl)cc3c2Cl)C1] | (R)-1-(1- acetylpiperidin-3-yl)- 3-((3,5-dichloro-1H- indol-2-yl)methyl)-1- methylurea | 396.11 | 397.1 |
| 177 | 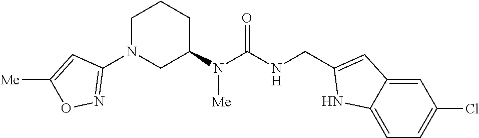 [SMILES: Cc1cc(N2CCC[C@@H](N(C)C(=O)NCc3cc4cc(Cl)ccc4[nH]3)C2)no1] | (R)-3-((5-chloro-1H- indol-2-yl)methyl)-1- methyl-1-(1-(5- methylisoxazol-3- yl)piperidin-3-yl)urea | 401.16 | 402.2 |
| 178 | 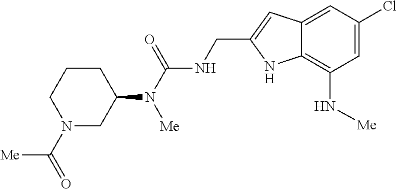 [SMILES: CNc1cc(Cl)cc2cc(CNC(=O)N(C)[C@@H]3CCCN(C(C)=O)C3)[nH]c12] | (R)-1-(1- acetylpiperidin-3-yl)- 3-((5-chloro-7- (methylamino)-1H- indol-2-yl)methyl)-1- methylurea | 391.18 | 392.2 |
| 179 | 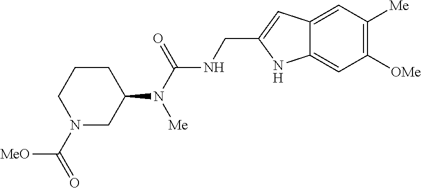 [SMILES: COC(=O)N1CCC[C@@H](N(C)C(=O)NCc2cc3cc(C)c(OC)cc3[nH]2)C1] | (R)-1-(1- acetylpiperidin-3-yl)- 3-((6-methoxy-5- methyl-1H-indol-2- yl)methyl)-1- methylurea | 372.22 | 373.3 |
| 182 | 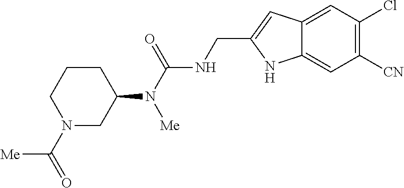 [SMILES: CC(=O)N1CCC[C@@H](N(C)C(=O)NCc2cc3cc(Cl)c(C#N)cc3[nH]2)C1] | (R)-1-(1- acetylpiperidin-3-yl)- 3-((5-chloro-6-cyano- 1H-indol-2- yl)methyl)-1- methylurea | 387.15 | 387.8 |
| 184 | 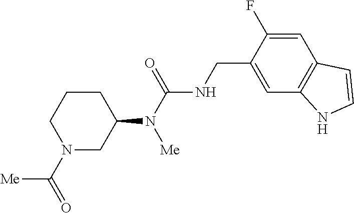 [SMILES: CC(=O)N1CCC[C@@H](N(C)C(=O)NCc2cc3[nH]ccc3cc2F)C1] | (R)-1-(1- acetylpiperidin-3-yl)- 3-((5-fluoro-1H-indol- 6-yl)methyl)-1- methylurea | 346.18 | 347.0 |
| 195 | 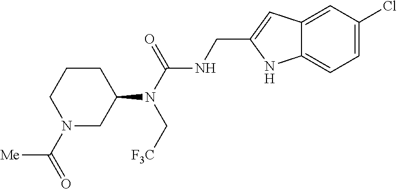 [SMILES: CC(=O)N1CCC[C@@H](N(CC(F)(F)F)C(=O)NCc2cc3cc(Cl)ccc3[nH]2)C1] | (R)-1-(1- acetylpiperidin-3-yl)- 3-((5-chloro-1H-indol- 2-yl)methyl)-1-(2,2,2- trifluoroethyl)urea | 430.14 | 431.0 |
| 212 | 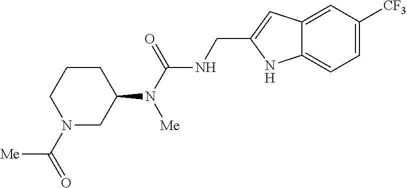 [SMILES: CC(=O)N1CCC[C@@H](N(C)C(=O)NCc2cc3cc(C(F)(F)F)ccc3[nH]2)C1] | (R)-1-(1- acetylpiperidin-3-yl)- 1-methyl-3-((5- (trifluoromethyl)-1H- indol-2-yl)methyl)urea | 396.18 | 397.2 |
| 214 | 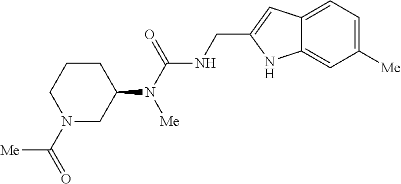 [SMILES: CC(=O)N1CCC[C@@H](N(C)C(=O)NCc2cc3ccc(C)cc3[nH]2)C1] | (R)-1-(1- acetylpiperidin-3-yl)- 1-methyl-3-((6- methyl-1H-indol-2- yl)methyl)urea | 342.21 | 343.2 |
| 217 | 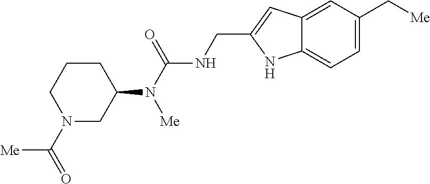 [SMILES: CCc1ccc2[nH]c(CNC(=O)N(C)[C@@H]3CCCN(C(C)=O)C3)cc2c1] | (R)-1-(1- acetylpiperidin-3-yl)- 3-((5-ethyl-1H-indol- 2-yl)methyl)-1- methylurea | 356.22 | 357.3 |
| 218 | 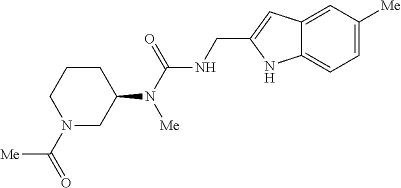 [SMILES: CC(=O)N1CCC[C@@H](N(C)C(=O)NCc2cc3cc(C)ccc3[nH]2)C1] | (R)-1-(1- acetylpiperidin-3-yl)- 1-methyl-3-((5- methyl-1H-indol-2- yl)methyl)urea | 342.21 | 343.2 |
| 219 | 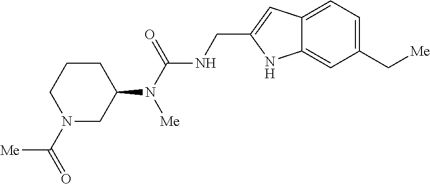 [SMILES: CCc1ccc2cc(CNC(=O)N(C)[C@@H]3CCCN(C(C)=O)C3)[nH]c2c1] | (R)-1-(1- acetylpiperidin-3-yl)- 3-((6-ethyl-1H-indol- 2-yl)methyl)-1- methylurea | 356.22 | 357.3 |
| 220 | 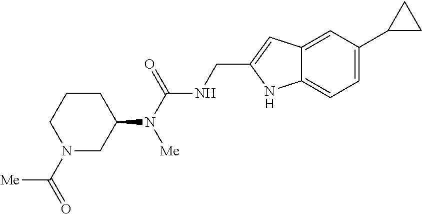 [SMILES: CC(=O)N1CCC[C@@H](N(C)C(=O)NCc2cc3cc(C4CC4)ccc3[nH]2)C1] | (R)-1-(1- acetylpiperidin-3-yl)- 3-((5-cyclopropyl-1H- indol-2-yl)methyl)-1- methylurea | 368.22 | 369.3 |
| 221 | 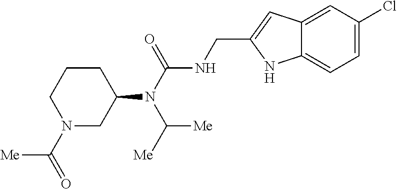 [SMILES: CC(=O)N1CCC[C@@H](N(C(=O)NCc2cc3cc(Cl)ccc3[nH]2)C(C)C)C1] | (R)-1-(1- acetylpiperidin-3-yl)- 3-((5-chloro-1H-indol- 2-yl)methyl)-1- isopropylurea | 390.18 | 391.3 |
| 222 | 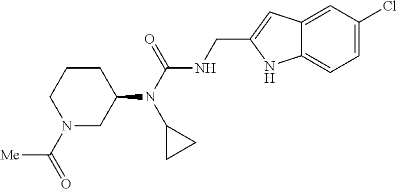 [SMILES: CC(=O)N1CCC[C@@H](N(C(=O)NCc2cc3cc(Cl)ccc3[nH]2)C2CC2)C1] | (R)-1-(1- acetylpiperidin-3-yl)- 3-((5-chloro-1H-indol- 2-yl)methyl)-1- cyclopropylurea | 388.17 | 389.4 |
| 223 | 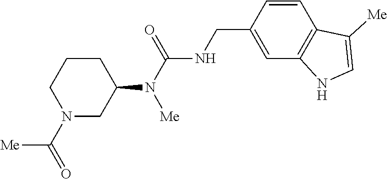 [SMILES: CC(=O)N1CCC[C@@H](N(C)C(=O)NCc2ccc3c(C)c[nH]c3c2)C1] | (R)-1-(1- acetylpiperidin-3-yl)- 1-methyl-3-((3- methyl-1H-indol-6- yl)methyl)urea | 342.21 | 343.2 |
| 224 | 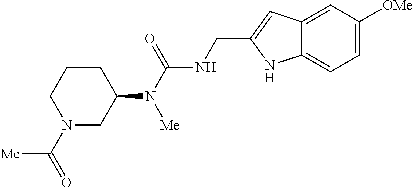 [SMILES: COc1ccc2[nH]c(CNC(=O)N(C)[C@@H]3CCCN(C(C)=O)C3)cc2c1] | (R)-1-(1- acetylpiperidin-3-yl)- 3-((5-methoxy-1H- indol-2-yl)methyl)-1- methylurea | 358.20 | 359.2 |
| 225 | 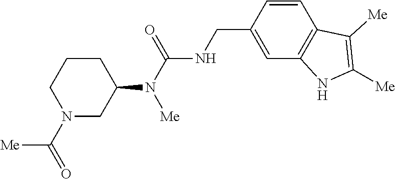 [SMILES: CC(=O)N1CCC[C@@H](N(C)C(=O)NCc2ccc3c(C)c(C)[nH]c3c2)C1] | (R)-1-(1- acetylpiperidin-3-yl)- 3-((2,3-dimethyl-1H- indol-6-yl)methyl)-1- methylurea | 356.22 | 357.3 |
| 226 | 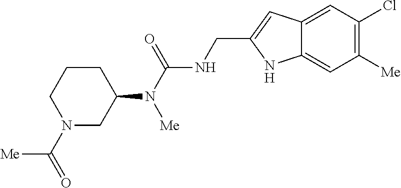 [SMILES: CC(=O)N1CCC[C@@H](N(C)C(=O)NCc2cc3cc(Cl)c(C)cc3[nH]2)C1] | (R)-1-(1- acetylpiperidin-3-yl)- 3-((5-chloro-6-methyl- 1H-indol-2- yl)methyl)-1- methylurea | 376.17 | 377.2 |
| 227 | 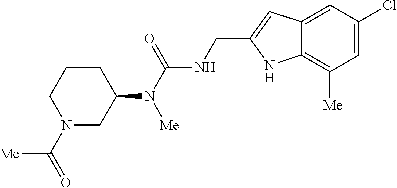 [SMILES: CC(=O)N1CCC[C@@H](N(C)C(=O)NCc2cc3cc(Cl)cc(C)c3[nH]2)C1] | (R)-1-(1- acetylpiperidin-3-yl)- 3-((5-chloro-7-methyl- 1H-indol-2- yl)methyl)-1- methylurea | 376.17 | 377.1 |
| 228 | 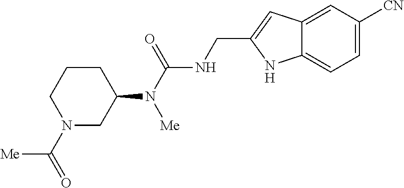 [SMILES: CC(=O)N1CCC[C@@H](N(C)C(=O)NCc2cc3cc(C#N)ccc3[nH]2)C1] | (R)-1-(1- acetylpiperidin-3-yl)- 3-((5-cyano-1H-indol- 2-yl)methyl)-1- methylurea | 353.19 | 354.2 |
| 229 | 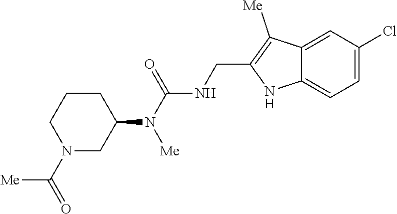 [SMILES: CC(=O)N1CCC[C@@H](N(C)C(=O)NCc2[nH]c3ccc(Cl)cc3c2C)C1] | (R)-1-(1- acetylpiperidin-3-yl)- 3-((5-chloro-3-methyl- 1H-indol-2- yl)methyl)-1- methylurea | 376.17 | 377.2 |
| 230 | 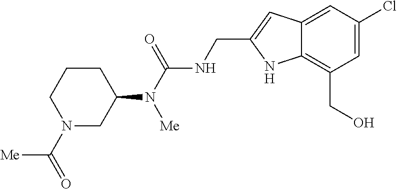 [SMILES: CC(=O)N1CCC[C@@H](N(C)C(=O)NCc2cc3cc(Cl)cc(CO)c3[nH]2)C1] | (R)-1-(1- acetylpiperidin-3-yl)- 3-((5-chloro-7- (hydroxymethyl)-1H- indol-2-yl)methyl)-1- methylurea | 392.16 | 319.2 [M − H]⁻ |
| 231 | 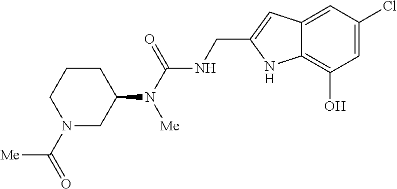 [SMILES: CC(=O)N1CCC[C@@H](N(C)C(=O)NCc2cc3cc(Cl)cc(O)c3[nH]2)C1] | (R)-1-(1- acetylpiperidin-3-yl)- 3-((5-chloro-7- hydroxy-1H-indol-2- yl)methyl)-1- methylurea | 378.15 | 379.1 |
| 233 | 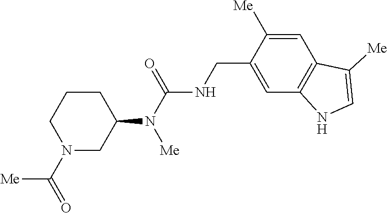 [SMILES: CC(=O)N1CCC[C@@H](N(C)C(=O)NCc2cc3[nH]cc(C)c3cc2C)C1] | (R)-1-(1- acetylpiperidin-3-yl)- 3-((3,5-dimethyl-1H- indol-6-yl)methyl)-1- methylurea | 356.22 | 357.3 |
| 235 | 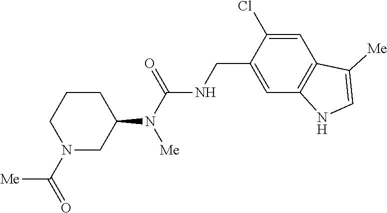 [SMILES: CC(=O)N1CCC[C@@H](N(C)C(=O)NCc2cc3[nH]cc(C)c3cc2Cl)C1] | (R)-1-(1- acetylpiperidin-3-yl)- 3-((5-chloro-3-methyl- 1H-indol-6- yl)methyl)-1- methylurea | 376.17 | 377.2 |

### Example 5: (R)-1-(1-acetylpiperidin-3-yl)-3-((5-chloro-1H-indol-2-yl)methyl)-1-methylurea (Compound 147)

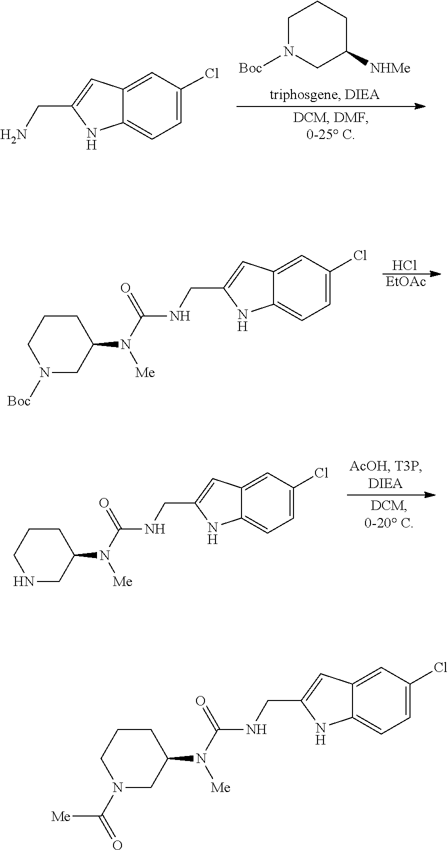

Step 1. 3 reactions were carried out in parallel. To a mixture of tert-butyl (R)-3-(methylamino)piperidine-1-carboxylate (1.00 g, 4.67 mmol, 1.00 eq) and (5-chloro-1H-indol-2-yl)methanamine hydrochloride (927 mg, 5.13 mmol, 1.10 eq) in DCM (10 mL) and DMF (1 mL) was added DIEA (1.21 g, 9.33 mmol, 2.00 eq). The resulting mixture was cooled to 0° C. and triphosgene (692 mg, 2.33 mmol, 0.5 eq) was added in portions, then the reaction mixture was allowed to warm to 25° C. and stir for 0.5 h. 3 reactions were combined for work up. The reaction mixture was quenched with H₂O (30 mL) and the aqueous phase was extracted with ethyl acetate (3×20 mL). The combined organic layers were washed with brine (20 mL), dried over Na₂SO₄, filtered and concentrated under reduced pressure. The resulting residue was purified by purified by column chromatography to give tert-butyl (R)-3-(3-((5-chloro-1H-indol-2-yl)methyl)-1-methylureido)piperidine-1-carboxylate. LC-MS (ESI) m/z: [M+H]⁺ calcd for C₂₁H₂₉ClN₄O₃. 420.19; found 421.3.

Step 2: To a solution of tert-butyl (R)-3-(3-((5-chloro-1H-indol-2-yl)methyl)-1-methylureido)piperidine-1-carboxylate (2.00 g, 4.75 mmol, 1.00 eq) in ethyl acetate (5 mL) was added HCl (4 M in EtOAc, 20.0 mL) and the reaction mixture was stirred at 25° C. After 0.5 h the reaction mixture was concentrated under reduced pressure and the resulting residue was purified by prep-HPLC to give (R)-3-((5-chloro-1H-indol-2-yl)methyl)-1-methyl-1-(piperidin-3-yl)urea hydrochloride. LC-MS (ESI) m/z: [M+H]⁺ calcd for C₁₆H₂₁ClN₄O: 320.14; found 321.2.

Step 3: To a solution of (R)-3-((5-chloro-1H-indol-2-yl)methyl)-1-methyl-1-(piperidin-3-yl)urea hydrochloride (850 mg, 1.90 mmol, 80% purity, 1.00 eq) in DCM (10 mL) at 0° C. was added AcOH (125 mg, 2.09 mmol, 1.10 eq), T3P (1.45 g, 2.28 mmol, 1.36 mL, 50 purity, 1.20 eq) and DIEA (737 mg, 5.71 mmol, 3.00 eq) in portions. The reaction mixture was warmed to 20° C. and stirred at 20° C. After 0.5 h the reaction mixture was quenched with H₂O (20 mL) and the aqueous phase was extracted with ethyl acetate (3×30 mL), the combined organic layers were washed with brine (20 mL), dried over Na₂SO₄, filtered and concentrated under reduced pressure. The resulting residue was purified by prep-HPLC to give (R)-1-(1-acetylpiperidin-3-yl)-3-((5-chloro-1H-indol-2-yl)methyl)-1-methylurea. LC-MS (ESI) m/z: [M+H]⁺ calcd for C₁₈H₂₃ClN₄O₂: 362.15; found 363.0. ¹H NMR (400 MHz, DMSO-d₆) δ 10.98 (s, 1H), 7.47 (s, 1H), 7.34 (d, J=8.8 Hz, 1H), 7.01-6.95 (m, 1H), 6.86 (t, J=5.8 Hz, 1H), 6.21 (s, 1H), 4.39-4.36 (m, 2H), 4.35-4.30 (m, 1H), 4.96-3.90 (m, 1H), 3.75-3.65 (m, 1H), 3.31-3.09 (m, 1H), 2.75 (d, J=8.4 Hz, 3H), 2.58-2.50 (m, 0.5H), 2.40-2.30 (m, 0.5H), 1.98 (d, J=9.2 Hz, 3H), 1.70-1.65 (m, 3H), 1.46-1.32 (m, 1H).

The following compounds in Table T-4 were synthesized using procedures similar to Compound 147 using the appropriate starting materials.

TABLE T-4

| | | | Exact | LCMS, |
|---|---|---|---|---|
| Compound | | | mass | found |
| No. | Structure | IUPAC name | (g/mol) | [M + H]⁺ |
| 148 | 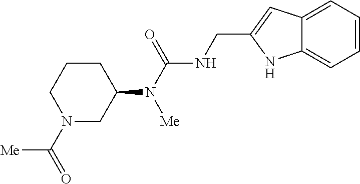 [SMILES: CC(=O)N1CCC[C@@H](N(C)C(=O)NCc2cc3ccccc3[nH]2)C1] | (R)-3-((1H-indol-2- yl)methyl)-1-(1- acetylpiperidin-3-yl)- 1-methylurea | 328.19 | 329.1 |

### Example 6: (R)-3-((5-chloro-1H-indol-2-yl)methyl)-1-methyl-1-(1-methylpiperidin-3-yl)urea (Compound 149)

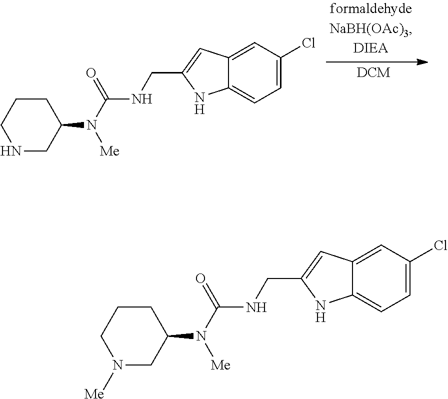 [SMILES: CN(C(=O)NCc1cc2cc(Cl)ccc2[nH]1)[C@@H]1CCCNC1 ; CN1CCC[C@@H](N(C)C(=O)NCc2cc3cc(Cl)ccc3[nH]2)C1]

To a mixture of formaldehyde (632 mg, 7.79 mmol, 37% purity, 10.0 eq) and (R)-3-((5-chloro-1H-indol-2-yl)methyl)-1-methyl-1-(piperidin-3-yl)urea hydrochloride (250 mg, 779 μmol, 1.00 eq) in DCM (6 mL) were added NaBH(OAc)₃ (495 mg, 2.34 mmol, 3.00 eq) and DIEA (503 mg, 3.90 mmol, 5.00 eq) in portions. The reaction mixture was stirred at 20° C. for 1 h. The reaction mixture was cooled to 0° C. and quenched with H₂O (10 mL). The aqueous phase was extracted with ethyl acetate (3×10 mL) then the combined organic layers were washed with brine (10 mL), dried over Na₂SO₄, filtered and concentrated under reduced pressure. The resulting residue was purified by prep-TLC to give (R)-3-((5-chloro-1H-indol-2-yl)methyl)-1-methyl-1-(1-methylpiperidin-3-yl)urea. LC-MS (ESI) m/z: [M+H]⁺ calcd for C₁₇H₂₃ClN₄O: 334.16; found 335.2. ¹H NMR (400 MHz, CDCl₃) δ 9.61 (s, 1H), 7.50 (s, 1H), 7.25 (s, 1H), 7.11-7.09 (m, 1H), 6.23 (s, 1H), 5.17 (s, 1H), 4.43 (d, J=6.0 Hz, 2H), 4.05 (t, J=9.2 Hz, 1H), 2.80 (d, J=10.4 Hz, 5H), 2.29 (s, 3H), 1.99-1.85 (m, 2H), 1.78-1.74 (m, 3H) 1.44-1.41 (m, 1H).

### Example 7: methyl (R)-3-(3-((5-chloro-1H-indol-2-yl)methyl)-1-methylureido)piperidine-1-carboxylate (Compound 158)

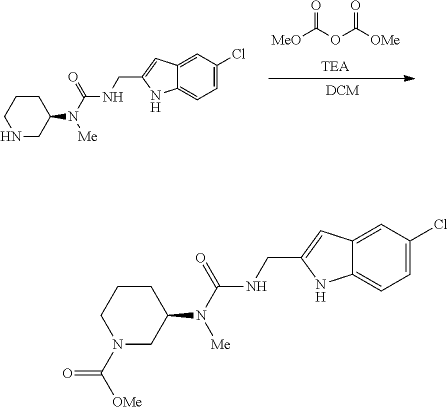 [SMILES: COC(=O)OC(=O)OC ; CN(C(=O)NCc1cc2cc(Cl)ccc2[nH]1)[C@@H]1CCCNC1 ; COC(=O)N1CCC[C@@H](N(C)C(=O)NCc2cc3cc(Cl)ccc3[nH]2)C1]

To a mixture of (R)-3-((5-chloro-1H-indol-2-yl)methyl)-1-methyl-1-(piperidin-3-yl)urea hydrochloride (80.0 mg, 223 μmol, 1.00 eq) and dimethyl dicarbonate (30.0 mg, 223 μmol, 1.00 eq) in DCM (1 mL) was added TEA (67.9 mg, 671 μmol, 3.00 eq) in one portion. The reaction mixture was stirred at 20° C. for 1 h. The reaction mixture was poured into water (15 mL) and stirred for 2 mins. The aqueous phase was extracted with ethyl acetate (2×15 mL). The combined organic phases were washed with brine (20 mL), dried over anhydrous Na₂SO₄, filtered and concentrated under reduced pressure. The resulting residue was purified by prep-TLC to give methyl (R)-3-(3-((5-chloro-1H-indol-2-yl)methyl)-1-methylureido)piperidine-1-carboxylate. LC-MS (ESI) m/z: [M+H]⁺ calcd for C₁₈H₂₃ClN₄O₃: 378.15; found 379.1. ¹H NMR (400 MHz, DMSO-d₆) δ 10.96 (s, 1H), 7.46 (d, J=1.6 Hz, 1H), 7.34 (d, J=8.4 Hz, 1H), 7.01-6.98 (m, 1H), 6.88 (t, J=5.6 Hz, 1H), 6.20 (s, 1H), 4.36 (d, J=5.6 Hz, 2H), 3.97-3.83 (m, 3H), 3.57 (s, 3H), 2.90-2.80 (m, 1H), 2.74 (s, 3H), 2.70-2.60 (m, 1H), 1.71-1.60 (m, 3H) 1.42-1.40 (m, 1H).

### Example 8: (R)-3-(3-((5-chloro-1H-indol-2-yl)methyl)-1-methylureido)-N-methylpiperidine-1-carboxamide (Compound 171)

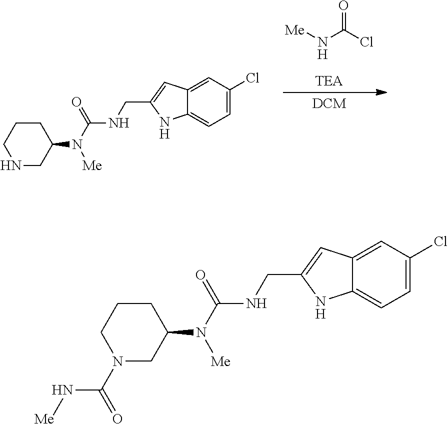 [SMILES: CNC(=O)Cl ; CN(C(=O)NCc1cc2cc(Cl)ccc2[nH]1)[C@@H]1CCCNC1 ; CNC(=O)N1CCC[C@@H](N(C)C(=O)NCc2cc3cc(Cl)ccc3[nH]2)C1]

To a solution of (R)-3-((5-chloro-1H-indol-2-yl)methyl)-1-methyl-1-(piperidin-3-yl)urea hydrochloride (150 mg, 419 μmol, 1.00 eq) in DCM (3 mL) was added dropwise TEA (127 mg, 1.26 mmol, 3 eq) followed by N-methylcarbamoyl chloride (51.0 mg, 545 μmol, 1.30 eq) portion wise. The reaction mixture was stirred at 20° C. After 1 h the reaction was poured into H₂O (20 mL) and stirred for 2 mins. The aqueous phase was extracted with ethyl acetate (3×15 mL). The combined organic layers were washed with brine (2×10 mL), dried with anhydrous Na₂SO₄, filtered and concentrated under reduced pressure. The resulting residue was purified by prep-HPLC to give (R)-3-(3-((5-chloro-1H-indol-2-yl)methyl)-1-methylureido)-N-methylpiperidine-1-carboxamide. LC-MS (ESI) m/z: [M+H]⁺ calcd for C₁₈H₂₄ClN₅O₂: 377.16; found 378.1. ¹H NMR (400 MHz, CDCl₃) δ 9.64 (s, 1H), 7.50 (s, 1H), 7.27-7.25 (m, 1H), 7.10-7.08 (m, 1H), 6.26 (s, 1H), 5.54 (s, 1H), 4.69-4.63 (m, 1H), 4.54-4.47 (m, 2H), 3.91-3.79 (m, 3H), 2.84 (d, J=5.6 Hz, 6H), 2.76-2.65 (m, 2H), 1.91-1.80 (m, 1H), 1.77-1.57 (m, 3H).

The following compounds in Table T-5 were synthesized using procedures similar to Compound 171 using the appropriate starting materials.

TABLE T-5

| | | | Exact | LCMS, |
|---|---|---|---|---|
| Compound | | | mass | found |
| No. | Structure | IUPAC name | (g/mol) | [M + H]⁺ |
| 173 | 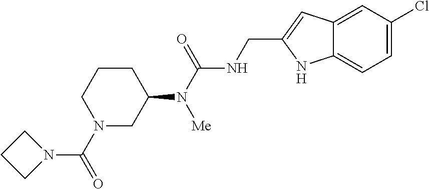 [SMILES: CN(C(=O)NCc1cc2cc(Cl)ccc2[nH]1)[C@@H]1CCCN(C(=O)N2CCC2)C1] | (R)-1-(1-(azetidine-1- carbonyl)piperidin-3-yl)- 3-((5-chloro-1H-indol-2- yl)methyl)-1-methylurea | 403.18 | 404.2 |
| 181 | 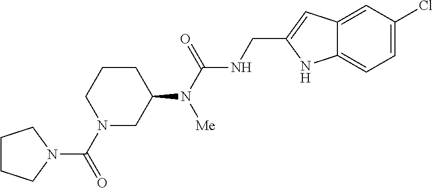 [SMILES: CN(C(=O)NCc1cc2cc(Cl)ccc2[nH]1)[C@@H]1CCCN(C(=O)N2CCCC2)C1] | (R)-3-((5-chloro-1H- indol-2-yl)methyl)-1- methyl-1-(1- (pyrrolidine-1- carbonyl)piperidin-3- yl)urea | 417.19 | 418.3 |
| 208 | 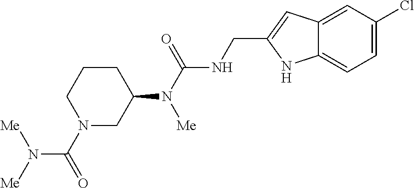 [SMILES: CN(C)C(=O)N1CCC[C@@H](N(C)C(=O)NCc2cc3cc(Cl)ccc3[nH]2)C1] | (R)-3-(3-((5-chloro-1H- indol-2-yl)methyl)-1- methylureido)-N,N- dimethylpiperidine-1- carboxamide | 391.18 | 392.4 |
| 209 | 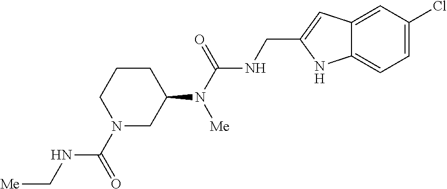 [SMILES: CCNC(=O)N1CCC[C@@H](N(C)C(=O)NCc2cc3cc(Cl)ccc3[nH]2)C1] | (R)-3-(3-((5-chloro-1H- indol-2-yl)methyl)-1- methylureido)-N- ethylpiperidine-1- carboxamide | 391.18 | 392.4 |
| 210 | 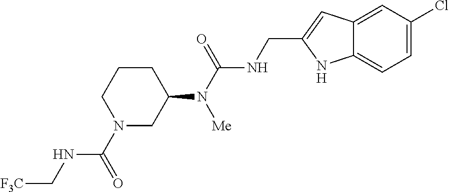 [SMILES: CN(C(=O)NCc1cc2cc(Cl)ccc2[nH]1)[C@@H]1CCCN(C(=O)NCC(F)(F)F)C1] | (R)-3-(3-((5-chloro-1H- indol-2-yl)methyl)-1- methylureido)-N-(2,2,2- trifluoroethyl)piperidine- 1-carboxamide | 445.15 | 446.1 |
| 211 | 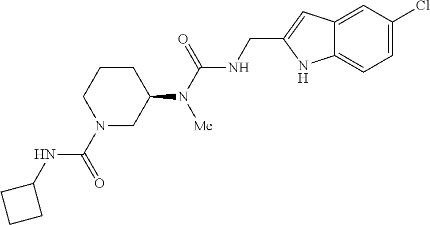 [SMILES: CN(C(=O)NCc1cc2cc(Cl)ccc2[nH]1)[C@@H]1CCCN(C(=O)NC2CCC2)C1] | (R)-3-(3-((5-chloro-1H- indol-2-yl)methyl)-1- methylureido)-N- cyclobutylpiperidine-1- carboxamide | 417.19 | 418.3 |
| 213 | 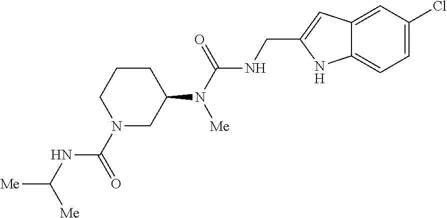 [SMILES: CC(C)NC(=O)N1CCC[C@@H](N(C)C(=O)NCc2cc3cc(Cl)ccc3[nH]2)C1] | (R)-3-(3-((5-chloro-1H- indol-2-yl)methyl)-1- methylureido)-N- isopropylpiperidine-1- carboxamide | 405.19 | 406.2 |
| 216 | 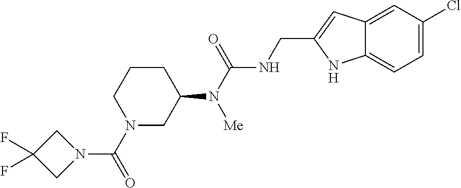 [SMILES: CN(C(=O)NCc1cc2cc(Cl)ccc2[nH]1)[C@@H]1CCCN(C(=O)N2CC(F)(F)C2)C1] | (R)-3-((5-chloro-1H- indol-2-yl)methyl)-1-(1- (3,3-difluoroazetidine-1- carbonyl)piperidin-3-yl)- 1-methylurea | 439.16 | 440.1 |

### BIOLOGICAL EXAMPLES

### Example B-1

CHO cells with doxycycline-inducible expression of human SLC6A19 and TMEM27 were maintained in T150 cell culture treated flasks using cell culture media, which consisted of 1×DMEM/F12 (Gibco, 11330057), 10% Tet System-Approved FBS (Gibco, A4736401), 100 U/mL Penicillin-Streptomycin (Gibco, 15140122), 1×GlutaMAX™ Supplement (Gibco, 35050061), 1 mM Sodium Pyruvate (Gibco, 11360070), 1.1 mg/mL Sodium Bicarbonate (Gibco, 25080094), 5 μg/mL Blasticidin (InvivoGen, ant-bl-1), 150 μg/mL Hygromycin B Gold (InvivoGen, ant-hg-5), and 500 μg/mL G418 (Sigma-Aldrich, G8168-100 mL). In preparation for the assay, this media was aspirated and 5 mL of TryμLE™ Express Enzyme (Gibco, 12605010) was added to each flask to initiate cell dissociation. The flasks were then incubated (humidified, 37° C. with 5% CO₂) for 2 minutes. After incubation, 10 mL of pre-warmed cell culture media was added to each flask. The cell suspension was collected and centrifuged for 5 minutes at 200xg.

The supernatant was aspirated and the pellet was resuspended in 5 mL pre-warmed cell culture media. The resuspended cells were counted using the Countess Cell Counting Chamber (Invitrogen) and the cell concentration of the suspension was readjusted to 1 million live cells/mL using pre-warmed cell culture media. 25 uL/well (25,000 cells/well) of readjusted cell suspension was dispensed into Poly-D-Lysine 384-well white/clear plates (Corning, 354660) using a MultiDrop Combi (Thermo Electron Corp). To induce expression of Human SLC6A19 and TMEM27, 25 uL/well of cell culture media containing 2 μg/mL doxycycline was added to the first 23 columns of each plate resulting in a final concentration of 1p g/mL doxycycline. 25 μL/well of cell culture media was added to the last column of each plate, to act as negative control wells for Human SLC6A19 and TMEM27 expression. Assay plates were then incubated (humidified, 37° C. with 5% CO₂) for 18 hours. After incubation, the media was aspirated and the wells were washed 4 times with pre-warmed HBSS (Sigma-Aldrich, H8264-1L) using an Apricot S-Pipette S2 (Apricot Designs), such that 5 uL/well HBSS was remaining at the end of each wash. 10 μL/well of test compound, a positive control compound to define 0% activity (i.e., 100% inhibition), or DMSO diluted in HBSS (Sigma-Aldrich, H₈₂₆₄-1L) was then added into each well using the Apricot S-Pipette S2 (Apricot Designs). At this step, the final in-well DMSO concentration in the assay plate is 0.5%. All compounds were plated in a duplicate 8-point dilution series that consisted of 3-fold dilutions. The plates were incubated with compound (humidified, 37° C. with 5% CO₂) for 1 hour. Afterwards, 15 uL/well of 3.2 mM hot:cold leucine solution (1:15000 ratio of L-[3,4,5-3H(N)] Leucine (Perkin Elmer, NET460001MC) to L-Leucine (Sigma-Aldrich, L8912-25G) diluted with HBSS (Sigma-Aldrich, H₈₂₆₄-1L)) was added using the Viaflo 384 (Integra), for a final concentration of 1.6 mM hot:cold leucine solution. The plates were then incubated (humidified, 37° C. with 5% CO₂) for 20 minutes to conduct the uptake reaction. Immediately after the incubation, the plates were washed 4 times with HBSS (Sigma-Aldrich, H8264-1L) using the ELx405 Microplate Washer (Biotek) to terminate the uptake reaction. After the washes, 50 uL/well Ultima Gold Scintillation Cocktail (Perkin Elmer, 6013326) was added to each well using the Viaflo 384 (Integra). The plates were then incubated (humidified, 37° C. with 5% CO₂) on a plate shaker at 200 rpm for 2 hours. After incubation, the plates were removed from the shaker and kept at room temperature for 15 minutes.

Liquid scintillation analysis was performed using the MicroBeta2 Microplate Counter (Perkin Elmer). Activity (%) was derived by normalizing the average of positive control compound treated wells to 0% and the average of DMSO (negative control) wells to 100%. Collaborative Drug Discovery software was used for graphing data.

TABLE B1

| | SLC6A19_Radioligand |
|---|---|
| Cmpd # | Uptake Assay IC50 (μM) |
| 111 | 1.32 |
| 112 | 0.367 |
| 113 | 0.494 |
| 114 | 0.847 |
| 115 | 0.688 |
| 116 | 0.515 |
| 117 | 0.608 |
| 118 | 1.62 |
| 119 | 0.629 |
| 120 | 0.574 |
| 121 | 0.28 |
| 134 | 3.77 |
| 135 | 0.162 |
| 137 | 0.359 |
| 147 | 0.183 |
| 163 | 0.145 |
| 171 | 0.15 |
| 187 | 1.23 |
| 188 | 0.656 |
| 189 | 0.264 |
| 190 | 0.483 |
| 191 | 0.151 |
| 192 | 0.558 |
| 193 | >50.0 |
| 194 | 0.197 |
| 195 | 0.102 |
| 196 | 0.64 |
| 197 | 0.219 |
| 198 | 0.232 |
| 199 | 0.434 |
| 200 | 0.35 |
| 201 | 0.205 |
| 202 | 0.168 |
| 203 | 2.17 |
| 204 | 0.256 |
| 205 | 0.444 |
| 206 | 0.441 |
| 207 | 0.715 |
| 208 | 0.418 |
| 209 | 0.126 |
| 210 | 0.892 |
| 211 | 1.36 |
| 212 | 0.259 |
| 213 | 0.804 |
| 214 | 1.29 |
| 215 | 0.637 |
| 216 | 0.807 |
| 217 | 0.574 |
| 218 | 0.273 |
| 219 | >10.0 |
| 220 | 0.794 |
| 221 | 2.41 |
| 222 | 0.193 |
| 232 | 0.23 |
| 233 | 15.6 |
| 234 | 0.367 |
| 235 | 0.673 |
| 236 | 0.975 |
| 237 | 0.434 |
| 238 | 0.283 |
| 239 | 0.209 |
| 240 | 0.154 |
| 241 | 0.387 |
| 242 | 1.04 |
| 243 | 0.262 |
| 244 | >10.0 |
| 245 | 0.459 |
| 246 | 1.16 |

### Example B2

Assay ready vials of CHO cells with doxycycline inducible expression of human SLC6A19 and TMEM27 were thawed, plated in T175 flasks containing DMEM/F12 media, 10% FBS, 100 Penicillin-Streptomycin, 1× UltraGlutamate, 1 mM Sodium Pyruvate, Sodium Bicarbonate (7.5 mL of 7.5% solution in 500 mL media final), 5 g/mL Blasticidin, 150 g/mL Hygromycin B Gold, and 500 g/mL G418, and incubated (humidified, 37° C. with 500 CO₂) for 48 hours. Media was then removed, flasks were washed with 20 mL of DPBS, and 5 mL of TryμLE™ Express was added to each flask to initiate cell dissociation. After 5 minutes, 10 mL of cell culture media was added to each flask and mixed. The cell suspension was collected and centrifuged for 5 minutes at 200×g. The supernatant was removed, and the pellet was resuspended in 10 mL of cell culture media. Resuspended cells were counted using a Vi-Cell. 30 μL/well (20,000 cells/well) of cell suspension was added to Poly-D-Lysine 384-well black/clear plates. Plates were incubated (humidified, 37° C. with 5% CO₂) for 24 hours. To induce expression of human SLC6A19 and TMEM27, 10 μL of assay culture media containing 4 μg/mL doxycycline was added to each well. Assay plates were then incubated (humidified, 37° C. with 5% CO₂) overnight. After incubation, the media was removed, and wells were washed 3 times with 40 μL of warmed DPBS. Next, 10 μL of warmed assay buffer (137 mM NaCl, 5 mM KCl, 1 mM CaCl2), 1 mM MgCl2, 10 mM HEPES, 10 mM glucose, pH 7.2) was added to each well. 10 μL/well of test compound or positive control compound to define 0% activity (i.e., 100% inhibition), was then added into each well. All compounds were plated in a duplicate 10-point dilution series that consisted of 2-fold dilutions. The plates were incubated (humidified, 37° C. with 5% CO₂) with compound for 20 minutes. Afterwards, 20 μL/well of 2 mM 13C6,15N-1-isoleucine was added to each well and plates were incubated again (humidified, 37° C. with 5% CO₂) for another 20 minutes. Assay buffer was then removed, and plates were washed. 70 μL/well of stop solution (75% acetonitrile, 25% HPLC grade water, 250 nM 1-leucine-1-13 C (RapidFire standard)) was added to each well. The plates were sealed and then placed in a −20° C. freezer for 20 minutes. The plates were thawed at room temperature for 20 minutes and then centrifuge for 20 minutes at 3000 rpm. Using an ABSciex API4000 mass spectrometer, processed samples were loaded onto the graphitic carbon SPE-column. The time for aspiration, load/wash, elution and re-equilibration time was 600, 1500, 3000 and 500 milliseconds, respectively. Peak areas were calculated using RapidFire Integrator software.

TABLE B2

| | SLC6A19_RapidFire |
|---|---|
| Cmpd # | Uptake Assay IC50 (μM) |
| 1 | 0.252 |
| 2 | 3.52 |
| 3 | 0.359 |
| 4 | 0.62 |
| 5 | 0.578 |
| 6 | 0.198 |
| 7 | 0.685 |
| 8 | 0.491 |
| 9 | 5.37 |
| 10 | 0.125 |
| 11 | 0.743 |
| 12 | 0.354 |
| 13 | 1.61 |
| 14 | 0.467 |
| 15 | 0.539 |
| 16 | >4.29 |
| 17 | 0.151 |
| 18 | 2.25 |
| 19 | 0.484 |
| 20 | 8.96 |
| 21 | 5.74 |
| 22 | 4.37 |
| 23 | 5.84 |
| 24 | 4.2 |
| 25 | 11.6 |
| 26 | 5.26 |
| 27 | 0.821 |
| 28 | >23.4 |
| 29 | 2.49 |
| 30 | 3.7 |
| 31 | 1.49 |
| 32 | 8.61 |
| 33 | 0.405 |
| 34 | 1.4 |
| 35 | 0.556 |
| 36 | 1.57 |
| 37 | 19.6 |
| 38 | 0.428 |
| 39 | 0.231 |
| 40 | 5.25 |
| 41 | 0.854 |
| 42 | 1.24 |
| 43 | 6.25 |
| 44 | 0.35 |
| 45 | 1.55 |
| 46 | 1.24 |
| 47 | 2.46 |
| 48 | 0.0442 |
| 49 | 0.371 |
| 50 | 4.77 |
| 51 | 4.8 |
| 52 | 0.366 |
| 53 | 1.63 |
| 54 | 0.401 |
| 55 | 0.571 |
| 56 | 2.46 |
| 57 | 8.64 |
| 58 | 4.05 |
| 59 | 0.529 |
| 60 | 7.78 |
| 61 | 0.542 |
| 62 | 1.35 |
| 63 | 0.935 |
| 64 | 0.651 |
| 65 | 0.973 |
| 66 | 4.24 |
| 67 | 0.887 |
| 68 | 0.417 |
| 69 | >2.81 |
| 70 | 3.06 |
| 71 | 6.39 |
| 72 | 1.37 |
| 73 | 0.897 |
| 74 | 2.54 |
| 75 | 1.75 |
| 76 | 1 |
| 77 | 6.57 |
| 78 | 1.76 |
| 79 | 5.63 |
| 80 | 5.65 |
| 81 | 1.41 |
| 82 | 3.81 |
| 83 | 4.79 |
| 84 | 0.176 |
| 85 | 0.369 |
| 86 | 0.559 |
| 87 | 1.67 |
| 88 | 4.08 |
| 89 | 1.08 |
| 90 | 13.5 |
| 91 | 1.81 |
| 92 | 8.99 |
| 93 | 1.63 |
| 94 | 2.75 |
| 95 | 3.24 |
| 96 | 0.933 |
| 97 | 1.34 |
| 98 | 3 |
| 99 | 0.745 |
| 100 | 0.38 |
| 101 | 2.93 |
| 102 | 1.8 |
| 103 | 2.24 |
| 104 | 6.78 |
| 105 | >27.0 |
| 106 | 1.77 |
| 107 | 1.25 |
| 108 | 2.28 |
| 109 | 1.81 |
| 110 | 8.56 |
| 119 | 0.839 |
| 120 | 0.949 |
| 121 | 0.405 |
| 122 | 8.6 |
| 123 | 0.427 |
| 124 | 0.256 |
| 125 | 0.259 |
| 126 | 1.44 |
| 127 | 0.679 |
| 128 | 1.82 |
| 129 | >31.8 |
| 130 | 12.1 |
| 131 | >45.4 |
| 132 | >31.0 |
| 133 | >7.64 |
| 135 | 0.326 |
| 136 | 3.95 |
| 137 | 0.273 |
| 138 | 0.643 |
| 139 | 0.517 |
| 140 | 1.24 |
| 141 | 0.67 |
| 142 | 1.31 |
| 143 | 1.09 |
| 144 | 0.284 |
| 145 | 0.909 |
| 146 | 0.553 |
| 147 | 0.195 |
| 148 | 1.08 |
| 149 | 10.1 |
| 150 | 0.0919 |
| 151 | 0.0404 |
| 152 | 0.171 |
| 153 | 0.504 |
| 154 | 0.173 |
| 155 | 0.537 |
| 156 | 0.282 |
| 157 | 0.194 |
| 158 | 0.113 |
| 159 | 0.179 |
| 160 | 0.708 |
| 161 | 0.493 |
| 162 | 6.78 |
| 163 | 0.0494 |
| 164 | 0.351 |
| 165 | 8.03 |
| 166 | 0.168 |
| 167 | 0.103 |
| 168 | 0.651 |
| 169 | 0.0978 |
| 170 | 0.0451 |
| 171 | 0.0806 |
| 172 | 10.9 |
| 173 | 0.314 |
| 174 | 2.79 |
| 175 | 20.9 |
| 176 | 0.0307 |
| 177 | 0.122 |
| 178 | 1.57 |
| 179 | 1.21 |
| 180 | 2.79 |
| 181 | 0.795 |
| 182 | 2.93 |
| 183 | 0.852 |
| 184 | 3.97 |
| 185 | 4.4 |
| 186 | 5.6 |
| 187 | 2.77 |
| 188 | 0.947 |
| 189 | 0.38 |
| 190 | 0.578 |
| 191 | 0.312 |
| 193 | >50.0 |
| 194 | 0.242 |
| 195 | 0.062 |
| 196 | 0.518 |
| 197 | 0.396 |
| 198 | 0.163 |
| 199 | 0.582 |
| 200 | 0.512 |
| 201 | 0.227 |
| 202 | 0.689 |
| 203 | 4.87 |
| 204 | 0.763 |
| 217 | 0.738 |
| 218 | 0.647 |
| 219 | 12.9 |
| 220 | 0.953 |
| 221 | 4.61 |
| 222 | 0.0633 |
| 223 | 4.67 |
| 224 | 1.65 |
| 225 | 2.96 |
| 226 | 0.209 |
| 227 | 0.327 |
| 228 | 2.21 |
| 229 | 0.108 |
| 230 | 12.8 |
| 231 | 1.06 |
| 232 | 0.0453 |
| 233 | 9.12 |
| 234 | 0.255 |
| 235 | 0.893 |
| 236 | 0.805 |
| 237 | 1.21 |
| 238 | 0.836 |
| 239 | 0.287 |
| 240 | 0.16 |
| 241 | 0.626 |
| 242 | 3.28 |
| 243 | 0.153 |
| 244 | 7.08 |
| 245 | 0.151 |

All publication, patent applications, patents, and other references mentioned herein are expressly incorporated by reference in their entireties, to the same extent as if each were incorporated by reference individually.

What is claimed is:

1. A compound of formula (I): 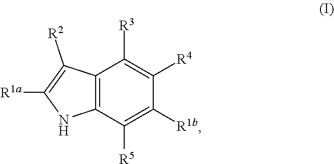
or a stereoisomer or tautomer thereof, or a pharmaceutically acceptable salt of any of the foregoing, wherein
one of R^{1a} and R^{1b} is 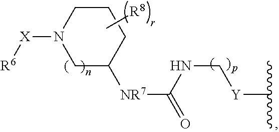
and
the other of R^{1a} and R^{1b} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein the C₁₋₆alkyl of R³ and R⁵ is optionally substituted with one or more —OH;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
the C₁₋₆alkyl, C₃₋₁₀cycloalkyl, and 3-10 membered heterocyclyl of R^{a} are each optionally substituted with one or more R^{b}, and
the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, or —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
X is absent or —C(O)—;
Y is absent or 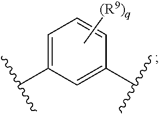
n is an integer from 1 to 2;
p is an integer from 1 to 4;
q is an integer from 0 to 4; and
r is an integer from 0 to 11.

2. The compound of claim 1, wherein the compound is a compound of formula (I-A): 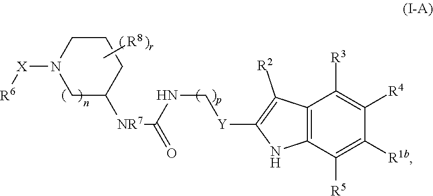
or a stereoisomer or tautomer thereof, or a pharmaceutically acceptable salt of any of the foregoing.

3. The compound of claim 1, wherein the compound is a compound of formula (I-B): 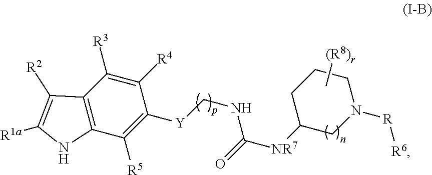
or a stereoisomer or tautomer thereof, or a pharmaceutically acceptable salt of any of the foregoing.

4. The compound of claim 1 or claim 2, wherein the compound is a compound of formula (I-C): 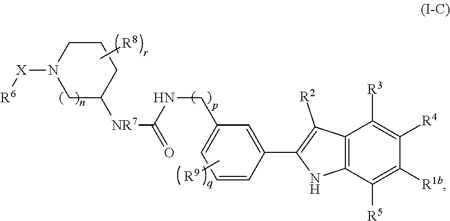
or a stereoisomer or tautomer thereof, or a pharmaceutically acceptable salt of any of the foregoing.

5. The compound of claim 1 or claim 2, wherein the compound is a compound of formula (I-D): 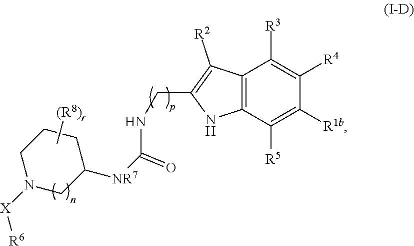
or a stereoisomer or tautomer thereof, or a pharmaceutically acceptable salt of any of the foregoing.

6. The compound of any one of claims 1-5, or a stereoisomer or tautomer thereof, or a pharmaceutically acceptable salt of any of the foregoing, wherein X is —C(O)—.

7. The compound of any one of claims 1-6, or a stereoisomer or tautomer thereof, or a pharmaceutically acceptable salt of any of the foregoing, wherein n is 1.

8. The compound of any one of claims 1-7, or a stereoisomer or tautomer thereof, or a pharmaceutically acceptable salt of any of the foregoing, wherein n is 2.

9. The compound of any one of claims 1-8, or a stereoisomer or tautomer thereof, or a pharmaceutically acceptable salt of any of the foregoing, wherein p is an integer from 1 to 3.

10. The compound of any one of claims 1-9, or a stereoisomer or tautomer thereof, or a pharmaceutically acceptable salt of any of the foregoing, wherein p is 1 or 2.

11. The compound of any one of claims 1-10, or a stereoisomer or tautomer thereof, or a pharmaceutically acceptable salt of any of the foregoing, wherein p is 1.

12. The compound of any one of claims 1-11, or a stereoisomer or tautomer thereof, or a pharmaceutically acceptable salt of any of the foregoing, wherein R⁷ is C₁₋₃alkyl, C₁₋₃cycloalkyl, or C₁₋₃haloalkyl.

13. The compound of any one of claims 1-12, or a stereoisomer or tautomer thereof, or a pharmaceutically acceptable salt of any of the foregoing, wherein R⁷ is C₁₋₃alkyl.

14. The compound of any one of claims 1-13, or a stereoisomer or tautomer thereof, or a pharmaceutically acceptable salt of any of the foregoing, wherein R⁴ is H, halo, —CN, C₁₋₆alkyl, C₁₋₆cycloalkyl, C₁₋₆haloalkyl.

15. The compound of any one of claims 1-14, or a stereoisomer or tautomer thereof, or a pharmaceutically acceptable salt of any of the foregoing, wherein R⁴ is H, halo, —CN, C₁₋₃alkyl, C₁₋₃cycloalkyl, C₁₋₃haloalkyl, or C₁₋₃alkoxy, wherein the C₁₋₃alkoxy of R² and R⁴ is optionally substituted with one or more R^{a}.

16. The compound of claim 2, or a stereoisomer or tautomer thereof, or a pharmaceutically acceptable salt of any of the foregoing, wherein R^{1b} is H, halo, —CN, H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}.

17. The compound of claim 3, or a stereoisomer or tautomer thereof, or a pharmaceutically acceptable salt of any of the foregoing, wherein R^{1a} is H, halo, —CN, H, halo, —CN, C₁₋₃alkyl, C₁₋₃haloalkyl, C₁₋₃cycloalkyl, or C₁₋₃alkoxy, wherein the C₁₋₃alkoxy of R^{1a} and R^{1b} is optionally substituted with one or more R^{a}.

18. The compound of any one of claims 1-17, or a stereoisomer or tautomer thereof, or a pharmaceutically acceptable salt of any of the foregoing, wherein R⁶ is C₁₋₃alkyl, C₁₋₃haloalkyl, C₁₋₃alkoxy, C₃₋₆cycloalkyl, —N(R^{w1})(R^{w2}), C₆₋₁₀aryl, 3-6 membered heterocyclyl, or 5-10 membered heteroaryl, wherein
the C₁₋₃alkyl, C₆₋₁₀aryl, and 5-10 membered heteroaryl of R⁶ are each independently optionally substituted with one or more R^{a}, and
the C₃₋₆cycloalkyl, and 3-6 membered heterocyclyl of R⁶ are each independently optionally substituted with one or more R^{b}.

19. The compound of any one of claims 1-18, or a stereoisomer or tautomer thereof, or a pharmaceutically acceptable salt of any of the foregoing, wherein R⁶ is 5-20 membered heteroaryl optionally substituted with one or more R^{a}.

20. The compound of claim 19, or a stereoisomer or tautomer thereof, or a pharmaceutically acceptable salt of any of the foregoing, wherein R⁶ is selected from the group consisting of

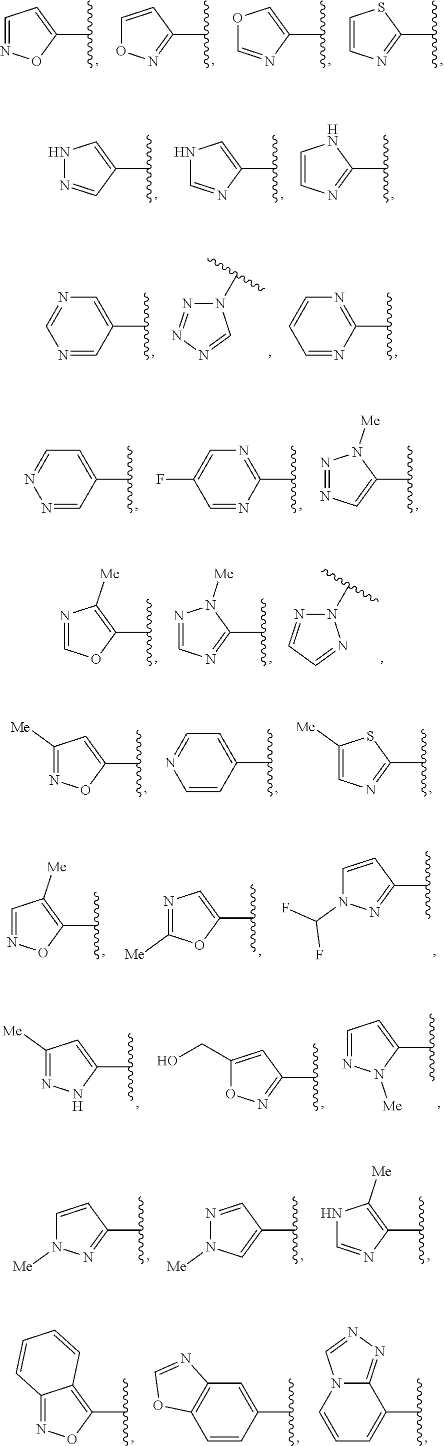

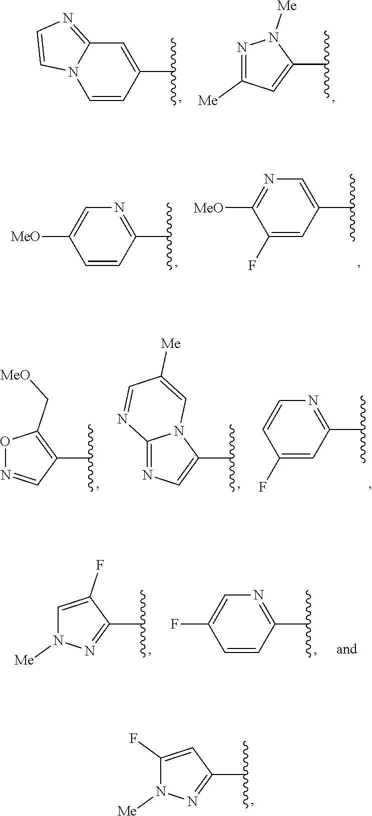

21. The compound of any one of claims 1-18, or a stereoisomer or tautomer thereof, or a pharmaceutically acceptable salt of any of the foregoing, wherein R⁶ is 3-10 membered heterocyclyl optionally substituted with one or more R^{b}.

22. The compound of claim 21, or a stereoisomer or tautomer thereof, or a pharmaceutically acceptable salt of any of the foregoing, wherein R⁶ is selected from the group consisting of

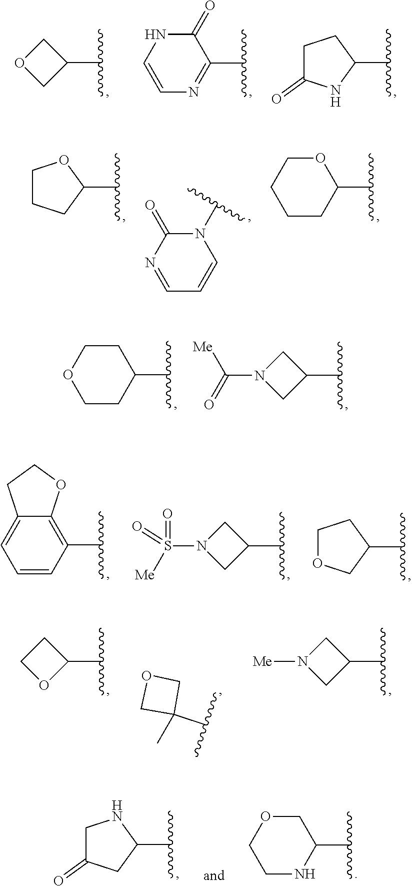

23. The compound of any one of claims 1-18, or a stereoisomer or tautomer thereof, or a pharmaceutically acceptable salt of any of the foregoing, wherein R⁶ is C₆₋₂₀aryl optionally substituted with one or more R^{a}.

24. The compound of claim 23, or a stereoisomer or tautomer thereof, or a pharmaceutically acceptable salt of any of the foregoing, wherein R⁶ is selected from the group consisting of

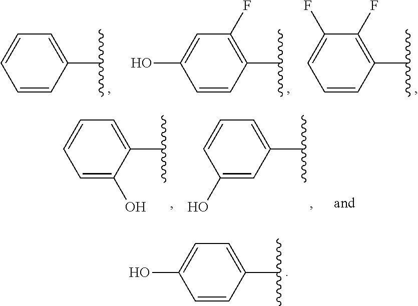

25. The compound of any one of claims 1-18, or a stereoisomer or tautomer thereof, or a pharmaceutically acceptable salt of any of the foregoing, wherein R⁶ is C₃₋₁₀cycloalkyl optionally substituted with one or more R^{b}.

26. The compound of claim 25, or a stereoisomer or tautomer thereof, or a pharmaceutically acceptable salt of any of the foregoing, wherein R⁶ is selected from the group consisting of

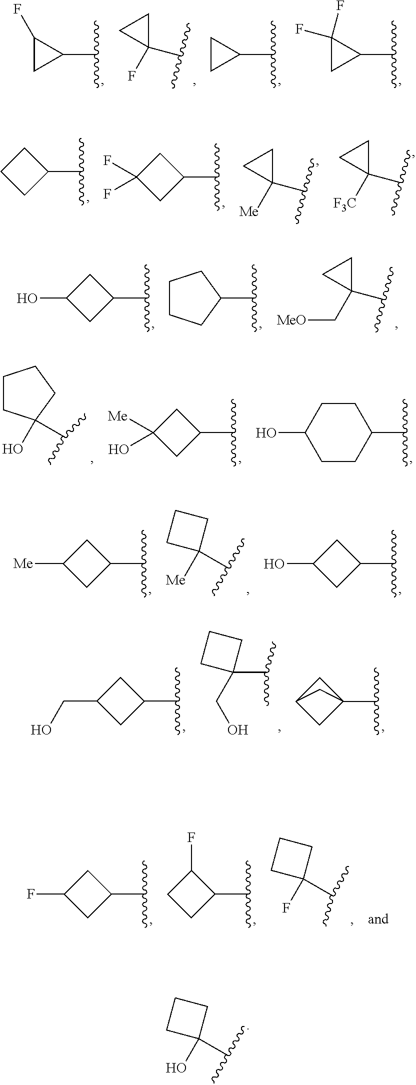

27. The compound of any one of claims 1-18, or a stereoisomer or tautomer thereof, or a pharmaceutically acceptable salt of any of the foregoing, wherein R⁶ is C₁₋₆alkyl optionally substituted with one or more R^{a}.

28. The compound of any one of claims 1-27, or a stereoisomer or tautomer thereof, or a pharmaceutically acceptable salt of any of the foregoing, wherein R² is H, halo, —CN, C₁₋₃alkyl, C₁₋₄cycloalkyl, C₁₋₃haloalkyl.

29. The compound of any one of claims 1-28, or a stereoisomer or tautomer thereof, or a pharmaceutically acceptable salt of any of the foregoing, wherein R³ and R⁵ are each independently H, —OH, halo, —N(R^{w1})(R^{w2}), or C₁₋₄alkyl, wherein the C₁₋₄alkyl is optionally substituted with one or more —OH.

30. The compound of any one of claims 1-29, or a stereoisomer or tautomer thereof, or a pharmaceutically acceptable salt of any of the foregoing, wherein R^{a} is independently C₁₋₆alkyl, C₆₋₂₀aryl, 5-20 membered heteroaryl, or 3-10 membered heterocyclyl, wherein the C₁₋₆alkyl, C₃₋₁₀cycloalkyl, and 3-10 membered heterocyclyl of R^{a} are each optionally substituted with one or more R^{b}, and the C₆₋₂₀aryl, and 5-20 membered heteroaryl of R^{a} are each optionally substituted with one or more R^{c}.

31. The compound of any one of claims 1-30, or a stereoisomer or tautomer thereof, or a pharmaceutically acceptable salt of any of the foregoing, wherein R^{b} is independently C₁₋₆alkyl optionally substituted with one or more R^{c}.

32. The compound of any one of claims 1-31, or a stereoisomer or tautomer thereof, or a pharmaceutically acceptable salt of any of the foregoing, wherein R is independently —OH, halo, —N(R^{w1})(R^{w2}), C₁₋₃alkyl, —C₁₋₃alkylN(R^{w1})(R^{w2}), or C₁₋₃alkoxy.

33. The compound of any one of claims 1-32, or a stereoisomer or tautomer thereof, or a pharmaceutically acceptable salt of any of the foregoing, wherein R^{w1} and R^{w2} are independently at each occurrence H, C₁₋₆alkyl, C₁₋₆haloalkyl, or C₃₋₁₀cycloalkyl.

34. The compound of claim 1, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from Table 1.

35. A process for preparing a compound of claim 1, or a stereoisomer or tautomer thereof, or a pharmaceutically acceptable salt of any of the foregoing, wherein the process comprises:
reacting a compound of formula (I-1): 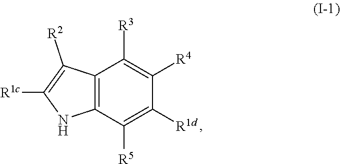
or a stereoisomer or tautomer thereof, or a pharmaceutically acceptable salt of any of the foregoing, wherein
one of R^{1c} and R^{1d} is 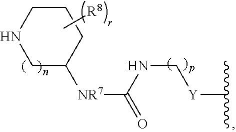
and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁷ is C₁₋₆alkyl, C₁₋₆cycloalkyl, or C₁₋₆haloalkyl;
R⁸ is independently at each occurrence, C₁₋₆alkyl;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
the C₁₋₆alkyl, C₃₋₁₀cycloalkyl, and 3-10 membered heterocyclyl of R^{a} are each optionally substituted with one or more R^{b}, and
the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Y is absent or 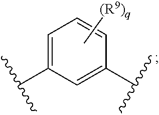
n is an integer from 1 to 2;
p is an integer from 1 to 4;
q is an integer from 0 to 4; and
r is an integer from 0 to 11;
with a compound of formula (I-2): 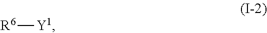
or a stereoisomer or tautomer thereof, or a pharmaceutically acceptable salt of any of the foregoing, wherein:
Y¹ is —C(O)OH or —C(O)-halo;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
the C₁₋₆alkyl, C₃₋₁₀cycloalkyl, and 3-10 membered heterocyclyl of R^{a} are each optionally substituted with one or more R^{b}, and
the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂; and
R^{z} is independently at each occurrence, C₁₋₆alkyl;
in the presence of one or more coupling reagents.

36. The process of claim 35, wherein the one or more coupling reagents comprises EDCI, HOBt and/or DIEA.

37. A process for preparing a compound of claim 1, or a stereoisomer or tautomer thereof, or a pharmaceutically acceptable salt of any of the foregoing, wherein the process comprises:
reacting a compound of formula (II-1): 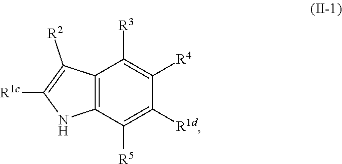
or a stereoisomer or tautomer thereof, or a pharmaceutically acceptable salt of any of the foregoing, wherein:
one of R^{1c} and R^{1d} is 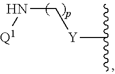
and
and the other of R^{1c} and R^{1d} is H, halo, —CN, C₁₋₆alkyl, C₁₋₆haloalkyl, C₁₋₆cycloalkyl, or C₁₋₆alkoxy, wherein the C₁₋₆alkoxy of R^{1a} and R^{1b} is optionally substituted with one or more R^{a};
R² and R⁴ are each independently H, halo, —CN, C₁₋₆alkyl, C₁₋₆cycloalkyl, C₁₋₆haloalkyl, or C₁₋₆alkoxy, wherein the C₁₋₆alkoxy of R² and R⁴ is optionally substituted with one or more R^{a};
R³ and R⁵ are each independently H, —OH, halo, —N(R^{w1})(R^{w2}), or C₁₋₆alkyl, wherein C₁₋₆alkyl of R³ and R⁵ is optionally substituted with one or more —OH;
R⁹ is independently at each occurrence, halo;
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
the C₁₋₆alkyl, C₃₋₁₀cycloalkyl, and 3-10 membered heterocyclyl of R^{a} are each optionally substituted with one or more R^{b}, and
the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl;
Q¹H or —C(O)-LG;
LG is a leaving group;
Y is absent or 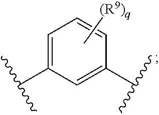
p is an integer from 1 to 4;
q is an integer from 0 to 4; and
with a compound of formula (I-2): 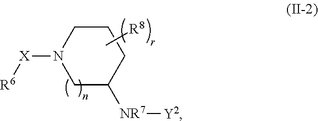
or a stereoisomer or tautomer thereof, or a pharmaceutically acceptable salt of any of the foregoing, wherein:
Y² is —H;
R⁶ is C₁₋₆alkyl, C₁₋₆haloalkyl, C₁₋₆alkoxy, C₃₋₁₀cycloalkyl, —N(R^{w1})(R^{w2}), C₆₋₂₀aryl, 3-10 membered heterocyclyl, or 5-20 membered heteroaryl, wherein
the C₁₋₆alkyl, C₆₋₂₀aryl, and 5-20 membered heteroaryl of R⁶ are each independently optionally substituted with one or more R^{a}, and
the C₃₋₁₀cycloalkyl, and 3-10 membered heterocyclyl of R⁶ are each independently optionally substituted with one or more R^{b};
R⁷ is C₁₋₆alkyl, C₁₋₆cycloalkyl, or C₁₋₆haloalkyl;
R⁸ is independently at each occurrence, C₁₋₆alkyl;
n is an integer from 1 to 2;
r is an integer from 0 to 11.
each R^{a} is independently —OH, halo, —CN, C₁₋₆alkyl, C₁₋₆alkoxy, —N(R^{w1})(R^{w2}), —OR^{x}, —C(O)R^{y}, —SO₂R^{z}, C₃₋₁₀cycloalkyl, C₆₋₂₀aryl, 5-20 membered heteroaryl, or 3-10 membered heterocyclyl, wherein
the C₁₋₆alkyl, C₃₋₁₀cycloalkyl, and 3-10 membered heterocyclyl of R^{a} are each optionally substituted with one or more R^{b}, and
the C₆₋₂₀aryl, and 5-20 membered heteroaryl of R^{a} are each optionally substituted with one or more R^{c};
each R^{b} is independently —OH, oxo, halo, C₁₋₆alkyl, C₁₋₆alkoxy, —C(O)R^{y}, or —SO₂R^{z}, wherein
the C₁₋₆alkyl is optionally substituted with one or more R^{c};
each R^{c} is independently —OH, halo, —N(R^{w1})(R^{w2}), C₁₋₆alkyl, —C₁₋₆alkylN(R^{w1})(R^{w2}), or C₁₋₆alkoxy;
R^{w1} and R^{w2} are independently at each occurrence H, C₁₋₆alkyl, C₁₋₆haloalkyl, C₃₋₁₀cycloalkyl, —C(O)R^{y};
R^{x} is independently at each occurrence, 3-10 membered heterocyclyl optionally substituted with one or more oxo or C₁₋₆alkyl;
R^{y} is independently at each occurrence, C₁₋₆alkyl, C₁₋₆haloalkyl; —NH₂, —NH(C₁₋₆alkyl), or —N(C₁₋₆alkyl)₂;
R^{z} is independently at each occurrence, C₁₋₆alkyl; and
X is absent or —C(O)—;
in the presence of one or more coupling reagents.

38. The process of claim 37, wherein the one or more coupling reagents comprises a base.

39. The process of claim 37, wherein the one or more coupling reagents comprises a carbonate ester.

40. The process of claim 39, wherein the carbonate ester is triphosgene.

41. The process of any one of claims 37-40, wherein LG is an alkoxy group optionally substituted with one or more halo.

42. A pharmaceutical composition comprising (i) a compound of any one of claims 1-34, or a stereoisomer or tautomer thereof, or a pharmaceutically acceptable salt of any of the foregoing, and (ii) one or more pharmaceutically acceptable excipients.

43. A method of modulating SLC6A19 in a cell, comprising exposing the cell to an effective amount of a compound of any one of claims 1-34, or a stereoisomer or tautomer thereof, or a pharmaceutically acceptable salt of any of the foregoing, or a pharmaceutical composition of claim 42.

44. A method of inhibiting SLC6A19 in a cell, comprising exposing the cell to an effective amount of a compound of any one of claims 1-34, or a stereoisomer or tautomer thereof, or a pharmaceutically acceptable salt of any of the foregoing, or a pharmaceutical composition of claim 42.

45. A method of reducing systemic phenylalanine, tyrosine, glutamine, or glycine levels in an individual in need thereof, comprising administering to the individual an effective amount of a compound of any one of claims 1-34, or a stereoisomer or tautomer thereof, or a pharmaceutically acceptable salt of any of the foregoing, or a pharmaceutical composition of claim 42.

46. A method of treating a SLC6A19-mediated disease, disorder, or condition in an individual in need thereof, comprising administering to the individual an effective amount of a compound of any one of claims 1-34, or a stereoisomer or tautomer thereof, or a pharmaceutically acceptable salt of any of the foregoing, or a pharmaceutical composition of claim 42.

47. The method of claim 46, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

48. The method of claim 46 or claim 47, wherein the disease, disorder, or condition is selected from the group consisting of phenylketonuria (PKU), chronic kidney disease (CKD), methabolic syndrome, and metabolic diseases.

49. The method of claim 46, wherein the disease, disorder, or condition is associated with abnormal levels of amino acids.

50. The method of claim 46, or claim 49, wherein the disease, disorder, or condition is associated with a genetic defect in phenylalanine hydroxylase.

51. A kit, comprising (i) a compound of any one of claims 1-34, or a stereoisomer or tautomer thereof, or a pharmaceutically acceptable salt of any of the foregoing, or a pharmaceutical composition of claim 43, and (ii) instructions for use in treating an SLC6A19-mediated disease, disorder, or condition in an individual in need thereof.

52. The kit of claim 51, wherein the disease, disorder, or condition is associated with abnormal levels of amino acids.

53. The kit of claim 51, or claim 52, wherein the disease, disorder, or condition is associated with a genetic defect in phenylalanine hydroxylase.

54. The kit of claim 51, wherein the disease, disorder, or condition is selected from the group consisting of phenylketonuria (PKU), chronic kidney disease (CKD), metabolic syndrome, metabolic diseases, hyperphenylalaninemia, tyrosinemia (Type I, II, or III), nonketotic hyperglycinemia, isovaleric acidemia, methylmalonic acidemia, propionic acidemia, maple syrup urine disease, DNAJC12 deficiency, urea cycle disorders, hyperammonemia, diabetes, nonalcoholic fatty liver disease, nonalcoholic steatohepatitis, obesity related disorders, and neurodevelopmental and autism-spectrum disorders.

55. The kit of claim 51, wherein the individual has a genetic defect in phenylalanine hydroxylase.
